(19) United States
(12) Patent Application Publication
(10) Pub. No.: US 2025/0099583 A1
(43) Pub. Date: Mar. 27, 2025

(54) PHARMACEUTICAL COMPOSITIONS OF ANTI-IGF-1R ANTIBODIES

(71) Applicant: Viridian Therapeutics, Inc., Waltham, MA (US)

(72) Inventors: Bernard Gaetan Huyghe, San Diego, CA (US); Kevin Bach, Avon, IN (US); Sean T. Nugent, Chatham, MA (US); Angela She, Waltham, MA (US); Yao Tang, Shanghai (CN); Jeremy Guo, Palo Alto, CA (US)

(21) Appl. No.: 18/827,383

(22) Filed: Sep. 6, 2024

(30) Foreign Application Priority Data
Sep. 6, 2023 (WO) PCT/CN2023/117341
Aug. 30, 2024 (CN) 202411218553.4

(51) Int. Cl. A61K 39/395; A61K 39/00; A61K 47/18; A61K 47/26; A61P 27/02; C07K 16/28

(52) U.S. Cl. CPC A61K 39/39591; A61K 47/183; A61K 47/26; A61P 27/02; C07K 16/2863; A61K 2039/505; A61K 2039/54

(57) ABSTRACT

Provided herein are pharmaceutical compositions of anti-IGF-1R antibodies and uses thereof.

### CROSS REFERENCE TO RELATED APPLICATIONS

This application claims priority to International Application No. PCT/CN2023/117341, filed Sep. 6, 2023, and Chinese Application No. 202411218553.4 filed on Aug. 30, 2024, each of which is hereby incorporated by reference in its entirety.

### REFERENCE TO SEQUENCE LISTING SUBMITTED ELECTRONICALLY

The instant application contains a Sequence Listing which has been submitted electronically in XML file format and is hereby incorporated by reference in its entirety. Said XML copy, created on Sep. 5, 2024, is named “VRD-010US1_SL.xml” and is 100 kilobytes in size.

### BACKGROUND

Developments in biotechnology have made it possible to produce a large variety of monoclonal antibodies for pharmaceutical applications. Antibodies are larger and more complex than traditional organic and inorganic drugs and therefore formulating antibodies for therapeutic administration poses special problems. One of the problems is maintaining stability of the antibody, which is a critical concern in ensuring product quality. Another problem when formulating antibodies for therapeutic use is viscosity, particularly when formulating the antibody at high concentrations. The unique amino acid sequences and third dimensional structures of antibodies causes to differences in physicochemical properties like isoelectric point, hydrophobicity, and surface charge distributions which can be both difficult to predict and greatly impact the behavior of the formulated antibody. The optimal pH, ionic strength, buffer type and excipients required for stabilization vary for each antibody based on its specific structural characteristics, and a “one-size-fits-all” formulation is not feasible.

Thyroid-associated ophthalmopathy (TAO), also known as thyroid eye disease (TED), Graves' ophthalmopathy or orbitopathy (GO), thyrotoxic exophthalmos, dysthyroid ophthalmopathy, and several other terms, is orbitopathy associated with thyroid dysfunction. TAO is divided into two types: active TAO and inactive TAO. Active TAO, which typically lasts 1-3 years, is characterized by an ongoing autoimmune/inflammatory response in the soft tissues of the orbit. Active TAO is responsible for the expansion and remodeling of the ocular soft tissues. The autoimmune/inflammatory response of active TAO spontaneously resolves and the condition transitions into inactive TAO. Inactive TAO is the term used to describe the long-term/permanent sequelae of active TAO. The cause of TAO is unknown. TAO is typically associated with Graves' hyperthyroidism but can also occur as part of other autoimmune conditions that affect the thyroid gland and produce pathology in orbital and periorbital tissue, and, rarely, the pretibial skin (pretibial myxedema) or digits (thyroid aeropathy). TAO is an autoimmune orbitopathy in which the orbital and periocular soft tissues are primarily affected with secondary effects on the eye and vision. In TAO, as a result of inflammation and expansion of orbital soft tissues, primarily eye muscles and adipose, the eyes are forced forward (bulge) out of the eye sockets—a phenomenon termed proptosis or exophthalmos. Although most cases of TAO do not result in loss of vision, this condition can cause vision-threatening exposure keratopathy, troublesome diplopia (double vision), and compressive dysthyroid optic neuropathy. TAO may precede, coincide with, or follow the systemic complications of dysthyroidism. The ocular manifestations of TAO include upper eyelid retraction, lid lag, swelling, redness (erythema), conjunctivitis, and bulging eyes (exophthalmos or proptosis), chemosis, periorbital edema, and altered ocular motility with significant functional, social, and cosmetic consequences. Many of the signs and symptoms of TAO, including proptosis and ocular congestion, result from expansion of the orbital adipose tissue and periocular muscles. The adipose tissue volume increases owing in part to new fat cell development (adipogenesis) within the orbital fat. The accumulation of hydrophilic glycosaminoglycans, primarily hyaluronic acid, within the orbital adipose tissue and the perimysial connective tissue between the extraocular muscle fibers, further expands the fat compartments and enlarges the extraocular muscle bodies. Hyaluronic acid is produced by fibroblasts residing within the orbital fat and extraocular muscles, and its synthesis in vitro is stimulated by several cytokines and growth factors, including IL-1beta, interferon-gamma, platelet-derived growth factor, thyroid stimulating hormone (TSH) and insulin-like growth factor I (IGF-I).

Antibodies that activate the insulin-like growth factor I receptor (IGF-IR) have also been detected and implicated in active TAO. Without being bound to any theory, it is believed that TSHR and IGF-IR form a physical and functional complex in orbital fibroblasts, and that blocking IGF-IR appears to attenuate both IGF-1 and TSH-dependent signaling. It has been suggested that blocking IGF-IR using an antibody antagonist might reduce both TSHR- and IGF-I-dependent signaling and therefore interrupt the pathological activities of autoantibodies acting as agonists on either receptor.

IGF-IR is a widely expressed heterotetrameric protein involved in the regulation of proliferation and metabolic function of many cell types. It is a tyrosine kinase receptor comprising two subunits. IGF-IRalpha contains a ligand-binding domain while IGF-IRbeta is involved in signaling and contains tyrosine phosphorylation sites.

Current therapies for hyperthyroidism due to Graves' disease are imperfect because therapies targeting the specific underlying pathogenic autoimmune mechanisms of the disease are lacking. Even more complex is the treatment of moderate-to-severe active TAO. Although recent years have witnessed a better understanding of its pathogenesis, TAO remains a therapeutic challenge and dilemma. Intravenous glucocorticoids (ivGCs) and oral glucocorticoids are used to treat patients with moderate-to-severe active TAO, but results are seldom satisfactory. Partial responses are frequent and relapses (rebound) after drug withdrawal are not uncommon. Adverse events do occur and many patients eventually require rehabilitative surgery conducted when their condition has transitioned to inactive TAO. Accordingly, there is still a need to provide alternative therapies for TAO and its related symptoms.

### SUMMARY

The present invention provides, among other things, stable formulations for anti-insulin like growth factor 1 receptor (IGF-1R) antibodies, particularly those described herein, suitable for intravenous, subcutaneous and other routes of administration. The present invention is, in part, based on the discovery of a particular pH range (i.e., 4.8-6.5, and particularly 4.8-6.0) that renders both conformational and colloidal stability of the antibody in the formulation. As a result, the present invention permits formulating the anti-IGF-1R antibodies, particularly those described herein, at a high concentration with high purity and long-term stability that allows safer and more efficacious clinical use.

In one aspect, the present invention provides, among other things, a stable formulation comprising an anti-IGF-1R antibody or antigen-binding fragment thereof at a concentration of 25-250 mg/ml, wherein the antibody comprises a heavy chain variable region comprising HCDR1 of SEQ ID NO: 53, an HCDR2 of SEQ ID NO: 54, an HCDR3 of SEQ ID NO: 55, a light chain variable region comprising a LCDR1 of SEQ ID NO: 56, a LCDR2 of SEQ ID NO: 57 and a LCDR3 of SEQ ID NO: 58, and wherein the formulation has a pH of 4.8-6.5.

In one aspect, the present invention provides, among other things, a stable formulation comprising an anti-IGF-1R antibody or antigen-binding fragment thereof at a concentration of 25-250 mg/ml, wherein the antibody comprises a heavy chain variable region comprising HCDR1 of SEQ ID NO: 53, an HCDR2 of SEQ ID NO: 54, an HCDR3 of SEQ ID NO: 55, a light chain variable region comprising a LCDR1 of SEQ ID NO: 56, a LCDR2 of SEQ ID NO: 57 and a LCDR3 of SEQ ID NO: 58, and wherein the formulation has a pH of 4.8-6.0.

In some embodiments, the heavy chain variable region is at least 90% identical to SEQ ID NO: 14 and the light chain variable region is at least 90% identical to SEQ ID NO: 13.

In some embodiments, the heavy chain variable region is at least 80% identical to SEQ ID NO: 14. In some embodiments, the heavy chain variable region is at least 82% identical to SEQ ID NO: 14. In some embodiments, the heavy chain variable region is at least 84% identical to SEQ ID NO: 14. In some embodiments, the heavy chain variable region is at least 86% identical to SEQ ID NO: 14. In some embodiments, the heavy chain variable region is at least 88% identical to SEQ ID NO: 14. In some embodiments, the heavy chain variable region is at least 90% identical to SEQ ID NO: 14. In some embodiments, the heavy chain variable region is at least 92% identical to SEQ ID NO: 14. In some embodiments, the heavy chain variable region is at least 94% identical to SEQ ID NO: 14. In some embodiments, the heavy chain variable region is at least 96% identical to SEQ ID NO: 14. In some embodiments, the heavy chain variable region is at least 98% identical to SEQ ID NO: 14. In some embodiments, the heavy chain variable region is at least 99% identical to SEQ ID NO: 14.

In some embodiments, the light chain variable region is at least 80% identical to SEQ ID NO: 13. In some embodiments, the light chain variable region is at least 82% identical to SEQ ID NO: 13. In some embodiments, the light chain variable region is at least 84% identical to SEQ ID NO: 13. In some embodiments, the light chain variable region is at least 86% identical to SEQ ID NO: 13. In some embodiments, the light chain variable region is at least 88% identical to SEQ ID NO: 13. In some embodiments, the light chain variable region is at least 90% identical to SEQ ID NO: 13. In some embodiments, the light chain variable region is at least 92% identical to SEQ ID NO: 13. In some embodiments, the light chain variable region is at least 94% identical to SEQ ID NO: 13. In some embodiments, the light chain variable region is at least 96% identical to SEQ ID NO: 13. In some embodiments, the light chain variable region is at least 98% identical to SEQ ID NO: 13. In some embodiments, the light chain variable region is at least 99% identical to SEQ ID NO: 13.

In some embodiments, the heavy chain variable region comprises SEQ ID NO: 14 and the light chain variable region comprises SEQ ID NO: 13.

In some embodiments, the antibody comprises a heavy chain of SEQ ID NO: 92 or SEQ ID NO: 94, and a light chain of SEQ ID NO: 93.

In some embodiments, the antibody comprises a heavy chain that is at least 80% identical to SEQ ID NO: 92. In some embodiments, the antibody comprises a heavy chain that is at least 82% identical to SEQ ID NO: 92. In some embodiments, the antibody comprises a heavy chain that is at least 84% identical to SEQ ID NO: 92. In some embodiments, the antibody comprises a heavy chain that is at least 85% identical to SEQ ID NO: 92. In some embodiments, the antibody comprises a heavy chain that is at least 86% identical to SEQ ID NO: 92. In some embodiments, the antibody comprises a heavy chain that is at least 88% identical to SEQ ID NO: 92. In some embodiments, the antibody comprises a heavy chain that is at least 90% identical to SEQ ID NO: 92. In some embodiments, the antibody comprises a heavy chain that is at least 92% identical to SEQ ID NO: 92. In some embodiments, the antibody comprises a heavy chain that is at least 94% identical to SEQ ID NO: 92. In some embodiments, the antibody comprises a heavy chain that is at least 95% identical to SEQ ID NO: 92. In some embodiments, the antibody comprises a heavy chain that is at least 96% identical to SEQ ID NO: 92. In some embodiments, the antibody comprises a heavy chain that is at least 97% identical to SEQ ID NO: 92. In some embodiments, the antibody comprises a heavy chain that is at least 98% identical to SEQ ID NO: 92. In some embodiments, the antibody comprises a heavy chain that is at least 99% identical to SEQ ID NO: 92.

In some embodiments, the antibody comprises a light chain that is at least 80% identical to SEQ ID NO: 93. In some embodiments, the antibody comprises a light chain that is at least 82% identical to SEQ ID NO: 93. In some embodiments, the antibody comprises a light chain that is at least 84% identical to SEQ ID NO: 93. In some embodiments, the antibody comprises a light chain that is at least 85% identical to SEQ ID NO: 93. In some embodiments, the antibody comprises a light chain that is at least 86% identical to SEQ ID NO: 93. In some embodiments, the antibody comprises a light chain that is at least 88% identical to SEQ ID NO: 93. In some embodiments, the antibody comprises a light chain that is at least 90% identical to SEQ ID NO: 93. In some embodiments, the antibody comprises a light chain that is at least 92% identical to SEQ ID NO: 93. In some embodiments, the antibody comprises a light chain that is at least 94% identical to SEQ ID NO: 93. In some embodiments, the antibody comprises a light chain that is at least 95% identical to SEQ ID NO: 93. In some embodiments, the antibody comprises a light chain that is at least 96% identical to SEQ ID NO: 93. In some embodiments, the antibody comprises a light chain that is at least 97% identical to SEQ ID NO: 93. In some embodiments, the antibody comprises a light chain that is at least 98% identical to SEQ ID NO: 93. In some embodiments, the antibody comprises a light chain that is at least 99% identical to SEQ ID NO: 93.

In some embodiments, the anti-IGF-1R antibody is present at a concentration of between 25-200 mg/ml. In some embodiments, the anti-IGF-1R antibody is present at a concentration of between 50-200 mg/ml. In some embodiments, the anti-IGF-1R antibody is present at a concentration of between 25-75 mg/ml. In some embodiments, the anti-IGF-1R antibody is present at a concentration of between 40-60 mg/ml. In some embodiments, the anti-IGF-1R antibody is present at a concentration of between 100-200 mg/ml. In some embodiments, the anti-IGF-1R antibody is present at a concentration of between 125-175 mg/ml. In some embodiments, the anti-IGF-1R antibody is present at a concentration of between 140-160 mg/ml. In some embodiments, the anti-IGF-1R antibody is present at a concentration of between 145-155 mg/ml.

In some embodiments, the anti-IGF-1R antibody is present at a concentration of 25 mg/ml. In some embodiments, the anti-IGF-1R antibody is present at a concentration of 50 mg/ml. In some embodiments, the anti-IGF-1R antibody is present at a concentration of 60 mg/ml. In some embodiments, the anti-IGF-1R antibody is present at a concentration of 70 mg/ml. In some embodiments, the anti-IGF-1R antibody is present at a concentration of 75 mg/ml. In some embodiments, the anti-IGF-1R antibody is present at a concentration of 100 mg/ml. In some embodiments, the anti-IGF-1R antibody is present at a concentration of 125 mg/ml. In some embodiments, the anti-IGF-1R antibody is present at a concentration of 150 mg/ml. In some embodiments, the anti-IGF-1R antibody is present at a concentration of 175 mg/ml. In some embodiments, the anti-IGF-1R antibody is present at a concentration of 200 mg/ml.

In some embodiments, the pH is between 4.8-6.5. In some embodiments, the pH is between 4.8-6.0. In some embodiments, the pH is between 5.0-6.0. In some embodiments, the pH is between 5.2-6.0. In some embodiments, the pH is between 5.2-5.8.

In some embodiments, formulation has a pH of 4.8. In some embodiments, formulation has a pH of 4.9. In some embodiments, formulation has a pH of 5.0. In some embodiments, formulation has a pH of 5.1. In some embodiments, formulation has a pH of 5.2. In some embodiments, formulation has a pH of 5.3. In some embodiments, formulation has a pH of 5.4. In some embodiments, formulation has a pH of 5.5. In some embodiments, formulation has a pH of 5.6. In some embodiments, formulation has a pH of 5.7. In some embodiments, formulation has a pH of 5.8. In some embodiments, formulation has a pH of 5.9. In some embodiments, formulation has a pH of 6.0. In some embodiments, formulation has a pH of 6.1. In some embodiments, formulation has a pH of 6.2. In some embodiments, formulation has a pH of 6.3. In some embodiments, formulation has a pH of 6.4. In some embodiments, formulation has a pH of 6.5.

In some embodiments, the formulation further comprises sucrose.

In some embodiments, sucrose is present at a concentration of between 1-20% (w/v). In some embodiments, sucrose is present at a concentration of between 3-15% (w/v). In some embodiments, sucrose is present at a concentration of between 4-10 (w/v). In some embodiments, sucrose is present at a concentration of between 6-10% (w/v). In some embodiments, sucrose is present at a concentration of between 1-20% (w/v).

In some embodiments, sucrose is present at a concentration of 1% (w/v). In some embodiments, sucrose is present at a concentration of 2% (w/v). In some embodiments, sucrose is present at a concentration of 4% (w/v). In some embodiments, sucrose is present at a concentration of 5% (w/v). In some embodiments, sucrose is present at a concentration of 6% (w/v). In some embodiments, sucrose is present at a concentration of 7% (w/v). In some embodiments, sucrose is present at a concentration of 8% (w/v). In some embodiments, sucrose is present at a concentration of 9% (w/v). In some embodiments, sucrose is present at a concentration of 10% (w/v). In some embodiments, sucrose is present at a concentration of 12% (w/v). In some embodiments, sucrose is present at a concentration of 14% (w/v). In some embodiments, sucrose is present at a concentration of 16% (w/v). In some embodiments, sucrose is present at a concentration of 18% (w/v). In some embodiments, sucrose is present at a concentration of 20% (w/v).

In some embodiments, the formulation further comprises a stabilizer. In some embodiments, a stabilizer is an anti-oxidant. In some embodiments, the stabilizer is methionine.

In some embodiments, methionine is present at a concentration of between 1-50 mM. In some embodiments, methionine is present at a concentration of between 5-40 mM. In some embodiments, methionine is present at a concentration of between 10-30 mM. In some embodiments, methionine is present at a concentration of between 10-20 mM. In some embodiments, methionine is present at a concentration of between 15-30 mM.

In some embodiments, methionine is present at a concentration of 5 mM. In some embodiments, methionine is present at a concentration of 10 mM. In some embodiments, methionine is present at a concentration of 15 mM. In some embodiments, methionine is present at a concentration of 20 mM. In some embodiments, methionine is present at a concentration of 25 mM. In some embodiments, methionine is present at a concentration of 30 mM. In some embodiments, methionine is present at a concentration of 35 mM. In some embodiments, methionine is present at a concentration of 40 mM.

In some embodiments, the composition further comprises a surfactant.

In some embodiments, a surfactant is polysorbate 80. In some embodiments, a surfactant is polysorbate 20.

In some embodiments, polysorbate 80 is present at a concentration of between 0.001-1% (w/v). In some embodiments, polysorbate 80 is present at a concentration of between 0.005-0.5% (w/v). In some embodiments, polysorbate 80 is present at a concentration of between 0.01-0.05% (w/v).

In some embodiments, polysorbate 80 is present at a concentration of 0.001% (w/v). In some embodiments, polysorbate 80 is present at a concentration of 0.005% (w/v). In some embodiments, polysorbate 80 is present at a concentration of 0.01% (w/v). In some embodiments, polysorbate 80 is present at a concentration of 0.02% (w/v). In some embodiments, polysorbate 80 is present at a concentration of 0.04% (w/v). In some embodiments, polysorbate 80 is present at a concentration of 0.05% (w/v). In some embodiments, polysorbate 80 is present at a concentration of 0.06% (w/v). In some embodiments, polysorbate 80 is present at a concentration of 0.08% (w/v). In some embodiments, polysorbate 80 is present at a concentration of 0.1% (w/v). In some embodiments, polysorbate 80 is present at a concentration of 0.2% (w/v).

In some embodiments, polysorbate 20 is present at a concentration of between 0.001-1% (w/v). In some embodiments, polysorbate 20 is present at a concentration of between 0.005-0.5% (w/v). In some embodiments, polysorbate 20 is present at a concentration of between 0.01-0.05% (w/v).

In some embodiments, polysorbate 20 is present at a concentration of 0.001% (w/v). In some embodiments, polysorbate 20 is present at a concentration of 0.005% (w/v). In some embodiments, polysorbate 20 is present at a concentration of 0.01% (w/v). In some embodiments, polysorbate 20 is present at a concentration of 0.02% (w/v). In some embodiments, polysorbate 20 is present at a concentration of 0.04% (w/v). In some embodiments, polysorbate 20 is present at a concentration of 0.05% (w/v). In some embodiments, polysorbate 20 is present at a concentration of 0.06% (w/v). In some embodiments, polysorbate 20 is present at a concentration of 0.08% (w/v). In some embodiments, polysorbate 20 is present at a concentration of 0.1% (w/v). In some embodiments, polysorbate 20 is present at a concentration of 0.2% (w/v).

In some embodiments, the formulation further comprises a buffering agent.

In some embodiments, the buffering agent is a histidine buffer. In some embodiments, the buffering agent is an acetate buffer. In some embodiments, the buffering agent is a glutamate buffer. In some embodiments, the buffering agent comprises a histidine buffer and an acetate buffer. In some embodiments, the buffering agent comprises a histidine buffer and a glutamate buffer.

In some embodiments, wherein the concentration of the buffering agent individually or combined is between 5-100 mM. In some embodiments, wherein the concentration of the buffering agent individually or combined is between 10-60 mM. In some embodiments, wherein the concentration of the buffering agent individually or combined is between 15-40 mM.

In some embodiments, the concentration of a histidine buffer is 5 mM. In some embodiments, the concentration of a histidine buffer is 10 mM. In some embodiments, the concentration of a histidine buffer is 15 mM. In some embodiments, the concentration of a histidine buffer is 20 mM. In some embodiments, the concentration of a histidine buffer is 25 mM. In some embodiments, the concentration of a histidine buffer is 30 mM. In some embodiments, the concentration of a histidine buffer is 35 mM. In some embodiments, the concentration of a histidine buffer is 40 mM. In some embodiments, the concentration of a histidine buffer is 45 mM. In some embodiments, the concentration of a histidine buffer is 50 mM. In some embodiments, the concentration of a histidine buffer is 55 mM. In some embodiments, the concentration of a histidine buffer is 60 mM. In some embodiments, the concentration of a histidine buffer is 65 mM. In some embodiments, the concentration of a histidine buffer is 70 mM. In some embodiments, the concentration of a histidine buffer is 75 mM. In some embodiments, the concentration of a histidine buffer is about 80 mM. In some embodiments, the concentration of a histidine buffer is 85 mM. In some embodiments, the concentration of a histidine buffer is 90 mM. In some embodiments, the concentration of a histidine buffer is 95 mM. In some embodiments, the concentration of a histidine buffer is 100 mM.

In some embodiments, the concentration of an acetate buffer is 5 mM. In some embodiments, the concentration of an acetate buffer is 10 mM. In some embodiments, the concentration of an acetate buffer is 15 mM. In some embodiments, the concentration of an acetate buffer is 20 mM. In some embodiments, the concentration of an acetate buffer is 25 mM. In some embodiments, the concentration of an acetate buffer is 30 mM. In some embodiments, the concentration of an acetate buffer is 35 mM. In some embodiments, the concentration of an acetate buffer is 40 mM. In some embodiments, the concentration of an acetate buffer is 45 mM. In some embodiments, the concentration of an acetate buffer is 50 mM. In some embodiments, the concentration of an acetate buffer is 55 mM. In some embodiments, the concentration of an acetate buffer is 60 mM. In some embodiments, the concentration of an acetate buffer is 65 mM. In some embodiments, the concentration of an acetate buffer is 70 mM. In some embodiments, the concentration of an acetate buffer is 75 mM. In some embodiments, the concentration of an acetate buffer is about 80 mM. In some embodiments, the concentration of an acetate buffer is 85 mM. In some embodiments, the concentration of an acetate buffer is 90 mM. In some embodiments, the concentration of an acetate buffer is 95 mM. In some embodiments, the concentration of an acetate buffer is 100 mM.

In some embodiments, the concentration of a glutamate buffer is 5 mM. In some embodiments, the concentration of a glutamate buffer is 10 mM. In some embodiments, the concentration of a glutamate buffer is 15 mM. In some embodiments, the concentration of a glutamate buffer is 20 mM. In some embodiments, the concentration of a glutamate buffer is 25 mM. In some embodiments, the concentration of a glutamate buffer is 30 mM. In some embodiments, the concentration of a glutamate buffer is 35 mM. In some embodiments, the concentration of a glutamate buffer is 40 mM. In some embodiments, the concentration of a glutamate buffer is 45 mM. In some embodiments, the concentration of a glutamate buffer is 50 mM. In some embodiments, the concentration of a glutamate buffer is 55 mM. In some embodiments, the concentration of a glutamate buffer is 60 mM. In some embodiments, the concentration of a glutamate buffer is 65 mM. In some embodiments, the concentration of a glutamate buffer is 70 mM. In some embodiments, the concentration of a glutamate buffer is 75 mM. In some embodiments, the concentration of a glutamate buffer is about 80 mM. In some embodiments, the concentration of a glutamate buffer is 85 mM. In some embodiments, the concentration of a glutamate buffer is 90 mM. In some embodiments, the concentration of a glutamate buffer is 95 mM. In some embodiments, the concentration of a glutamate buffer is 100 mM.

In some embodiments, the buffering agent does not comprise succinate.

In one aspect, the present invention provides, among other things, a stable formulation comprising: an anti-IGF-1R antibody comprising at a concentration of 50 mg/ml; a histidine buffer at concentration of 20 mM; methionine at a concentration of 10 mM; polysorbate 80 at a concentration of 0.02% (w/v); sucrose at a concentration of 8% (w/v); and pH of 5.5; wherein the antibody comprises an HCDR1 of SEQ ID NO: 53, a HCDR2 of SEQ ID NO: 54, an HCDR3 of SEQ ID NO: 55, a LCDR1 of SEQ ID NO: 56, a LCDR2 of SEQ ID NO: 57 and a LCDR3 of SEQ ID NO: 58.

In one aspect, the present invention provides, among other things, a stable formulation comprising: an anti-IGF-1R antibody comprising at a concentration of 50 mg/ml; a histidine buffer at concentration of 20 mM; methionine at a concentration of 10 mM; polysorbate 80 at a concentration of 0.02% (w/v); sucrose at a concentration of 8% (w/v); and pH of 5.5; wherein the antibody comprises a heavy chain variable region of SEQ ID NO: 14 and a light chain variable region of SEQ ID NO: 13.

In one aspect, the present invention provides, among other things, a stable formulation comprising: an anti-IGF-1R antibody comprising at a concentration of 50 mg/ml; a histidine buffer at concentration of 20 mM; methionine at a concentration of 10 mM; polysorbate 80 at a concentration of 0.02% (w/v); sucrose at a concentration of 8% (w/v); and pH of 5.5; wherein the antibody comprises a heavy chain comprising SEQ ID NO: 94, and a light chain comprising SEQ ID NO: 93.

In one aspect, the present invention provides, among other things, a stable formulation comprising: an anti-IGF-1R antibody comprising at a concentration of 50 mg/ml; a histidine buffer at concentration of 20 mM; methionine at a concentration of 10 mM; polysorbate 80 at a concentration of 0.02% (w/v); sucrose at a concentration of 8% (w/v); and pH of 5.5; wherein the antibody comprises a heavy chain comprising SEQ ID NO: 92, and a light chain comprising SEQ ID NO: 93.

In one aspect, the present invention provides, among other things, a stable formulation comprising: an anti-IGF-1R antibody comprising at a concentration of 150 mg/ml; a histidine buffer at concentration of 20 mM; methionine at a concentration of 10 mM; polysorbate 80 at a concentration of 0.04% (w/v); sucrose at a concentration of 8% (w/v); and pH of 5.5; wherein the antibody comprises an HCDR1 of SEQ ID NO: 53, an HCDR2 of SEQ ID NO: 54, an HCDR3 of SEQ ID NO: 55, a LCDR1 of SEQ ID NO: 56, a LCDR2 of SEQ ID NO: 57 and a LCDR3 of SEQ ID NO: 58.

In some embodiments, antibody comprises a heavy chain variable region that is at least 90% identical to SEQ ID NO: 14 and a light chain variable region that is at least 90% identical to SEQ ID NO: 13.

In some embodiments, the heavy chain variable region comprises SEQ ID NO: 14 and the light chain variable region comprises SEQ ID NO: 13.

In one aspect, the present invention provides, among other things, a stable formulation comprising: an anti-IGF-1R antibody comprising at a concentration of 150 mg/ml; a histidine buffer at concentration of 20 mM; methionine at a concentration of 10 mM; polysorbate 80 at a concentration of 0.04% (w/v); sucrose at a concentration of 8% (w/v); and pH of 5.5; wherein the antibody comprises a heavy chain variable region of SEQ ID NO: 14 and a light chain variable region of SEQ ID NO: 13.

In some embodiments, anti-IGF-1R antibody comprises a heavy chain comprising SEQ ID NO: 92, and a light chain comprising SEQ ID NO: 93.

In some embodiments, anti-IGF-1R antibody comprises a heavy chain comprising SEQ ID NO: 94, and a light chain comprising SEQ ID NO: 93.

In one aspect, the present invention provides, among other things, a stable formulation comprising: an anti-IGF-1R antibody comprising at a concentration of 150 mg/ml; a histidine buffer at concentration of 20 mM; methionine at a concentration of 10 mM; polysorbate 80 at a concentration of 0.04% (w/v); sucrose at a concentration of 8% (w/v); and pH of 5.5; wherein the antibody comprises a heavy chain comprising SEQ ID NO: 92, and a light chain comprising SEQ ID NO: 93.

In one aspect, the present invention provides, among other things, a stable formulation comprising: an anti-IGF-1R antibody comprising at a concentration of 150 mg/ml; a histidine buffer at concentration of 20 mM; methionine at a concentration of 10 mM; polysorbate 80 at a concentration of 0.04% (w/v); sucrose at a concentration of 8% (w/v); and pH of 5.5; wherein the antibody comprises a heavy chain comprising SEQ ID NO: 94, and a light chain comprising SEQ ID NO: 93.

In some embodiments, less than 5% of the IGF-1R antibody exists as HMW species in the formulation upon storage at 40° C. for at least 4 weeks. In some embodiments, less than 4% of the IGF-1R antibody exists as HMW species in the formulation upon storage at 40° C. for at least 4 weeks. In some embodiments, less than 3% of the IGF-1R antibody exists as HMW species in the formulation upon storage at 40° C. for at least 4 weeks. In some embodiments, less than 2.5% of the IGF-1R antibody exists as HMW species in the formulation upon storage at 40° C. for at least 4 weeks. In some embodiments, less than 2% of the IGF-1R antibody exists as HMW species in the formulation upon storage at 40° C. for at least 4 weeks. In some embodiments, less than 1.5% of the IGF-1R antibody exists as HMW species in the formulation upon storage at 40° C. for at least 4 weeks. In some embodiments, less than 1% of the IGF-1R antibody exists as HMW species in the formulation upon storage at 40° C. for at least 4 weeks.

In some embodiments, at least 90% of the IGF-1R antibody exists as monomer in the formulation upon storage at 40° C. for at least 4 weeks. In some embodiments, at least 92% of the IGF-1R antibody exists as monomer in the formulation upon storage at 40° C. for at least 4 weeks. In some embodiments, at least 93% of the IGF-1R antibody exists as monomer in the formulation upon storage at 40° C. for at least 4 weeks. In some embodiments, at least 94% of the IGF-1R antibody exists as monomer in the formulation upon storage at 40° C. for at least 4 weeks. In some embodiments, at least 95% of the IGF-1R antibody exists as monomer in the formulation upon storage at 40° C. for at least 4 weeks. In some embodiments, at least 96% of the IGF-1R antibody exists as monomer in the formulation upon storage at 40° C. for at least 4 weeks. In some embodiments, at least 97% of the IGF-1R antibody exists as monomer in the formulation upon storage at 40° C. for at least 4 weeks. In some embodiments, at least 98% of the IGF-1R antibody exists as monomer in the formulation upon storage at 40° C. for at least 4 weeks. In some embodiments, at least 99% of the IGF-1R antibody exists as monomer in the formulation upon storage at 40° C. for at least 4 weeks.

In some embodiments, wherein less than 10% of the IGF-1R antibody exists as HMW species in the formulation upon storage at 5° C. for at least 9 months. In some embodiments, wherein less than 8% of the IGF-1R antibody exists as HMW species in the formulation upon storage at 5° C. for at least 9 months. In some embodiments, wherein less than 7% of the IGF-1R antibody exists as HMW species in the formulation upon storage at 5° C. for at least 9 months. In some embodiments, wherein less than 6% of the IGF-1R antibody exists as HMW species in the formulation upon storage at 5° C. for at least 9 months. In some embodiments, wherein less than 5% of the IGF-1R antibody exists as HMW species in the formulation upon storage at 5° C. for at least 9 months. In some embodiments, wherein less than 4% of the IGF-1R antibody exists as HMW species in the formulation upon storage at 5° C. for at least 9 months. In some embodiments, wherein less than 3% of the IGF-1R antibody exists as HMW species in the formulation upon storage at 5° C. for at least 9 months.

In some embodiments, less than 1% of the IGF-1R antibody exists as HMW species upon storage at 25° C. for at least 6 months. In some embodiments, less than 2% of the IGF-1R antibody exists as HMW species upon storage at 25° C. for at least 6 months. In some embodiments, less than 3% of the IGF-1R antibody exists as HMW species upon storage at 25° C. for at least 6 months. In some embodiments, less than 4% of the IGF-1R antibody exists as HMW species upon storage at 25° C. for at least 6 months. In some embodiments, less than 5% of the IGF-1R antibody exists as HMW species upon storage at 25° C. for at least 6 months.

In some embodiments, the % of HMW species or monomer is measured by size exclusion chromatography (SEC).

In some embodiments, the Tm onset is higher than 50° C. as measured by DSC. In some embodiments, the Tm onset is higher than 55° C. as measured by DSC. In some embodiments, the Tm onset is higher than 58° C. as measured by DSC. In some embodiments, the Tm onset is higher than 59° C. as measured by DSC. In some embodiments, the Tm onset is higher than 60° C. as measured by DSC. In some embodiments, the Tm onset is higher than 61° C. as measured by DSC. In some embodiments, the Tm onset is higher than 62° C. as measured by DSC.

In some embodiments, the acidic peak increases by less than 40% upon storage at 40° C. for at least four weeks as measured by iCIEF. In some embodiments, the acidic peak increases by less than 38% upon storage at 40° C. for at least four weeks as measured by iCIEF. In some embodiments, the acidic peak increases by less than 36% upon storage at 40° C. for at least four weeks as measured by iCIEF. In some embodiments, the acidic peak increases by less than 35% upon storage at 40° C. for at least four weeks as measured by iCIEF. In some embodiments, the acidic peak increases by less than 32% upon storage at 40° C. for at least four weeks as measured by iCIEF. In some embodiments, the acidic peak increases by less than 30% upon storage at 40° C. for at least four weeks as measured by iCIEF. In some embodiments, the acidic peak increases by less than 25% upon storage at 40° C. for at least four weeks as measured by iCIEF.

In some embodiments, wherein the composition clarity is 20 NTU or less. In some embodiments, wherein the composition clarity is 18 NTU or less. In some embodiments, wherein the composition clarity is 15 NTU or less. In some embodiments, wherein the composition clarity is 13 NTU or less. In some embodiments, wherein the composition clarity is 12 NTU or less. In some embodiments, wherein the composition clarity is 10 NTU or less. In some embodiments, wherein the composition clarity is 8 NTU or less.

In some embodiments, the formulation is free of particle upon storage at 40° C. for at least two weeks. In some embodiments, the formulation is free of particle upon storage at 40° C. for at least three weeks. In some embodiments, the formulation is free of particle upon storage at 40° C. for at least four weeks. In some embodiments, the formulation is free of particle upon storage at 40° C. for at least six weeks. In some embodiments, the formulation is free of particle upon storage at 40° C. for at least two months. In some embodiments, the formulation is free of particle upon storage at 40° C. for at least four months. In some embodiments, the formulation is free of particle upon storage at 40° C. for at least six months.

In some embodiments, the formulation is free of particle upon storage at 25° C. for at least two weeks. In some embodiments, the formulation is free of particle upon storage at 25° C. for at least three weeks. In some embodiments, the formulation is free of particle upon storage at 25° C. for at least four weeks. In some embodiments, the formulation is free of particle upon storage at 25° C. for at least six weeks. In some embodiments, the formulation is free of particle upon storage at 25° C. for at least two months. In some embodiments, the formulation is free of particle upon storage at 25° C. for at least four months. In some embodiments, the formulation is free of particle upon storage at 25° C. for at least six months.

In some embodiments, the decrease in purity is less than 8% upon storage at 40° C. for at least four weeks. In some embodiments, the decrease in purity is less than 5% upon storage at 40° C. for at least four weeks. In some embodiments, the decrease in purity is less than 4% upon storage at 40° C. for at least four weeks. In some embodiments, the decrease in purity is less than 3.5% upon storage at 40° C. for at least four weeks. In some embodiments, the decrease in purity is less than 3% upon storage at 40° C. for at least four weeks. In some embodiments, the decrease in purity is less than 2% upon storage at 40° C. for at least four weeks. In some embodiments, the decrease in purity is less than 1% upon storage at 40° C. for at least four weeks.

In some embodiments, the osmolality is between 280-380 mOsmol/kg, or between 340 and 420 mOsmol/kg.

In some embodiments, the formulation is stored at 25 to 25° C.

In some embodiments, the formulation is stored at 2-8° C.

In some embodiments, the formulation is suitable for intravenous administration. In some embodiments, the formulation is suitable for subcutaneous administration. In some embodiments, the formulation is suitable for intramuscular administration.

In some embodiments, wherein the formulation is a liquid pharmaceutical composition. In some embodiments, wherein the formulation is a lyophilized pharmaceutical composition.

In one aspect, the present invention provides, among other things, a dosage form comprising the stable formulation of the present invention in a vial. In one aspect, the present invention provides, among other things, a dosage form comprising the stable formulation of the present invention in a pre-filled syringe. In one aspect, the present invention provides, among other things, a dosage form comprising the stable formulation of the present invention in an autoinjector.

In one aspect, the present invention provides, among other things, use of the stable formulation for treatment of thyroid associated ophthalmopathy in a subject.

In some embodiments, the stable formulation is administered intravenously.

In some embodiments, the stable formulation is administered subcutaneously.

In one aspect, the present invention provides, among other things, use of the stable formulation for reducing Clinical Activity Score (CAS) of thyroid-associated ophthalmopathy (TAO) in a subject, comprising administering the stable formulation.

In some embodiments, the treated subject has reduced diplopia.

In one aspect, the present invention provides, among other things, use of the stable formulation for treating or reducing the severity of thyroid-associated ophthalmopathy (TAO) in a subject comprising administering the stable formulation, wherein treatment with said pharmaceutical composition reduces proptosis by at least 2 mm in an eye; is not accompanied by a deterioration of 2 mm or more in the other (or fellow eye); and reduces the CAS in said subject to either one (1) or zero (0).

In one aspect, the present invention provides, among other things, use of the stable formulation for improving the quality of life in a subject with thyroid-associated ophthalmopathy (TAO, also called Graves' Ophthalmopathy/Graves' Orbitopathy), comprising administering the stable formulation of the present invention.

In some embodiments, the quality of life is measured by the Graves' Ophthalmopathy Quality of Life (GO-QoL) assessment. In some embodiments, the quality of life is measured by the Visual Functioning or Appearance subscale thereof.

In some embodiments, the treatment results in an improvement on the Functioning subscale of the GO-QoL.

In some embodiments, the treatment results in an improvement on the Appearance subscale of the GO-QoL.

In one aspect, the present invention provides, among other things, use of the stable formulation for treating or reducing the severity of diplopia in a subject with thyroid-associated ophthalmopathy (TAO), comprising administering the stable formulation of the present invention.

In some embodiments, the diplopia is constant diplopia.

In some embodiments, the diplopia is inconstant diplopia.

In some embodiments, the diplopia is intermittent diplopia.

In some embodiments, the formulations comprise an anti-IGF-1R antibody, or antigen-binding fragment thereof, at a concentration of 25-75 mg/ml and a pH of 4.5-6.0. In some embodiments, the anti-IGF-1R antibody, or antigen-binding fragment thereof, comprises a heavy chain HCDR1 comprising SEQ ID NO: 53, an HCDR2 comprising SEQ ID NO: 54, an HCDR3 comprising SEQ ID NO: 55, a light chain comprising a LCDR1 comprising SEQ ID NO: 56, a LCDR2 comprising SEQ ID NO: 57 and a LCDR3 comprising SEQ ID NO: 58.

In some embodiments, the formulation comprises: (i) an anti-IGF-1R antibody at a concentration of 45-55 mg/ml, wherein the antibody comprises a heavy chain HCDR1 comprising SEQ ID NO: 53, an HCDR2 comprising SEQ ID NO: 54, an HCDR3 comprising SEQ ID NO: 55, a light chain comprising a LCDR1 comprising SEQ ID NO: 56, a LCDR2 comprising SEQ ID NO: 57 and a LCDR3 comprising SEQ ID NO: 58, (ii) a buffer at a concentration of about 10-60 mM; (iii) sucrose at a concentration of about 1-20% (w/v); (iv) a stabilizer at a concentration of about 1-15 mM; and (v) a surfactant at a concentration of about 0.001-1% (w/v) and wherein the pharmaceutical composition has a pH of 4.5-6.0.

In some embodiments, the pH is 5-6. In some embodiments, the pH is 5.5. In some embodiments, the pH is 4.5. In some embodiments, the pH is 6.0. In some embodiments, the pH is about 5 to about 6. In some embodiments, the pH is about 5.5. In some embodiments, the pH is about 4.5. In some embodiments, the pH is about 6.0.

In some embodiments, the formulation comprises an anti-IGF-1R antibody at a concentration of 45-55 mg/ml; a histidine buffer at concentration of 20 mM; methionine at a concentration of 10 mM; polysorbate 80 at a concentration of 0.02% (w/v); sucrose at a concentration of 8% (w/v); and pH of 5.5; wherein the antibody comprises a heavy chain HCDR1 comprising SEQ ID NO: 53, an HCDR2 comprising SEQ ID NO: 54, an HCDR3 comprising SEQ ID NO: 55, a light chain comprising a LCDR1 comprising SEQ ID NO: 56, a LCDR2 comprising SEQ ID NO: 57 and a LCDR3 comprising SEQ ID NO: 58.

In some embodiments, the anti-IGF-1R antibody comprises a heavy chain variable region comprising an amino acid sequence that is at least 90% (e.g., at least 91%, at least 92%, at least 93%, at least 94%, at least 95%, at least 96%, at least 97%, at least 98%, or at least 99%) identical to SEQ ID NO: 14, and a light chain variable region comprising an amino acid sequence that is at least 90% (e.g., at least 91%, at least 92%, at least 93%, at least 94%, at least 95%, at least 96%, at least 97%, at least 98%, or at least 99%) identical to SEQ ID NO: 13, provided that the heavy chain HCDR1 comprises the amino acid sequence of SEQ ID NO: 53, an HCDR2 comprises the amino acid sequence of SEQ ID NO: 54, an HCDR3 comprises the amino acid sequence of SEQ ID NO: 55, a light chain comprising a LCDR1 comprises the amino acid sequence of SEQ ID NO: 56, a LCDR2 comprises the amino acid sequence of SEQ ID NO: 57 and a LCDR3 comprises the amino acid sequence of SEQ ID NO: 58.

In some embodiments, the anti-IGF-1R antibody comprises a heavy chain variable region comprising SEQ ID NO: 14, and a light chain variable region comprising SEQ ID NO: 13.

In some embodiments, the anti-IGF-1R antibody comprises a heavy chain comprising SEQ ID NO: 92, and a light chain comprising SEQ ID NO: 93.

In some embodiments, the anti-IGF-1R antibody is present at a concentration of 50 mg/ml. In some embodiments, the formulation further comprises sucrose. In some embodiments, sucrose is present at a concentration of about 1-20% (w/v). In some embodiments, the sucrose is present at a concentration of about 8% (w/v).

In some embodiments the formulation further comprises a stabilizer. In some embodiments, the stabilizer is methionine. methionine is present at a concentration of about 1-15 mM. In some embodiments, methionine is present at a concentration of about 10 mM.

In some embodiments, the formulation further comprises a surfactant. In some embodiments, the surfactant is polysorbate 80. In some embodiments, polysorbate 80 is present at a concentration of about 0.001-1% (w/v). In some embodiments, polysorbate 80 is present at a concentration of about 0.02% (w/v).

In some embodiments, the formulation further comprises a buffering agent. In some embodiments, the buffering agent is a histidine buffer. In some embodiments, the concentration of a histidine buffer is between about 10-60 mM. In some embodiments, the concentration of a histidine buffer is about 20 mM.

In some embodiments, the anti-IGF-1R antibody of the formulation is present in the formulation as one or more anti-IGF-1R antibody variant species. In some embodiments, the anti-IGF-1R antibody variant species are characterized as monomer species, high molecular weight species (HMWS), or charge variants. In some embodiments, the amount of anti-IGF-1R antibody monomer is (i) 95% or greater as measured by the main peak in size exclusion chromatography (SEC), and (ii) the amount of high molecular weight species (HMWS) is 5% or less as measured by SEC.

In some embodiments, the formulation comprises an acidic peak, a main peak and a basic peak as measured by imaged capillary isoelectric focusing (iCIEF).

In some embodiments, the formulation is characterized by a charge variant profile as measured by iCIEF comprising (i) 25% or greater main peak species, (ii) 70% or less acidic species, and (iii) 15% or less basic species.

In some embodiments, the acidic peak in the stable formulation, measured by iCIEF, increases in area percent by less than 40% (e.g., less than 38%, 36%, 34%, 32% or 30%) after storage for 4 weeks at a temperature of 40° C., relative to the initial value.

In some embodiments, the pharmaceutical composition main peak, measured by iCIEF, decreases in area percent by less than 35% (e.g., less than 34%, less than 33%, less than 32%, less than 31%, less than 30%, less than 29%, less than 28%, less than 27%, less than 26%, less than 25%, than less than 24%, less than 23%, less than 22%, less than 21% or less than 20%) after 4 weeks at a temperature of 40° C., relative to the initial value.

In some embodiments, the formulation basic peak, measured by iCIEF, decreases in area percent by less than 6.5% (e.g., less than 6%, less than 5%, less than 4%, less than 3%, less than 2%, or less than 1%) after 4 weeks at a temperature of 40° C., relative to the initial value.

In some embodiments, the composition clarity of the stable formulation is 18 NTU or less. In some embodiments, the osmolality of the stable formulation is between 280-380 mOsmol/kg, or between 340-420 mOsmol/kg. In some embodiments, the osmolality of the stable formulation is between 280-380 mOsmol/kg. In some embodiments, the osmolality of the stable formulation is between 340-420 mOsmol/kg.

In some embodiments, the formulation is stored at −25° C. to 25° C. In some embodiments, the stable formulation is stored at 2-8° C. In some embodiments, the formulation is stored at 2-6° C. In some embodiments, the formulation is stored at 4-6° C.

In some embodiments, the formulation is suitable for intravenous, subcutaneous, or intramuscular administration. In some embodiments, the stable formulation is a liquid pharmaceutical composition or a lyophilized pharmaceutical composition. Further disclosed herein are dosage forms of the stable formulation in a vial, pre-filled syringe or autoinjector.

In some embodiments, pharmaceutical compositions are provided comprising the formulations provided for herein. In some embodiments, the pharmaceutical composition comprises: (i) an anti-IGF-1R antibody, or antigen-binding fragment thereof; (ii) a buffer at a concentration of about 10-60 mM; (iii) sucrose at a concentration of about 1-20% (w/v); (iv) a stabilizer at a concentration of about 1-15 mM; and (v) a surfactant at a concentration of about 0.001-1% (w/v).

In some embodiments, the antibody, or antigen binding fragment thereof, comprises: a VL sequence as set forth in SEQ ID NO: 1, 3, 5, 7, 9, 11, 13, 15, 79, or 86; a VH sequence as set forth in SEQ ID NO: 2, 4, 6, 8, 10, 12, 14, 16, 80, or 83; a LCDR sequence as set forth in SEQ ID NO: 17, 18, 19, 23, 24, 25, 29, 30, 31, 35, 36, 37, 41, 42, 43, 47, 48, 49, 53, 54, 55, 59, 60, 61, or 81, or a HCDR sequence as set forth in SEQ ID NO: 20, 21, 22, 26, 27, 28, 32, 33, 34, 38, 39, 40, 44, 45, 46, 50, 51, 52, 56, 57, 58, 62, 63, or 64; and any combination or variant thereof.

In some embodiments, the antibody, or antigen binding fragment thereof, comprises: (i) a heavy chain variable region comprising heavy chain CDR1, CDR2, and CDR3 sequences, wherein the heavy chain CDR1 sequence has the amino acid sequence of SEQ ID NO: 20; the heavy chain CDR2 has the amino acid sequence of SEQ ID NO: 21; and the heavy chain CDR3 sequence has the amino acid sequence of SEQ ID NO: 22; or variants of any of the foregoing; and (ii) a light chain variable region comprising light chain CDR1, CDR2, and CDR3 sequences, wherein the light chain CDR1 sequence has the amino acid sequence SEQ ID NO: 17; the light chain CDR2 sequence has the amino acid sequence of SEQ ID NO: 18; and the light chain CDR3 sequence has the amino acid sequence of SEQ ID NO: 19; or variants of any of the foregoing. In some embodiments, the antibody, or antigen binding fragment thereof, comprises: (i) a heavy chain variable region comprising heavy chain CDR1, CDR2, and CDR3 sequences, wherein the heavy chain CDR1 sequence has the amino acid sequence of SEQ ID NO: 26; the heavy chain CDR2 has the amino acid sequence of SEQ ID NO: 27; and the heavy chain CDR3 sequence has the amino acid sequence of SEQ ID NO: 28; or variants of any of the foregoing; and (ii) a light chain variable region comprising light chain CDR1, CDR2, and CDR3 sequences, wherein the light chain CDR1 sequence has the amino acid sequence SEQ ID NO: 23; the light chain CDR2 sequence has the amino acid sequence of SEQ ID NO: 24; and the light chain CDR3 sequence has the amino acid sequence of SEQ ID NO: 25; or variants of any of the foregoing. In some embodiments, the antibody, or antigen binding fragment thereof, comprises: (i) a heavy chain variable region comprising heavy chain CDR1, CDR2, and CDR3 sequences, wherein the heavy chain CDR1 sequence has the amino acid sequence of SEQ ID NO: 32; the heavy chain CDR2 has the amino acid sequence of SEQ ID NO: 33; and the heavy chain CDR3 sequence has the amino acid sequence of SEQ ID NO: 34; or variants of any of the foregoing; and (ii) a light chain variable region comprising light chain CDR1, CDR2, and CDR3 sequences, wherein the light chain CDR1 sequence has the amino acid sequence SEQ ID NO: 29; the light chain CDR2 sequence has the amino acid sequence of SEQ ID NO: 30; and the light chain CDR3 sequence has the amino acid sequence of SEQ ID NO: 31; or variants of any of the foregoing. In some embodiments, the antibody, or antigen binding fragment thereof, comprises: (i) a heavy chain variable region comprising heavy chain CDR1, CDR2, and CDR3 sequences, wherein the heavy chain CDR1 sequence has the amino acid sequence of SEQ ID NO: 38; the heavy chain CDR2 has the amino acid sequence of SEQ ID NO: 39; and the heavy chain CDR3 sequence has the amino acid sequence of SEQ ID NO: 40; or variants of any of the foregoing; and (ii) a light chain variable region comprising light chain CDR1, CDR2, and CDR3 sequences, wherein the light chain CDR1 sequence has the amino acid sequence SEQ ID NO: 35; the light chain CDR2 sequence has the amino acid sequence of SEQ ID NO: 36; and the light chain CDR3 sequence has the amino acid sequence of SEQ ID NO: 37; or variants of any of the foregoing. In some embodiments, the antibody, or antigen binding fragment thereof, comprises: (i) a heavy chain variable region comprising heavy chain CDR1, CDR2, and CDR3 sequences, wherein the heavy chain CDR1 sequence has the amino acid sequence of SEQ ID NO: 44; the heavy chain CDR2 has the amino acid sequence of SEQ ID NO: 45; and the heavy chain CDR3 sequence has the amino acid sequence of SEQ ID NO: 46; or variants of any of the foregoing; and (ii) a light chain variable region comprising light chain CDR1, CDR2, and CDR3 sequences, wherein the light chain CDR1 sequence has the amino acid sequence SEQ ID NO: 41; the light chain CDR2 sequence has the amino acid sequence of SEQ ID NO: 42; and the light chain CDR3 sequence has the amino acid sequence of SEQ ID NO: 43; or variants of any of the foregoing. In some embodiments, the antibody, or antigen binding fragment thereof, comprises: (i) a heavy chain variable region comprising heavy chain CDR1, CDR2, and CDR3 sequences, wherein the heavy chain CDR1 sequence has the amino acid sequence of SEQ ID NO: 50; the heavy chain CDR2 has the amino acid sequence of SEQ ID NO: 51; and the heavy chain CDR3 sequence has the amino acid sequence of SEQ ID NO: 52; or variants of any of the foregoing; and (ii) a light chain variable region comprising light chain CDR1, CDR2, and CDR3 sequences, wherein the light chain CDR1 sequence has the amino acid sequence SEQ ID NO: 47; the light chain CDR2 sequence has the amino acid sequence of SEQ ID NO: 48; and the light chain CDR3 sequence has the amino acid sequence of SEQ ID NO: 49; or variants of any of the foregoing. In some embodiments, the antibody, or antigen binding fragment thereof, comprises: (i) a heavy chain variable region comprising heavy chain CDR1, CDR2, and CDR3 sequences, wherein the heavy chain CDR1 sequence has the amino acid sequence of SEQ ID NO: 56; the heavy chain CDR2 has the amino acid sequence of SEQ ID NO: 57; and the heavy chain CDR3 sequence has the amino acid sequence of SEQ ID NO: 58; or variants of any of the foregoing; and (ii) a light chain variable region comprising light chain CDR1, CDR2, and CDR3 sequences, wherein the light chain CDR1 sequence has the amino acid sequence SEQ ID NO: 53; the light chain CDR2 sequence has the amino acid sequence of SEQ ID NO: 54; and the light chain CDR3 sequence has the amino acid sequence of SEQ ID NO: 55; or variants of any of the foregoing. In some embodiments, the antibody, or antigen binding fragment thereof, comprises: (i) a heavy chain variable region comprising heavy chain CDR1, CDR2, and CDR3 sequences, wherein the heavy chain CDR1 sequence has the amino acid sequence of SEQ ID NO: 62; the heavy chain CDR2 has the amino acid sequence of SEQ ID NO: 63; and the heavy chain CDR3 sequence has the amino acid sequence of SEQ ID NO: 64; or variants of any of the foregoing; and (ii) a light chain variable region comprising light chain CDR1, CDR2, and CDR3 sequences, wherein the light chain CDR1 sequence has the amino acid sequence SEQ ID NO: 59; the light chain CDR2 sequence has the amino acid sequence of SEQ ID NO: 60; and the light chain CDR3 sequence has the amino acid sequence of SEQ ID NO: 61; or variants of any of the foregoing. In some embodiments, the antibody, or antigen binding fragment thereof, comprises: (i) a heavy chain variable region comprising heavy chain CDR1, CDR2, and CDR3 sequences, wherein the heavy chain CDR1 sequence has the amino acid sequence of SEQ ID NO: 38; the heavy chain CDR2 has the amino acid sequence of SEQ ID NO: 39; and the heavy chain CDR3 sequence has the amino acid sequence of SEQ ID NO: 40; or variants of any of the foregoing; and (ii) a light chain variable region comprising light chain CDR1, CDR2, and CDR3 sequences, wherein the light chain CDR1 sequence has the amino acid sequence SEQ ID NO: 35; the light chain CDR2 sequence has the amino acid sequence of SEQ ID NO: 36; and the light chain CDR3 sequence has the amino acid sequence of SEQ ID NO: 81; or variants of any of the foregoing.

In some embodiments, the antibody comprises a VL sequence as set forth in SEQ ID NO: 1, 3, 5, 7, 9, 11, 13, 15, 79, or 86, or a variant thereof.

In some embodiments, the antibody comprises a VH sequence as set forth in SEQ ID NO: 2, 4, 6, 8, 10, 12, 14, 16, 80, or 83, or a variant thereof.

In some embodiments, the antibody comprises a V_{L} and V_{H} of SEQ ID NO:1 and SEQ ID NO: 2; SEQ ID NO: 3 and SEQ ID NO: 4; SEQ ID NO: 5 and SEQ ID NO: 6; SEQ ID NO: 7 and SEQ ID NO: 8; SEQ ID NO: 9 and SEQ ID NO: 10; SEQ ID NO: 11 and SEQ ID NO: 12; SEQ ID NO: 3 and SEQ ID NO: 83; SEQ ID NO: 13 and SEQ ID NO: 14; SEQ ID NO: 15 and SEQ ID NO: 16; SEQ ID NO: 79 and SEQ ID NO: 80; SEQ ID NO: 86 and SEQ ID NO: 14; SEQ ID NO: 98 and SEQ ID NO: 99, respectively, or a variant thereof, wherein the CDRs of the variant are constant.

In some embodiments, the antibody comprises a V_{L} and V_{H} of SEQ ID NO: 13 and SEQ ID NO: 14 or a variant thereof, wherein the CDRs of the variant are constant.

In some embodiments, the antibody comprises a light chain comprising the amino acid sequence of SEQ ID NO: 93 and a heavy chain comprises an amino acid sequence of ID NO: 92; or a light chain comprising the amino acid sequence of SEQ ID NO: 93 and a heavy chain comprises an amino acid sequence of ID NO: 94.

In some embodiments, the antibody comprises: a V_{L} and V_{H} of SEQ ID NO:98 and SEQ ID NO: 99, or a variant thereof, wherein the CDRs of the variant are constant; or a light chain comprising the amino acid sequence of SEQ ID NO: 3 and a heavy chain comprises an amino acid sequence of ID NO: 83.

In some embodiments, the variant has at least 90%, 91%, 92%, 93%, 94%, 95%, 96%, 97%, 98%, or 99% identity to a V_{L} and V_{H} of SEQ ID NO: 1 and SEQ ID NO: 2; SEQ ID NO: 3 and SEQ ID NO: 4; SEQ ID NO: 5 and SEQ ID NO: 6; SEQ ID NO: 7 and SEQ ID NO: 8; SEQ ID NO: 9 and SEQ ID NO: 10; SEQ ID NO: 11 and SEQ ID NO: 12; SEQ ID NO: 3 and SEQ ID NO: 83; SEQ ID NO: 13 and SEQ ID NO: 14; SEQ ID NO: 15 and SEQ ID NO: 16; SEQ ID NO: 79 and SEQ ID NO: 80; SEQ ID NO: 86 and SEQ ID NO: 14; SEQ ID NO: 98 and SEQ ID NO: 99, respectively, or a variant thereof, wherein the CDRs of the variant are constant.

In some embodiments, the antibody, or antigen-binding fragment thereof, is present at a concentration of about 1-300 mg/mL. In some embodiments, the antibody, or antigen-binding fragment thereof, is present at a concentration of about 1-200 mg/mL. In some embodiments, the antibody, or antigen-binding fragment thereof, is present at a concentration of about 20-150 mg/mL. In some embodiments, the antibody, or antigen-binding fragment thereof, is present at a concentration of about 25 mg/mL. In some embodiments, the antibody, or antigen-binding fragment thereof, is present at a concentration of about 50 mg/mL. In some embodiments, the antibody, or antigen-binding fragment thereof, is present at a concentration of about 150 mg/mL.

In some embodiments, the buffer is histidine buffer, histidine HCl buffer, glycine buffer, Tris/glycine buffer, acetate buffer, sodium acetate buffer, potassium acetate buffer, magnesium acetate buffer, phosphate buffer, citrate buffer, or succinate buffer. In some embodiments, the buffer is histidine buffer. In some embodiments, the histidine buffer is at a concentration of about 10-60 mM. In some embodiments, the histidine buffer is at a concentration of about 10-40 mM. In some embodiments, the histidine buffer is at a concentration of about 15-30 mM. In some embodiments, the histidine buffer is at a concentration of about 20 mM.

In some embodiments, the sucrose is at a concentration of about 1-15% (w/v). In some embodiments, the sucrose is at a concentration of about 5-10% (w/v). In some embodiments, the sucrose is at a concentration of about 8% (w/v).

In some embodiments, the stabilizer, or the anti-oxidant, is methionine, L-methionine, arginine, glycine, histidine, or proline. In some embodiments, the stabilizer, or the anti-oxidant, is methionine, or L-methionine. In some embodiments, the methionine, or L-methionine, is present at a concentration of about 1-15 mM. In some embodiments, the methionine, or L-methionine, is present at a concentration of about 7-13 mM. In some embodiments, the methionine, or L-methionine, is present at a concentration of about 10 mM.

In some embodiments, the surfactant is a polysorbate, or a poloxamer. In some embodiments, the polysorbate is polysorbate 80 (PS80), or polysorbate 20 (PS20). In some embodiments, the polysorbate is polysorbate 80 (PS80), or polysorbate 20 (PS20). In some embodiments, the polysorbate is polysorbate 80 (PS80). In some embodiments, the polysorbate 80 is at a concentration of about 0.001-1% (w/v). In some embodiments, the polysorbate 80 is at a concentration of about 0.01-0.1% (w/v). In some embodiments, the polysorbate 80 is at a concentration of about 0.01-0.05% (w/v). In some embodiments, the polysorbate 80 is at a concentration of about 0.01% (w/v). In some embodiments, the polysorbate 80 is at a concentration of about 0.02% (w/v).

In some embodiments, the pH of the pharmaceutical composition is about 4.5 to about 6.0. In some embodiments, the pH of the pharmaceutical composition is about 5.0 to about 6.0. In some embodiments, the pH of the pharmaceutical composition is about 5.5. In some embodiments, the pH of the pharmaceutical composition is about 6.0.

In some embodiments, the pharmaceutical composition comprises: (i) an anti-IGF-1R antibody, or antigen-binding fragment thereof, at a concentration of about 10-300 mg/mL; (ii) a histidine buffer at a concentration of about 10-60 mM; (iii) sucrose at a concentration of about 1-20% (w/v); (iv) methionine, or L-methionine, at a concentration of about 1-15 mM; and (v) a polysorbate at a concentration of about 0.001-1% (w/v).

In some embodiments, the pharmaceutical composition comprises: (i) an anti-IGF-1R antibody, or antigen-binding fragment thereof, comprising a light chain comprising the amino acid sequence of SEQ ID NO: 1 and a heavy chain comprising the amino acid sequence of SEQ ID NO: 2; a light chain comprising the amino acid sequence of SEQ ID NO: 3 and a heavy chain comprising the amino acid sequence of SEQ ID NO: 4; a light chain comprising the amino acid sequence of SEQ ID NO: 5 and a heavy chain comprising the amino acid sequence of SEQ ID NO: 6; a light chain comprising the amino acid sequence of SEQ ID NO: 7 and a heavy chain comprising the amino acid sequence of SEQ ID NO: 8; a light chain comprising the amino acid sequence of SEQ ID NO: 9 and a heavy chain comprising the amino acid sequence of SEQ ID NO: 10; a light chain comprising the amino acid sequence of SEQ ID NO: 11 and a heavy chain comprising the amino acid sequence of SEQ ID NO: 12; a light chain comprising the amino acid sequence of SEQ ID NO: 93 and a heavy chain comprising the amino acid sequence of SEQ ID NO: 92; a light chain comprising the amino acid sequence of SEQ ID NO: 15 and a heavy chain comprising the amino acid sequence of SEQ ID NO: 16; a light chain comprising the amino acid sequence of SEQ ID NO: 79 and a heavy chain comprising the amino acid sequence of SEQ ID NO: 80; a light chain comprising the amino acid sequence of SEQ ID NO: 3 and a heavy chain comprising the amino acid sequence of SEQ ID NO: 83; a light chain comprising the amino acid sequence of SEQ ID NO: 100 and a heavy chain comprising the amino acid sequence of SEQ ID NO: 101; or a variant thereof, at a concentration of about 10-200 mg/mL; (ii) a histidine buffer at a concentration of about 10-40 mM; (iii) sucrose at a concentration of about 1-10% (w/v); (iv) methionine, or L-methionine, at a concentration of about 1-15 mM; and (v) a polysorbate at a concentration of about 0.01-0.1% (w/v), wherein the pH of the pharmaceutical compositions is about 5.0 to about 6.5.

In some embodiments, the variant antibody has at least 85% homology to a sequence of SEQ ID NO: 1-72, 78-83, or 85-86. In some embodiments, the variant has at least 90%, 91%, 92%, 93%, 94%, 95%, 96%, 97%, 98%, or 99% homology to a sequence of SEQ ID NO: 1-72, 78-83, or 85-86. In some embodiments, the variant has at least 85% identity to a sequence of SEQ ID NO: 1-72, 78-83, or 85-86. In some embodiments, the variant has at least 90%, 91%, 92%, 93%, 94%, 95%, 96%, 97%, 98%, or 99% identify to a sequence of SEQ ID NO: 1-72, 78-83, or 85-86. In some embodiments, the variant has CDRs that are constant as compared to the reference sequence.

In some embodiments, the antibody, or antigen-binding fragment thereof, is present at a concentration of about 20-150 mg/mL. In some embodiments, the antibody, or antigen-binding fragment thereof, is present at a concentration of about 25 mg/mL. In some embodiments, the antibody, or antigen-binding fragment thereof, is present at a concentration of about 50 mg/mL. In some embodiments, the antibody, or antigen-binding fragment thereof, is present at a concentration of about 150 mg/mL.

In some embodiments, the histidine buffer is at a concentration of about 15-30 mM. In some embodiments, the histidine buffer is at a concentration of about 20 mM.

In some embodiments, the sucrose is at a concentration of about 5-10% (w/v). In some embodiments, the sucrose is at a concentration of about 8% (w/v).

In some embodiments, the methionine, or L-methionine, is present at a concentration of about 1-15 mM. In some embodiments, the methionine, or L-methionine, is present at a concentration of about 7-13 mM. In some embodiments, the methionine, or L-methionine, is present at a concentration of about 10 mM.

In some embodiments, the polysorbate is polysorbate 80 (PS80), or polysorbate 20 (PS20). In some embodiments, the polysorbate is polysorbate 80 (PS80). In some embodiments, the polysorbate 80 is at a concentration of about 0.01-0.05% (w/v). In some embodiments, the polysorbate 80 is at a concentration of about 0.01% (w/v). In some embodiments, the polysorbate 80 is at a concentration of about 0.02% (w/v).

In some embodiments, the pH of the pharmaceutical composition is about 5.5 to about 6.0. In some embodiments, the pH of the pharmaceutical composition is about 5.5. In some embodiments, the pH of the pharmaceutical composition is about 6.0.

In some embodiments, the pharmaceutical composition is suitable for intravenous, subcutaneous, or intramuscular administration.

In some embodiments, the pharmaceutical composition is a liquid pharmaceutical composition.

In some embodiments, the pharmaceutical composition is a lyophilized pharmaceutical composition.

In some embodiments, the pharmaceutical composition main peak, measured by SEC-UPLC, decreases in area percent by less than 3% after 4 weeks at a temperature of 40° C., relative to the initial value. In some embodiments, the pharmaceutical composition main peak, measured by SEC-UPLC, decreases in area percent by less than 2% after 4 weeks at a temperature of 40° C., relative to the initial value. In some embodiments, the pharmaceutical composition main peak, measured by SEC-UPLC, decreases in area percent by less than 1.3% after 4 weeks at a temperature of 40° C., relative to the initial value.

In some embodiments, the dosage form comprises the pharmaceutical composition in a container. In some embodiments, the container is a plastic vial or glass vial. In some embodiments, the container is a glass vial with a volume of 2 mL, 6 mL, or 10 mL. In some embodiments, the container is a pre-filled syringe. In some embodiments, the container is an autoinjector.

In some embodiments, the kit comprises the pharmaceutical composition or the dosage form and instructions for use.

In some embodiments, the method of treating thyroid associated ophthalmopathy in a subject comprises administering a pharmaceutical composition to the subject. In some embodiments, the pharmaceutical composition is administered intravenously. In some embodiments, the pharmaceutical composition is administered subcutaneously.

Further disclosed herein are methods of treating or reducing the severity of thyroid-associated ophthalmopathy (TAO), or a symptom thereof. In some embodiments, treating or reducing the severity of TAO, or a symptom thereof, comprises administering to a subject a pharmaceutical composition. In some embodiments, reducing proptosis in an eye in a subject with TAO comprising administering to a subject a pharmaceutical composition. In some embodiments, treating thyroid eye disease in a subject comprises administering to a subject a pharmaceutical composition. In some embodiments, reducing CAS of TAO in a subject comprises administering to a subject a pharmaceutical composition. In some embodiments, a) reducing proptosis by at least 2 mm and b) reducing the CAS in a subject with TAO comprises administering to a subject a pharmaceutical composition. In some embodiments, proptosis is reduced by at least 2 mm. In some embodiments, proptosis is reduced by at least 3 mm. In some embodiments, proptosis is reduced by at least 4 mm. In some embodiments, the CAS of the subject is reduced by at least 2 points. In some embodiments, the CAS of the subject is reduced to one (1). In some embodiments, the CAS of the subject is reduced to zero (0).

In some embodiments, treating or reducing the severity of TAO in a subject comprises administering to a subject a pharmaceutical composition, wherein treatment with said pharmaceutical composition (i) reduces proptosis by at least 2 mm in an eye; (ii) is not accompanied by a deterioration of 2 mm or more in the other (or fellow eye); and (iii) reduces the CAS in said subject to either one (1) or zero (0).

In some embodiments, improving the quality of life in a subject with thyroid-associated ophthalmopathy (TAO, also called Graves' Ophthalmopathy/Graves' Orbitopathy) comprising administering to a subject a pharmaceutical composition. In some embodiments, the quality of life is measured by the Graves' Ophthalmopathy Quality of Life (GO-QoL) assessment, or either the Visual Functioning or Appearance subscale thereof. In some embodiments, the treatment results in an improvement of greater than or equal to 8 points on the GO-QoL. In some embodiments, the treatment results in an improvement on the Functioning subscale of the GO-QoL. In some embodiments, the treatment results in an improvement on the Appearance subscale of the GO-QoL.

In some embodiments, treating or reducing the severity of diplopia in a subject with TAO comprises administering to a subject a pharmaceutical composition. In some embodiments, the diplopia is constant diplopia. In some embodiments, the diplopia is inconstant diplopia. In some embodiments, the diplopia is intermittent diplopia.

### DETAILED DESCRIPTION

Provided herein are formulations and pharmaceutical compositions of antibodies that bind and modulate the activity of IGF-1R. The antibodies can be used, for example, to treat thyroid eye disease, such as, but not limited to thyroid-associated ophthalmopathy (TAO). The formulations and compositions provided for herein demonstrate unexpected stability of the antibodies provided for herein that allow, for example, high concentrations of antibodies to be stable.

As used herein, the term “pharmaceutical composition” refers to a medicinal or pharmaceutical formulation that contains an active ingredient as well as one or more excipients and/or diluents to facilitate the active ingredient to be used in methods of administration. The pharmaceutical composition of the present disclosure includes pharmaceutically acceptable components that are compatible with the anti-IGF-1R antibody. As used herein, the terms “pharmaceutical compositions” and “stable formulations” are used interchangeably.

In some embodiments, the anti-IGF-1R antibody is VRDN-2700, VRDN-03100, VRDN-02100, VRDN-02200, VRDN-02300, VRDN-02400, VRDN-02500, VRDN-01100, VRDN-02600, or VRDN-02301, the sequence of which are provided for herein. In some embodiments, more than one anti-IGF-1R antibody is present. In some embodiments, the anti-IGF-1R antibody is a biosimilar of any of anti-IGF-1R antibodies provided for herein. In some embodiments, the Fc domain of the antibody is not fucosylated. In some embodiments, the Fc domain of the antibody is not glycosylated. As provided for herein, in some embodiments, the Fc domain comprises a mutation as compared to wild-type.

As used herein, the term “pharmaceutically acceptable carrier” refers to an excipient or diluent in a pharmaceutical composition. The pharmaceutically acceptable carrier must be compatible with the other ingredients of the formulation and not deleterious to the recipient. The nature of the carrier differs with the mode of administration. For example, for intravenous administration, an aqueous solution carrier is generally used; for oral administration, a solid carrier is generally used.

TAO is an autoimmune condition most commonly associated with Graves' disease and hyperthyroidism but can also be found in patients who are euthyroid or hypothyroid. Orbitopathy in TAO is driven by Thyroid Stimulating Hormone Receptor (TSHR) agonistic autoantibodies and crosstalk between TSHR and IGF-1R. Pathological remodeling of the orbit and periorbital tissues results in varied presentations which may include dry eyes, increased lacrimation, local irritation, eyelid retraction and eventually proptosis, diplopia, and optic nerve compression, with ensuing vision loss. A person with TAO can also be said to be suffering from thyroid eye disease (TED).

The underlying pathology of TAO is the activation of an inflammatory cascade within the orbit, primarily due to recruitment of fibrocytes and immune cells. Over-expression of IGF-1R has been demonstrated within the orbit of TAO patients, and it has been surmised that IGF-1R inhibitory antibodies may disrupt the IGF-1R and TSHR cross-talk and dampen the inflammatory cascade. Indeed, IGF-1R antagonism has been demonstrated to robustly relieve much of the inflammatory symptomology that affects TAO patients.

As used herein, “Thyroid-associated Ophthalmopathy” (TAO), “Thyroid Eye Disease” (TED), “Graves' Ophthalmopathy” or “Graves' Orbitopathy” (GO) refer to the same disorder or condition and are used interchangeably. They all refer to the inflammatory orbital pathology associated with some autoimmune thyroid disorders, most commonly with “Graves' Disease” (GD), but sometimes with other diseases, e.g., Hashimoto's thyroiditis.

The terms “proptosis” and “exophthalmos” (also known as exophthalmos, exophthalmia, or exorbitism) refer to the forward projection, displacement, bulging, or protrusion of an organ. As used herein, the terms refer to the forward projection, displacement, bulging, or protrusion of the eye anteriorly out of the orbit. Proptosis and exophthalmos are considered by some of skill in the art to have the same meaning and are often used interchangeably, while others attribute subtle differences to their meanings. Exophthalmos is used by some to refer to severe proptosis; or to refer to endocrine-related proptosis. Yet others use the term exophthalmos when describing proptosis associated with the eye, in, for example, subjects with TAO (TED or GO).

As used herein, the terms “proptosis” and “exophthalmos” are used interchangeably and refer to the forward projection, displacement, bulging, or protrusion of the eye anteriorly out of the orbit. Owing to the rigid bony structure of the orbit with only anterior opening for expansion, any increase in orbital soft tissue contents taking place from the side or from behind will displace the eyeball forward. Proptosis or exophthalmos can be the result of a several disease processes including infections, inflammations, tumors, trauma, metastases, endocrine lesions, vascular diseases & extra orbital lesions. TAO (TED or GO) is currently recognized as the most common cause of proptosis in adults. Exophthalmos can be either bilateral, as is often seen in TAO (TED or GO), or unilateral (as is often seen in an orbital tumor). Based on the technologies currently available, or that will become available in the future, one of skill in the art would be capable of determining the best modality for diagnosing and evaluating the extent of proptosis or exophthalmos.

As used herein, the term “antibody” refers to any form of antibody that exhibits the desired biological activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, chimeric antibodies and camelized single domain antibodies. “Parental antibodies” are antibodies obtained by exposure of an immune system to an antigen prior to modification of the antibodies for an intended use, such as humanization of an antibody for use as a human therapeutic antibody.

An “Fc” region contains two heavy chain fragments comprising the C_{H}1 and C_{H}2 domains of an antibody. The two heavy chain fragments are held together by two or more disulfide bonds and by hydrophobic interactions of the C_{H}3 domains.

In some embodiments, the antibody comprises a Fc domain. In some embodiments, the Fc domain comprises a mutation to extend the half-life of the antibody. In some embodiments, the Fc domain comprises a mutation such as those described in U.S. Pat. No. 7,670,600, which is hereby incorporated by reference in its entirety. In some embodiment, the constant region comprises a mutation at position at amino acid residue 428 relative to a wild-type human IgG constant domain, numbered according to the EU numbering index of Kabat. Without being bound to any particular theory, an antibody comprising a mutation that corresponds to residue 428 can have an increased half-life compared to the half-life of an IgG having the wild-type human IgG constant domain. In some embodiments, the mutation is a substitution of the native residue with a threonine, leucine, phenylalanine or serine. In some embodiments, the antibody further comprises one or more amino acid substitutions relative to the corresponding wild-type human IgG constant domain at one or more of amino acid residues 251-256, 285-290, 308-314, 385-389, and 429-436, numbered according to the Kabat EU numbering index. The specific mutations or substitutions at these positions are described in U.S. Pat. No. 7,670,600, which is hereby incorporated by reference in its entirety.

In some embodiments, the Fc region comprises a S228P, L235E, M252Y, S254T, T256E, M428L, N434S, L234F, P331S mutation, or any combination thereof. In some embodiments, the Fc region comprises a M252Y, S254T, and T256E mutations. A non-limiting example of a Fc region comprising the M252Y, S254T, and T256E mutations (collectively, “YTE Mutations”) can be found in a sequence of SEQ ID NO: 89. In some embodiments, the Fc region comprising the YTE Mutations comprises a sequence of SEQ ID NO: 90, which differs from SEQ ID NO: 89 by the presence of a C-terminal lysine (K) residue. The numbering of the Fc region can be according to the Kabat numbering system for the Fc region.

In some embodiments, the Fc region comprises a S228P and a L235E mutation. In some embodiments, the antibody comprises a L234F, L235E, and P331S mutations. In some embodiments, the Fc region comprises M252Y, S254T, T256E, S228P and L235E mutations. In some embodiments, the Fc region comprises S228P, L235E, M428L, and N434S mutations. In some embodiments, the Fc region comprises the M428L and N434S mutations. In some embodiments, the Fc region comprises the L234F, L235E, P331S, M252Y, S254T, and T256E mutations. Mutations in the Fc region are also described in US2007041972A1, EP2235059B1, U.S. Pat. No. 8,394,925, and Mueller et al, Mol Immunol 1997 April; 34(6):441-52, each of which is incorporated by reference in its entirety. The numbering referenced herein refers to the Kabat numbering system for the Fc region.

In some embodiments, the Fc region comprises the sequence selected from:

(SEQ ID NO: 75)
APELLGGPSVFLFPPKPKDTLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVMHEALHNHY
TQKSLSLSPGK;
(SEQ ID NO: 76)
APELLGGPSVFLFPPKPKDTLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPPSREEMTKNQVSLTCL
VKGFYPSDIAVEWESNGQPENNYKTTPPVLDSDGSFFLYSKLTVDKSRWQQGNVFSCSVMHEALHNHY
TQKSLSLSPGK;
or
(SEQ ID NO: 77)
APPVAGPSVFLFPPKPKDTLMISRTPEVTCVVVDVSHEDPEVQFNWYVDGVEVHNAKTKPREEQFNSTFR
VVSVLTVVHQDWLNGKEYKCKVSNKGLPAPIEKTISKTKGQPREPQVYTLPPSREEMTKNQVSLTCLVK
GFYPSDIAVEWESNGQPENNYKTTPPMLDSDGSFFLYSKLTVDKSRWQQGNVFSCSVMHEALHNHYTQ
KSLSLSPG;
or
(SEQ ID NO: 84)
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKRVEPKSCDKTHTCPPCPAPELLGGPSVFLFPPKPKDTLYITREPEV
TCVVVDVSHEDPEVKFNWYVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPG
(SEQ ID NO: 87)
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGKEYKCKVS
NKALPAPIEKTISKAKGQPREPQVYTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSLSPGK
(SEQ ID NO: 88)
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGKEYKCKVS
NKALPAPIEKTISKAKGQPREPQVYTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSLSPG
(SEQ ID NO: 89)
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELLGGPSVFLFPPKPKDTLYITREPEV
TCVVVDVSHEDPEVKFNWYVDGVEVHNAKTKPREEQYNSTYRVVSVLTVLHQDWLNGKEYKCKVSN
KALPAPIEKTISKAKGQPREPQVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPG
(SEQ ID NO: 90)
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELLGGPSVFLFPPKPKDTLYITREPEV
TCVVVDVSHEDPEVKFNWYVDGVEVHNAKTKPREEQYNSTYRVVSVLTVLHQDWLNGKEYKCKVSN
KALPAPIEKTISKAKGQPREPQVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPGK

In some embodiments, the anti-IGF-1R antibody is a variant antibody. Typically, a variant antibody or antigen binding fragment of the antibodies provided herein retain at least 10% of its IGF-1R binding activity (when compared to a parental antibody that is modified) when that activity is expressed on a molar basis. In some embodiments, a variant antibody (or antigen fragment thereof), or antigen binding fragment of an antibody provided herein, retains at least 20%, 50%, 70%, 80%, 90%, 95% or 100% or more of the IGF-1R binding affinity as the parental antibody. As described herein, it is also intended that an antibody, or antigen binding fragment thereof, can include conservative or non-conservative amino acid substitutions, which can also be referred to as “conservative variants” or “function conserved variants” of the antibody, that do not substantially alter its biologic activity.

The term “monoclonal antibody”, as used herein, refers to population of substantially homogeneous antibodies, i.e., the antibody molecules comprising the population are identical in amino acid sequence except for possible naturally occurring mutations that may be present in minor amounts. The modifier “monoclonal” indicates the character of the antibody as being obtained from a substantially homogeneous population of antibodies and is not to be construed as requiring production of the antibody by any particular method. For example, the monoclonal antibodies to be used as provided for herein may be made by the hybridoma method first described by Kohler et al. (1975) Nature 256:495 or may be made by recombinant DNA methods (see, e.g., U.S. Pat. No. 4,816,567). The “monoclonal antibodies” may also be isolated from phage antibody libraries using the techniques described in Clackson et al. (1991) Nature 352:624-628 and Marks et al. (1991) J. Mol. Biol. 222:581-597, for example. See also Presta (2005) J. Allergy Clin. Immunol. 116:731.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2^{nd} ed. Raven Press, N.Y. (1989).

The variable regions of each light/heavy chain pair form the antibody binding site. Thus, in general, an intact antibody has two binding sites. Typically, the variable domains of both the heavy and light chains comprise three hypervariable regions, also called complementarity determining regions (CDRs), located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, unless otherwise specified, in accordance with the definitions of Sequences of Proteins of Immunological Interest, Kabat, et al.; National Institutes of Health, Bethesda, Md.; 5^{th} ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

As used herein, the term “hypervariable region” refers to the amino acid residues of an antibody that are responsible for antigen-binding. The hypervariable region comprises amino acid residues from a “complementarity determining region” or “CDR” (i.e. residues 24-34 (CDRL1), 50-56 (CDRL2) and 89-97 (CDRL3) in the light chain variable domain and residues 31-35 (CDRH1), 50-65 (CDRH2) and 95-102 (CDRH3) in the heavy chain variable domain; Kabat et al. (1991) Sequences of Proteins of Immunological Interest, 5th Ed. Public Health Service, National Institutes of Health, Bethesda, Md.) and/or those residues from a “hypervariable loop” (i.e. residues 26-32 (CDRL1), 50-52 (CDRL2) and 91-96 (CDRL3) in the light chain variable domain and 26-32 (CDRH1), 53-55 (CDRH2) and 96-101 (CDRH3) in the heavy chain variable domain; Chothia and Lesk (1987) J. Mol. Biol. 196:901-917). As used herein, the term “framework” or “FR” residues refers to those variable domain residues other than the hypervariable region residues defined herein as CDR residues. CDRs provide the majority of contact residues for the binding of the antibody to the antigen or epitope. CDRs of interest can be derived from donor antibody variable heavy and light chain sequences, and include analogs of the naturally occurring CDRs, which analogs also share or retain the same antigen binding specificity and/or neutralizing ability as the donor antibody from which they were derived.

”The term “homolog” means protein sequences having between 40% and 100% sequence homology or identity to a reference sequence. Percent identity between two peptide chains can be determined by pair wise alignment using the default settings of the AlignX module of Vector NTI v.9.0.0 (Invitrogen Corp., Carslbad, Calif.). In some embodiments, the antibody, or antigenic binding fragment thereof has, at least 50, 60, 70, 80, 90, 91, 92, 93, 94, 95, 96, 97, 98, or 99% homology or identity to a sequence described herein. In some embodiments, the antibody has conservative substitutions as compared to a sequence described herein. Exemplary conservative substitutions are illustrated in Table 1 and are encompassed within the scope of the disclosed subject matter. The conservative substitution may reside in the framework regions, or in antigen-binding sites, as long they do not adversely affect the properties of the antibody. Substitutions may be made to improve antibody properties, for example stability or affinity. Conservative substitutions will produce molecules having functional and chemical characteristics similar to those molecules into which such modifications are made. Exemplary amino acid substitutions are shown in Table 1, below.

TABLE 1
Exemplary Conservative Substitutions

| Original Residue | Exemplary Conservative Substitutions |
|---|---|
| Ala | Val, Leu, Ile |
| Arg | Lys, Gln, Asn |
| Asn | Gln |
| Asp | Glu |
| Cys | Ser, Ala |
| Gln | Asn |
| Gly | Pro, Ala |
| His | Asn, Gln, Lys, Arg |
| Ile | Leu, Val, Met, Ala, Phe |
| Leu | Ile, Val, Met, Ala, Phe |
| Lys | Arg, Gln, Asn |
| Met | Leu, Phe, Ile |
| Phe | Leu, Val, Ile, Ala, Tyr |
| Pro | Ala |
| Ser | Thr, Ala, Cys |
| Thr | Ser |
| Trp | Tyr, Phe |
| Tyr | Trp, Phe, Thr, Ser |
| Val | Ile, Met, Leu, Phe, Ala |

In some embodiments, variants of the proteins and peptides provided herein are provided. In some embodiments, a variant comprises a substitution, deletions, or insertion. In some embodiments, the variant comprises 1, 2, 3, 4, 5, 6, 7, 8, 9, or 10 (e.g., 1-10) substitutions. As described herein, the substitutions can be conservative substitutions. In some embodiments, the substitution is non-conservative. In some embodiments, the variant comprises 1, 2, 3, 4, 5, 6, 7, 8, 9, or 10 (e.g., 1-10) deletions. In some embodiments, the variant comprises 1, 2, 3, 4, 5, 6, 7, 8, 9, or 10 (e.g., 1-10) insertions. In some embodiments, the substitutions, deletions, or insertions are present in the CDRs provided for herein. In some embodiments, the substitutions, deletions, or insertions are not present in the CDRs provided for herein. In some embodiments, the variant antibodies have CDRs that are constant or unchanged as compared to the reference sequence. Put another way, the light chain or heavy chain can have changes as compared to the reference sequence in the framework regions of the light and heavy chains, but no changes in the CDRs.

As described herein the production of antibodies with a known sequence is routine and can be done by any method. Accordingly, in some embodiments, a nucleic acid encoding an antibody or fragment thereof is provided. In some embodiments, the nucleic acid encodes a sequence provided for herein. The antibodies can also be used in injectable pharmaceutical compositions. As also described herein, the antibodies can be isolated antibodies or engineered antibodies.

The nucleic acid sequence encoding an antibody described herein can be genomic DNA or cDNA, or RNA (e.g., mRNA) which encodes at least one of the variable regions described herein. A convenient alternative to the use of chromosomal gene fragments as the source of DNA encoding the V region antigen-binding segment is the use of cDNA for the construction of chimeric immunoglobulin genes, e.g., as reported by Liu et al. (Proc. Natl. Acad. Sci., USA 84:3439 (1987) and J. Immunology 139:3521 (1987), which references are hereby entirely incorporated herein by reference. The use of cDNA requires that gene expression elements appropriate for the host cell be combined with the gene in order to achieve synthesis of the desired protein. The use of cDNA sequences is advantageous over genomic sequences (which contain introns), in that cDNA sequences can be expressed in bacteria or other hosts which lack appropriate RNA splicing systems.

For example, a cDNA encoding a V region antigen-binding segment able to detect, bind, to or neutralize an IGF-1R antigen can be provided using known methods based on the use of the amino acid sequences provided herein. Because the genetic code is degenerate, more than one codon can be used to encode a particular amino acid (Watson, et al., infra). Using the genetic code, one or more different oligonucleotides can be identified, each of which would be capable of encoding the amino acid. The probability that a particular oligonucleotide will, in fact, constitute the actual XXX-encoding sequence can be estimated by considering abnormal base pairing relationships and the frequency with which a particular codon is actually used (to encode a particular amino acid) in eukaryotic or prokaryotic cells expressing an antibody or fragment. Such “codon usage rules” are disclosed by Lathe, et al., J. Molec. Biol. 183:1 12 (1985). Using the “codon usage rules” of Lathe, a single oligonucleotide, or a set of oligonucleotides, that contains a theoretical “most probable” nucleotide sequence capable of encoding an antibody variable or constant region sequences is identified.

As used herein and unless otherwise indicated, the term “about” is intended to mean ±5% of the value it modifies. Thus, about 100 means 95 to 105.

The term “purified” with referenced to an antibody refers to an antibody that is substantially free of other material that associates with the molecule in its natural environment. For instance, a purified protein is substantially free of the cellular material or other proteins from the cell or tissue from which it is derived. The term refers to preparations where the isolated protein is sufficiently pure to be analyzed, or at least 70% to 80% (w/w) pure, at least 80%-90% (w/w) pure, 90-95% pure; and at least 95%, 96%, 97%, 98%, 99%, or 100% (w/w) pure. In some embodiments, the antibody is purified.

As used herein, the term “stable”, or a grammatical equivalent thereof, refers to the ability of the antibody to maintain all or the majority of its intended biological activity and/or physiochemical integrity over time. Typically, an antibody described herein has been formulated such that the formulation is capable of stabilizing, or alternatively slowing or preventing the degradation, of the antibody formulated therewith. In the context of a formulation, a stable formulation is one in which the antibody therein retains its substantial physical and/or chemical integrity upon storage and during processes, such, as but not limited, storage at a specific temperature, freeze/thaw, thermal stress (e.g., at about 40° C. or higher), mechanical mixing, or lyophilization. The “stable” formulation comprising the anti-IGF-1R antibody retains some or all of the biological activity against the protein IGF-1R and for the treatment of TAO.

In some embodiments, antibody stability in a formulation may be measured by formation of high molecular weight (HMW) aggregates, loss of activity, generation of peptide fragments and shift of charge profiles. In some embodiments, the shift in charge profiles may be measured through iCIEF the relative percentages of main peak, acid peak and/or basic peak. For example, a stable formulation can be one in which the change in measured acidic peak increases by less than 40% (e.g., less than 38%, 36%, 34%, 32%, 30%, 28%, 26%, 24%, 22% or less than 20%) relative to an initial value following storage at 40° C. for four weeks as measured by iCIEF and quantified using chromatographic software. In some embodiments, a stable formulation is one in which the main peak percentage decreases by less than 35% (e.g., less than 34%, less than 33%, less than 32%, less than 31%, less than 30%, less than 29%, less than 28%, less than 27%, less than 26%, less than 25%, than less than 24%, less than 23%, less than 22%, less than 21% or less than 20%) relative to an initial value following storage at 40° C. for four weeks as measured by iCIEF and quantified using chromatographic software. In some embodiments, the stable formulation basic peak, measured by iCIEF, decreases in area percent by less than 6.5% (e.g., less than 6%, less than 5%, less than 4%, less than 3%, less than 2%, or less than 1%) after 4 weeks at a temperature of 40° C., relative to the initial value.

In some embodiments, a stable formulation is characterized by the amount of high molecular weight (HMW) aggregates. In some embodiments, the amount of high molecular weight species (HMWS) in the stable formulation is less 10% (e.g., less than 9%, less than 8%, less than 7%, less than 6%, less than 5%, less than 4% less than 3%, less than 2% or less than 1%) as measured by SEC. In some embodiments, the amount of high molecular weight species (HMWS) in the stable formulation is less than 5% as measured by SEC. In some embodiments, the stability based on the HMW or HMWS is based on the composition that is stored at 40 C for a period of time, such as 4 weeks.

Any numerical values used in this application are meant to cover any variations within the standard deviation or normal fluctuations appreciated by one of ordinary skill in the relevant art.

### Anti-IGF-IR Antibodies

Non-limiting examples of anti-IGF-1R antibodies are provided herein. In some embodiments, the antibody is a recombinant antibody that binds to an IGF-1R protein. In some embodiments, the IGF-1R protein is a human IGF-1R protein. In some embodiments, the IGF-1R protein that is recognized by the antibodies is in its native conformation (non-denatured) conformation. In some embodiments, the antibody does not specifically bind to a denatured IGF-1R protein. As used herein, the term “recombinant antibody” refers to an antibody that is not naturally occurring. In some embodiments, the term “recombinant antibody” refers to an antibody that is not isolated from a human subject.

In some embodiments, the antibody comprises one or more peptides having the following sequences, or a variant thereof, in Table 2, below.

TABLE 2
IGF-1R Light Chain and Heavy Chain Sequences

| AB ID NO. | LC Sequence | HC Sequence |
|---|---|---|
| VRDN-03100 | EIVLTQSPATLSLSPGERATLSCR | QVELVESGGGVVQPGRSQRLSCAASGFTFSSY |
| | ASQSVSSYLAWYQQKPGQAPRLL | GMHWVRQAPGKGLEWVAIIWFDGSSTYYADS |
| | IYDASKRATGIPARFSGSGSGTDF | VRGRFTISRDNSKNTLYLQMNSLRAEDTAVYF |
| | TLTISSLEPEDFAVYYCQQRSKWP | CARELGRRYFDLWGRGTLVSVSSASTKGPSVF |
| | PWTFGQGTKVESKRTVAAPSVFI | PLAPSSKSTSGGTAALGCLVKDYFPEPVTVSW |
| | FPPSDEQLKSGTASVVCLLNNFYP | NSGALTSGVHTFPAVLQSSGLYSLSSVVTVPSS |
| | REAKVQWKVDNALQSGNSQESV | SLGTQTYICNVNHKPSNTKVDKKVEPKSCDKT |
| | TEQDSKDSTYSLSSTLTLSKADYE | HTCPPCPAPELLGGPSVFLFPPKPKDTLMISRTP |
| | KHKVYACEVTHQGLSSPVTKSFN | EVTCVVVDVSHEDPEVKFNWYVDGVEVHNA |
| | RGEC (SEQ ID NO: 1) | KTKPREEQYNSTYRVVSVLTVLHQDWLNGKE |
| | | YKCKVSNKALPAPIEKTISKAKGQPREPQVYTL |
| | | PPSRDELTKNQVSLTCLVKGFYPSDIAVEWESN |
| | | GQPENNYKTTPPVLDSDGSFFLYSKLTVDKSR |
| | | WQQGNVFSCSVMHEALHNHYTQKSLSLSPGK |
| | | (SEQ ID NO: 2) |
| VRDN-02100 | DIVMTQSPLSLPVTPGEPASISCRS | QVQLQESGPGLVKPSETLSLTCTVSGYSITGGY |
| | SQSIVHSNGNTYLQWYLQKPGQS | LWNWIRQPPGKGLEWIGYISYDGTNNYKPSLK |
| | PQLLIYKVSNRLYGVPDRFSGSGS | DRVTISRDTSKNQFSLKLSSVTAADTAVYYCA |
| | GTDFTLKISRVEAEDVGVYYCFQ | RYGRVFFDYWGQGTLVTVSSASTKGPSVFPLA |
| | GSHVPWTFGQGTKVEIKRTVAAP | PSSKSTSGGTAALGCLVKDYFPEPVTVSWNSG |
| | SVFIFPPSDEQLKSGTASVVCLLN | ALTSGVHTFPAVLQSSGLYSLSSVVTVPSSSLG |
| | NFYPREAKVQWKVDNALQSGNS | TQTYICNVNHKPSNTKVDKRVEPKSCDKTHTC |
| | QESVTEQDSKDSTYSLSSTLTLSK | PPCPAPELLGGPSVFLFPPKPKDTLMISRTPEVT |
| | ADYEKHKVYACEVTHQGLSSPV | CVVVDVSHEDPEVKFNWYVDGVEVHNAKTK |
| | TKSFNRGEC (SEQ ID NO: 3)f | PREEQYNSTYRVVSVLTVLHQDWLNGKEYKC |
| | | KVSNKALPAPIEKTISKAKGQPREPQVYTLPPS |
| | | REEMTKNQVSLTCLVKGFYPSDIAVEWESNGQ |
| | | PENNYKTTPPVLDSDGSFFLYSKLTVDKSRWQ |
| | | QGNVFSCSVMHEALHNHYTQKSLSLSPGK |
| | | (SEQ ID NO: 4) |
| VRDN-02200 | SSELTQDPAVSVALGQTVRITCQ | EVQLVQSGAEVKKPGSSVKVSCKASGGTFSSY |
| | GDSLRSYYATWYQQKPGQAPILV | AISWVRQAPGQGLEWMGGIIPIFGTANYAQKF |
| | IYGENKRPSGIPDRFSGSSSGNTAS | QGRVTITADKSTSTAYMELSSLRSEDTAVYYC |
| | LTITGAQAEDEADYYCKSRDGSG | ARAPLRFLEWSTQDHYYYYYMDVWGKGTTV |
| | QHLVFGGGTKLTVLGQPKAAPSV | TVSSASTKGPSVFPLAPSSKSTSGGTAALGCLV |
| | TLFPPSSEELQANKATLVCLISDF | KDYFPEPVTVSWNSGALTSGVHTFPAVLQSSG |
| | YPGAVTVAWKADSSPVKAGVET | LYSLSSVVTVPSSSLGTQTYICNVNHKPSNTKV |
| | TTPSKQSNNKYAASSYLSLTPEQ | DKKVEPKSCDKTHTCPPCPAPELLGGPSVFLFP |
| | WKSHRSYSCQVTHEGSTVEKTV | PKPKDTLMISRTPEVTCVVVDVSHEDPEVKFN |
| | APAECS (SEQ ID NO: 5) | WYVDGVEVHNAKTKPREEQYNSTYRVVSVLT |
| | | VLHQDWLNGKEYKCKVSNKALPAPIEKTISKA |
| | | KGQPREPQVYTLPPSREEMTKNQVSLTCLVKG |
| | | FYPSDIAVEWESNGQPENNYKTTPPVLDSDGSF |
| | | FLYSKLTVDKSRWQQGNVFSCSVMHEALHNH |
| | | YTQKSLSLSPGK (SEQ ID NO: 6) |
| VRDN-02300 | DIQMTQFPSSLSASVGDRVTITCR | EVQLLESGGGLVQPGGSLRLSCTASGFTFSSYA |
| | ASQGIRNDLGWYQQKPGKAPKR | MNWVRQAPGKGLEWVSAISGSGGTTFYADSV |
| | LIYAASRLHRGVPSRFSGSGSGTE | KGRFTISRDNSRTTLYLQMNSLRAEDTAVYYC |
| | FTLTISSLQPEDFATYYCLQHNSY | AKDLGWSDSYYYYYGMDVWGQGTTVTVSSA |
| | PCSFGQGTKLEIKRTVAAPSVFIFP | STKGPSVFPLAPCSRSTSESTAALGCLVKDYFP |
| | PSDEQLKSGTASVVCLLNNFYPR | EPVTVSWNSGALTSGVHTFPAVLQSSGLYSLSS |
| | EAKVQWKVDNALQSGNSQESVT | VVTVPSSNFGTQTYTCNVDHKPSNTKVDKTVE |
| | EQDSKDSTYSLSSTLTLSKADYEK | RKCCVECPPCPAPPVAGPSVFLFPPKPKDTLMI |
| | HKVYACEVTHQGLSSPVTKSFNR | SRTPEVTCVVVDVSHEDPEVQFNWYVDGVEV |
| | GEC (SEQ ID NO: 7) | HNAKTKPREEQFNSTFRVVSVLTVVHQDWLN |
| | | GKEYKCKVSNKGLPAPIEKTISKTKGQPREPQV |
| | | YTLPPSREEMTKNQVSLTCLVKGFYPSDIAVE |
| | | WESNGQPENNYKTTPPMLDSDGSFFLYSKLTV |
| | | DKSRWQQGNVFSCSVMHEALHNHYTQKSLSL |
| | | SPG (SEQ ID NO: 8) |
| VRDN-02400 | DVVMTQSPLSLPVTPGEPASISCR | QVQLQESGPGLVKPSGTLSLTCAVSGGSISSSN |
| | SSQSLLHSNGYNYLDWYLQKPG | WWSWVRQPPGKGLEWIGEIYHSGSTNYNPSL |
| | QSPQLLIYLGSNRASGVPDRESGS | KSRVTISVDKSKNQFSLKLSSVTAADTAVYYC |
| | GSGTDFTLKISRVEAEDVGVYYC | ARWTGRTDAFDIWGQGTMVTVSSASTKGPSV |
| | MQGTHWPLTFGQGTKVEIKRTV | FPLAPSSKSTSGGTAALGCLVKDYFPEPVTVSW |
| | AAPSVFIFPPSDEQLKSGTASVVC | NSGALTSGVHTFPAVLQSSGLYSLSSVVTVPSS |
| | LLNNFYPREAKVQWKVDNALQS | SLGTQTYICNVNHKPSNTKVDKKVEPKSCDKT |
| | GNSQESVTEQDSKDSTYSLSSTLT | HTCPPCPAPELLGGPSVFLFPPKPKDTLMISRTP |
| | LSKADYEKHKVYACEVTHQGLS | EVTCVVVDVSHEDPEVKFNWYVDGVEVHNA |
| | SPVTKSFNRGEC (SEQ ID NO: 9) | KTKPREEQYNSTYRVVSVLTVLHQDWLNGKE |
| | | YKCKVSNKALPAPIEKTISKAKGQPREPQVYTL |
| | | PPSRDELTKNQVSLTCLVKGFYPSDIAVEWESN |
| | | GQPENNYKTTPPVLDSDGSFFLYSKLTVDKSR |
| | | WQQGNVFSCSVMHEALHNHYTQKSLSLSPGK |
| | | (SEQ ID NO: 10) |
| VRDN-02500 | EIVLTQSPGTLSVSPGERATLSCR | EVQLVQSGGGLVKPGGSLRLSCAASGFTFSSFA |
| | ASQSIGSSLHWYQQKPGQAPRLLI | MHWVRQAPGKGLEWISVIDTRGATYYADSVK |
| | KYASQSLSGIPDRFSGSGSGTDFT | GRFTISRDNAKNSLYLQMNSLRAEDTAVYYCA |
| | LTISRLEPEDFAVYYCHQSSRLPH | RLGNFYYGMDVWGQGTTVTVSSASTKGPSVF |
| | TFGQGTKVEIKRTVAAPSVFIFPPS | PLAPSSKSTSGGTAALGCLVKDYFPEPVTVSW |
| | DEQLKSGTASVVCLLNNFYPREA | NSGALTSGVHTFPAVLQSSGLYSLSSVVTVPSS |
| | KVQWKVDNALQSGNSQESVTEQ | SLGTQTYICNVNHKPSNTKVDKKVEPKSCDKT |
| | DSKDSTYSLSSTLTLSKADYEKH | HTCPPCPAPELLGGPSVFLFPPKPKDTLMISRTP |
| | KVYACEVTHQGLSSPVTKSFNRG | EVTCVVVDVSHEDPEVKFNWYVDGVEVHNA |
| | EC (SEQ ID NO: 11) | KTKPREEQYNSTYRVVSVLTVLHQDWLNGKE |
| | | YKCKVSNKALPAPIEKTISKAKGQPREPQVYTL |
| | | PPSRDELTKNQVSLTCLVKGFYPSDIAVEWESN |
| | | GQPENNYKTTPPVLDSDGSFFLYSKLTVDKSR |
| | | WQQGNVFSCSVMHEALHNHYTQKSLSLSPGK |
| | | (SEQ ID NO: 12) |
| VRDN-02700 | DIVMTQSPLSLPVTPGEPASISCRS | QVQLQESGPGLVKPSETLSLTCTVSGYSITGGY |
| | SQSIVHSNGNTYLQWYLQKPGQS | LWNWIRQPPGKGLEWIGYISYDGTNNYKPSLK |
| | PQLLIYKVSNRLYGVPDRFSGSGS | DRVTISRDTSKNQFSLKLSSVTAADTAVYYCA |
| | GTDFTLKISRVEAEDVGVYYCFQ | RYGRVFFDYWGQGTLVTVSSASTKGPSVFPLA |
| | GSHVPWTFGQGTKVEIKRTVAAP | PSSKSTSGGTAALGCLVKDYFPEPVTVSWNSG |
| | SVFIFPPSDEQLKSGTASVVCLLN | ALTSGVHTFPAVLQSSGLYSLSSVVTVPSSSLG |
| | NFYPREAKVQWKVDNALQSGNS | TQTYICNVNHKPSNTKVDKRVEPKSCDKTHTC |
| | QESVTEQDSKDSTYSLSSTLTLSK | PPCPAPELLGGPSVFLFPPKPKDTLYITREPEVT |
| | ADYEKHKVYACEVTHQGLSSPV | CVVVDVSHEDPEVKFNWYVDGVEVHNAKTK |
| | TKSFNRGEC (SEQ ID NO: 3) | PREEQYNSTYRVVSVLTVLHQDWLNGKEYKC |
| | | KVSNKALPAPIEKTISKAKGQPREPQVYTLPPS |
| | | REEMTKNQVSLTCLVKGFYPSDIAVEWESNGQ |
| | | PENNYKTTPPVLDSDGSFFLYSKLTVDKSRWQ |
| | | QGNVFSCSVMHEALHNHYTQKSLSLSPG (SEQ |
| | | ID NO: 83) |
| VRDN-01100 | DVVMTQTPLSLPVSLGDPASISCR | QVQLVQSGAEVVKPGASVKLSCKASGYTFTSY |
| | SSQSIVHSNVNTYLEWYLQKPGQ | WMHWVKQRPGQGLEWIGEINPSNGRTNYNQK |
| | SPRLLIYKVSNRFSGVPDRFSGSG | FQGKATLTVDKSSSTAYMQLSSLTSEDSAVYY |
| | AGTDFTLRISRVEAEDLGIYYCFQ | FARGRPDYYGSSKWYFDVWGQGTTVTVSSAS |
| | GSHVPPTFGGGTKLEIKRTVAAPS | TKGPSVFPLAPSSKSTSGGTAALGCLVKDYFPE |
| | VFIFPPSDEQLKSGTASVVCLLNN | PVTVSWNSGALTSGVHTFPAVLQSSGLYSLSS |
| | FYPREAKVQWKVDNALQSGNSQ | VVTVPSSSLGTQTYICNVNHKPSNTKVDKKVE |
| | ESVTEQDSKDSTYSLSSTLTLSKA | PKSCDKTHTCPPCPAPELLGGPSVFLFPPKPKD |
| | DYEKHKVYACEVTHQGLSSPVT | TLMISRTPEVTCVVVDVSHEDPEVKFNWYVDG |
| | KSFNRGEC (SEQ ID NO: 93). | VEVHNAKTKPREEQYNSTYRVVSVLTVLHQD |
| | | WLNGKEYKCKVSNKALPAPIEKTISKAKGQPR |
| | | EPQVYTLPPSRDELTKNQVSLTCLVKGFYPSDI |
| | | AVEWESNGQPENNYKTTPPVLDSDGSFFLYSK |
| | | LTVDKSRWQQGNVFSCSVMHEALHNHYTQKS |
| | | LSLSPGK (SEQ ID NO: 92) |

In some embodiments, the antibody comprises one or more peptides having the following sequences, or a variant thereof, in Table 3, below.

TABLE 3
IGF-1R antibody Variable Light Chain and Variable Heavy Chain Sequences

| AB ID NO. | VL Sequence | VH Sequence |
|---|---|---|
| VRDN-01100 | DVVMTQTPLSLPVSLGDPASISCRSSQSI | QVQLVQSGAEVVKPGASVKLSCKASG |
| | VHSNVNTYLEWYLQKPGQSPRLLIYKVS | YTFTSYWMHWVKQRPGQGLEWIGEIN |
| | NRFSGVPDRFSGSGAGTDFTLRISRVEAE | PSNGRTNYNQKFQGKATLTVDKSSSTA |
| | DLGIYYCFQGSHVPPTFGGGTKLEIKR | YMQLSSLTSEDSAVYYFARGRPDYYGS |
| | (SEQ ID NO: 13) | SKWYFDVWGQGTTVTVSS (SEQ ID |
| | | NO: 14) |
| VRDN-02600 | DIQMTQSPLSLSASVGDRVTITCQASRDI | EVQLLESGGGLVQPGGSLRLSCAASGFT |
| | RNYLNWYQQKPGKAPKLLIYDASSLQT | FSIYRMQWVRQAPGKGLEWVSGISPSG |
| | GVPSRFGGSGSGTDFSFTIGSLQPEDIAT | GTTWYADSVKGRFTISRDNSKNTLYLQ |
| | YYCQQFDSLPHTFGQGTKLEIK (SEQ ID | MNSLRAEDTAVYYCARWSGGSGYAFD |
| | NO: 15) | IWGQGTMVTVSS (SEQ ID NO: 16) |
| VRDN-02301 | DIQMTQFPSSLSASVGDRVTITCRASQGI | EVQLLESGGGLVQPGGSLRLSCTASGFT |
| | RNDLGWYQQKPGKAPKRLIYAASRLHR | FSSYAMNWVRQAPGKGLEWVSAISGS |
| | GVPSRFSGSGSGTEFTLTISSLQPEDFATY | GGTTFYADSVKGRFTISRDNSRTTLYLQ |
| | YCLQHNSYPSSFGQGTKLEIK (SEQ ID | MNSLRAEDTAVYYCAKDLGWSDSYYY |
| | NO: 79) | YYGMDVWGQGTTVTVSS (SEQ ID NO: |
| | | 80) |
| VRDN-01101 | DVVMTQTPLSLPVSLGDPASISCRSSQSI | QVQLVQSGAEVVKPGASVKLSCKASG |
| | VHSNVNTYLEWYLQKPGQSPKLLIYKVS | YTFTSYWMHWVKQRPGQGLEWIGEIN |
| | NRFSGVPDRFSGSGAGTDFTLRISRVEAE | PSNGRTNYNQKFQGKATLTVDKSSSTA |
| | DLGIYYCFQGSHVPPTFGGGTKLEIKR | YMQLSSLTSEDSAVYYFARGRPDYYGS |
| | (SEQ ID NO: 86) | SKWYFDVWGQGTTVTVSS (SEQ ID |
| | | NO: 14) |
| VRDN-2700 | DIVMTQSPLSLPVTPGEPASISCRSSQSIV | QVQLQESGPGLVKPSETLSLTCTVSGYS |
| | HSNGNTYLQWYLQKPGQSPQLLIYKVS | ITGGYLWNWIRQPPGKGLEWIGYISYD |
| | NRLYGVPDRFSGSGSGTDFTLKISRVEAE | GTNNYKPSLKDRVTISRDTSKNQFSLKL |
| | DVGVYYCFQGSHVPWTFGQGTKVEIKR | SSVTAADTAVYYCARYGRVFFDYWGQ |
| | (SEQ ID NO: 98) | GTLVTVSS (SEQ ID NO: 99) |
| VRDN- | DVVMTQTPLSLPVSLGDPASISCRSSQSI | QVQLVQSGAEVVKPGASVKLSSKASGY |
| 01100A or | VHSNVNTYLEWYLQKPGQSPKLLIYKVS | TFTSYWMHWVKQRPGQGLEWIGEINPS |
| 01110B | NRFSGVPDRFSGSGAGTDFTLRISRVEAE | NGRTNYNQKFQGKATLTVDKSSSTAY |
| | DLGIYYCFQGSHVPPTFGGGTKLEIKR | MQLSSLTSEDSAVYYFARGRPDYYGSS |
| | (SEQ ID NO: 86) | KWYFDVWGQGTTVTVSS (SEQ ID NO: |
| | | 91) |

The column that is indicated as the antibody sequence comprises the VH and VL chains of the antibody. In instances where the VH chain is illustrated with a Fc sequence, the Fc sequence can be modified or substituted for a different Fc region as provided for herein. However, in some embodiments, the antibody can comprise the VH and VL sequence as provided for in the tables provided for herein.

In some embodiments, the variable light chain as set forth in SEQ ID NO: 13 does not have the C-terminal arginine residue. This is illustrated for example, in the following sequence:

(SEQ ID NO: 97)
DVVMTQTPLSLPVSLGDPASISCRSSQSIVHSNVNTYLEWYLQKPGQSP
RLLIYKVSNRFSGVPDRFSGSGAGTDFTLRISRVEAEDLGIYYCFQGSH
VPPTFGGGTKLEIK.

Thus, in some embodiments, where the variable light chain comprises the sequence of SEQ ID NO: 13, it can be substituted with a sequence of SEQ ID NO: 97.

In some embodiments, the heavy chain variable region as set forth in SEQ ID NO: 14 can comprises a C22S substitution. This is illustrated in the following sequence:

(SEQ ID NO: 96)
QVQLVQSGAEVVKPGASVKLSSKASGYTFTSYWMHWVKQRPGQGLEWIG
EINPSNGRTNYNQKFQGKATLTVDKSSSTAYMQLSSLTSEDSAVYYFAR
GRPDYYGSSKWYFDVWGQGTTVTVSS.

Accordingly, in some embodiments, the antibody comprises a VH sequence of SEQ ID NO: 96 and a VL sequence of SEQ ID NO: 13 or SEQ ID NO: 97.

In some embodiments, the antibody comprises a VH of SEQ ID NO: 14 and a VL sequence of SEQ ID NO: 97.

In some embodiments, the antibody comprises a VL of SEQ ID NO: 98 and a VH of SEQ ID NO: 99. In some embodiments, the antibody comprises a VL of SEQ ID NO: 98 and a VH of SEQ ID NO: 99 with a Fc region comprising the M252Y, S254T, and T256E mutations. In some embodiments, the antibody comprises a VL of SEQ ID NO: 98 and a VH of SEQ ID NO: 99 with a Fc region comprising the M428L and N434S mutations.

As provided for herein, the heavy chain can be linked to a Fc region, including those with mutations that can affect the half-life of the antibody. Non-limiting mutations in the Fc region are provided for herein.

In the tables provided for herein, the LC and HC may be illustrated with the VH and VL domains with or without constant regions. The constant regions can be replaced as provided for herein. The VH and VL regions can be used to form an antibody as provided for herein.

In some embodiments, an antibody, or antigen binding fragment thereof is provided, wherein the antibody or antibody fragment comprises a CDR selected from Tables 4-7, below.

TABLE 4
IGF-1R Antibody Kabat CDRs

| Ab ID No | LCDR1 | LCDR2 | LCDR3 | HCDR1 | HCDR2 | HCDR3 |
|---|---|---|---|---|---|---|
| VRDN- | RASQSV | DASKRAT | QQRSKWPP | SYGMH | IIWFDGSSTY | ELGRRYFD |
| 03100 | SSYLA | (SEQ ID | WT (SEQ ID | (SEQ ID | YADSVRG | L (SEQ ID |
| | (SEQ ID | NO: 18) | NO: 19) | NO: 20) | (SEQ ID NO: | NO: 22) |
| | NO: 17) | | | | 21) | |
| VRDN- | RSSQSIV | KVSNRLY | FQGSHVPW | GGYLWN | YISYDGTNNY | YGRVFFDY |
| 02100/2700 | HSNGNT | (SEQ ID | T (SEQ ID | (SEQ ID | KPSLKD (SEQ | (SEQ ID |
| | YLQWY | NO: 24) | NO: 25) | NO: 26) | ID NO: 27) | NO: 28) |
| | LQ (SEQ | | | | | |
| | ID NO: 23) | | | | | |
| VRDN- | QGDSLR | GENKRPS | KSRDGSGQ | SYAIS | GIIPIFGTANY | APLRFLEW |
| 02200 | SYYAT | (SEQ ID | HLV (SEQ | (SEQ ID | AQKFQG | STQDHYYY |
| | (SEQ ID | NO: 30) | ID NO: 31) | NO: 32) | (SEQ ID NO: | YYMDV |
| | NO: 29) | | | | 33) | (SEQ ID |
| | | | | | | NO: 34) |
| VRDN- | RASQGI | AASRLHR | LQHNSYPC | SYAMN | AISGSGGTTF | DLGWSDSY |
| 02300 | RNDLG | (SEQ ID | S (SEQ ID | (SEQ ID | YADSVKG | YYYYGMD |
| | (SEQ ID | NO: 36) | NO: 37) | NO: 38) | (SEQ ID NO: | V (SEQ ID |
| | NO: 35) | | | | 39) | NO: 40) |
| VRDN- | RSSQSL | LGSNRA | MQGTHWP | SSSNWWS | EIYHSGSTNY | WTGRTDAF |
| 02400 | LHSNGY | (SEQ ID | LT (SEQ ID | (SEQ ID | NPSLKS (SEQ | DI (SEQ ID |
| | NYLD | NO: 42) | NO: 43) | NO: 44) | ID NO: 45) | NO: 46) |
| | (SEQ ID | | | | | |
| | NO: 41) | | | | | |
| VRDN- | RASQSI | YASQSLS | HQSSRLPH | SFAMH | VIDTRGATYY | LGNFYYGM |
| 02500 | GSSLH | (SEQ ID | T (SEQ ID | (SEQ ID | ADSVKG | DV (SEQ ID |
| | (SEQ ID | NO: 48) | NO: 49) | NO: 50) | (SEQ ID NO: | NO: 52) |
| | NO: 47) | | | | 51) | |
| VRDN- | RSSQSIV | KVSNRFS | FQGSHVPP | SYWMH | GEINPSNGRT | GRPDYYGS |
| 1100/1100A/ | HSNVNT | (SEQ ID | T (SEQ ID | (SEQ ID | NYNQKFQG | SKWYFDV |
| 1100B | YLE | NO: 54) | NO: 55) | NO: 56) | (SEQ ID NO: | (SEQ ID |
| | (SEQ ID | | | | 57) | NO: 58) |
| | NO: 53) | | | | | |
| VRDN-2600 | QASRDI | DASSLQT | QQFDSLPH | IYRMQ | GISPSGGTTW | WSGGSGYA |
| | RNYLN | (SEQ ID | T (SEQ ID | (SEQ ID | YADSVK | FDI (SEQ ID |
| | (SEQ ID | NO: 60) | NO: 61) | NO: 62) | (SEQ ID NO: | NO: 64) |
| | NO: 59) | | | | 63) | |
| VRDN-2301 | RASQGI | AASRLHR | LQHNSYPS | SYAMN | AISGSGGTTF | DLGWSDSY |
| | RNDLG | (SEQ ID | S (SEQ ID | (SEQ ID | YADSVKG | YYYYGMD |
| | (SEQ ID | NO: 36) | NO: 81) | NO: 38) | (SEQ ID NO: | V (SEQ ID |
| | NO: 35) | | | | 39) | NO: 40) |

TABLE 5
IGF-1R Antibody IMGT CDRs

| Ab ID No | LCDR1 | LCDR2 | LCDR3 | HCDR1 | HCDR2 | HCDR3 |
|---|---|---|---|---|---|---|
| VRDN- | QSIVHSN | KVS (SEQ | FQGSHVPP | GYTFTSY | INPSNGRT | ARGRPDYY |
| 1100/1100A/ | VNTY | ID NO: | T (SEQ ID | W (SEQ ID | (SEQ ID NO: | GSSKWYFD |
| 1100B | (SEQ ID | 103) | NO: 55) | NO: 104) | 105) | V (SEQ ID |
| | NO: 102) | | | | | NO: 106) |
| VRDN- | QSIVHSN | KVS (SEQ | FQGSHVPW | GYSITGGY | ISYDGTN | ARYGRVFF |
| 02100/2700 | GNTY | ID NO: | T (SEQ ID | L (SEQ ID | (SEQ ID NO: | DY (SEQ ID |
| | (SEQ ID | 103) | NO: 25) | NO: 108) | 109) | NO: 110) |
| | 107) | | | | | |

TABLE 6
IGF-1R Antibody Chothia CDRs

| Ab ID No | LCDR1 | LCDR2 | LCDR3 | HCDR1 | HCDR2 | HCDR3 |
|---|---|---|---|---|---|---|
| VRDN- | RSSQSIVH | KVSNRFS | FQGSHVPP | GYTFTSY | NPSNGR (SEQ | GRPDYYGS |
| 1100/1100A/ | SNVNTYL | (SEQ ID | T (SEQ ID | (SEQ ID | ID NO: 112) | SKWYFDV |
| 1100B | E (SEQ ID | NO: 54) | NO: 55) | NO: 111) | | (SEQ ID |
| | NO: 53) | | | | | NO: 58) |
| VRDN- | RSSQSIVH | KVSNRLY | FQGSHVPW | GYSITGGY | SYDGT (SEQ | YGRVFFDY |
| 02100/2700 | SNGNTYL | (SEQ ID | T (SEQ ID | (SEQ ID | ID NO: 112) | (SEQ ID |
| | QWYLQ | NO: 24) | NO: 25) | NO: 108) | | NO: 28) |
| | (SEQ ID | | | | | |
| | NO: 23) | | | | | |

TABLE 7
IGF-1R Antibody North CDRs

| Ab ID No | LCDR1 | LCDR2 | LCDR3 | HCDR1 | HCDR2 | HCDR3 |
|---|---|---|---|---|---|---|
| VRDN- | RSSQSIVH | YKVSNRF | FQGSHVPP | KASGYTF | EINPSNGRTN | ARGRPDYY |
| 1100/1100A/ | SNVNTYL | S (SEQ ID | T (SEQ ID | TSYWMH | (SEQ ID NO: | GSSKWYFD |
| 1100B | E (SEQ ID | NO: 113) | NO: 55) | (SEQ ID | 115) | V (SEQ ID |
| | NO: 53) | | | NO: 114) | | NO: 106) |
| VRDN- | RSSQSIVH | YKVSNRL | FQGSHVPW | TVSGYSIT | YISYDGTNN | ARYGRVFF |
| 02100/2700 | SNGNTYL | Y (SEQ ID | T (SEQ ID | GGYLWN | (SEQ ID NO: | DY (SEQ ID |
| | QWYLQ | NO: 118) | NO: 25) | (SEQ ID | 117) | NO: 110) |
| | (SEQ ID | | | NO: 116) | | |
| | NO: 23) | | | | | |

In some embodiments, an antibody, or antibody binding fragment thereof, comprises a heavy or light chain CDR having a sequence of SEQ ID Nos: 17-64 and 81. In some embodiments, an antibody, or antibody binding fragment thereof, comprises a light chain CDR having a sequence of SEQ ID NO: 17, 18, 19, 23, 24, 25, 29, 30, 31, 35, 36, 37, 41, 42, 43, 47, 48, 49, 53, 54, 55, 59, 60, 61, or 81. In some embodiments, an antibody, or antibody binding fragment thereof, comprises a heavy chain CDR having a sequence of SEQ ID NO: 20, 21, 22, 26, 27, 28, 32, 33, 34, 38, 39, 40, 44, 45, 46, 50, 51, 52, 56, 57, 58, 62, 63, or 64.

In some embodiments, an antibody, or antibody binding fragment thereof, comprises a light chain having a LCDR1, a LCDR2, and a LCDR3, wherein the LCDR1 has a sequence of SEQ ID NO: 17, 23, 29, 35, 41, 47, 53, or 59 the LCDR2 has a sequence of SEQ ID NO: 18, 24, 30, 36, 42, 48, 54, or 60 and the LCDR3 has a sequence of SEQ ID NO: 19, 25, 31, 37, 43, 49, 55, 61, or 81.

In some embodiments, an antibody, or antibody binding fragment thereof, comprises a heavy chain having a HCDR1, a HCDR2, and a HCDR3, wherein the HCDR1 has a sequence of SEQ ID NO: 20, 26, 32, 38, 44, 50, 56, or 62 the HCDR2 has a sequence of SEQ ID NO: 21, 27, 33, 39, 45, 51, 57, or 63 and the HCDR3 has a sequence of SEQ ID NO: 22, 28, 34, 40, 46, 52, 58, or 64.

The different CDR motifs can be combined in any combination including those not depicted in the table above. For example, the following embodiments are provided as non-limiting examples of such combinations.

In some embodiments, an antibody, or antigen binding fragment thereof, comprises: (i) a light chain variable region comprising light chain CDR1, CDR2, and CDR3 sequences, wherein the light chain CDR1 sequence has the amino acid sequence of SEQ ID NO: 17; the light chain CDR2 has the amino acid sequence of SEQ ID NO: 18; and the light chain CDR3 sequence has the amino acid sequence of SEQ ID NO: 19; and (ii) a heavy chain variable region comprising heavy chain CDR1, CDR2, and CDR3 sequences, wherein the heavy chain CDR1 sequence has the amino acid sequence of SEQ ID NO: 20; the heavy chain CDR2 sequence has the amino acid sequence of SEQ ID NO: 21; and the heavy chain CDR3 sequence has the amino acid sequence of SEQ ID NO: 22; or variants of any of the foregoing.

In some embodiments, an antibody, or antigen binding fragment thereof, comprises: (i) a light chain variable region comprising light chain CDR1, CDR2, and CDR3 sequences, wherein the light chain CDR1 sequence has the amino acid sequence of SEQ ID NO: 23; the light chain CDR2 has the amino acid sequence of SEQ ID NO: 24; and the light chain CDR3 sequence has the amino acid sequence of SEQ ID NO: 25; and (ii) a heavy chain variable region comprising heavy chain CDR1, CDR2, and CDR3 sequences, wherein the heavy chain CDR1 sequence has the amino acid sequence of SEQ ID NO: 26; the heavy chain CDR2 sequence has the amino acid sequence of SEQ ID NO: 27; and the heavy chain CDR3 sequence has the amino acid sequence of SEQ ID NO: 28; or variants of any of the foregoing.

In some embodiments, an antibody, or antigen binding fragment thereof, comprises: (i) a light chain variable region comprising light chain CDR1, CDR2, and CDR3 sequences, wherein the light chain CDR1 sequence has the amino acid sequence of SEQ ID NO: 29; the light chain CDR2 has the amino acid sequence of SEQ ID NO: 30; and the light chain CDR3 sequence has the amino acid sequence of SEQ ID NO: 31; and (ii) a heavy chain variable region comprising heavy chain CDR1, CDR2, and CDR3 sequences, wherein the heavy chain CDR1 sequence has the amino acid sequence of SEQ ID NO: 32; the heavy chain CDR2 sequence has the amino acid sequence of SEQ ID NO: 33; and the heavy chain CDR3 sequence has the amino acid sequence of SEQ ID NO: 34; or variants of any of the foregoing.

In some embodiments, an antibody, or antigen binding fragment thereof, comprises: (i) a light chain variable region comprising light chain CDR1, CDR2, and CDR3 sequences, wherein the light chain CDR1 sequence has the amino acid sequence of SEQ ID NO: 35; the light chain CDR2 has the amino acid sequence of SEQ ID NO: 36; and the light chain CDR3 sequence has the amino acid sequence of SEQ ID NO: 37; and (ii) a heavy chain variable region comprising heavy chain CDR1, CDR2, and CDR3 sequences, wherein the heavy chain CDR1 sequence has the amino acid sequence of SEQ ID NO: 38; the heavy chain CDR2 sequence has the amino acid sequence of SEQ ID NO: 39; and the heavy chain CDR3 sequence has the amino acid sequence of SEQ ID NO: 40; or variants of any of the foregoing.

In some embodiments, an antibody, or antigen binding fragment thereof, comprises: (i) a light chain variable region comprising light chain CDR1, CDR2, and CDR3 sequences, wherein the light chain CDR1 sequence has the amino acid sequence of SEQ ID NO: 41; the light chain CDR2 has the amino acid sequence of SEQ ID NO: 42; and the light chain CDR3 sequence has the amino acid sequence of SEQ ID NO: 43; and (ii) a heavy chain variable region comprising heavy chain CDR1, CDR2, and CDR3 sequences, wherein the heavy chain CDR1 sequence has the amino acid sequence of SEQ ID NO: 44; the heavy chain CDR2 sequence has the amino acid sequence of SEQ ID NO: 45; and the heavy chain CDR3 sequence has the amino acid sequence of SEQ ID NO: 46; or variants of any of the foregoing.

In some embodiments, an antibody, or antigen binding fragment thereof, comprises: (i) a light chain variable region comprising light chain CDR1, CDR2, and CDR3 sequences, wherein the light chain CDR1 sequence has the amino acid sequence of SEQ ID NO: 47; the light chain CDR2 has the amino acid sequence of SEQ ID NO: 48; and the light chain CDR3 sequence has the amino acid sequence of SEQ ID NO: 49; and (ii) a heavy chain variable region comprising heavy chain CDR1, CDR2, and CDR3 sequences, wherein the heavy chain CDR1 sequence has the amino acid sequence of SEQ ID NO: 50; the heavy chain CDR2 sequence has the amino acid sequence of SEQ ID NO: 51; and the heavy chain CDR3 sequence has the amino acid sequence of SEQ ID NO: 52; or variants of any of the foregoing.

In some embodiments, an antibody, or antigen binding fragment thereof, comprises: (i) a light chain variable region comprising light chain CDR1, CDR2, and CDR3 sequences, wherein the light chain CDR1 sequence has the amino acid sequence of SEQ ID NO: 53; the light chain CDR2 has the amino acid sequence of SEQ ID NO: 54; and the light chain CDR3 sequence has the amino acid sequence of SEQ ID NO: 55; and (ii) a heavy chain variable region comprising heavy chain CDR1, CDR2, and CDR3 sequences, wherein the heavy chain CDR1 sequence has the amino acid sequence of SEQ ID NO: 56; the heavy chain CDR2 sequence has the amino acid sequence of SEQ ID NO: 57; and the heavy chain CDR3 sequence has the amino acid sequence of SEQ ID NO: 58; or variants of any of the foregoing.

In some embodiments, an antibody, or antigen binding fragment thereof, comprises: (i) a light chain variable region comprising light chain CDR1, CDR2, and CDR3 sequences, wherein the light chain CDR1 sequence has the amino acid sequence of SEQ ID NO: 59; the light chain CDR2 has the amino acid sequence of SEQ ID NO: 60; and the light chain CDR3 sequence has the amino acid sequence of SEQ ID NO: 61; and (ii) a heavy chain variable region comprising heavy chain CDR1, CDR2, and CDR3 sequences, wherein the heavy chain CDR1 sequence has the amino acid sequence of SEQ ID NO: 62; the heavy chain CDR2 sequence has the amino acid sequence of SEQ ID NO: 63; and the heavy chain CDR3 sequence has the amino acid sequence of SEQ ID NO: 64; or variants of any of the foregoing.

In some embodiments, an antibody, or antigen binding fragment thereof, comprises: (i) a light chain variable region comprising light chain CDR1, CDR2, and CDR3 sequences, wherein the light chain CDR1 sequence has the amino acid sequence of SEQ ID NO: 35; the light chain CDR2 has the amino acid sequence of SEQ ID NO: 36; and the light chain CDR3 sequence has the amino acid sequence of SEQ ID NO: 81; and (ii) a heavy chain variable region comprising heavy chain CDR1, CDR2, and CDR3 sequences, wherein the heavy chain CDR1 sequence has the amino acid sequence of SEQ ID NO: 38; the heavy chain CDR2 sequence has the amino acid sequence of SEQ ID NO: 39; and the heavy chain CDR3 sequence has the amino acid sequence of SEQ ID NO: 40; or variants of any of the foregoing.

In some embodiments, an antibody, or antibody binding fragment thereof, comprises a light chain having a LCDR1, a LCDR2, and a LCDR3, wherein the LCDR1 has a sequence of SEQ ID NO: 102, the LCDR2 has a sequence of SEQ ID NO: 103, and the LCDR3 has a sequence of SEQ ID NO: 55. In some embodiments, an antibody, or antibody binding fragment thereof, comprises a heavy chain having a HCDR1, a HCDR2, and a HCDR3, wherein the HCDR1 has a sequence of SEQ ID NO: 104, the HCDR2 has a sequence of SEQ ID NO: 105, and the HCDR3 has a sequence of SEQ ID NO: 106. In some embodiments, an antibody, or antibody binding fragment thereof, comprises: (i) a light chain having a LCDR1, a LCDR2, and a LCDR3, wherein the LCDR1 has a sequence of SEQ ID NO: 102, the LCDR2 has a sequence of SEQ ID NO: 103, and the LCDR3 has a sequence of SEQ ID NO: 55, and (ii) a heavy chain having a HCDR1, a HCDR2, and a HCDR3, wherein the HCDR1 has a sequence of SEQ ID NO: 104, the HCDR2 has a sequence of SEQ ID NO: 105, and the LCDR3 has a sequence of SEQ ID NO: 106.

In some embodiments, an antibody, or antibody binding fragment thereof, comprises a light chain having a LCDR1, a LCDR2, and a LCDR3, wherein the LCDR1 has a sequence of SEQ ID NO: 53, the LCDR2 has a sequence of SEQ ID NO: 54, and the LCDR3 has a sequence of SEQ ID NO: 55. In some embodiments, an antibody, or antibody binding fragment thereof, comprises a heavy chain having a HCDR1, a HCDR2, and a HCDR3, wherein the HCDR1 has a sequence of SEQ ID NO: 111, the HCDR2 has a sequence of SEQ ID NO: 112, and the HCDR3 has a sequence of SEQ ID NO: 58. In some embodiments, an antibody, or antibody binding fragment thereof, comprises: (i) a light chain having a LCDR1, a LCDR2, and a LCDR3, wherein the LCDR1 has a sequence of SEQ ID NO: 53, the LCDR2 has a sequence of SEQ ID NO: 54, and the LCDR3 has a sequence of SEQ ID NO: 55, and (ii) a heavy chain having a HCDR1, a HCDR2, and a HCDR3, wherein the HCDR1 has a sequence of SEQ ID NO: 111, the HCDR2 has a sequence of SEQ ID NO: 112, and the HCDR3 has a sequence of SEQ ID NO: 58.

In some embodiments, an antibody, or antibody binding fragment thereof, comprises a light chain having a LCDR1, a LCDR2, and a LCDR3, wherein the LCDR1 has a sequence of SEQ ID NO: 53, the LCDR2 has a sequence of SEQ ID NO: 113, and the LCDR3 has a sequence of SEQ ID NO: 55. In some embodiments, an antibody, or antibody binding fragment thereof, comprises a heavy chain having a HCDR1, a HCDR2, and a HCDR3, wherein the HCDR1 has a sequence of SEQ ID NO: 114, the HCDR2 has a sequence of SEQ ID NO: 115, and the HCDR3 has a sequence of SEQ ID NO: 106. In some embodiments, an antibody, or antibody binding fragment thereof, comprises: (i) a light chain having a LCDR1, a LCDR2, and a LCDR3, wherein the LCDR1 has a sequence of SEQ ID NO: 53, the LCDR2 has a sequence of SEQ ID NO: 113, and the LCDR3 has a sequence of SEQ ID NO: 55, and (ii) a heavy chain having a HCDR1, a HCDR2, and a HCDR3, wherein the HCDR1 has a sequence of SEQ ID NO: 114, the HCDR2 has a sequence of SEQ ID NO: 115, and the HCDR3 has a sequence of SEQ ID NO: 106.

In some embodiments, an antibody, or antibody binding fragment thereof, comprises a light chain having a LCDR1, a LCDR2, and a LCDR3, wherein the LCDR1 has a sequence of SEQ ID NO: 107, the LCDR2 has a sequence of SEQ ID NO: 103, and the LCDR3 has a sequence of SEQ ID NO: 25. In some embodiments, an antibody, or antibody binding fragment thereof, comprises a heavy chain having a HCDR1, a HCDR2, and a HCDR3, wherein the HCDR1 has a sequence of SEQ ID NO: 108, the HCDR2 has a sequence of SEQ ID NO: 109, and the HCDR3 has a sequence of SEQ ID NO: 110. In some embodiments, an antibody, or antibody binding fragment thereof, comprises: (i) a light chain having a LCDR1, a LCDR2, and a LCDR3, wherein the LCDR1 has a sequence of SEQ ID NO: 107, the LCDR2 has a sequence of SEQ ID NO: 103, and the LCDR3 has a sequence of SEQ ID NO: 25, and (ii) a heavy chain having a HCDR1, a HCDR2, and a HCDR3, wherein the HCDR1 has a sequence of SEQ ID NO: 108, the HCDR2 has a sequence of SEQ ID NO: 109, and the HCDR3 has a sequence of SEQ ID NO: 110.

In some embodiments, an antibody, or antibody binding fragment thereof, comprises a light chain having a LCDR1, a LCDR2, and a LCDR3, wherein the LCDR1 has a sequence of SEQ ID NO: 23, the LCDR2 has a sequence of SEQ ID NO: 24, and the LCDR3 has a sequence of SEQ ID NO: 25. In some embodiments, an antibody, or antibody binding fragment thereof, comprises a heavy chain having a HCDR1, a HCDR2, and a HCDR3, wherein the HCDR1 has a sequence of SEQ ID NO: 108, the HCDR2 has a sequence of SEQ ID NO: 112, and the HCDR3 has a sequence of SEQ ID NO: 28. In some embodiments, an antibody, or antibody binding fragment thereof, comprises: (i) a light chain having a LCDR1, a LCDR2, and a LCDR3, wherein the LCDR1 has a sequence of SEQ ID NO: 23, the LCDR2 has a sequence of SEQ ID NO: 24, and the LCDR3 has a sequence of SEQ ID NO: 25, and (ii) a heavy chain having a HCDR1, a HCDR2, and a HCDR3, wherein the HCDR1 has a sequence of SEQ ID NO: 108, the HCDR2 has a sequence of SEQ ID NO: 112, and the LCDR3 has a sequence of SEQ ID NO: 28.

In some embodiments, an antibody, or antibody binding fragment thereof, comprises a light chain having a LCDR1, a LCDR2, and a LCDR3, wherein the LCDR1 has a sequence of SEQ ID NO: 23, the LCDR2 has a sequence of SEQ ID NO: 118, and the LCDR3 has a sequence of SEQ ID NO: 25. In some embodiments, an antibody, or antibody binding fragment thereof, comprises a heavy chain having a HCDR1, a HCDR2, and a HCDR3, wherein the HCDR1 has a sequence of SEQ ID NO: 116, the HCDR2 has a sequence of SEQ ID NO: 117, and the LCDR3 has a sequence of SEQ ID NO: 110. In some embodiments, an antibody, or antibody binding fragment thereof, comprises: (i) a light chain having a LCDR1, a LCDR2, and a LCDR3, wherein the LCDR1 has a sequence of SEQ ID NO: 25, the LCDR2 has a sequence of SEQ ID NO: 118, and the LCDR3 has a sequence of SEQ ID NO: 25, and (ii) a heavy chain having a HCDR1, a HCDR2, and a HCDR3, wherein the HCDR1 has a sequence of SEQ ID NO: 116, the HCDR2 has a sequence of SEQ ID NO: 117, and the LCDR3 has a sequence of SEQ ID NO: 110.

In some embodiments, the light chain variable region CDR1 is replaced with any of the other light chain CDR1 sequences. In some embodiments, the light chain variable region CDR2 is replaced with any of the other light chain CDR2 sequences. In some embodiments, the light chain variable region CDR3 is replaced with any of the other light chain CDR3 sequences. In some embodiments, the heavy chain variable region CDR1 is replaced with any of the other light chain CDR1 sequences. In some embodiments, the heavy chain variable region CDR2 is replaced with any of the other light chain CDR2 sequences. In some embodiments, the heavy chain variable region CDR3 is replaced with any of the other light chain CDR3 sequences.

In some embodiments, the antibody, or antigen binding fragment thereof, or protein is provided that comprises a peptide having a sequence as set forth in any of SEQ ID Nos: 1, 3, 5, 7, 9, 11, 13, 15, 79, or 86, and 2, 4, 6, 8, 10, 12, 14, 16, 80, or 83.

In some embodiments, the antibody, or antigen binding fragment thereof, comprises a sequence of, or a variant of any of the foregoing.

In some embodiments, the antibody, or antigen binding fragment thereof, comprises a sequence of SEQ ID Nos: 65, or a variant of any of the foregoing. In some embodiments, the antibody, or antigen binding fragment thereof, comprises a sequence of SEQ ID Nos: 66, or a variant of any of the foregoing. In some embodiments, the antibody, or antigen binding fragment thereof, comprises a sequence of SEQ ID Nos: 67, or a variant of any of the foregoing. In some embodiments, the antibody, or antigen binding fragment thereof, comprises a sequence of SEQ ID Nos: 68, or a variant of any of the foregoing. In some embodiments, the antibody, or antigen binding fragment thereof, comprises a sequence of SEQ ID Nos: 69, or a variant of any of the foregoing. In some embodiments, the antibody, or antigen binding fragment thereof, comprises a sequence of SEQ ID Nos: 70, or a variant of any of the foregoing. In some embodiments, the antibody, or antigen binding fragment thereof, comprises a sequence of SEQ ID Nos: 71, or a variant of any of the foregoing. In some embodiments, the antibody, or antigen binding fragment thereof, comprises a sequence of SEQ ID Nos: 72, or a variant of any of the foregoing. In some embodiments, the antibody, or antigen binding fragment thereof, comprises a sequence of SEQ ID Nos: 78, or a variant of any of the foregoing. In some embodiments, the antibody, or antigen binding fragment thereof, comprises a sequence of SEQ ID Nos: 82, or a variant of any of the foregoing. In some embodiments, the antibody, or antigen binding fragment thereof, comprises a sequence of SEQ ID Nos: 85, or a variant of any of the foregoing.

In some embodiment, the V_{L} and/or V_{H} sequences are as provided herein. In some embodiments, the V_{L} sequences are provided as elements of the light chain (LC). In some embodiments, the V_{L} sequences that are provided as elements of the light chain (LC) are underlined in the LC sequence. In some embodiments, the V_{H} sequences that are provided as elements of the heavy chain (LC) are underlined in the HC sequence.

In some embodiments, an antibody, or antigen binding fragment thereof, comprises a V_{L} peptide as set forth in SEQ ID Nos: 1, 3, 5, 7, 9, 11, 13, 15, 79, or 86, or any combination thereof. The V_{L} peptide can comprise a variant of any of these sequences as provided for herein.

In some embodiments, an antibody, or antigen binding fragment thereof, comprises a V_{H} peptide as set forth in SEQ ID Nos: 2, 4, 6, 8, 10, 12, 14, 16, 80, or 83, or any combination thereof. The V_{H} peptide can comprise a variant of any of these sequences as provided for herein.

In some embodiments, an antibody, or antigen binding fragment thereof, comprises a V_{H} peptide and a V_{L} peptide, wherein the wherein the V_{H} peptide comprises a sequence as set forth in SEQ ID NO: 2, 4, 6, 8, 10, 12, 14, 16, 80, or 83 and the V_{L} peptide comprises a sequence as set forth in SEQ ID NO: 1, 3, 5, 7, 9, 11, 13, 15, 79, or 86.

In some embodiments, an antibody, or antigen binding fragment thereof, comprises a V_{H} peptide and a V_{L} peptide, wherein the V_{H} peptide comprises a sequence as set forth in SEQ ID NO: 2 and the V_{L} peptide comprises a sequence as set forth in SEQ ID NO: 1. In some embodiments, an antibody, or antigen binding fragment thereof, comprises a V_{H} peptide and a V_{L} peptide, wherein the V_{H} peptide comprises a sequence as set forth in SEQ ID NO: 4 and the V_{L} peptide comprises a sequence as set forth in SEQ ID NO: 3. In some embodiments, an antibody, or antigen binding fragment thereof, comprises a V_{H} peptide and a V_{L} peptide, wherein the V_{H} peptide comprises a sequence as set forth in SEQ ID NO: 6 and the V_{L} peptide comprises a sequence as set forth in SEQ ID NO: 5. In some embodiments, an antibody, or antigen binding fragment thereof, comprises a V_{H} peptide and a V_{L} peptide, wherein the V_{H} peptide comprises a sequence as set forth in SEQ ID NO: 8 and the V_{L} peptide comprises a sequence as set forth in SEQ ID NO: 7. In some embodiments, an antibody, or antigen binding fragment thereof, comprises a V_{H} peptide and a V_{L} peptide, wherein the V_{H} peptide comprises a sequence as set forth in SEQ ID NO: 10 and the V_{L} peptide comprises a sequence as set forth in SEQ ID NO: 9. In some embodiments, an antibody, or antigen binding fragment thereof, comprises a V_{H} peptide and a V_{L} peptide, wherein the V_{H} peptide comprises a sequence as set forth in SEQ ID NO: 12 and the V_{L} peptide comprises a sequence as set forth in SEQ ID NO: 11. In some embodiments, an antibody, or antigen binding fragment thereof, comprises a V_{H} peptide and a V_{L} peptide, wherein the V_{H} peptide comprises a sequence as set forth in SEQ ID NO: 14 and the V_{L} peptide comprises a sequence as set forth in SEQ ID NO: 13. In some embodiments, an antibody, or antigen binding fragment thereof, comprises a V_{H} peptide and a V_{L} peptide, wherein the V_{H} peptide comprises a sequence as set forth in SEQ ID NO: 16 and the V_{L} peptide comprises a sequence as set forth in SEQ ID NO: 15. In some embodiments, an antibody, or antigen binding fragment thereof, comprises a V_{H} peptide and a V_{L} peptide, wherein the V_{H} peptide comprises a sequence as set forth in SEQ ID NO: 80 and the V_{L} peptide comprises a sequence as set forth in SEQ ID NO: 79. In some embodiments, an antibody, or antigen binding fragment thereof, comprises a V_{H} peptide and a V_{L} peptide, wherein the V_{H} peptide comprises a sequence as set forth in SEQ ID NO: 83 and the V_{L} peptide comprises a sequence as set forth in SEQ ID NO: 3. In some embodiments, an antibody, or antigen binding fragment thereof, comprises a V_{H} peptide and a V_{L} peptide, wherein the V_{H} peptide comprises a sequence as set forth in SEQ ID NO: 14 and the V_{L} peptide comprises a sequence as set forth in SEQ ID NO: 86.

In some embodiments, an antibody, or antigen binding fragment thereof, comprises a LC peptide as set forth in SEQ ID Nos: 1, 3, 5, 7, 9, or 11, or any combination thereof. The LC peptide can comprise a variant of any of these sequences as provided for herein.

In some embodiments, an antibody, or antigen binding fragment thereof, comprises a HC peptide as set forth in SEQ ID Nos: 2, 4, 6, 8, 10, 12, or 83, or any combination thereof. The HC peptide can comprise a variant of any of these sequences as provided for herein.

In some embodiments, an antibody, or antigen binding fragment thereof, comprises a HC peptide and a LC peptide, wherein the wherein the HC peptide comprises a sequence as set forth in SEQ ID NO: 2, 4, 6, 8, 10, 12, or 83 and the LC peptide comprises a sequence as set forth in SEQ ID NO: 1, 3, 5, 7, 9, or 11. In some embodiments, an antibody, or antigen binding fragment thereof, comprises a HC peptide and a LC peptide, wherein the HC peptide comprises a sequence as set forth in SEQ ID NO: 2 and the LC peptide comprises a sequence as set forth in SEQ ID NO: 1. In some embodiments, an antibody, or antigen binding fragment thereof, comprises a HC peptide and a LC peptide, wherein the HC peptide comprises a sequence as set forth in SEQ ID NO: 4 and the LC peptide comprises a sequence as set forth in SEQ ID NO: 3. In some embodiments, the HC peptide comprising the sequence as set forth in SEQ ID NO: 4 has an additional C terminal lysine (K) residue. In some embodiments, an antibody, or antigen binding fragment thereof, comprises a HC peptide and a LC peptide, wherein the HC peptide comprises a sequence as set forth in SEQ ID NO: 6 and the LC peptide comprises a sequence as set forth in SEQ ID NO: 5. In some embodiments, an antibody, or antigen binding fragment thereof, comprises a HC peptide and a LC peptide, wherein the HC peptide comprises a sequence as set forth in SEQ ID NO: 8 and the LC peptide comprises a sequence as set forth in SEQ ID NO: 7. In some embodiments, an antibody, or antigen binding fragment thereof, comprises a HC peptide and a LC peptide, wherein the HC peptide comprises a sequence as set forth in SEQ ID NO: 10 and the LC peptide comprises a sequence as set forth in SEQ ID NO: 9. In some embodiments, an antibody, or antigen binding fragment thereof, comprises a HC peptide and a LC peptide, wherein the HC peptide comprises a sequence as set forth in SEQ ID NO: 12 and the LC peptide comprises a sequence as set forth in SEQ ID NO: 11. In some embodiments, an antibody, or antigen binding fragment thereof, comprises a HC peptide and a LC peptide, wherein the HC peptide comprises a sequence as set forth in SEQ ID NO: 83 and the LC peptide comprises a sequence as set forth in SEQ ID NO: 3.

In addition to these specific combinations any of the V_{H} peptides and the V_{L} peptides can be combined with one another.

In addition to these specific combinations any of the HC peptides and the LC peptides can be combined with one another.

In some embodiments, the antibody comprises a sequence, or antigen binding fragment of ATCC clone PTA-7444. The sequence of the antibody produced by ATCC clone PTA-7444 is hereby incorporated by reference in its entirety, which includes the antigen binding fragments thereof.

In some embodiments, the antibody comprises a heavy and a light chain, wherein the heavy chain comprises a sequence of:

(SEQ ID NO: 92)
QVQLVQSGAEVVKPGASVKLSCKASGYTFTSYWMHWVKQRPGQGLE
WIGEINPSNGRTNYNQKFQGKATLTVDKSSSTAYMQLSSLTSEDSAVYY
FARGRPDYYGSSKWYFDVWGQGTTVTVSSASTKGPSVFPLAPSSKSTSG
GTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELL
GGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEWE
SNGQPENNYKTTPPVLDSDGSFFLYSKLTVDKSRWQQGNVFSCSVMHEA
LHNHYTQKSLSLSPGK;

(SEQ ID NO: 93)
DVVMTQTPLSLPVSLGDPASISCRSSQSIVHSNVNTYLEWYLQKPGQSP
RLLIYKVSNRFSGVPDRFSGSGAGTDFTLRISRVEAEDLGIYYCFQGSH
VPPTFGGGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC.

In some embodiments, the antibody comprises a heavy and a light chain, wherein the heavy chain comprises a sequence of:

(SEQ ID NO: 94)
QVQLVQSGAEVVKPGASVKLSCKASGYTFTSYWMHWVKQRPGQGLEWIG
EINPSNGRTNYNQKFQGKATLTVDKSSSTAYMQLSSLTSEDSAVYYFAR
GRPDYYGSSKWYFDVWGQGTTVTVSSASTKGPSVFPLAPSSKSTSGGTA
ALGCLVKDYFPEPVTVSWNSGALTSGVHTFPAVLQSSGLYSLSSVVTVP
SSSLGTQTYICNVNHKPSNTKVDKKVEPKSCDKTHTCPPCPAPELLGGP
SVFLFPPKPKDTLYITREPEVTCVVVDVSHEDPEVKFNWYVDGVEVHNA
KTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALHN
HYTQKSLSLSPG;

and the light chain comprises a sequence of

(SEQ ID NO: 93)
DVVMTQTPLSLPVSLGDPASISCRSSQSIVHSNVNTYLEWYLQKPGQSP
RLLIYKVSNRFSGVPDRFSGSGAGTDFTLRISRVEAEDLGIYYCFQGSH
VPPTFGGGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC.

In some embodiments, the heavy chain of SEQ ID NO: 94 comprises a C-terminal lysine residue that is added to the C-terminus of SEQ ID NO: 94.

In some embodiments, the antibody comprises a heavy and a light chain, wherein the heavy chain comprises a sequence of:

(SEQ ID NO: 95)
QVQLVQSGAEVVKPGASVKLSSKASGYTFTSYWMHWVKQRPGQGLEWIG
EINPSNGRTNYNQKFQGKATLTVDKSSSTAYMQLSSLTSEDSAVYYFAR
GRPDYYGSSKWYFDVWGQGTTVTVSSASTKGPSVFPLAPSSKSTSGGTA
ALGCLVKDYFPEPVTVSWNSGALTSGVHTFPAVLQSSGLYSLSSVVTVP
SSSLGTQTYICNVNHKPSNTKVDKKVEPKSCDKTHTCPPCPAPELLGGP
SVFLFPPKPKDTLYITREPEVTCVVVDVSHEDPEVKFNWYVDGVEVHNA
KTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALHN
HYTQKSLSLSPG;

and the light chain comprises a sequence of SEQ ID NO: 93

In some embodiments, the heavy chain of SEQ ID NO: 95 comprises a C-terminal lysine residue that is added to the C-terminus of SEQ ID NO: 95.

In some embodiments, the antibody comprises a heavy chain and light chain, wherein the heavy chain comprises a sequence of SEQ ID NO: 83 and the and the light chain comprises a sequence of SEQ ID NO: 3.

In some embodiments, the antibody comprises a VH sequence of SEQ ID NO: 96 and a VL sequence of SEQ ID NO: 13 or SEQ ID NO: 97. In some embodiments, the antibody comprises a VH of SEQ ID NO: 14 and a VL sequence of SEQ ID NO: 97.

In some embodiments, the IGF-1R antibody comprises a heavy chain that is at least 85% identical to SEQ ID NO: 92. In some embodiments, the IGF-1R antibody comprises a heavy chain that is at least 90% identical to SEQ ID NO: 92. In some embodiments, the IGF-1R antibody comprises a heavy chain that is at least 91% identical to SEQ ID NO: 92. In some embodiments, the IGF-1R antibody comprises a heavy chain that is at least 92% identical to SEQ ID NO: 92. In some embodiments, the IGF-1R antibody comprises a heavy chain that is at least 93% identical to SEQ ID NO: 92. In some embodiments, the IGF-1R antibody comprises a heavy chain that is at least 94% identical to SEQ ID NO: 92. In some embodiments, the IGF-1R antibody comprises a heavy chain that is at least 95% identical to SEQ ID NO: 92. In some embodiments, the IGF-1R antibody comprises a heavy chain that is at least 96% identical to SEQ ID NO: 92. In some embodiments, the IGF-1R antibody comprises a heavy chain that is at least 97% identical to SEQ ID NO: 92. In some embodiments, the IGF-1R antibody comprises a heavy chain that is at least 98% identical to SEQ ID NO: 92. In some embodiments, the IGF-1R antibody comprises a heavy chain that is at least 99% identical to SEQ ID NO: 92. In some embodiments, the IGF-1R antibody comprises a light chain that is at least 85% identical to SEQ ID NO: 93. In some embodiments, the IGF-1R antibody comprises a light chain that is at least 90% identical to SEQ ID NO: 93. In some embodiments, the IGF-1R antibody comprises a light chain that is at least 91% identical to SEQ ID NO: 93. In some embodiments, the IGF-1R antibody comprises a light chain that is at least 92% identical to SEQ ID NO: 93. In some embodiments, the IGF-1R antibody comprises a light chain that is at least 93% identical to SEQ ID NO: 93. In some embodiments, the IGF-1R antibody comprises a light chain that is at least 94% identical to SEQ ID NO: 93. In some embodiments, the IGF-1R antibody comprises a light chain that is at least 95% identical to SEQ ID NO: 93. In some embodiments, the IGF-1R antibody comprises a light chain that is at least 96% identical to SEQ ID NO: 93. In some embodiments, the IGF-1R antibody comprises a light chain that is at least 97% identical to SEQ ID NO: 93. In some embodiments, the IGF-1R antibody comprises a light chain that is at least 98% identical to SEQ ID NO: 93. In some embodiments, the IGF-1R antibody comprises a light chain that is at least 99% identical to SEQ ID NO: 93.

In some embodiments, the IGF-1R antibody comprises a V_{H} sequence that is at least 85% identical to SEQ ID NO: 91. In some embodiments, the IGF-1R antibody comprises a V_{H} sequence that is at least 90% identical to SEQ ID NO: 91. In some embodiments, the IGF-1R antibody comprises a V_{H} sequence that is at least 91% identical to SEQ ID NO: 91. In some embodiments, the IGF-1R antibody comprises a V_{H} sequence that is at least 92% identical to SEQ ID NO: 91. In some embodiments, the IGF-1R antibody comprises a V_{H} sequence that is at least 93% identical to SEQ ID NO: 91. In some embodiments, the IGF-1R antibody comprises a V_{H} sequence that is at least 94% identical to SEQ ID NO: 91. In some embodiments, the IGF-1R antibody comprises a V_{H} sequence that is at least 95% identical to SEQ ID NO: 91. In some embodiments, the IGF-1R antibody comprises a V_{H} sequence that is at least 96% identical to SEQ ID NO: 91. In some embodiments, the IGF-1R antibody comprises a V_{H} sequence that is at least 97% identical to SEQ ID NO: 91. In some embodiments, the IGF-1R antibody comprises a V_{H} sequence that is at least 98% identical to SEQ ID NO: 91. In some embodiments, the IGF-1R antibody comprises a V_{H} sequence that is at least 99% identical to SEQ ID NO: 91.

In some embodiments, the IGF-1R antibody comprises a V_{L} sequence that is at least 85% identical to SEQ ID NO: 86. In some embodiments, the IGF-1R antibody comprises a V_{L} sequence that is at least 90% identical to SEQ ID NO: 86. In some embodiments, the IGF-1R antibody comprises a V_{L} sequence that is at least 91% identical to SEQ ID NO: 86. In some embodiments, the IGF-1R antibody comprises a V_{L} sequence that is at least 92% identical to SEQ ID NO: 86. In some embodiments, the IGF-1R antibody comprises a V_{L} sequence that is at least 93% identical to SEQ ID NO: 86. In some embodiments, the IGF-1R antibody comprises a V_{L} sequence that is at least 94% identical to SEQ ID NO: 86. In some embodiments, the IGF-1R antibody comprises a V_{L} sequence that is at least 95% identical to SEQ ID NO: 86. In some embodiments, the IGF-1R antibody comprises a V_{L} sequence that is at least 96% identical to SEQ ID NO: 86. In some embodiments, the IGF-1R antibody comprises a V_{L} sequence that is at least 97% identical to SEQ ID NO: 86. In some embodiments, the IGF-1R antibody comprises a V_{L} sequence that is at least 98% identical to SEQ ID NO: 86. In some embodiments, the IGF-1R antibody comprises a V_{L} sequence that is at least 99% identical to SEQ ID NO: 86.

In some embodiments, the IGF-1R antibody comprises a heavy chain that is at least 85% identical to SEQ ID NO: 94. In some embodiments, the IGF-1R antibody comprises a heavy chain that is at least 90% identical to SEQ ID NO: 94. In some embodiments, the IGF-1R antibody comprises a heavy chain that is at least 91% identical to SEQ ID NO: 94. In some embodiments, the IGF-1R antibody comprises a heavy chain that is at least 92% identical to SEQ ID NO: 94. In some embodiments, the IGF-1R antibody comprises a heavy chain that is at least 93% identical to SEQ ID NO: 94. In some embodiments, the IGF-1R antibody comprises a heavy chain that is at least 94% identical to SEQ ID NO: 94. In some embodiments, the IGF-1R antibody comprises a heavy chain that is at least 95% identical to SEQ ID NO: 94. In some embodiments, the IGF-1R antibody comprises a heavy chain that is at least 96% identical to SEQ ID NO: 94. In some embodiments, the IGF-1R antibody comprises a heavy chain that is at least 97% identical to SEQ ID NO: 94. In some embodiments, the IGF-1R antibody comprises a heavy chain that is at least 98% identical to SEQ ID NO: 94. In some embodiments, the IGF-1R antibody comprises a heavy chain that is at least 99% identical to SEQ ID NO: 94.

In some embodiments, the IGF-1R antibody comprises a heavy chain that is at least 85% identical to SEQ ID NO: 83. In some embodiments, the IGF-1R antibody comprises a heavy chain that is at least 90% identical to SEQ ID NO: 83. In some embodiments, the IGF-1R antibody comprises a heavy chain that is at least 91% identical to SEQ ID NO: 83. In some embodiments, the IGF-1R antibody comprises a heavy chain that is at least 92% identical to SEQ ID NO: 83. In some embodiments, the IGF-1R antibody comprises a heavy chain that is at least 93% identical to SEQ ID NO: 83. In some embodiments, the IGF-1R antibody comprises a heavy chain that is at least 94% identical to SEQ ID NO: 83. In some embodiments, the IGF-1R antibody comprises a heavy chain that is at least 95% identical to SEQ ID NO: 83. In some embodiments, the IGF-1R antibody comprises a heavy chain that is at least 96% identical to SEQ ID NO: 83. In some embodiments, the IGF-1R antibody comprises a heavy chain that is at least 97% identical to SEQ ID NO: 83. In some embodiments, the IGF-1R antibody comprises a heavy chain that is at least 98% identical to SEQ ID NO: 83. In some embodiments, the IGF-1R antibody comprises a heavy chain that is at least 99% identical to SEQ ID NO: 83.

In some embodiments, the IGF-1R antibody comprises a light chain that is at least 85% identical to SEQ ID NO: 3. In some embodiments, the IGF-1R antibody comprises a light chain that is at least 90% identical to SEQ ID NO: 3. In some embodiments, the IGF-1R antibody comprises a light chain that is at least 91% identical to SEQ ID NO: 3. In some embodiments, the IGF-1R antibody comprises a light chain that is at least 92% identical to SEQ ID NO: 3. In some embodiments, the IGF-1R antibody comprises a light chain that is at least 93% identical to SEQ ID NO: 3. In some embodiments, the IGF-1R antibody comprises a light chain that is at least 94% identical to SEQ ID NO: 3. In some embodiments, the IGF-1R antibody comprises a light chain that is at least 95% identical to SEQ ID NO: 3. In some embodiments, the IGF-1R antibody comprises a light chain that is at least 96% identical to SEQ ID NO: 3. In some embodiments, the IGF-1R antibody comprises a light chain that is at least 97% identical to SEQ ID NO: 3. In some embodiments, the IGF-1R antibody comprises a light chain that is at least 98% identical to SEQ ID NO: 3. In some embodiments, the IGF-1R antibody comprises a light chain that is at least 99% identical to SEQ ID NO: 3.

In some embodiments, the IGF-1R antibody comprises a V_{H} sequence that is at least 85% identical to SEQ ID NO: 99. In some embodiments, the IGF-1R antibody comprises a V_{H} sequence that is at least 90% identical to SEQ ID NO: 99. In some embodiments, the IGF-1R antibody comprises a V_{H} sequence that is at least 91% identical to SEQ ID NO: 99. In some embodiments, the IGF-1R antibody comprises a V_{H} sequence that is at least 92% identical to SEQ ID NO: 99. In some embodiments, the IGF-1R antibody comprises a V_{H} sequence that is at least 93% identical to SEQ ID NO: 99. In some embodiments, the IGF-1R antibody comprises a V_{H} sequence that is at least 94% identical to SEQ ID NO: 99. In some embodiments, the IGF-1R antibody comprises a V_{H} sequence that is at least 95% identical to SEQ ID NO: 99. In some embodiments, the IGF-1R antibody comprises a V_{H} sequence that is at least 96% identical to SEQ ID NO: 99. In some embodiments, the IGF-1R antibody comprises a V_{H} sequence that is at least 97% identical to SEQ ID NO: 99. In some embodiments, the IGF-1R antibody comprises a V_{H} sequence that is at least 98% identical to SEQ ID NO: 99. In some embodiments, the IGF-1R antibody comprises a V_{H} sequence that is at least 99% identical to SEQ ID NO: 99.

In some embodiments, the IGF-1R antibody comprises a V_{L} sequence that is at least 85% identical to SEQ ID NO: 98. In some embodiments, the IGF-1R antibody comprises a V_{L} sequence that is at least 90% identical to SEQ ID NO: 98. In some embodiments, the IGF-1R antibody comprises a V_{L} sequence that is at least 91% identical to SEQ ID NO: 98. In some embodiments, the IGF-1R antibody comprises a V_{L} sequence that is at least 92% identical to SEQ ID NO: 98. In some embodiments, the IGF-1R antibody comprises a V_{L} sequence that is at least 93% identical to SEQ ID NO: 98. In some embodiments, the IGF-1R antibody comprises a V_{L} sequence that is at least 94% identical to SEQ ID NO: 98. In some embodiments, the IGF-1R antibody comprises a V_{L} sequence that is at least 95% identical to SEQ ID NO: 98. In some embodiments, the IGF-1R antibody comprises a V_{L} sequence that is at least 96% identical to SEQ ID NO: 98. In some embodiments, the IGF-1R antibody comprises a V_{L} sequence that is at least 97% identical to SEQ ID NO: 98. In some embodiments, the IGF-1R antibody comprises a V_{L} sequence that is at least 98% identical to SEQ ID NO: 98. In some embodiments, the IGF-1R antibody comprises a V_{L} sequence that is at least 99% identical to SEQ ID NO: 98.

### Pharmaceutical Compositions/Stable Formulations

The present disclosure is directed to anti-IGF-1R antibody compositions or stable formulation comprising IGF-1R antibodies. Throughout the specification, the terms “pharmaceutical compositions” and “stable formulations” are used interchangeably.

Throughout the present disclosure, all expressions of percentage, ratio, and the like are “by weight” unless otherwise indicated. As used herein, “by weight” is synonymous with the term “by mass,” and indicates that a ratio or percentage defined herein is done according to weight rather than volume, thickness, or some other measure.

As described herein, the term “anti-IGF-1R antibody composition” refers to a composition comprising one or more anti-IRG-1R antibodies, or one or more antigen binding fragments thereof.

### Antibody Concentration

In some embodiments, an anti-IGF-1R antibody, or antigen binding fragment thereof, is present in the pharmaceutical composition at an amount of from 1 mg/ml to 300 mg/ml, 1 mg/ml to 200 mg, 10 mg/ml to 200 mg, 20 mg/ml to 150 mg/ml, 25 mg/ml to 150 mg/ml, 25 mg/ml to 125 mg/ml, 25 mg/ml to 100 mg/ml, 25 mg/ml to 75 mg/ml, 30 mg/ml to 60 mg/ml, or 45 mg/ml to 55 mg/ml. In some embodiments, an anti-IGF-1R antibody, or antigen binding fragment thereof, is present in the pharmaceutical composition at an amount of a 20 mg/ml, 25 mg/ml, 30 mg/ml, 40 mg/ml, 45 mg/ml, 50 mg/ml, 55 mg/ml, 60 mg/ml, 65 mg/ml, 70 mg/ml, 75 mg/ml, 80 mg/ml, 85 mg/ml, 90 mg/ml, 95 mg/ml, 100 mg/ml, 125 mg/ml, 150 mg/ml, 175 mg/ml, 200 mg/ml, 225 mg/ml, 250 mg/ml, 275 mg/ml, or 300 mg/ml. In some embodiments, an anti-IGF-1R antibody, or antigen binding fragment thereof, is present in the pharmaceutical composition at an amount of 25 mg/ml, 50 mg/ml, or 150 mg/ml. In some embodiments, an anti-IGF-1R antibody, or antigen binding fragment thereof, is present in the pharmaceutical composition at an amount of 20 mg/ml. In some embodiments, an anti-IGF-1R antibody, or antigen binding fragment thereof, is present in the pharmaceutical composition at an amount of 25 mg/ml. In some embodiments, an anti-IGF-1R antibody, or antigen binding fragment thereof, is present in the pharmaceutical composition at an amount of 30 mg/ml. In some embodiments, an anti-IGF-1R antibody, or antigen binding fragment thereof, is present in the pharmaceutical composition at an amount of 40 mg/ml. In some embodiments, an anti-IGF-1R antibody, or antigen binding fragment thereof, is present in the pharmaceutical composition at an amount of 45 mg/ml. In some embodiments, an anti-IGF-1R antibody, or antigen binding fragment thereof, is present in the pharmaceutical composition at an amount of 50 mg/ml. In some embodiments, an anti-IGF-1R antibody, or antigen binding fragment thereof, is present in the pharmaceutical composition at an amount of 55 mg/ml. In some embodiments, an anti-IGF-1R antibody, or antigen binding fragment thereof, is present in the pharmaceutical composition at an amount of 60 mg/ml. In some embodiments, an anti-IGF-1R antibody, or antigen binding fragment thereof, is present in the pharmaceutical composition at an amount of 65 mg/ml. In some embodiments, an anti-IGF-1R antibody, or antigen binding fragment thereof, is present in the pharmaceutical composition at an amount of 70 mg/ml. In some embodiments, an anti-IGF-1R antibody, or antigen binding fragment thereof, is present in the pharmaceutical composition at an amount of 75 mg/ml. In some embodiments, an anti-IGF-1R antibody, or antigen binding fragment thereof, is present in the pharmaceutical composition at an amount of 80 mg/ml. In some embodiments, an anti-IGF-1R antibody, or antigen binding fragment thereof, is present in the pharmaceutical composition at an amount of 85 mg/ml. In some embodiments, an anti-IGF-1R antibody, or antigen binding fragment thereof, is present in the pharmaceutical composition at an amount of 90 mg/ml. In some embodiments, an anti-IGF-1R antibody, or antigen binding fragment thereof, is present in the pharmaceutical composition at an amount of 95 mg/ml. In some embodiments, an anti-IGF-1R antibody, or antigen binding fragment thereof, is present in the pharmaceutical composition at an amount of 100 mg/ml. In some embodiments, an anti-IGF-1R antibody, or antigen binding fragment thereof, is present in the pharmaceutical composition at an amount of 125 mg/ml. In some embodiments, an anti-IGF-1R antibody, or antigen binding fragment thereof, is present in the pharmaceutical composition at an amount of 150 mg/ml. In some embodiments, an anti-IGF-1R antibody, or antigen binding fragment thereof, is present in the pharmaceutical composition at an amount of 175 mg/ml. In some embodiments, an anti-IGF-1R antibody, or antigen binding fragment thereof, is present in the pharmaceutical composition at an amount of 200 mg/ml. In some embodiments, an anti-IGF-1R antibody, or antigen binding fragment thereof, is present in the pharmaceutical composition at an amount of 225 mg/ml. In some embodiments, an anti-IGF-1R antibody, or antigen binding fragment thereof, is present in the pharmaceutical composition at an amount of 250 mg/ml. In some embodiments, an anti-IGF-1R antibody, or antigen binding fragment thereof, is present in the pharmaceutical composition at an amount of 275 mg/ml. In some embodiments, an anti-IGF-1R antibody, or antigen binding fragment thereof, is present in the pharmaceutical composition at an amount of 300 mg/ml.

In some embodiments, an anti-IGF-1R antibody, or antigen binding fragment thereof, is present at a concentration of greater than 20 mg/ml. In some embodiments, an anti-IGF-1R antibody, or antigen binding fragment thereof, is present at a concentration of greater than 25 mg/ml. In some embodiments, an anti-IGF-1R antibody, or antigen binding fragment thereof, is present at a concentration of greater than 40 mg/ml. In some embodiments, an anti-IGF-1R antibody, or antigen binding fragment thereof, is present at a concentration of greater than 50 mg/ml. In some embodiments, an anti-IGF-1R antibody, or antigen binding fragment thereof, is present at a concentration of greater than 75 mg/ml. In some embodiments, an anti-IGF-1R antibody, or antigen binding fragment thereof, is present at a concentration of greater than 90 mg/ml. In some embodiments, an anti-IGF-1R antibody, or antigen binding fragment thereof, is present at a concentration of greater than 100 mg/ml. In some embodiments, an anti-IGF-1R antibody, or antigen binding fragment thereof, is present at a concentration of greater than 120 mg/ml. In some embodiments, an anti-IGF-1R antibody, or antigen binding fragment thereof, is present at a concentration of greater than 140 mg/ml. In some embodiments, an anti-IGF-1R antibody, or antigen binding fragment thereof, is present at a concentration of greater than 160 mg/ml. In some embodiments, an anti-IGF-1R antibody, or antigen binding fragment thereof, is present at a concentration of greater than 180 mg/ml. In some embodiments, an anti-IGF-1R antibody, or antigen binding fragment thereof, is present at a concentration of greater than 200 mg/ml.

In some embodiments, an anti-IGF-1R antibody, or antigen binding fragment thereof, is present at a concentration of 1 mg/ml to 300 mg/ml. In some embodiments, an anti-IGF-1R antibody, or antigen binding fragment thereof, is present at a concentration of 1 mg/ml to 200 mg/ml. In some embodiments, an anti-IGF-1R antibody, or antigen binding fragment thereof, is present at a concentration of 10 mg/ml to 200 mg/ml. In some embodiments, an anti-IGF-1R antibody, or antigen binding fragment thereof, is present at a concentration of 20 mg/ml to 150 mg/ml. In some embodiments, an anti-IGF-1R antibody, or antigen binding fragment thereof, is present at a concentration of 25 mg/ml to 150 mg/ml. In some embodiments, an anti-IGF-1R antibody, or antigen binding fragment thereof, is present at a concentration of 25 mg/ml to 125 mg/ml. In some embodiments, an anti-IGF-1R antibody, or antigen binding fragment thereof, is present at a concentration of 25 mg/ml to 100 mg/ml., In some embodiments, an anti-IGF-1R antibody, or antigen binding fragment thereof, is present at a concentration of 25 mg/ml to 75 mg/ml. In some embodiments, an anti-IGF-1R antibody, or antigen binding fragment thereof, is present at a concentration of 30 mg/ml to 60 mg/ml. In some embodiments, an anti-IGF-1R antibody, or antigen binding fragment thereof, is present at a concentration of 45 mg/ml to 55 mg/ml. In some embodiments, an anti-IGF-1R antibody, or antigen binding fragment thereof, is present at a concentration of 100 mg/ml to 300 mg/ml. In some embodiments, an anti-IGF-1R antibody, or antigen binding fragment thereof, is present at a concentration of 105 mg/ml to 180 mg/ml. In some embodiments, an anti-IGF-1R antibody, or antigen binding fragment thereof, is present at a concentration of 120 mg/ml to 160 mg/ml. In some embodiments, an anti-IGF-1R antibody, or antigen binding fragment thereof, is present at a concentration of 125 mg/ml to 175 mg/ml. In some embodiments, an anti-IGF-1R antibody, or antigen binding fragment thereof, is present at a concentration of 110 mg/ml to 140 mg/ml. In some embodiments, an anti-IGF-1R antibody, or antigen binding fragment thereof, is present at a concentration of 165 mg/ml to 185 mg/ml.

### Buffer

In some embodiments, the pharmaceutical compositions comprise a buffer (e.g., histidine, acetate, phosphate or citrate buffer) and/or a stabilizer agent (e.g., human albumin), etc., or a combination thereof. In some embodiments, the pharmaceutical compositions comprise one or more pharmaceutically acceptable carriers, including, e.g., ion exchangers, alumina, aluminum stearate, lecithin, serum proteins, such as human serum albumin, buffer substances such as phosphates, sucrose, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, polyethylene-polyoxypropylene-block polymers, and polyethylene glycol, or a combination thereof.

Various buffers can be present in the anti-IGF-1R antibody compositions of the present disclosure. In some embodiments, the pharmaceutical composition comprises a pharmaceutically acceptable buffer, wherein the pharmaceutically acceptable buffer is phosphoric acid buffer, citric acid buffer, acetic acid buffer, citrate buffer, ascorbic acid buffer, glutamic acid buffer, lactic acid buffer, maleic acid buffer, trometamol buffer, and gluconic acid buffer, acetate buffer, succinate buffer, phosphate buffer, histidine buffer or any combination thereof. In some embodiments, the buffer is histidine buffer, histidine HCl buffer, glycine buffer, Tris/glycine buffer, acetate buffer, sodium acetate buffer, potassium acetate buffer, magnesium acetate buffer, phosphate buffer, or citrate buffer. In some embodiments, the buffer is histidine buffer. In some embodiments, the buffer is histidine HCl buffer. In some embodiments, the buffer is glycine buffer. In some embodiments, the buffer is Tris/glycine buffer. In some embodiments, the buffer is acetate buffer. In some embodiments, the buffer is sodium acetate buffer. In some embodiments, the buffer is potassium acetate buffer. In some embodiments, the buffer is magnesium acetate buffer. In some embodiments, the buffer is phosphate buffer. In some embodiments, the buffer is citrate buffer. In some embodiments, the buffer is not succinate buffer.

Unless stated otherwise, a buffer comprises both acid and its conjugate base sufficient to adjust and maintain the pH. One of ordinary skill in the art will be able to ascertain an acid and its conjugate base used in the specific buffer. In some embodiments, a histidine buffer comprises histidine and histidine hydrochloride. In some embodiments, a histidine buffer comprises L-histidine and L-histidine-HCl. In some embodiments, an acetate buffer (acetate acid buffer) comprises acetic acid and sodium acetate. In some embodiments, a glutamic acid comprises glutamic acid and sodium glutamate.

In some embodiments, the pharmaceutically acceptable buffer is phosphoric acid buffer. In some embodiments, the pharmaceutically acceptable buffer is 5 mM phosphoric acid buffer to 100 mM phosphoric acid buffer. In some embodiments, the pharmaceutically acceptable buffer is 10 mM to 70 mM phosphoric acid buffer. In some embodiments, the pharmaceutically acceptable buffer is 10 mM to 50 mM phosphoric acid buffer. In some embodiments, the pharmaceutically acceptable buffer is 5 mM to 60 mM phosphoric acid buffer. In some embodiments, the pharmaceutically acceptable buffer is 10 mM to 40 mM phosphoric acid buffer. In some embodiments, the pharmaceutically acceptable buffer is 15 mM to 30 mM phosphoric acid buffer. In some embodiments, the pharmaceutically acceptable buffer is 15 mM to 25 mM phosphoric acid buffer. In some embodiments, the pharmaceutically acceptable buffer is 5 mM phosphoric acid buffer. In some embodiments, the pharmaceutically acceptable buffer is 10 mM phosphoric acid buffer. In some embodiments, the pharmaceutically acceptable buffer is 15 mM phosphoric acid buffer. In some embodiments, the pharmaceutically acceptable buffer is 20 mM phosphoric acid buffer. In some embodiments, the pharmaceutically acceptable buffer is 25 mM phosphoric acid buffer. In some embodiments, the pharmaceutically acceptable buffer is 30 mM phosphoric acid buffer. In some embodiments, the pharmaceutically acceptable buffer is 35 mM phosphoric acid buffer. In some embodiments, the pharmaceutically acceptable buffer is 40 mM phosphoric acid buffer. In some embodiments, the pharmaceutically acceptable buffer is 45 mM phosphoric acid buffer. In some embodiments, the pharmaceutically acceptable buffer is 50 mM phosphoric acid buffer. In some embodiments, the pharmaceutically acceptable buffer is 55 mM phosphoric acid buffer. In some embodiments, the pharmaceutically acceptable buffer is 60 mM phosphoric acid buffer. In some embodiments, the pharmaceutically acceptable buffer is 75 mM phosphoric acid buffer. In some embodiments, the pharmaceutically acceptable buffer is 80 mM phosphoric acid buffer. In some embodiments, the pharmaceutically acceptable buffer is 85 mM phosphoric acid buffer. In some embodiments, the pharmaceutically acceptable buffer is 90 mM phosphoric acid buffer. In some embodiments, the pharmaceutically acceptable buffer is 95 mM phosphoric acid buffer. In some embodiments, the pharmaceutically acceptable buffer is 100 mM phosphoric acid buffer.

In some embodiments, the pharmaceutically acceptable buffer is a citric acid buffer. In some embodiments, the pharmaceutically acceptable buffer is 5 mM citric acid buffer to 100 mM citric acid buffer. In some embodiments, the pharmaceutically acceptable buffer is 10 mM to 70 mM citric acid buffer. In some embodiments, the pharmaceutically acceptable buffer is 10 mM to 50 mM citric acid buffer. In some embodiments, the pharmaceutically acceptable buffer is 5 mM to 60 mM citric acid buffer. In some embodiments, the pharmaceutically acceptable buffer is 10 mM to 40 mM citric acid buffer. In some embodiments, the pharmaceutically acceptable buffer is 15 mM to 30 mM citric acid buffer. In some embodiments, the pharmaceutically acceptable buffer is 15 mM to 25 mM citric acid buffer. In some embodiments, the pharmaceutically acceptable buffer is 5 mM citric acid buffer. In some embodiments, the pharmaceutically acceptable buffer is 10 mM citric acid buffer. In some embodiments, the pharmaceutically acceptable buffer is 15 mM citric acid buffer. In some embodiments, the pharmaceutically acceptable buffer is 20 mM citric acid buffer. In some embodiments, the pharmaceutically acceptable buffer is 25 mM citric acid buffer. In some embodiments, the pharmaceutically acceptable buffer is 30 mM citric acid buffer. In some embodiments, the pharmaceutically acceptable buffer is 35 mM citric acid buffer. In some embodiments, the pharmaceutically acceptable buffer is 40 mM citric acid buffer. In some embodiments, the pharmaceutically acceptable buffer is 45 mM citric acid buffer. In some embodiments, the pharmaceutically acceptable buffer is 50 mM citric acid buffer. In some embodiments, the pharmaceutically acceptable buffer is 55 mM citric acid buffer. In some embodiments, the pharmaceutically acceptable buffer is 60 mM citric acid buffer. In some embodiments, the pharmaceutically acceptable buffer is 75 mM citric acid buffer. In some embodiments, the pharmaceutically acceptable buffer is 80 mM citric acid buffer. In some embodiments, the pharmaceutically acceptable buffer is 85 mM citric acid buffer. In some embodiments, the pharmaceutically acceptable buffer is 90 mM citric acid buffer. In some embodiments, the pharmaceutically acceptable buffer is 95 mM citric acid buffer. In some embodiments, the pharmaceutically acceptable buffer is 100 mM citric acid buffer.

In some embodiments, the pharmaceutically acceptable buffer is acetic acid buffer (acetate buffer). In some embodiments, the pharmaceutically acceptable buffer is 5 mM acetic acid buffer to 100 mM acetic acid buffer. In some embodiments, the pharmaceutically acceptable buffer is 10 mM to 70 mM acetic acid buffer. In some embodiments, the pharmaceutically acceptable buffer is 10 mM to 50 mM acetic acid buffer. In some embodiments, the pharmaceutically acceptable buffer is 5 mM to 60 mM acetic acid buffer. In some embodiments, the pharmaceutically acceptable buffer is 10 mM to 40 mM acetic acid buffer. In some embodiments, the pharmaceutically acceptable buffer is 15 mM to 30 mM acetic acid buffer. In some embodiments, the pharmaceutically acceptable buffer is 15 mM to 25 mM acetic acid buffer. In some embodiments, the pharmaceutically acceptable buffer is 5 mM acetic acid buffer. In some embodiments, the pharmaceutically acceptable buffer is 10 mM acetic acid buffer. In some embodiments, the pharmaceutically acceptable buffer is 15 mM acetic acid buffer. In some embodiments, the pharmaceutically acceptable buffer is 20 mM acetic acid buffer. In some embodiments, the pharmaceutically acceptable buffer is 25 mM acetic acid buffer. In some embodiments, the pharmaceutically acceptable buffer is 30 mM acetic acid buffer. In some embodiments, the pharmaceutically acceptable buffer is 35 mM acetic acid buffer. In some embodiments, the pharmaceutically acceptable buffer is 40 mM acetic acid buffer. In some embodiments, the pharmaceutically acceptable buffer is 45 mM acetic acid buffer. In some embodiments, the pharmaceutically acceptable buffer is 50 mM acetic acid buffer. In some embodiments, the pharmaceutically acceptable buffer is 55 mM acetic acid buffer. In some embodiments, the pharmaceutically acceptable buffer is 60 mM acetic acid buffer. In some embodiments, the pharmaceutically acceptable buffer is 75 mM acetic acid buffer. In some embodiments, the pharmaceutically acceptable buffer is 80 mM acetic acid buffer. In some embodiments, the pharmaceutically acceptable buffer is 85 mM acetic acid buffer. In some embodiments, the pharmaceutically acceptable buffer is 90 mM acetic acid buffer. In some embodiments, the pharmaceutically acceptable buffer is 95 mM acetic acid buffer. In some embodiments, the pharmaceutically acceptable buffer is 100 mM acetic acid buffer.

In some embodiments, the pharmaceutically acceptable buffer is 5 mM succinic acid buffer to 100 mM succinic acid buffer. In some embodiments, the pharmaceutically acceptable buffer is 10 mM to 70 mM succinic acid buffer. In some embodiments, the pharmaceutically acceptable buffer is 10 mM to 50 mM succinic acid buffer. In some embodiments, the pharmaceutically acceptable buffer is 5 mM to 60 mM succinic acid buffer. In some embodiments, the pharmaceutically acceptable buffer is 10 mM to 40 mM succinic acid buffer. In some embodiments, the pharmaceutically acceptable buffer is 15 mM to 30 mM succinic acid buffer. In some embodiments, the pharmaceutically acceptable buffer is 15 mM to 25 mM succinic acid buffer. In some embodiments, the pharmaceutically acceptable buffer is 5 mM succinic acid buffer. In some embodiments, the pharmaceutically acceptable buffer is 10 mM succinic acid buffer. In some embodiments, the pharmaceutically acceptable buffer is 15 mM succinic acid buffer. In some embodiments, the pharmaceutically acceptable buffer is 20 mM succinic acid buffer. In some embodiments, the pharmaceutically acceptable buffer is 25 mM succinic acid buffer. In some embodiments, the pharmaceutically acceptable buffer is 30 mM succinic acid buffer. In some embodiments, the pharmaceutically acceptable buffer is 35 mM succinic acid buffer. In some embodiments, the pharmaceutically acceptable buffer is 40 mM succinic acid buffer. In some embodiments, the pharmaceutically acceptable buffer is 45 mM succinic acid buffer. In some embodiments, the pharmaceutically acceptable buffer is 50 mM succinic acid buffer. In some embodiments, the pharmaceutically acceptable buffer is 55 mM succinic acid buffer. In some embodiments, the pharmaceutically acceptable buffer is 60 mM succinic acid buffer. In some embodiments, the pharmaceutically acceptable buffer is 75 mM succinic acid buffer. In some embodiments, the pharmaceutically acceptable buffer is 80 mM succinic acid buffer. In some embodiments, the pharmaceutically acceptable buffer is 85 mM succinic acid buffer. In some embodiments, the pharmaceutically acceptable buffer is 90 mM succinic acid buffer. In some embodiments, the pharmaceutically acceptable buffer is 95 mM succinic acid buffer. In some embodiments, the pharmaceutically acceptable buffer is 100 mM succinic acid buffer.

In some embodiments, the pharmaceutically acceptable buffer 5 mM citrate buffer to 100 mM citrate buffer. In some embodiments, the pharmaceutically acceptable buffer is 10 mM to 70 mM citrate buffer. In some embodiments, the pharmaceutically acceptable buffer is 10 mM to 50 mM citrate buffer. In some embodiments, the pharmaceutically acceptable buffer is 5 mM to 60 mM citrate buffer. In some embodiments, the pharmaceutically acceptable buffer is 10 mM to 40 mM citrate buffer. In some embodiments, the pharmaceutically acceptable buffer is 15 mM to 30 mM citrate buffer. In some embodiments, the pharmaceutically acceptable buffer is 15 mM to 25 mM citrate buffer. In some embodiments, the pharmaceutically acceptable buffer is 5 mM citrate buffer. In some embodiments, the pharmaceutically acceptable buffer is 10 mM citrate buffer. In some embodiments, the pharmaceutically acceptable buffer is 15 mM citrate buffer. In some embodiments, the pharmaceutically acceptable buffer is 20 mM citrate buffer. In some embodiments, the pharmaceutically acceptable buffer is 25 mM citrate buffer. In some embodiments, the pharmaceutically acceptable buffer is 30 mM citrate buffer. In some embodiments, the pharmaceutically acceptable buffer is 35 mM citrate buffer. In some embodiments, the pharmaceutically acceptable buffer is 40 mM citrate buffer. In some embodiments, the pharmaceutically acceptable buffer is 45 mM citrate buffer. In some embodiments, the pharmaceutically acceptable buffer is 50 mM citrate buffer. In some embodiments, the pharmaceutically acceptable buffer is 55 mM citrate buffer. In some embodiments, the pharmaceutically acceptable buffer is 60 mM citrate buffer. In some embodiments, the pharmaceutically acceptable buffer is 75 mM citrate buffer. In some embodiments, the pharmaceutically acceptable buffer is 80 mM citrate buffer. In some embodiments, the pharmaceutically acceptable buffer is 85 mM citrate buffer. In some embodiments, the pharmaceutically acceptable buffer is 90 mM citrate buffer. In some embodiments, the pharmaceutically acceptable buffer is 95 mM citrate buffer. In some embodiments, the pharmaceutically acceptable buffer is 100 mM citrate buffer.

In some embodiments, the pharmaceutically acceptable buffer is 5 mM glutamic acid buffer to 100 mM glutamic acid buffer. In some embodiments, the pharmaceutically acceptable buffer is 10 mM to 70 mM glutamic acid buffer. In some embodiments, the pharmaceutically acceptable buffer is 10 mM to 50 mM glutamic acid buffer. In some embodiments, the pharmaceutically acceptable buffer is 5 mM to 60 mM glutamic acid buffer. In some embodiments, the pharmaceutically acceptable buffer is 10 mM to 40 mM glutamic acid buffer. In some embodiments, the pharmaceutically acceptable buffer is 15 mM to 30 mM glutamic acid buffer. In some embodiments, the pharmaceutically acceptable buffer is 15 mM to 25 mM glutamic acid buffer. In some embodiments, the pharmaceutically acceptable buffer is 5 mM glutamic acid buffer. In some embodiments, the pharmaceutically acceptable buffer is 10 mM glutamic acid buffer. In some embodiments, the pharmaceutically acceptable buffer is 15 mM glutamic acid buffer. In some embodiments, the pharmaceutically acceptable buffer is 20 mM glutamic acid buffer. In some embodiments, the pharmaceutically acceptable buffer is 25 mM glutamic acid buffer. In some embodiments, the pharmaceutically acceptable buffer is 30 mM glutamic acid buffer. In some embodiments, the pharmaceutically acceptable buffer is 35 mM glutamic acid buffer. In some embodiments, the pharmaceutically acceptable buffer is 40 mM glutamic acid buffer. In some embodiments, the pharmaceutically acceptable buffer is 45 mM glutamic acid buffer. In some embodiments, the pharmaceutically acceptable buffer is 50 mM glutamic acid buffer. In some embodiments, the pharmaceutically acceptable buffer is 55 mM glutamic acid buffer. In some embodiments, the pharmaceutically acceptable buffer is 60 mM glutamic acid buffer. In some embodiments, the pharmaceutically acceptable buffer is 75 mM glutamic acid buffer. In some embodiments, the pharmaceutically acceptable buffer is 80 mM glutamic acid buffer. In some embodiments, the pharmaceutically acceptable buffer is 85 mM glutamic acid buffer. In some embodiments, the pharmaceutically acceptable buffer is 90 mM glutamic acid buffer. In some embodiments, the pharmaceutically acceptable buffer is 95 mM glutamic acid buffer. In some embodiments, the pharmaceutically acceptable buffer is 100 mM glutamic acid buffer.

In some embodiments, the pharmaceutically acceptable buffer is 5 mM acetate buffer to 100 mM acetate buffer. In some embodiments, the pharmaceutically acceptable buffer is 10 mM to 70 mM acetate buffer. In some embodiments, the pharmaceutically acceptable buffer is 10 mM to 50 mM acetate buffer. In some embodiments, the pharmaceutically acceptable buffer is 5 mM to 60 mM acetate buffer. In some embodiments, the pharmaceutically acceptable buffer is 10 mM to 40 mM acetate buffer. In some embodiments, the pharmaceutically acceptable buffer is 15 mM to 30 mM acetate buffer. In some embodiments, the pharmaceutically acceptable buffer is 15 mM to about 25 mM acetate buffer. In some embodiments, the pharmaceutically acceptable buffer is 5 mM acetate buffer. In some embodiments, the pharmaceutically acceptable buffer is 10 mM acetate buffer. In some embodiments, the pharmaceutically acceptable buffer is 15 mM acetate buffer. In some embodiments, the pharmaceutically acceptable buffer is 20 mM acetate buffer. In some embodiments, the pharmaceutically acceptable buffer is 25 mM acetate buffer. In some embodiments, the pharmaceutically acceptable buffer is 30 mM acetate buffer. In some embodiments, the pharmaceutically acceptable buffer is 35 mM acetate buffer. In some embodiments, the pharmaceutically acceptable buffer is 40 mM acetate buffer. In some embodiments, the pharmaceutically acceptable buffer is 45 mM acetate buffer. In some embodiments, the pharmaceutically acceptable buffer is 50 mM acetate buffer. In some embodiments, the pharmaceutically acceptable buffer is 55 mM acetate buffer. In some embodiments, the pharmaceutically acceptable buffer is 60 mM acetate buffer. In some embodiments, the pharmaceutically acceptable buffer is 75 mM acetate buffer. In some embodiments, the pharmaceutically acceptable buffer is 80 mM acetate buffer. In some embodiments, the pharmaceutically acceptable buffer is 85 mM acetate buffer. In some embodiments, the pharmaceutically acceptable buffer is 90 mM acetate buffer. In some embodiments, the pharmaceutically acceptable buffer is 95 mM acetate buffer. In some embodiments, the pharmaceutically acceptable buffer is 100 mM acetate buffer.

In some embodiments, the acetate buffer is a potassium acetate buffer, sodium acetate buffer or magnesium acetate buffer. In some embodiments, the acetate buffer is a potassium acetate buffer. In some embodiments, the potassium acetate buffer is provided at an acetate buffer concentration provided above. In some embodiments, the acetate buffer is a sodium acetate buffer. In some embodiments, the sodium acetate buffer is provided at an acetate buffer concentration provided above. In some embodiments, the acetate buffer is a magnesium acetate buffer. In some embodiments, the magnesium acetate buffer is provided at an acetate buffer concentration provided above.

In some embodiments, the pharmaceutically acceptable buffer is 5 mM succinate buffer to 100 mM succinate buffer. In some embodiments, the pharmaceutically acceptable buffer is 10 mM to 70 mM succinate buffer. In some embodiments, the pharmaceutically acceptable buffer is 10 mM to 50 mM succinate buffer. In some embodiments, the pharmaceutically acceptable buffer is 5 mM to 60 mM succinate buffer. In some embodiments, the pharmaceutically acceptable buffer is 10 mM to 40 mM succinate buffer. In some embodiments, the pharmaceutically acceptable buffer is 15 mM to 30 mM succinate buffer. In some embodiments, 15 mM to about 25 mM succinate buffer. In some embodiments, the pharmaceutically acceptable buffer is 5 mM succinate buffer. In some embodiments, the pharmaceutically acceptable buffer is 10 mM succinate buffer. In some embodiments, the pharmaceutically acceptable buffer is 15 mM succinate buffer. In some embodiments, the pharmaceutically acceptable buffer is 20 mM succinate buffer. In some embodiments, the pharmaceutically acceptable buffer is 25 mM succinate buffer. In some embodiments, the pharmaceutically acceptable buffer is 30 mM succinate buffer. In some embodiments, the pharmaceutically acceptable buffer is 35 mM succinate buffer. In some embodiments, the pharmaceutically acceptable buffer is 40 mM succinate buffer. In some embodiments, the pharmaceutically acceptable buffer is 45 mM succinate buffer. In some embodiments, the pharmaceutically acceptable buffer is 50 mM succinate buffer. In some embodiments, the pharmaceutically acceptable buffer is 55 mM succinate buffer. In some embodiments, the pharmaceutically acceptable buffer is 60 mM succinate buffer. In some embodiments, the pharmaceutically acceptable buffer is 75 mM succinate buffer. In some embodiments, the pharmaceutically acceptable buffer is 80 mM succinate buffer. In some embodiments, the pharmaceutically acceptable buffer is 85 mM succinate buffer. In some embodiments, the pharmaceutically acceptable buffer is 90 mM succinate buffer. In some embodiments, the pharmaceutically acceptable buffer is 95 mM succinate buffer. In some embodiments, the pharmaceutically acceptable buffer is 100 mM succinate buffer.

In some embodiments, the succinate buffer is sodium succinate buffer. In some embodiments, the sodium succinate buffer is provided at a concentration succinate buffer concentration provided above.

In some embodiments, the pharmaceutically acceptable buffer is 5 mM phosphate buffer to 100 mM phosphate buffer. In some embodiments, the pharmaceutically acceptable buffer is 10 mM to 70 mM phosphate buffer. In some embodiments, the pharmaceutically acceptable buffer is 10 mM to 50 mM phosphate buffer. In some embodiments, the pharmaceutically acceptable buffer is 5 mM to 60 mM phosphate buffer. In some embodiments, the pharmaceutically acceptable buffer is 10 mM to 40 mM phosphate buffer. In some embodiments, the pharmaceutically acceptable buffer is 15 mM to 30 mM phosphate buffer. In some embodiments, the pharmaceutically acceptable buffer is 15 mM to 25 mM phosphate buffer. In some embodiments, the pharmaceutically acceptable buffer is 5 mM phosphate buffer. In some embodiments, the pharmaceutically acceptable buffer is 10 mM phosphate buffer. In some embodiments, the pharmaceutically acceptable buffer is 15 mM phosphate buffer. In some embodiments, the pharmaceutically acceptable buffer is 20 mM phosphate buffer. In some embodiments, the pharmaceutically acceptable buffer is 25 mM phosphate buffer. In some embodiments, the pharmaceutically acceptable buffer is 30 mM phosphate buffer. In some embodiments, the pharmaceutically acceptable buffer is 35 mM phosphate buffer. In some embodiments, the pharmaceutically acceptable buffer is 40 mM phosphate buffer. In some embodiments, the pharmaceutically acceptable buffer is 45 mM phosphate buffer. In some embodiments, the pharmaceutically acceptable buffer is 50 mM phosphate buffer. In some embodiments, the pharmaceutically acceptable buffer is 55 mM phosphate buffer. In some embodiments, the pharmaceutically acceptable buffer is 60 mM phosphate buffer. In some embodiments, the pharmaceutically acceptable buffer is 75 mM phosphate buffer. In some embodiments, the pharmaceutically acceptable buffer is 80 mM phosphate buffer. In some embodiments, the pharmaceutically acceptable buffer is 85 mM phosphate buffer. In some embodiments, the pharmaceutically acceptable buffer is 90 mM phosphate buffer. In some embodiments, the pharmaceutically acceptable buffer is 95 mM phosphate buffer. In some embodiments, the pharmaceutically acceptable buffer is 100 mM phosphate buffer.

In some embodiments, the phosphate buffer is sodium phosphate. In some embodiments, sodium phosphate buffer is present at a phosphate buffer concentration provided above.

In some embodiments, the pharmaceutically acceptable buffer is 5 mM to 100 mM histidine buffer. In some embodiments, the pharmaceutically acceptable buffer is 10 mM to 70 mM histidine buffer. In some embodiments, the pharmaceutically acceptable buffer is 10 mM to 50 mM histidine buffer. In some embodiments, the pharmaceutically acceptable buffer is 5 mM to 60 mM histidine buffer. In some embodiments, the pharmaceutically acceptable buffer is 10 mM to 40 mM histidine buffer. In some embodiments, the pharmaceutically acceptable buffer is 15 mM to 30 mM histidine buffer. In some embodiments, the pharmaceutically acceptable buffer is 15 mM to about 25 mM histidine buffer. In some embodiments, the pharmaceutically acceptable buffer is 5 mM histidine buffer. In some embodiments, the pharmaceutically acceptable buffer is 10 mM histidine buffer. In some embodiments, the pharmaceutically acceptable buffer is 15 mM histidine buffer. In some embodiments, the pharmaceutically acceptable buffer is 20 mM histidine buffer. In some embodiments, the pharmaceutically acceptable buffer is 25 mM histidine buffer. In some embodiments, the pharmaceutically acceptable buffer is 30 mM histidine buffer. In some embodiments, the pharmaceutically acceptable buffer is 35 mM histidine buffer. In some embodiments, the pharmaceutically acceptable buffer is 40 mM histidine buffer. In some embodiments, the pharmaceutically acceptable buffer is 45 mM histidine buffer. In some embodiments, the pharmaceutically acceptable buffer is 50 mM histidine buffer. In some embodiments, the pharmaceutically acceptable buffer is 55 mM histidine buffer. In some embodiments, the pharmaceutically acceptable buffer is 60 mM histidine buffer. In some embodiments, the pharmaceutically acceptable buffer is 75 mM histidine buffer. In some embodiments, the pharmaceutically acceptable buffer is 80 mM histidine buffer. In some embodiments, the pharmaceutically acceptable buffer is 85 mM histidine buffer. In some embodiments, the pharmaceutically acceptable buffer is 90 mM histidine buffer. In some embodiments, the pharmaceutically acceptable buffer is 95 mM histidine buffer. In some embodiments, the pharmaceutically acceptable buffer is 100 mM histidine buffer.

### Uncharged Excipients and Sugars

In some embodiments, anti-IGF-1R antibody compositions of the present disclosure comprise an uncharged excipient. The term “excipient” refers to a pharmacologically inactive substance formulated with an antibody or antigen binding fragment thereof as described herein. In some embodiments, the uncharged excipient can assist in the prevention of denaturation or otherwise assist in stabilizing the antibody or antigen binding fragment thereof. Examples of excipients are known in the art. Examples can be taken, e.g., from the handbook: Gennaro, Alfonso R.: “Remington's Pharmaceutical Sciences”, Mack Publishing Company, Easton, Pa., 1990.

In some embodiments, the uncharged excipient is fructose, glucose, mannose, sorbose, xylose, lactose, maltose, sucrose, dextran, pullulan, dextrin, cyclodextrins, soluble starch, trehalose, sorbitol, erythritol, isomalt, lactitol, maltitol, xylitol, glycerol, lactitol, hydroxyethyl starch, water-soluble glucans, or a combination thereof. In some embodiments, the uncharged excipient is sucrose. In some embodiments, the uncharged excipient is trehaolse. In some embodiments, the uncharged excipient is mannitol.

In some embodiments, the uncharged excipient is present in anti-IGF-1R antibody compositions in an amount of from 0.5% (w/v) to 20% (w/v) in the pharmaceutical composition, for example, from 0.5% to 20%, from 1% to 20%, from 1% to 15%, from 1% to 10%, from 2% to 10%, from 3% to 10%, from 4% to 10%, from 5% to 10%, from 6% to 10%, from 7% to 10%, from 7.5% to 10%, from 7.5% to 8.5%, from 7.5% to 8%. In some embodiments the uncharged excipient is present at a concentration of 0.5%, 1%, 1.5%, 2%, 2.5%, 3%, 3.5%, 4%, 4.5%, 6%, 6.5%, 7%, 7.5%, 8%, 8.5%, 9%, 9.5%, or 10% (w/v). In some embodiments, an uncharged excipient is present at a concentration of 0.5% (w/v) to 20% (w/v). In some embodiments, an uncharged excipient is present at a concentration of 0.5% (w/v) to 20% (w/v). In some embodiments, an uncharged excipient is present at a concentration of 1% (w/v) to 20% (w/v). In some embodiments, an uncharged excipient is present at a concentration of 1% (w/v) to 15% (w/v). In some embodiments, an uncharged excipient is present at a concentration of 1% (w/v) to 10% (w/v). In some embodiments, an uncharged excipient is present at a concentration of 2% (w/v) to 10% (w/v). In some embodiments, an uncharged excipient is present at a concentration of 3% (w/v) to 10% (w/v). In some embodiments, an uncharged excipient is present at a concentration of 4% (w/v) to 10% (w/v). In some embodiments, an uncharged excipient is present at a concentration of 5% (w/v) to 10% (w/v). In some embodiments, an uncharged excipient is present at a concentration of 6% (w/v) to 10% (w/v). In some embodiments, an uncharged excipient is present at a concentration of 7% (w/v) to 10% (w/v). In some embodiments, an uncharged excipient is present at a concentration of 7.5% (w/v) to 10% (w/v). In some embodiments, an uncharged excipient is present at a concentration of 7.5% (w/v) to about 8.5% (w/v). In some embodiments, an uncharged excipient is present at a concentration of 7.5% (w/v) to 8% (w/v).

In some embodiments the uncharged excipient is present at a concentration of 0.5% (w/v). In some embodiments the uncharged excipient is present at a concentration of 1% (w/v). In some embodiments the uncharged excipient is present at a concentration of 1.5% (w/v). In some embodiments the uncharged excipient is present at a concentration of 2% (w/v). In some embodiments the uncharged excipient is present at a concentration of 2.5% (w/v). In some embodiments the uncharged excipient is present at a concentration of 3% (w/v). In some embodiments the uncharged excipient is present at a concentration of 3.5% (w/v). In some embodiments the uncharged excipient is present at a concentration of 4% (w/v). In some embodiments the uncharged excipient is present at a concentration of 4.5% (w/v). In some embodiments the uncharged excipient is present at a concentration of 5% (w/v). In some embodiments the uncharged excipient is present at a concentration of 5.5% (w/v). In some embodiments the uncharged excipient is present at a concentration of 6% (w/v). In some embodiments the uncharged excipient is present at a concentration of 6.5% (w/v). In some embodiments the uncharged excipient is present at a concentration of 7% (w/v). In some embodiments the uncharged excipient is present at a concentration of 7.5% (w/v). In some embodiments the uncharged excipient is present at a concentration of 8% (w/v). In some embodiments the uncharged excipient is present at a concentration of 8.5% (w/v). In some embodiments the uncharged excipient is present at a concentration of 9% (w/v). In some embodiments the uncharged excipient is present at a concentration of 9.5% (w/v). In some embodiments the uncharged excipient is present at a concentration of 10% (w/v).

In some embodiments, the sucrose is present in anti-IGF-1R antibody compositions in an amount of from 0.5% (w/v) to 20% (w/v) in the pharmaceutical composition, for example, from 0.5% to 20%, from 1% to 20%, from 1% to 15%, from 1% to 10%, from 2% to 10%, from 3% to 10%, from 4% to 10%, from 5% to 10%, from 6% to 10%, from 7% to 10%, from 7.5% to 10%, from 7.5% to 8.5%, from 7.5% to 8%. In some embodiments, sucrose is present at a concentration of 0.5% (w/v) to 20% (w/v). In some embodiments, sucrose is present at a concentration of 0.5% (w/v) to 20% (w/v). In some embodiments, sucrose is present at a concentration of 1% (w/v) to 20% (w/v). In some embodiments, sucrose is present at a concentration of 1% (w/v) to 15% (w/v). In some embodiments, sucrose is present at a concentration of 1% (w/v) to 10% (w/v). In some embodiments, sucrose is present at a concentration of 2% (w/v) to 10% (w/v). In some embodiments, sucrose is present at a concentration of 3% (w/v) to 10% (w/v). In some embodiments, sucrose is present at a concentration of 4% (w/v) to 10% (w/v). In some embodiments, sucrose is present at a concentration of 5% (w/v) to 10% (w/v). In some embodiments, sucrose is present at a concentration of 6% (w/v) to 10% (w/v). In some embodiments, sucrose is present at a concentration of 7% (w/v) to 10% (w/v). In some embodiments, sucrose is present at a concentration of 7.5% (w/v) to 10% (w/v). In some embodiments, sucrose is present at a concentration of 7.5% (w/v) to about 8.5% (w/v). In some embodiments, sucrose is present at a concentration of 7.5% (w/v) to 8% (w/v).

In some embodiments the sucrose is present at a concentration of 0.5%, 1%, 1.5%, 2%, 2.5%, 3%, 3.5%, 4%, 4.5%, 6%, 6.5%, 7%, 7.5%, 8%, 8.5%, 9%, 9.5%, or 10% (w/v). In some embodiments the sucrose is present at a concentration of 0.5% (w/v). In some embodiments the sucrose is present at a concentration of 1% (w/v). In some embodiments the sucrose is present at a concentration of 1.5% (w/v). In some embodiments the sucrose is present at a concentration of 2% (w/v). In some embodiments the sucrose is present at a concentration of 2.5% (w/v). In some embodiments the sucrose is present at a concentration of 3% (w/v). In some embodiments the sucrose is present at a concentration of 3.5% (w/v). In some embodiments the sucrose is present at a concentration of 4% (w/v). In some embodiments the sucrose is present at a concentration of 4.5% (w/v). In some embodiments the sucrose is present at a concentration of 5% (w/v). In some embodiments the sucrose is present at a concentration of 5.5% (w/v). In some embodiments the sucrose is present at a concentration of 6% (w/v). In some embodiments the sucrose is present at a concentration of 6.5% (w/v). In some embodiments the sucrose is present at a concentration of 7% (w/v). In some embodiments the sucrose is present at a concentration of 7.5% (w/v). In some embodiments the sucrose is present at a concentration of 8% (w/v). In some embodiments the sucrose is present at a concentration of 8.5% (w/v). In some embodiments the sucrose is present at a concentration of 9% (w/v). In some embodiments the sucrose is present at a concentration of 9.5% (w/v). In some embodiments the sucrose is present at a concentration of 10% (w/v).

### Stabilizer

In some embodiments, the pharmaceutical compositions comprise a stabilizer. In some embodiments, the stabilizer is an amino acid. In some embodiments, the stabilizer comprises methionine, L-methionine, arginine, L-arginine, glycine, L-glycine, histidine, L-histidine, proline, or L-proline. In some embodiments, the stabilizer is methionine, L-methionine, arginine, L-arginine, glycine, L-glycine, histidine, L-histidine, proline, or L-proline. In some embodiments, the stabilizer is methionine. In some embodiments, the stabilizer is L-methionine. In some embodiments, the stabilizer is arginine. In some embodiments, the stabilizer is L-arginine. In some embodiments, the stabilizer is glycine. In some embodiments, the stabilizer is L-glycine. In some embodiments, the stabilizer is histidine. In some embodiments, the stabilizer is L-histidine. In some embodiments, the stabilizer is proline. In some embodiments, the stabilizer is L-proline.

In some embodiments, anti-IGF-1R antibody compositions of the present disclosure comprise methionine.

Methionine, as used herein, includes the free base form of methionine, as well as any and all salts thereof. In some embodiments, methionine includes a pharmaceutically acceptable salt thereof, e.g., methionine hydrochloride. Methionine, as used herein, also includes all enantiomers (e.g., L-methionine and S-methionine), and any combination of enantiomers (e.g., 50% L-methionine and 50% S-methionine; 90%-100% L-methionine and 10%-0% S-methionine, and the like). In some embodiments, the term “methionine” includes greater than 99% L-methionine and less than 1% S-methionine. In some embodiments, the term “methionine” includes an enantiomerically pure L-methionine. In some embodiments, methionine is a pharmaceutical grade methionine.

In some embodiments, anti-IGF-1R antibody compositions of the present disclosure comprise arginine.

Arginine, as used herein, includes the free base form of arginine, as well as any and all salts thereof. In some embodiments, arginine includes a pharmaceutically acceptable salt thereof, e.g., arginine hydrochloride. Arginine, as used herein, also includes all enantiomers (e.g., L-arginine and S-arginine), and any combination of enantiomers (e.g., 50% L-arginine and 50% S-arginine; 90%-100% L-arginine and 10%-0% S-arginine, etc.). In some embodiments, the term “arginine” includes greater than 99% L-arginine and less than 1% S-arginine. In some embodiments, the term “arginine” includes an enantiomerically pure L-arginine. In some embodiments, arginine is a pharmaceutical grade arginine.

In some embodiments, anti-IGF-1R antibody compositions of the present disclosure comprise glycine

Glycine, as used herein, includes the free base form of arginine, as well as any and all salts thereof. In some embodiments, arginine includes a pharmaceutically acceptable salt thereof, e.g., glycine hydrochloride. Glycine, as used herein, also includes all enantiomers (e.g., L-glycine and S-glycine), and any combination of enantiomers (e.g., 50% L-glycine and 50% S-glycine; 90%-100% L-glycine and 10%-0% S-glycine, etc.). In some embodiments, the term “glycine” includes greater than 99% L-glycine and less than 1% S-glycine. In some embodiments, the term “glycine” includes an enantiomerically pure L-glycine. In some embodiments, glycine is a pharmaceutical grade glycine.

In some embodiments, anti-IGF-1R antibody compositions of the present disclosure comprise histidine.

Histidine, as used herein, includes the free base form of histidine, as well as any and all salts thereof. In some embodiments, histidine includes a pharmaceutically acceptable salt thereof, e.g., histidine hydrochloride. Histidine, as used herein, also includes all enantiomers (e.g., L-histidine and S-histidine), and any combination of enantiomers (e.g., 50% L-histidine and 50% S-histidine; 90%-100% L-histidine and 10%-0% S-histidine, etc.). In some embodiments, the term “histidine” includes greater than 99% L-histidine and less than 1% S-histidine. In some embodiments, the term “histidine” includes an enantiomerically pure L-histidine. In some embodiments, histidine is a pharmaceutical grade histidine.

In some embodiments, anti-IGF-1R antibody compositions of the present disclosure comprise proline.

Proline, as used herein, includes the free base form of proline, as well as any and all salts thereof. In some embodiments, proline includes a pharmaceutically acceptable salt thereof, e.g., proline hydrochloride. Proline, as used herein, also includes all enantiomers (e.g., L-proline and S-proline), and any combination of enantiomers (e.g., 50% L-proline and 50% S-proline; 90%-100% L-proline and 10%-0% S-proline, etc.). In some embodiments, the term “proline” includes greater than 99% L-proline and less than 1% S-proline. In some embodiments, the term “proline” includes an enantiomerically pure L-proline. In some embodiments, proline is a pharmaceutical grade proline.

Various concentrations of a stabilizer can be present in the anti-IGF-1R antibody compositions of the present disclosure. In some embodiments, the pharmaceutical composition comprises greater than 1 mM stabilizer, greater than 2 mM stabilizer, greater than 3 mM stabilizer, or greater than 4 mM stabilizer. In other aspects, the pharmaceutical composition comprises up to 5 mM stabilizer, up to 6 mM stabilizer, up to 7 mM stabilizer, up to 8 mM stabilizer, up to 9 mM stabilizer, up to 10 mM stabilizer, up to 11 mM stabilizer, up to 12 mM stabilizer, up to 13 mM stabilizer, up to 15 mM stabilizer, up to 20 mM stabilizer, or up to 200 mM stabilizer. In some embodiments, a stable formulation comprises greater than 1 mM stabilizer. In some embodiments, a stable formulation comprises greater than 2 mM stabilizer. In some embodiments, a stable formulation comprises greater than 3 mM stabilizer. In some embodiments, a stable formulation comprises greater than 4 mM stabilizer. In other aspects, a stable formulation comprises up to 5 mM stabilizer. In some embodiments, a stable formulation comprises up to 6 mM stabilizer. In some embodiments, a stable formulation comprises up to 7 mM stabilizer. In some embodiments, a stable formulation comprises up to 8 mM stabilizer. In some embodiments, a stable formulation comprises up to 9 mM stabilizer. In some embodiments, a stable formulation comprises up to 10 mM stabilizer. In some embodiments, a stable formulation comprises up to 11 mM stabilizer. In some embodiments, a stable formulation comprises up to 12 mM stabilizer. In some embodiments, a stable formulation comprises up to 13 mM stabilizer. In some embodiments, a stable formulation comprises up to 15 mM stabilizer. In some embodiments, a stable formulation comprises up to 20 mM stabilizer. In some embodiments, a stable formulation comprises up to 0 mM stabilizer. In some embodiments, a stable formulation comprises up to 200 mM stabilizer.

In other aspects, the pharmaceutical composition comprises 1 mM to 200 mM, 1 mM to 150 mM, 1 mM to 100 mM, 1 mM to 50 mM, 1 mM to 25 mM, 1 mM to 15 mM, 7 mM to 13 mM, 9 mM to 11 mM, or 10 mM stabilizer. In some embodiments, the pharmaceutical composition comprises 10 mM stabilizer. In some embodiments, the pharmaceutical composition comprises 10 mM stabilizer. In some embodiments, a stable formulation comprises 1 mM to 200 mM stabilizer. In some embodiments, a stable formulation comprises 1 mM to 150 mM stabilizer. In some embodiments, a stable formulation comprises 1 mM to 100 mM stabilizer. In some embodiments, a stable formulation comprises 1 mM to 50 mM stabilizer. In some embodiments, a stable formulation comprises 1 mM to 25 mM stabilizer. In some embodiments, a stable formulation comprises 1 mM to 15 mM stabilizer. In some embodiments, a stable formulation comprises 7 mM to 13 mM stabilizer. In some embodiments, a stable formulation comprises 9 mM to 11 mM stabilizer. In some embodiments, a stable formulation comprises 5 mM stabilizer. In some embodiments, a stable formulation comprises 10 mM stabilizer. In some embodiments, a stable formulation comprises 15 mM stabilizer. In some embodiments, a stable formulation comprises 20 mM stabilizer. In some embodiments, a stable formulation comprises 25 mM stabilizer. In some embodiments, a stable formulation comprises 30 mM stabilizer. In some embodiments, a stable formulation comprises 35 mM stabilizer. In some embodiments, a stable formulation comprises 40 mM stabilizer.

Various concentrations of methionine can be present in the anti-IGF-1R antibody compositions of the present disclosure. In some embodiments, the pharmaceutical composition comprises greater than 1 mM methionine, greater than 2 mM methionine, greater than 3 mM methionine, or greater than 4 mM methionine. In other aspects, the pharmaceutical composition comprises up to 5 mM methionine, up to 6 mM methionine, up to 7 mM methionine, up to 8 mM methionine, up to 9 mM methionine, up to 10 mM methionine, up to 11 mM methionine, up to 12 mM methionine, up to 13 mM methionine, up to 14 mM methionine, up to 15 mM methionine, or up to 200 mM methionine. In other aspects, the pharmaceutical composition comprises 1 mM to 200 mM, 1 mM to 150 mM, 1 mM to 100 mM, 1 mM to 50 mM, 1 mM to 25 mM, 1 mM to 15 mM, 7 mM to 13 mM, 9 mM to 11 mM, or 10 mM methionine. In some embodiments, the pharmaceutical composition comprises 5 mM methionine. In some embodiments, the pharmaceutical composition comprises 10 mM methionine. In some embodiments, a stable formulation comprises greater than 1 mM methionine. In some embodiments, a stable formulation comprises greater than 2 mM methionine. In some embodiments, a stable formulation comprises greater than 3 mM methionine. In some embodiments, a stable formulation comprises greater than 4 mM methionine. In other aspects, a stable formulation comprises up to 5 mM methionine. In some embodiments, a stable formulation comprises up to 6 mM methionine. In some embodiments, a stable formulation comprises up to 7 mM methionine. In some embodiments, a stable formulation comprises up to 8 mM methionine. In some embodiments, a stable formulation comprises up to 9 mM methionine. In some embodiments, a stable formulation comprises up to 10 mM methionine. In some embodiments, a stable formulation comprises up to 11 mM methionine. In some embodiments, a stable formulation comprises up to 12 mM methionine. In some embodiments, a stable formulation comprises up to 13 mM methionine. In some embodiments, a stable formulation comprises up to 15 mM methionine. In some embodiments, a stable formulation comprises up to 15 mM methionine. In some embodiments, a stable formulation comprises up to 20 mM methionine. In some embodiments, a stable formulation comprises up to 200 mM methionine. In some embodiments, a stable formulation comprises 1 mM to 200 mM methionine. In some embodiments, a stable formulation comprises 1 mM to 150 mM methionine. In some embodiments, a stable formulation comprises 1 mM to 100 mM methionine. In some embodiments, a stable formulation comprises 1 mM to 50 mM methionine. In some embodiments, a stable formulation comprises 1 mM to 25 mM methionine. In some embodiments, a stable formulation comprises 1 mM to 15 mM methionine. In some embodiments, a stable formulation comprises 7 mM to 13 mM methionine. In some embodiments, a stable formulation comprises 9 mM to 11 mM methionine. In some embodiments, a stable formulation comprises 5 mM methionine. In some embodiments, a stable formulation comprises 10 mM methionine. In some embodiments, a stable formulation comprises 15 mM methionine. In some embodiments, a stable formulation comprises 20 mM methionine. In some embodiments, a stable formulation comprises 25 mM methionine. In some embodiments, a stable formulation comprises 30 mM methionine. In some embodiments, a stable formulation comprises 35 mM methionine. In some embodiments, a stable formulation comprises 40 mM methionine. In some embodiments, methionine is added in an amount sufficient to maintain osmolality of the pharmaceutical composition. In some embodiments, methionine is added in an amount sufficient to achieve a hyper-tonic solution.

Various concentrations of L-methionine can be present in the anti-IGF-1R antibody compositions of the present disclosure. In some embodiments, the pharmaceutical composition comprises greater than 1 mM L-methionine, greater than 2 mM L-methionine, greater than 3 mM L-methionine, or greater than 4 mM L-methionine. In other aspects, the pharmaceutical composition comprises up to 5 mM L-methionine, up to 6 mM L-methionine, up to 7 mM L-methionine, up to 8 mM L-methionine, up to 9 mM L-methionine, up to 10 mM L-methionine, up to 11 mM L-methionine, up to 12 mM L-methionine, up to 13 mM L-methionine, up to 14 mM L-methionine, up to 15 mM L-methionine, or up to 200 mM L-methionine. In other aspects, the pharmaceutical composition comprises 1 mM to 200 mM, 1 mM to 150 mM, 1 mM to 100 mM, 1 mM to 50 mM, 1 mM to 25 mM, 1 mM to 15 mM, 7 mM to 13 mM, 9 mM to 11 mM, or 10 mM L-methionine. In some embodiments, the pharmaceutical composition comprises 10 mM L-methionine. In some embodiments, the pharmaceutical composition comprises 10 mM L-methionine. In some embodiments, a stable formulation comprises greater than 1 mM L-methionine. In some embodiments, a stable formulation comprises greater than 2 mM L-methionine. In some embodiments, a stable formulation comprises greater than 3 mM L-methionine. In some embodiments, a stable formulation comprises greater than 4 mM L-methionine. In other aspects, a stable formulation comprises up to 5 mM L-methionine. In some embodiments, a stable formulation comprises up to 6 mM L-methionine. In some embodiments, a stable formulation comprises up to 7 mM L-methionine. In some embodiments, a stable formulation comprises up to 8 mM L-methionine. In some embodiments, a stable formulation comprises up to 9 mM L-methionine. In some embodiments, a stable formulation comprises up to 10 mM L-methionine. In some embodiments, a stable formulation comprises up to 11 mM L-methionine. In some embodiments, a stable formulation comprises up to 12 mM L-methionine. In some embodiments, a stable formulation comprises up to 13 mM L-methionine. In some embodiments, a stable formulation comprises up to 15 mM L-methionine. In some embodiments, a stable formulation comprises up to 15 mM L-methionine. In some embodiments, a stable formulation comprises up to 20 mM L-methionine. In some embodiments, a stable formulation comprises up to 200 mM L-methionine. In some embodiments, a stable formulation comprises 1 mM to 200 mM L-methionine. In some embodiments, a stable formulation comprises 1 mM to 150 mM L-methionine. In some embodiments, a stable formulation comprises 1 mM to 100 mM L-methionine. In some embodiments, a stable formulation comprises 1 mM to 50 mM L-methionine. In some embodiments, a stable formulation comprises 1 mM to 25 mM L-methionine. In some embodiments, a stable formulation comprises 1 mM to 15 mM L-methionine. In some embodiments, a stable formulation comprises 7 mM to 13 mM L-methionine. In some embodiments, a stable formulation comprises 9 mM to 11 mM L-methionine. In some embodiments, a stable formulation comprises 5 mM L-methionine. In some embodiments, a stable formulation comprises 10 mM L-methionine. In some embodiments, a stable formulation comprises 15 mM L-methionine. In some embodiments, a stable formulation comprises 20 mM L-methionine. In some embodiments, a stable formulation comprises 25 mM L-methionine. In some embodiments, a stable formulation comprises 30 mM L-methionine. In some embodiments, a stable formulation comprises 35 mM L-methionine. In some embodiments, a stable formulation comprises 40 mM L-methionine.

Various concentrations of arginine, such as L-arginine, can be present in the anti-IGF-1R antibody compositions of the present disclosure. In some embodiments, the pharmaceutical composition comprises greater than 1 mM arginine, greater than 2 mM arginine, greater than 3 mM arginine, or greater than 4 mM arginine. In other aspects, the pharmaceutical composition comprises up to 5 mM arginine, up to 6 mM arginine, up to 7 mM arginine, up to 8 mM arginine, up to 9 mM arginine, up to 10 mM arginine, or up to 200 mM arginine. In other aspects, the pharmaceutical composition comprises 1 mM to 200 mM, 1 mM to 150 mM, 1 mM to 100 mM, 1 mM to 50 mM, 1 mM to 25 mM, 1 mM to 10 mM, 2 mM to 8 mM, 4 mM to 6 mM, or 5 mM arginine.

Various concentrations of L-arginine can be present in the anti-IGF-1R antibody compositions of the present disclosure. In some embodiments, the pharmaceutical composition comprises greater than 1 mM L-arginine, greater than 2 mM L-arginine, greater than 3 mM L-arginine, or greater than 4 mM L-arginine. In other aspects, the pharmaceutical composition comprises up to 5 mM L-arginine, up to 6 mM L-arginine, up to 7 mM L-arginine, up to 8 mM arginine, up to 9 mM L-arginine, up to 10 mM L-arginine, or up to 200 mM L-arginine. In other aspects, the pharmaceutical composition comprises 1 mM to 200 mM, 1 mM to 150 mM, 1 mM to 100 mM, 1 mM to 50 mM, 1 mM to 25 mM, 1 mM to 10 mM, 2 mM to 8 mM, 4 mM to 6 mM, or 5 mM L-arginine.

Various concentrations of glycine can be present in the anti-IGF-1R antibody compositions of the present disclosure. In some embodiments, the pharmaceutical composition comprises greater than 1 mM glycine, greater than 2 mM glycine, greater than 3 mM glycine, or greater than 4 mM glycine. In other aspects, the pharmaceutical composition comprises up to 5 mM glycine, up to 6 mM glycine, up to 7 mM glycine, up to 8 mM glycine, up to 9 mM glycine, up to 10 mM glycine, or up to 200 mM glycine. In other aspects, the pharmaceutical composition comprises 1 mM to 200 mM, 1 mM to 150 mM, 1 mM to 100 mM, 1 mM to 50 mM, 1 mM to 25 mM, 1 mM to 10 mM, 2 mM to 8 mM, 4 mM to 6 mM, or 5 mM glycine.

Various concentrations of L-glycine can be present in the anti-IGF-1R antibody compositions of the present disclosure. In some embodiments, the pharmaceutical composition comprises greater than 1 mM L-glycine, greater than 2 mM L-glycine, greater than 3 mM L-glycine, or greater than 4 mM L-glycine. In other aspects, the pharmaceutical composition comprises up to 5 mM L-glycine, up to 6 mM L-glycine, up to 7 mM L-glycine, up to 8 mM L-glycine, up to 9 mM L-glycine, up to 10 mM L-glycine, or up to 200 mM L-glycine. In other aspects, the pharmaceutical composition comprises 1 mM to 200 mM, 1 mM to 150 mM, 1 mM to 100 mM, 1 mM to 50 mM, 1 mM to 25 mM, 1 mM to 10 mM, 2 mM to 8 mM, 4 mM to 6 mM, or 5 mM L-glycine.

Various concentrations of proline can be present in the anti-IGF-1R antibody compositions of the present disclosure. In some embodiments, the pharmaceutical composition comprises greater than 1 mM proline, greater than 2 mM proline, greater than 3 mM proline, or greater than 4 mM proline. In other aspects, the pharmaceutical composition comprises up to 5 mM proline, up to 6 mM proline, up to 7 mM proline, up to 8 mM proline, up to 9 mM proline, up to 10 mM proline, or up to 200 mM proline. In other aspects, the pharmaceutical composition comprises 1 mM to 200 mM, 1 mM to 150 mM, 1 mM to 100 mM, 1 mM to 50 mM, 1 mM to 25 mM, 1 mM to 10 mM, 2 mM to 8 mM, 4 mM to 6 mM, or 5 mM proline.

Various concentrations of L-proline can be present in the anti-IGF-1R antibody compositions of the present disclosure. In some embodiments, the pharmaceutical composition comprises greater than 1 mM L-proline, greater than 2 mM L-proline, greater than 3 mM L-proline, or greater than 4 mM L-proline. In other aspects, the pharmaceutical composition comprises up to 5 mM L-proline, up to 6 mM L-proline, up to 7 mM L-proline, up to 8 mM L-proline, up to 9 mM L-proline, up to 10 mM L-proline, or up to 200 mM L-proline. In other aspects, the pharmaceutical composition comprises 1 mM to 200 mM, 1 mM to 150 mM, 1 mM to 100 mM, 1 mM to 50 mM, 1 mM to 25 mM, 1 mM to 10 mM, 2 mM to 8 mM, 4 mM to 6 mM, or 5 mM L-proline.

### Surfactants

One of the major stresses that proteins (e.g., antibodies) may encounter is interfacial stress (e.g., from air/water interfaces in liquid compositions, or ice/water interfaces during freezing/thawing.) Surfactants are typically used to stabilize proteins in biopharmaceutical compositions while under stress or long-term storage to prevent or minimize aggregation and/or particle formation. Examples of a surfactant include, but are not limited to, anionic surfactants (e.g., ammonium lauryl sulfate, sodium lauryl sulfate, sodium laureth sulfate, sodium myreth sulfate, diocytl sodium sulfosuccinate, perfluorooctanesulfonate, perfluorobutanesulfonate, alkyl-aryl ether phosphates, alkyl ether phosphates, carboxylates, sodium lauroyl sarcosinate, perfluorononanoate, perfluorooctanoate); cationic surfactants (e.g., octenidine dihydrochloride, cetrimonium bromide, cetylpyridinium chloride, benzalkonium chloride, benzethonium chloride, dimethyldioctadecylammonium chloride, and dioctadecyldimethylammonium bromide); zwitterionic (amphoteric) surfactants (e.g., 3-[(3-cholamidopropyl)dimethylammonio]-1-propanesulfonate, cocamidopropyl hydroxysultaine, phosphatidylserine, phosphatidylethanolamine, phosphatidylcholine, sphingomyelins, lauryldimethylamine oxide and myristamine oxide); non-ionic surfactants (e.g., polysorbates or Brij series); ethoxylates (e.g., fatty alcohol ethoxylate (e.g., octaethylene glycol monododecyl ether and pentaethylene glycol monododecyl ether), alkylphenolethoxylates (e.g., nonoxynols and Triton X-100); fatty acid ethoxylates, ethoxylated amines and/or fatty acid amides (e.g., poly ethoxylated tallow amine, cocamide monoethanol amine, and cocamide diethanolamine); terminally blocked ethoxylates (e.g., poloxamers); fatty acid esters of polyhydroxy compounds; fatty acid esters of glycerol (e.g., glycerol monostearate and glycerol monolaurate); fatty acid esters of sorbitol (e.g., Spans such as sorbitan monolaurate, sorbitan monostearate, and sorbitan tristearate, and Tweens such as Tween 20, Tween 40, Tween 60, and Tween 80); fatty acid esters of sucrose; alkyl poly glucosides (e.g., decyl glucoside, lauryl glucoside, and octyl glucoside); of a combination thereof.

In some embodiments, the surfactant is polysorbate 80 (PS80). PS80 is also known as polyoxyethylene (80) sorbitan monooleate.

In some embodiments, PS80 is present at a concentration of 0.001 to 1% (w/v), 0.001% to 0.5% (w/v), 0.01% to 0.5% (w/v), 0.01% to 0.1% (w/v), 0.01% to 0.05% (w/v), 0.01% to 0.04% (w/v), 0.01% (w/v), 0.02% (w/v), 0.03% (w/v), 0.04% (w/v), 0.05% (w/v), 0.06% (w/v), 0.07% (w/v), 0.08% (w/v), 0.09% (w/v), or 0.1% (w/v). In some embodiments, PS80 is present at a concentration of 0.001 to 1% (w/v). In some embodiments, PS80 is present at a concentration of 0.001% to 0.5% (w/v). In some embodiments, PS80 is present at a concentration of 0.01% to 0.5% (w/v). In some embodiments, PS80 is present at a concentration of 0.01% to 0.1% (w/v). In some embodiments, PS80 is present at a concentration of 0.01% to 0.05% (w/v). In some embodiments, PS80 is present at a concentration of 0.01% to 0.04% (w/v). In some embodiments, PS80 is present at a concentration of 0.01% (w/v). In some embodiments, PS80 is present at a concentration of 0.02% (w/v). In some embodiments, PS80 is present at a concentration of 0.03% (w/v). In some embodiments, PS80 is present at a concentration of 0.04% (w/v). In some embodiments, PS80 is present at a concentration of 0.05% (w/v). In some embodiments, PS80 is present at a concentration of 0.06% (w/v). In some embodiments, PS80 is present at a concentration of 0.07% (w/v). In some embodiments, PS80 is present at a concentration of 0.08% (w/v). In some embodiments, PS80 is present at a concentration of 0.09% (w/v). In some embodiments, PS80 is present at a concentration of about 0.1% (w/v).

In some embodiments, the surfactant is polysorbate 20 (PS20). PS20 is also known as polyoxyethylene (20) sorbitan monolaurate.

In some embodiments, PS20 is present at a concentration of 0.001 to 1% (w/v), 0.001% to 0.5% (w/v), 0.01% to 0.5% (w/v), 0.01% to 0.1% (w/v), 0.01% to 0.05% (w/v), 0.01% to 0.04% (w/v), 0.01% (w/v), 0.02% (w/v), 0.03% (w/v), 0.04% (w/v), 0.05% (w/v), 0.06% (w/v), 0.07% (w/v), 0.08% (w/v), 0.09% (w/v), or 0.1% (w/v). In some embodiments, PS20 is present at a concentration of 0.001 to 1% (w/v). In some embodiments, PS20 is present at a concentration of 0.001% to 0.5% (w/v). In some embodiments, PS20 is present at a concentration of 0.01% to 0.5% (w/v). In some embodiments, PS20 is present at a concentration of 0.01% to 0.1% (w/v). In some embodiments, PS20 is present at a concentration of 0.01% to 0.05% (w/v) In some embodiments, PS20 is present at a concentration of 0.01% (w/v). In some embodiments, PS20 is present at a concentration of 0.02% (w/v). In some embodiments, PS20 is present at a concentration of 0.03% (w/v). In some embodiments, PS20 is present at a concentration of 0.04% (w/v). In some embodiments, PS20 is present at a concentration of 0.05% (w/v). In some embodiments, PS20 is present at a concentration of 0.06% (w/v). In some embodiments, PS20 is present at a concentration of 0.07% (w/v). In some embodiments, PS20 is present at a concentration of 0.08% (w/v). In some embodiments, PS20 is present at a concentration of 0.09% (w/v). In some embodiments, PS20 is present at a concentration of about 0.1% (w/v).

In some embodiments, the surfactant is a poloxamer. Poloxamers are nonionic triblock copolymers composed of a central hydrophobic chain of polyoxypropylene (polypropylene oxide)) flanked by two hydrophilic chains of polyoxyethylene (poly(ethylene oxide)). Examples of a poloxamer include, but are not limited to, poloxamer 188, poloxamer 407, poloxamer 184, poloxamer 124, or a combination thereof.

pH

The pH of antibody formulation is crucial for ensuring their stability and efficacy. Low pH can cause antibodies to unfold and aggregate, especially during processes like freeze-thaw cycles. Maintaining an optimal pH is essential to minimize aggregation, which can cause unwanted immunogenic responses in patients. Therefore, selecting the appropriate pH and buffer system is vital in antibody formulation, and is specific to each antibody.

In some embodiments, the pH of the pharmaceutical composition is 4.8 to 6.5. In some embodiments, the pH of the pharmaceutical composition is 4.8 to 6.0. In some embodiments, the pH of the pharmaceutical composition is 4.5 to 6.0. In some embodiments, the pH of the pharmaceutical composition is 5.0 to 6.0. In some embodiments, the pH of the pharmaceutical composition is 4.5 to 5.9, 4.5 to 5.8, 4.5 to 5.7, 4.5 to 5.6, 4.5 to 5.5, 4.6 to 5.9, 4.6 to 5.8, 4.6 to 5.7, 4.6 to 5.6, 4.6 to 5.5, 4.7 to 6.0, 4.7 to 5.9, 4.7 to 5.8, 4.7 to 5.7, 4.7 to 5.6, 4.7 to 5.5, 4.8 to 6.5, 4.8 to 6.0, 4.8 to 5.9, 4.8 to 5.8, 4.8 to 5.7, 4.8 to 5.6, 4.8 to 5.5, 4.9 to 6.0, 4.9 to 5.9, 4.9 to 5.8, 4.9 to 5.7, 4.9 to 5.6, 4.9 to 5.5, 5.0 to 5.9, 5.0 to 5.8, 5.0 to 5.7, 5.0 to 5.6, 5.0 to 5.5, 5.1 to 5.9, 5.1 to 5.8, 5.1 to 5.7, 5.1 to 5.6, 5.1 to 5.5, 5.2 to 5.9, 5.2 to 5.8, 5.2 to 5.7, 5.2 to 5.6, 5.2 to 5.5, 5.3 to 5.9, 5.3 to 5.8, 5.3 to 5.7, 5.3 to 5.6, 5.3 to 5.5, 5.4 to 5.9, 5.4 to 5.8, 5.4 to 5.7, 5.4 to 5.6, 5.4 to 5.5. In some embodiments, the pH of a stable formulation is 4.5 to 6.5. In some embodiments, the pH of a stable formulation is 4.8 to 6.5. In some embodiments, the pH of a stable formulation is 4.8 to 6.0. In some embodiments, the pH of a stable formulation is 5.0 to 6.0. In some embodiments, the pH of a stable formulation is 5.2 to 6.0. In some embodiments, the pH of a stable formulation is 5.2 to 6.5. In some embodiments, the pH of a stable formulation is 4.8 to 5.5. In some embodiments, the pH of a stable formulation is 5.5 to 6.5. In some embodiments, the pH of a stable formulation is 5.0 to 5.5. In some embodiments, the pH of a stable formulation is 5.5 to 6.0.

In some embodiments, the pH of the pharmaceutical composition is 4.5. In some embodiments, the pH of the pharmaceutical composition is 5.0. In some embodiments, the pH of the pharmaceutical composition is 5.5. In some embodiments, the pH of the pharmaceutical composition is 6.0. In some embodiments, the pH of the pharmaceutical composition is 4.5-6.0 (e.g., 4.5, 4.6, 4.7, 4.8, 4.9, 5.0, 5.1, 5.2, 5.3, 5.4, 5.5, 5.6, 5.7, 5.8, 5.9, 6.0) In some embodiments, the pH of the pharmaceutical composition is between 5.0-6.0 (e.g., 5.0, 5.1, 5.2, 5.3, 5.4, 5.5, 5.6, 5.7, 5.8, 5.9, or 6.0). In some embodiments, the pH of the pharmaceutical composition is 4.5. In some embodiments, the pH of the pharmaceutical composition is 4.6. In some embodiments, the pH of the pharmaceutical composition is 4.7. In some embodiments, the pH of the pharmaceutical composition is 4.8. In some embodiments, the pH of the pharmaceutical composition is 4.9. In some embodiments, the pH of the pharmaceutical composition is 5.0. In some embodiments, the pH of the pharmaceutical composition is 5.1. In some embodiments, the pH of the pharmaceutical composition is 5.2. In some embodiments, the pH of the pharmaceutical composition is 5.3. In some embodiments, the pH of the pharmaceutical composition is 5.4. In some embodiments, the pH of the pharmaceutical composition is 5.5. In some embodiments, the pH of the pharmaceutical composition is 5.6. In some embodiments, the pH of the pharmaceutical composition is 5.7. In some embodiments, the pH of the pharmaceutical composition is 5.8. In some embodiments, the pH of the pharmaceutical composition is 5.9. In some embodiments, the pH of the pharmaceutical composition is 6.0. In some embodiments, the pH of a stable formulation is 6.1. In some embodiments, the pH of a stable formulation is 6.2. In some embodiments, the pH of a stable formulation is 6.3. In some embodiments, the pH of a stable formulation is 6.4. In some embodiments, the pH of a stable formulation is 6.5.

### Stability of Antibody Formulations

### Monomer, High Molecular Weight (HMW) Species, Low Molecular Weight (LMW) Species

In some embodiments, a stable formulation is characterized by the amount of high molecular weight (HMW) aggregates. In some embodiments, the amount of high molecular weight (HMW) species, monomer, and low molecular weight (LMW) species are measured by SEC or SEC-UPLC. In some embodiments, the amount of high molecular weight species (HMWS) in the stable formulation is less 10% (e.g., less than 9%, less than 8%, less than 7%, less than 6%, less than 5%, less than 4% less than 3%, less than 2% or less than 1%) as measured by SEC. In some embodiments, the amount of high molecular weight species (HMWS) in the stable formulation is less than 5% as measured by SEC. In some embodiments, the stability based on the HMW or HMWS is based on the composition that is stored at 40 C for a period of time, such as 4 weeks.

In some embodiments, less than 5% of the IGF-1R antibody exists as HMW species in the formulation upon storage at 5° C. for at least 9 months. In some embodiments, less than 4% of the IGF-1R antibody exists as HMW species in the formulation upon storage at 5° C. for at least 9 months. In some embodiments, less than 3% of the IGF-1R antibody exists as HMW species in the formulation upon storage at 5° C. for at least 9 months. In some embodiments, less than 2% of the IGF-1R antibody exists as HMW species in the formulation upon storage at 5° C. for at least 9 months. In some embodiments, less than 1.5% of the IGF-1R antibody exists as HMW species in the formulation upon storage at 5° C. for at least 9 months. In some embodiments, less than 1% of the IGF-1R antibody exists as HMW species in the formulation upon storage at 5° C. for at least 9 months.

In some embodiments, less than 5% of the IGF-1R antibody exists as HMW species in the formulation upon storage at 25° C. for at least 6 months. In some embodiments, less than 4% of the IGF-1R antibody exists as HMW species in the formulation upon storage at 25° C. for at least 6 months. In some embodiments, less than 3% of the IGF-1R antibody exists as HMW species in the formulation upon storage at 25° C. for at least 6 months. In some embodiments, less than 2% of the IGF-1R antibody exists as HMW species in the formulation upon storage at 25° C. for at least 6 months. In some embodiments, less than 1.5% of the IGF-1R antibody exists as HMW species in the formulation upon storage at 25° C. for at least 6 months. In some embodiments, less than 1% of the IGF-1R antibody exists as HMW species in the formulation upon storage at 25° C. for at least 6 months.

In some embodiments, more than 90% of the IGF-1R antibody exists as monomer species in the formulation upon storage at 5° C. for at least 9 months. In some embodiments, more than 95% of the IGF-1R antibody exists as monomer species in the formulation upon storage at 5° C. for at least 9 months. In some embodiments, more than 96% of the IGF-1R antibody exists as monomer species in the formulation upon storage at 5° C. for at least 9 months. In some embodiments, more than 97% of the IGF-1R antibody exists as monomer species in the formulation upon storage at 5° C. for at least 9 months. In some embodiments, more than 98% of the IGF-1R antibody exists as monomer species in the formulation upon storage at 5° C. for at least 9 months. In some embodiments, more than 99% of the IGF-1R antibody exists as monomer species in the formulation upon storage at 5° C. for at least 9 months.

In some embodiments, more than 90% of the IGF-1R antibody exists as monomer species in the formulation upon storage at 25° C. for at least 6 months. In some embodiments, more than 95% of the IGF-1R antibody exists as monomer species in the formulation upon storage at 25° C. for at least 6 months. In some embodiments, more than 96% of the IGF-1R antibody exists as monomer species in the formulation upon storage at 25° C. for at least 6 months. In some embodiments, more than 97% of the IGF-1R antibody exists as monomer species in the formulation upon storage at 25° C. for at least 6 months. In some embodiments, more than 98% of the IGF-1R antibody exists as monomer species in the formulation upon storage at 25° C. for at least 6 months. In some embodiments, more than 99% of the IGF-1R antibody exists as monomer species in the formulation upon storage at 25° C. for at least 6 months.

In some embodiments, less than 5% of the IGF-1R antibody exists as LMW species in the formulation upon storage at 5° C. for at least 9 months. In some embodiments, less than 4% of the IGF-1R antibody exists as LMW species in the formulation upon storage at 5° C. for at least 9 months. In some embodiments, less than 3% of the IGF-1R antibody exists as LMW species in the formulation upon storage at 5° C. for at least 9 months. In some embodiments, less than 2% of the IGF-1R antibody exists as LMW species in the formulation upon storage at 5° C. for at least 9 months. In some embodiments, less than 1.5% of the IGF-1R antibody exists as LMW species in the formulation upon storage at 5° C. for at least 9 months. In some embodiments, less than 1% of the IGF-1R antibody exists as LMW species in the formulation upon storage at 5° C. for at least 9 months.

In some embodiments, less than 5% of the IGF-1R antibody exists as LMW species in the formulation upon storage at 25° C. for at least 6 months. In some embodiments, less than 4% of the IGF-1R antibody exists as LMW species in the formulation upon storage at 25° C. for at least 6 months. In some embodiments, less than 3% of the IGF-1R antibody exists as LMW species in the formulation upon storage at 25° C. for at least 6 months. In some embodiments, less than 2% of the IGF-1R antibody exists as LMW species in the formulation upon storage at 25° C. for at least 6 months. In some embodiments, less than 1.5% of the IGF-1R antibody exists as LMW species in the formulation upon storage at 25° C. for at least 6 months. In some embodiments, less than 1% of the IGF-1R antibody exists as LMW species in the formulation upon storage at 25° C. for at least 6 months.

In some embodiments, % of monomers is determined by size-exclusion chromatography (SEC). In some embodiments, % of HMW is determined by size-exclusion chromatography (SEC). In some embodiments, % of LMW is determined by size-exclusion chromatography (SEC). Size exclusion ultra-performance liquid chromatography (SEC-UPLC) is a purity analysis method that identifies, separates, and purifies proteins based on the size of the molecule. SEC works by inhibiting the access of large molecules to the stationary particles lining the inner surface of SEC column. This results in larger particles eluting quickly while small molecules retain longer in the SEC column due to their hydrophobic interactions with the adsorbing materials. Herein, the separation of pure monomers from aggregates, high molecular weight (HMW), and fragments, low molecular weight (LMW), is pertinent for determining stability and aggregation of the relevant compound. Retaining the highest percent monomer over time and under stress conditions indicates greater stability of the compound.

### Thermal and Conformational Stability

In some embodiments, the Tm onset is higher than 56° C. as measured by DSC. In some embodiments, the Tm onset is higher than 57° C. as measured by DSC. In some embodiments, the Tm onset is higher than 58° C. as measured by DSC. In some embodiments, the Tm onset is higher than 59° C. as measured by DSC. In some embodiments, the Tm onset is higher than 60° C. as measured by DSC. In some embodiments, the Tm onset is higher than 61° C. as measured by DSC. In some embodiments, the Tm onset is higher than 62° C. as measured by DSC. In some embodiments, the Tm onset is higher than 63° C. as measured by DSC. In some embodiments, the Tm onset is higher than 64° C. as measured by DSC.

Differential Scanning calorimeter is a thermoanalytical technique measuring the difference in the amount of heat required to increase the temperature of a sample and reference as a function of temperature. There are several characteristics of a sample this technique can quantify, herein the glass transition was measured. Glass transitions can occur as the temperature of an amorphous solid increases. These transitions appear as a step in the baseline of the recorded DSC signal due to the sample undergoing a change in heat capacity. Modulated DSC superimposes the underlying heating rate by a sinusoidal temperature variation. This allows the separation of overlapping DSC effects by calculating the reversing and the non-reversing signals. The reversing heat flow is related to the changes in specific heat capacity (glass transition) while the non-reversing heat flow corresponds to time-dependent phenomena. This technique informs on the thermal stability of protein samples and assesses conformational differences between them.

Imaged Capillary Isoelectric Focusing (iCIEF)

Imaged Capillary Isoelectric Focusing (iCIEF) is used to monitor the distribution of the charge variants. The isoelectric point (pI), being an intrinsic property of a specific protein, is the pH at which the protein molecule does not carry net electrical charge. Under an external electric field, the charge variants move along a continuous pH gradient formed by carrier ampholytes and stop at where the pH equals its pI. This technique used to characterize and quantify protein charge variants.

In some embodiments, the shift in charge profiles may be measured through iCIEF the relative percentages of main peak, acid peak and/or basic peak. For example, a stable formulation can be one in which the change in measured acidic peak increases by less than 40% (e.g., less than 38%, 36%, 34%, 32%, 30%, 28%, 26%, 24%, 22% or less than 20%) relative to an initial value following storage at 40° C. for four weeks as measured by iCIEF and quantified using chromatographic software. In some embodiments, a stable formulation is one in which the main peak percentage decreases by less than 35% (e.g., less than 34%, less than 33%, less than 32%, less than 31%, less than 30%, less than 29%, less than 28%, less than 27%, less than 26%, less than 25%, than less than 24%, less than 23%, less than 22%, less than 21% or less than 20%) relative to an initial value following storage at 40° C. for four weeks as measured by iCIEF and quantified using chromatographic software. In some embodiments, the stable formulation basic peak, measured by iCIEF, decreases in area percent by less than 6.5% (e.g., less than 6%, less than 5%, less than 4%, less than 3%, less than 2%, or less than 1%) after 4 weeks at a temperature of 40° C., relative to the initial value.

In some embodiments, the acidic peak increases by less than 50% upon storage at 40° C. for at least four weeks as measured by iCIEF. In some embodiments, the acidic peak increases by less than 45% upon storage at 40° C. for at least four weeks as measured by iCIEF. In some embodiments, the acidic peak increases by less than 40% upon storage at 40° C. for at least four weeks as measured by iCIEF. In some embodiments, the acidic peak increases by less than 35% upon storage at 40° C. for at least four weeks as measured by iCIEF. In some embodiments, the acidic peak increases by less than 30% upon storage at 40° C. for at least four weeks as measured by iCIEF.

In some embodiments, the main peak decreases by less than 50% upon storage at 40° C. for at least four weeks as measured by iCIEF. In some embodiments, the main peak decreases by less than 45% upon storage at 40° C. for at least four weeks as measured by iCIEF. In some embodiments, the main peak decreases by less than 40% upon storage at 40° C. for at least four weeks as measured by iCIEF. In some embodiments, the main peak decreases by less than 35% upon storage at 40° C. for at least four weeks as measured by iCIEF. In some embodiments, the main peak decreases by less than 30% upon storage at 40° C. for at least four weeks as measured by iCIEF.

In some embodiments, the basic peak decreases by less than 10% upon storage at 40° C. for at least four weeks as measured by iCIEF. In some embodiments, the basic peak decreases by less than 8% upon storage at 40° C. for at least four weeks as measured by iCIEF. In some embodiments, the basic peak decreases by less than 7% upon storage at 40° C. for at least four weeks as measured by iCIEF. In some embodiments, the basic peak decreases by less than 6.5% upon storage at 40° C. for at least four weeks as measured by iCIEF. In some embodiments, the basic peak decreases by less than 6% upon storage at 40° C. for at least four weeks as measured by iCIEF. In some embodiments, the basic peak decreases by less than 5.5% upon storage at 40° C. for at least four weeks as measured by iCIEF. In some embodiments, the basic peak decreases by less than 5% upon storage at 40° C. for at least four weeks as measured by iCIEF.

### Purity

In some embodiments, the decrease in purity is less than 5%, less than 4%, less than 3% or less than 2.5% upon storage at 5° C. for at least three months. In some embodiments, the decrease in purity is less than 5% upon storage at 5° C. for at least three months. In some embodiments, the decrease in purity is less than 4%, upon storage at 5° C. for at least three months. In some embodiments, the decrease in purity is less than 3%, upon storage at 5° C. for at least three months. In some embodiments, the decrease in purity is less than 2%, upon storage at 5° C. for at least three months. In some embodiments, the decrease in purity is less than 1%, upon storage at 5° C. for at least three months.

In some embodiments, the decrease in purity is less than 5%, less than 4%, less than 3% or less than 2.5% upon storage at 25° C. for at least three months. In some embodiments, the decrease in purity is less than 5% upon storage at 25° C. for at least three months. In some embodiments, the decrease in purity is less than 4%, upon storage at 25° C. for at least three months. In some embodiments, the decrease in purity is less than 3%, upon storage at 25° C. for at least three months. In some embodiments, the decrease in purity is less than 2%, upon storage at 25° C. for at least three months. In some embodiments, the decrease in purity is less than 1%, upon storage at 25° C. for at least three months.

In some embodiments, the decrease in purity is less than 5%, less than 4%, less than 3% or less than 2.5% upon storage at 40° C. for at least four weeks. In some embodiments, the decrease in purity is less than 5% upon storage at 40° C. for at least four weeks. In some embodiments, the decrease in purity is less than 4%, upon storage at 40° C. for at least four weeks. In some embodiments, the decrease in purity is less than 3%, upon storage at 40° C. for at least four weeks. In some embodiments, the decrease in purity is less than 2%, upon storage at 40° C. for at least four weeks. In some embodiments, the decrease in purity is less than 1%, upon storage at 40° C. for at least four weeks.

Non-reduced Capillary Electrophoresis-Sodium Dodecyl Sulfate (CE-SDS-NR) is a capillary electrophoresis method that allows protein separation by size driven by a high-voltage direct current electric field. This technique is a purity analysis method that separates proteins based on their electrophoretic mobility, where proteins of smaller sizes move faster, and larger sizes move slower.

In some embodiments, caliper sodium dodecyl sulfate (Caliper-SDS) is used to assess the purity of the stable formulation. In some embodiments, caliper sodium dodecyl sulfate reduced (Caliper-SDS-R) is used to assess the purity of the stable formulation. In some embodiments, caliper sodium dodecyl sulfate non-reduced (Caliper-SDS-NR) is used to assess the purity of the stable formulation. In some embodiments, capillary sodium dodecyl sulfate (CE-SDS) is used to assess the purity of the stable formulation.

### Appearance/Aggregation

Appearance is a visual confirmation of a sample compared to a standard. The appearance of all samples is assessed for visible particles, clarity and color. This is routinely examined against black and white backgrounds using a Clarity Detector to shine light directly onto the samples. This technique measures stability.

In some embodiments, the stable formulation clarity is 24 NTU or less. In some embodiments, the stable formulation clarity is 20 NTU or less. In some embodiments, the stable formulation clarity is 20 NTU or less. In some embodiments, the stable formulation clarity is 18 NTU or less. In some embodiments, the stable formulation clarity is 16 NTU or less.

In some embodiments, the stable formulation has less than or equal to five visible particles upon storage at 5° C. for at least 9 months. In some embodiments, the stable formulation has less than or equal to five visible particles upon storage at 25° C. for at least six months. In some embodiments, the stable formulation has less than or equal to five visible particles upon storage at 40° C. for at least two weeks.

In some embodiments, the stable formulation is free of particles upon storage at 5° C. for at least 9 months. In some embodiments, the stable formulation is free of particles upon storage at 25° C. for at least six months. In some embodiments, the stable formulation is free of particles upon storage at 40° C. for at least two weeks.

### Exemplary Anti-IGF-IR Antibody Formulation

In some embodiments, the pharmaceutical composition comprising an antibody provided for herein, or a variant thereof, comprises sucrose, methionine, PS80 or PS20, and histidine (e.g., L-histidine) at a pH of 5 to 6, such as a pH of 5.5. In some embodiments, the sucrose is at 7 to 9% (w/v), such as 8%. In some embodiments, methionine is at a concentration of 5 to 15 mM, such as 10 mM. In some embodiments, PS80 or PS20 is at a concentration of 0.01 to 0.05% (w/v), such as 0.2%. In some embodiments, a histidine buffer, is at a concentration of 15 to 25 mM, such as 20 mM.

In some embodiments, a pharmaceutical composition comprises: (i) an anti-IGF-1R antibody, or antigen-binding fragment thereof; (ii) a buffer at a concentration of 10-60 mM; (iii) sucrose at a concentration of 1-20% (w/v); (iv) a stabilizer, or an anti-oxidant, at a concentration of 1-15 mM; and (v) a surfactant at a concentration of 0.001-1% (w/v). In some embodiments, the pharmaceutical composition further comprises a pH of 4.5 to 6.0. In some embodiments, the antibody is such as those provided herein (e.g., antibodies described in Tables 2-7).

In some embodiments, a pharmaceutical composition comprises (i) an anti-IGF-1R antibody, or antigen-binding fragment thereof (e.g., an anti-IGF-1R antibody, described in Tables 2-7), at a concentration of 10-300 mg/mL; (ii) a histidine buffer at a concentration of 10-60 mM; (iii) sucrose at a concentration of 1-20% (w/v); (iv) methionine, or L-methionine, at a concentration of 1-15 mM; and (v) a polysorbate at a concentration of 0.001-1% (w/v). In some embodiments, the pharmaceutical composition further comprises a pH of 4.5 to 6.0. In some embodiments, the pH of the formulation is 4.8. In some embodiments, the pH of the formulation is 5.5.

In some embodiments, a pharmaceutical composition comprises (i) an anti-IGF-1R antibody, or antigen-binding fragment thereof (e.g., an anti-IGF-1R antibody, described in Tables 2-7 or a variant thereof), at a concentration of 10-200 mg/mL; (ii) a histidine buffer at a concentration of 10-40 mM; (iii) sucrose at a concentration of 1-10% (w/v); (iv) methionine, or L-methionine, at a concentration of 1-15 mM; and (v) a polysorbate at a concentration of 0.01-0.1% (w/v).

In some embodiments, a pharmaceutical composition comprises: (i) an anti-IGF-1R antibody, or antigen-binding fragment thereof (e.g., an anti-IGF-1R antibody, described in Tables 2-7 or a variant thereof), at a concentration of 25 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.04% (w/v).

In some embodiments, a pharmaceutical composition comprises: (i) an anti-IGF-1R antibody, or antigen-binding fragment thereof ((e.g., an anti-IGF-1R antibody, described in Tables 2-7 or a variant thereof), at a concentration of 25 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.02% (w/v).

In some embodiments, a pharmaceutical composition comprises: (i) an anti-IGF-1R antibody, or antigen-binding fragment thereof (e.g., an anti-IGF-1R antibody, described in Tables 2-7 or a variant thereof), at a concentration of 25 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.04% (w/v).

In some embodiments, a pharmaceutical composition comprises: (i) an anti-IGF-1R antibody, or antigen-binding fragment thereof (e.g., an anti-IGF-1R antibody, described in Tables 2-7 or a variant thereof), at a concentration of 25 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.02% (w/v).

In some embodiments, a pharmaceutical composition comprises: (i) an anti-IGF-1R antibody, or antigen-binding fragment thereof (e.g., an anti-IGF-1R antibody, described in Tables 2-7 or a variant thereof), at a concentration of 50 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.04% (w/v).

In some embodiments, a pharmaceutical composition comprises: (i) an anti-IGF-1R antibody, or antigen-binding fragment thereof (e.g., an anti-IGF-1R antibody, described in Tables 2-7 or a variant thereof), at a concentration of 50 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.02% (w/v).

In some embodiments, a pharmaceutical composition comprises: (i) an anti-IGF-1R antibody, or antigen-binding fragment (e.g., an anti-IGF-1R antibody, described in Tables 2-7 or a variant thereof), at a concentration of 50 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.04% (w/v).

In some embodiments, a pharmaceutical composition comprises: (i) an anti-IGF-1R antibody, or antigen-binding fragment thereof (e.g., an anti-IGF-1R antibody, described in Tables 2-7 or a variant thereof), at a concentration of 50 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.02% (w/v).

In some embodiments, a pharmaceutical composition comprises: (i) an anti-IGF-1R antibody, or antigen-binding fragment thereof (e.g., an anti-IGF-1R antibody, described in Tables 2-7 or a variant thereof), at a concentration of 100 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.04% (w/v).

In some embodiments, a pharmaceutical composition comprises: (i) an anti-IGF-1R antibody, or antigen-binding fragment thereof (e.g., an anti-IGF-1R antibody, described in Tables 2-7 or a variant thereof), at a concentration of 100 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.02% (w/v).

In some embodiments, a pharmaceutical composition comprises: (i) an anti-IGF-1R antibody, or antigen-binding fragment thereof (e.g., an anti-IGF-1R antibody, described in Tables 2-7 or a variant thereof), at a concentration of 100 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.04% (w/v).

In some embodiments, a pharmaceutical composition comprises: (i) an anti-IGF-1R antibody, or antigen-binding fragment thereof (e.g., an anti-IGF-1R antibody, described in Tables 2-7 or a variant thereof), at a concentration of 100 mg/mL; (ii) a histidine buffer at a concentration of 20 mM; (iii) sucrose at a concentration of 8% (w/v); (iv) L-methionine at a concentration of 10 mM; and (v) a polysorbate at a concentration of about 0.02% (w/v).

In some embodiments, a pharmaceutical composition comprises: (i) an anti-IGF-1R antibody, or antigen-binding fragment thereof (e.g., an anti-IGF-1R antibody, described in Tables 2-7 or a variant thereof), at a concentration of 150 mg/mL; (ii) a histidine buffer at a concentration of 20 mM; (iii) sucrose at a concentration of about 8% (w/v); (iv) methionine at a concentration of 10 mM; and (v) a polysorbate at a concentration of 0.04% (w/v).

In some embodiments, a pharmaceutical composition comprises: (i) an anti-IGF-1R antibody, or antigen-binding fragment thereof (e.g., an anti-IGF-1R antibody, described in Tables 2-7 or a variant thereof), at a concentration of 150 mg/mL; (ii) a histidine buffer at a concentration of 20 mM; (iii) sucrose at a concentration of about 8% (w/v); (iv) methionine at a concentration of 10 mM; and (v) a polysorbate at a concentration of 0.02% (w/v).

In some embodiments, a pharmaceutical composition comprises: (i) an anti-IGF-1R antibody, or antigen-binding fragment thereof (e.g., an anti-IGF-1R antibody, described in Tables 2-7 or a variant thereof), at a concentration of 150 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.04% (w/v).

In some embodiments, a pharmaceutical composition comprises: (i) an anti-IGF-1R antibody, or antigen-binding fragment thereof (e.g., an anti-IGF-1R antibody, described in Tables 2-7 or a variant thereof), at a concentration of 150 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.02% (w/v).

In some embodiments, a pharmaceutical composition comprises: (i) an anti-IGF-1R antibody, or antigen-binding fragment thereof, comprising a light chain comprising the amino acid sequence of SEQ ID NO: 1 and a heavy chain comprising the amino acid sequence of SEQ ID NO: 2, or a variant thereof, at a concentration of 25 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 1 and a heavy chain comprising the amino acid sequence of SEQ ID NO: 2, or a variant thereof, at a concentration of 25 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 1 and a heavy chain comprising the amino acid sequence of SEQ ID NO: 2, or a variant thereof, at a concentration of 25 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 1 and a heavy chain comprising the amino acid sequence of SEQ ID NO: 2, or a variant thereof, at a concentration of 25 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 3 and a heavy chain comprising the amino acid sequence of SEQ ID NO: 4, or a variant thereof, at a concentration of about 25 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 3 and a heavy chain comprising the amino acid sequence of SEQ ID NO: 4, or a variant thereof, at a concentration of 25 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 3 and a heavy chain comprising the amino acid sequence of SEQ ID NO: 4, or a variant thereof, at a concentration of 25 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 3 and a heavy chain comprising the amino acid sequence of SEQ ID NO: 4, or a variant thereof, at a concentration of 25 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 5 and a heavy chain comprising the amino acid sequence of SEQ ID NO: 6, or a variant thereof, at a concentration of 25 mg/mL; (ii) a histidine buffer at a concentration of 20 mM; (iii) sucrose at a concentration of 8% (w/v); (iv) methionine at a concentration of 10 mM; and (v) a polysorbate at a concentration of about 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 5 and a heavy chain comprising the amino acid sequence of SEQ ID NO: 6, or a variant thereof, at a concentration of 25 mg/mL; (ii) a histidine buffer at a concentration of 20 mM; (iii) sucrose at a concentration of 8% (w/v); (iv) methionine at a concentration of about 10 mM; and (v) a polysorbate at a concentration of about 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 5 and a heavy chain comprising the amino acid sequence of SEQ ID NO: 6, or a variant thereof, at a concentration of 25 mg/mL; (ii) a histidine buffer at a concentration of 20 mM; (iii) sucrose at a concentration of 8% (w/v); (iv) L-methionine at a concentration of 10 mM; and (v) a polysorbate at a concentration of about 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 5 and a heavy chain comprising the amino acid sequence of SEQ ID NO: 6, or a variant thereof, at a concentration of 25 mg/mL; (ii) a histidine buffer at a concentration of 20 mM; (iii) sucrose at a concentration of 8% (w/v); (iv) L-methionine at a concentration of 10 mM; and (v) a polysorbate at a concentration of about 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 7 and a heavy chain comprising the amino acid sequence of SEQ ID NO: 8, or a variant thereof, at a concentration of 25 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 7 and a heavy chain comprising the amino acid sequence of SEQ ID NO: 8, or a variant thereof, at a concentration of 25 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.02% (w/v); wherein the pharmaceutical composition is at a pH of 5.5. In some embodiments, a pharmaceutical composition a stable formulation comprises: (i) an anti-IGF-1R antibody, or antigen-binding fragment thereof, comprising a light chain comprising the amino acid sequence of SEQ ID NO: 7 and a heavy chain comprising the amino acid sequence of SEQ ID NO: 8, or a variant thereof, at a concentration of 25 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 7 and a heavy chain comprising the amino acid sequence of SEQ ID NO: 8, or a variant thereof, at a concentration of 25 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 9 and a heavy chain comprising the amino acid sequence of SEQ ID NO: 10, or a variant thereof, at a concentration of 25 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 9 and a heavy chain comprising the amino acid sequence of SEQ ID NO: 10, or a variant thereof, at a concentration of 25 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 9 and a heavy chain comprising the amino acid sequence of SEQ ID NO: 10, or a variant thereof, at a concentration of 25 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 9 and a heavy chain comprising the amino acid sequence of SEQ ID NO: 10, or a variant thereof, at a concentration of 25 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 11 and a heavy chain comprising the amino acid sequence of SEQ ID NO: 12, or a variant thereof, at a concentration of 25 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 11 and a heavy chain comprising the amino acid sequence of SEQ ID NO: 12, or a variant thereof, at a concentration of 25 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 11 and a heavy chain comprising the amino acid sequence of SEQ ID NO: 12, or a variant thereof, at a concentration of 25 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 11 and a heavy chain comprising the amino acid sequence of SEQ ID NO: 12, or a variant thereof, at a concentration of 25 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a V_{L} of SEQ ID NO: 13 and a V_{H} of SEQ ID NO: 14; or a light chain of SEQ ID NO: 93 and a heavy chain of SEQ ID NO: 92; or a light chain of SEQ ID NO: 93 and a heavy chain of SEQ ID NO: 94, or a variant thereof, at a concentration of 25 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a V_{L} of SEQ ID NO: 13 and a V_{H} of SEQ ID NO: 14; or a light chain of SEQ ID NO: 93 and a heavy chain of SEQ ID NO: 92; or a light chain of SEQ ID NO: 93 and a heavy chain of SEQ ID NO: 94, or a variant thereof, at a concentration of 25 mg/ml; (ii) a histidine buffer at a concentration of 20 mM; (iii) sucrose at a concentration of 8% (w/v); (iv) methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a V_{L} of SEQ ID NO: 13 and a V_{H} of SEQ ID NO: 14; or a light chain of SEQ ID NO: 93 and a heavy chain of SEQ ID NO: 92; or a light chain of SEQ ID NO: 93 and a heavy chain of SEQ ID NO: 94, or a variant thereof, at a concentration of 25 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a V_{L} of SEQ ID NO: 13 and a V_{H} of SEQ ID NO: 14; or a light chain of SEQ ID NO: 93 and a heavy chain of SEQ ID NO: 92; or a light chain of SEQ ID NO: 93 and a heavy chain of SEQ ID NO: 94, or a variant thereof, at a concentration of 25 mg/ml; (ii) a histidine buffer at a concentration of 20 mM; (iii) sucrose at a concentration of 8% (w/v); (iv) L-methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 15 and a heavy chain comprising the amino acid sequence of SEQ ID NO: 16, or a variant thereof, at a concentration of 25 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 15 and a heavy chain comprising the amino acid sequence of SEQ ID NO: 16, or a variant thereof, at a concentration of 25 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 15 and a heavy chain comprising the amino acid sequence of SEQ ID NO: 16, or a variant thereof, at a concentration of 25 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 15 and a heavy chain comprising the amino acid sequence of SEQ ID NO: 16, or a variant thereof, at a concentration of 25 mg/ml; (ii) a histidine buffer at a concentration of 20 mM; (iii) sucrose at a concentration of 8% (w/v); (iv) L-methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 79 and a heavy chain comprising the amino acid sequence of SEQ ID NO: 80, or a variant thereof, at a concentration of 25 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 79 and a heavy chain comprising the amino acid sequence of SEQ ID NO: 80, or a variant thereof, at a concentration of 25 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 79 and a heavy chain comprising the amino acid sequence of SEQ ID NO: 80, or a variant thereof, at a concentration of 25 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 79 and a heavy chain comprising the amino acid sequence of SEQ ID NO: 80, or a variant thereof, at a concentration of 25 mg/ml; (ii) a histidine buffer at a concentration of 20 mM; (iii) sucrose at a concentration of 8% (w/v); (iv) L-methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 3 and a heavy chain comprising the amino acid sequence of SEQ ID NO: 83, or a variant thereof, at a concentration of 25 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 3 and a heavy chain comprising the amino acid sequence of SEQ ID NO: 83, or a variant thereof, at a concentration of 25 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 3 and a heavy chain comprising the amino acid sequence of SEQ ID NO: 83, or a variant thereof, at a concentration of 25 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 3 and a heavy chain comprising the amino acid sequence of SEQ ID NO: 83, or a variant thereof, at a concentration of 25 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 100 and a heavy chain comprising the amino acid sequence of SEQ ID NO: 101, or a variant thereof, at a concentration of 25 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 100 and a heavy chain comprising the amino acid sequence of SEQ ID NO: 101, or a variant thereof, at a concentration of 25 mg/mL; (ii) a histidine buffer at a concentration of about 20 mM; (iii) sucrose at a concentration of 8% (w/v); (iv) methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 100 and a heavy chain comprising the amino acid sequence of SEQ ID NO: 101, or a variant thereof, at a concentration of 25 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 100 and a heavy chain comprising the amino acid sequence of SEQ ID NO: 101, or a variant thereof, at a concentration of 25 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.02% (w/v); wherein the pharmaceutical composition is at a pH of 5.5.

In some embodiments, a pharmaceutical composition comprises: (i) an anti-IGF-1R antibody, or antigen-binding fragment thereof, comprising a light chain comprising the amino acid sequence of SEQ ID NO: 1 and a heavy chain comprising the amino acid sequence of SEQ ID NO: 2, or a variant thereof, at a concentration of 50 mg/mL; (ii) a histidine buffer at a concentration of 20 mM; (iii) sucrose at a concentration of about 8% (w/v); (iv) methionine at a concentration of 10 mM; and (v) a polysorbate at a concentration of 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 1 and a heavy chain comprising the amino acid sequence of SEQ ID NO: 2, or a variant thereof, at a concentration of 50 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 1 and a heavy chain comprising the amino acid sequence of SEQ ID NO: 2, or a variant thereof, at a concentration of 50 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 1 and a heavy chain comprising the amino acid sequence of SEQ ID NO: 2, or a variant thereof, at a concentration of 50 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 3 and a heavy chain comprising the amino acid sequence of SEQ ID NO: 4, or a variant thereof, at a concentration of 50 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 3 and a heavy chain comprising the amino acid sequence of SEQ ID NO: 4, or a variant thereof, at a concentration of 50 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 3 and a heavy chain comprising the amino acid sequence of SEQ ID NO: 4, or a variant thereof, at a concentration of 50 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 3 and a heavy chain comprising the amino acid sequence of SEQ ID NO: 4, or a variant thereof, at a concentration of 50 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 5 and a heavy chain comprising the amino acid sequence of SEQ ID NO: 6, or a variant thereof, at a concentration of 50 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 5 and a heavy chain comprising the amino acid sequence of SEQ ID NO: 6, or a variant thereof, at a concentration of 50 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 5 and a heavy chain comprising the amino acid sequence of SEQ ID NO: 6, or a variant thereof, at a concentration of 50 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 5 and a heavy chain comprising the amino acid sequence of SEQ ID NO: 6, or a variant thereof, at a concentration of 50 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 7 and a heavy chain comprising the amino acid sequence of SEQ ID NO: 8, or a variant thereof, at a concentration of 50 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 7 and a heavy chain comprising the amino acid sequence of SEQ ID NO: 8, or a variant thereof, at a concentration of 50 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 7 and a heavy chain comprising the amino acid sequence of SEQ ID NO: 8, or a variant thereof, at a concentration of 50 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 7 and a heavy chain comprising the amino acid sequence of SEQ ID NO: 8, or a variant thereof, at a concentration of 50 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 9 and a heavy chain comprising the amino acid sequence of SEQ ID NO: 10, or a variant thereof, at a concentration of 50 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 9 and a heavy chain comprising the amino acid sequence of SEQ ID NO: 10, or a variant thereof, at a concentration of 50 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 9 and a heavy chain comprising the amino acid sequence of SEQ ID NO: 10, or a variant thereof, at a concentration of 50 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 9 and a heavy chain comprising the amino acid sequence of SEQ ID NO: 10, or a variant thereof, at a concentration of 50 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 11 and a heavy chain comprising the amino acid sequence of SEQ ID NO: 12, or a variant thereof, at a concentration of 50 mg/mL; (ii) a histidine buffer at a concentration of 20 mM; (iii) sucrose at a concentration of about 8% (w/v); (iv) methionine at a concentration of about 10 mM; and (v) a polysorbate at a concentration of about 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 11 and a heavy chain comprising the amino acid sequence of SEQ ID NO: 12, or a variant thereof, at a concentration of 50 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 11 and a heavy chain comprising the amino acid sequence of SEQ ID NO: 12, or a variant thereof, at a concentration of 50 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 11 and a heavy chain comprising the amino acid sequence of SEQ ID NO: 12, or a variant thereof, at a concentration of 50 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a V_{L} of SEQ ID NO: 13 and a V_{H} of SEQ ID NO: 14; or a light chain of SEQ ID NO: 93 and a heavy chain of SEQ ID NO: 92; or a light chain of SEQ ID NO: 93 and a heavy chain of SEQ ID NO: 94, or a variant thereof, at a concentration of 50 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a V_{L} of SEQ ID NO: 13 and a V_{H} of SEQ ID NO: 14; or a light chain of SEQ ID NO: 93 and a heavy chain of SEQ ID NO: 92; or a light chain of SEQ ID NO: 93 and a heavy chain of SEQ ID NO: 94, or a variant thereof, at a concentration of 50 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a V_{L} of SEQ ID NO: 13 and a V_{H} of SEQ ID NO: 14; or a light chain of SEQ ID NO: 93 and a heavy chain of SEQ ID NO: 92; or a light chain of SEQ ID NO: 93 and a heavy chain of SEQ ID NO: 94, or a variant thereof, at a concentration of 50 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a V_{L} of SEQ ID NO: 13 and a V_{H} of SEQ ID NO: 14; or a light chain of SEQ ID NO: 93 and a heavy chain of SEQ ID NO: 92; or a light chain of SEQ ID NO: 93 and a heavy chain of SEQ ID NO: 94, or a variant thereof, at a concentration of 50 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 15 and a heavy chain comprising the amino acid sequence of SEQ ID NO: 16, or a variant thereof, at a concentration of 50 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 15 and a heavy chain comprising the amino acid sequence of SEQ ID NO: 16, or a variant thereof, at a concentration of 50 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 15 and a heavy chain comprising the amino acid sequence of SEQ ID NO: 16, or a variant thereof, at a concentration of 50 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 15 and a heavy chain comprising the amino acid sequence of SEQ ID NO: 16, or a variant thereof, at a concentration of 50 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 79 and a heavy chain comprising the amino acid sequence of SEQ ID NO: 80, or a variant thereof, at a concentration of 50 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 79 and a heavy chain comprising the amino acid sequence of SEQ ID NO: 80, or a variant thereof, at a concentration of 50 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 79 and a heavy chain comprising the amino acid sequence of SEQ ID NO: 80, or a variant thereof, at a concentration of 50 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 79 and a heavy chain comprising the amino acid sequence of SEQ ID NO: 80, or a variant thereof, at a concentration of 50 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 3 and a heavy chain comprising the amino acid sequence of SEQ ID NO: 83, or a variant thereof, at a concentration of 50 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 3 and a heavy chain comprising the amino acid sequence of SEQ ID NO: 83, or a variant thereof, at a concentration of 50 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 3 and a heavy chain comprising the amino acid sequence of SEQ ID NO: 83, or a variant thereof, at a concentration of 50 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 3 and a heavy chain comprising the amino acid sequence of SEQ ID NO: 83, or a variant thereof, at a concentration of 50 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 100 and a heavy chain comprising the amino acid sequence of SEQ ID NO: 101, or a variant thereof, at a concentration of 50 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 100 and a heavy chain comprising the amino acid sequence of SEQ ID NO: 101, or a variant thereof, at a concentration of 50 mg/mL; (ii) a histidine buffer at a concentration of 20 mM; (iii) sucrose at a concentration of 8% (w/v); (iv) methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 100 and a heavy chain comprising the amino acid sequence of SEQ ID NO: 101, or a variant thereof, at a concentration of 50 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.04% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 100 and a heavy chain comprising the amino acid sequence of SEQ ID NO: 101, or a variant thereof, at a concentration of 50 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.02% (w/v); wherein the pharmaceutical composition is at a pH of 5.5.

In some embodiments, a pharmaceutical composition comprises: (i) an anti-IGF-1R antibody, or antigen-binding fragment thereof, comprising a light chain variable region comprising the amino acid sequence of SEQ ID NO: 98 and a heavy chain variable region comprising the amino acid sequence of SEQ ID NO: 99, or a variant thereof, at a concentration of 50 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain variable region comprising the amino acid sequence of SEQ ID NO: 98 and a heavy chain variable region comprising the amino acid sequence of SEQ ID NO: 99, or a variant thereof, at a concentration of 150 mg/mL; (ii) a histidine buffer at a concentration of 20 mM; (iii) sucrose at a concentration of 8% (w/v); (iv) L-methionine at a concentration of 10 mM; and (v) a polysorbate at a concentration of about 0.04% (w/v); wherein the pharmaceutical composition is at a pH of 5.5.

In some embodiments, a pharmaceutical composition comprises: (i) an anti-IGF-1R antibody, or antigen-binding fragment thereof, comprising a light chain comprising the amino acid sequence of SEQ ID NO: 3 and a heavy chain comprising the amino acid sequence of SEQ ID NO: 83, or a variant thereof, at a concentration of 50 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 3 and a heavy chain comprising the amino acid sequence of SEQ ID NO: 83, or a variant thereof, at a concentration of 150 mg/mL; (ii) a histidine buffer at a concentration of 20 mM; (iii) sucrose at a concentration of 8% (w/v); (iv) L-methionine at a concentration of 10 mM; and (v) a polysorbate at a concentration of about 0.04% (w/v); wherein the pharmaceutical composition is at a pH of 5.5.

In some embodiments, a pharmaceutical composition comprises: (i) an anti-IGF-1R antibody, or antigen-binding fragment thereof, comprising a light chain variable region comprising the amino acid sequence of SEQ ID NO: 86 and a heavy chain variable region comprising the amino acid sequence of SEQ ID NO: 91, or a variant thereof, at a concentration of 50 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain variable region comprising the amino acid sequence of SEQ ID NO: 86 and a heavy chain variable region comprising the amino acid sequence of SEQ ID NO: 91, or a variant thereof, at a concentration of 150 mg/mL; (ii) a histidine buffer at a concentration of 20 mM; (iii) sucrose at a concentration of 8% (w/v); (iv) L-methionine at a concentration of 10 mM; and (v) a polysorbate at a concentration of about 0.04% (w/v); wherein the pharmaceutical composition is at a pH of 5.5.

In some embodiments, a pharmaceutical composition comprises: (i) an anti-IGF-1R antibody, or antigen-binding fragment thereof, comprising a light chain comprising the amino acid sequence of SEQ ID NO: 93 and a heavy chain comprising the amino acid sequence of SEQ ID NO: 92, or a variant thereof, at a concentration of 50 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 93 and a heavy chain comprising the amino acid sequence of SEQ ID NO: 92, or a variant thereof, at a concentration of 150 mg/mL; (ii) a histidine buffer at a concentration of 20 mM; (iii) sucrose at a concentration of 8% (w/v); (iv) L-methionine at a concentration of 10 mM; and (v) a polysorbate at a concentration of about 0.04% (w/v); wherein the pharmaceutical composition is at a pH of 5.5.

In some embodiments, a pharmaceutical composition comprises: (i) an anti-IGF-1R antibody, or antigen-binding fragment thereof, comprising a light chain comprising the amino acid sequence of SEQ ID NO: 93 and a heavy chain comprising the amino acid sequence of SEQ ID NO: 94, or a variant thereof, at a concentration of 50 mg/mL; (ii) a histidine buffer at a concentration of 20 mM; (iii) sucrose at a concentration of 8% (w/v); (iv) L-methionine at a concentration of 10 mM; and (v) a polysorbate at a concentration of 0.02% (w/v); wherein the pharmaceutical composition is at a pH of 5.5. In some embodiments, a pharmaceutical composition comprises: (i) an anti-IGF-1R antibody, or antigen-binding fragment thereof, comprising a light chain comprising the amino acid sequence of SEQ ID NO: 93 and a heavy chain comprising the amino acid sequence of SEQ ID NO: 94, or a variant thereof, at a concentration of 150 mg/mL; (ii) a histidine buffer at a concentration of 20 mM; (iii) sucrose at a concentration of 8% (w/v); (iv) L-methionine at a concentration of 10 mM; and (v) a polysorbate at a concentration of about 0.04% (w/v); wherein the pharmaceutical composition is at a pH of 5.5.

### Administration Mode

In some embodiments, the pharmaceutical composition is suitable for intravenous, subcutaneous, or intramuscular administration. In some embodiments, the pharmaceutical composition is suitable for intravenous administration. In some embodiments, the pharmaceutical composition is suitable for subcutaneous administration. In some embodiments, the pharmaceutical composition is suitable for intramuscular administration.

In some embodiments, the pharmaceutical composition is a liquid pharmaceutical composition. In some embodiments, the pharmaceutical composition is a lyophilized pharmaceutical composition.

In some embodiments, a dosage form in a container is provided. In some embodiments, the dosage form comprises a pharmaceutical composition such as those provided herein. In some embodiments, the container is a plastic vial or a glass vial.

In some embodiments, a kit, comprising a pharmaceutical composition, such as those provided herein, is provided.

In some embodiments, to prepare pharmaceutical or sterile compositions of the anti-IGF-1R antibodies or other proteins provided herein, the antibody or antigen binding fragment thereof or other proteins provided herein are admixed with a pharmaceutically acceptable carrier or excipient. See, e.g., Remingto's Pharmaceutical Sciences and U.S. Pharmacopeia: National Formulary, Mack Publishing Company, Easton, PA (1984).

Pharmaceutical composition of therapeutic and diagnostic agents may be prepared by mixing with acceptable carriers, excipients, or stabilizers in the form of, e.g., lyophilized powders, slurries, aqueous solutions or suspensions (see, e.g., Hardman, et al. (2001) Goodman and Gilman's The Pharmacological Basis of Therapeutics, McGraw-Hill, New York, NY; Gennaro (2000) Remington: The Science and Practice of Pharmacy, Lippincott, Williams, and Wilkins, New York, NY; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, NY). In some embodiments, the antibodies are diluted to an appropriate concentration in a sodium acetate solution pH 5.0 to 6.0, and NaCl or sucrose is added for tonicity. Additional agents, such as polysorbate 20 or polysorbate 80, may be added to enhance stability.

Toxicity and therapeutic efficacy of the antibody compositions, administered alone or in combination with another agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index (LD₅₀/ED₅₀). In particular aspects, antibodies exhibiting high therapeutic indices are desirable. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration.

In some embodiments, a composition is administered to a subject in accordance with the Physician” Desk Reference 2003 (Thomson Healthcare; 5⁷^{ₜ}h edition (Nov. 1, 2002)).

The mode of administration can vary. Suitable routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal, or intra-arterial.

In some embodiments, the pharmaceutical composition can be administered by an invasive route such as by injection. In some embodiments, the pharmaceutical composition is administered intravenously, subcutaneously, intramuscularly, intraarterially, intra-articularly (e.g., in arthritis joints), or by inhalation, aerosol delivery. Administration by non-invasive routes (e.g., orally; for example, in a pill, capsule or tablet) is also within the scope of the present embodiments.

In some embodiments, the pharmaceutical composition can be administered directly to the eye, the anterior chamber of the eye, the vitreous chamber of the eye, the suprachoroidal space, or the retro-orbital sinus. In some embodiments, administration to the eye, the anterior chamber of the eye, the vitreous chamber of the eye, the suprachoroidal space, or the retro-orbital sinus is via an injection. In some embodiments, the injection is an intravitreal injection, intraorbital injection, retro-orbital injection, suprachoroidal injection, or intracameral injection. In some embodiments, the injection is an intravitreal injection. In some embodiments, the injection is an, intraorbital injection. In some embodiments, the injection is a retro-orbital injection. In some embodiments, the injection is a suprachoroidal injection. In some embodiments, the injection is an intracameral injection.

In some embodiments, the anti-IGF-1R antibody, or antigen binding fragment thereof, is administered in combination with at least one additional therapeutic agent, such as, but not limited to any therapeutic used to treat thyroid eye disease. For example, in some embodiments, the anti-IGF-1R antibody, or antigen binding fragment thereof, is administered in combination with at least one additional therapeutic agent, such as, but not limited to a therapeutic used to treat thyroid eye disease or a condition related to the same. Examples of such treatments and therapeutics include, but are not limited to anti-thyroid medications, diabetes medications, beta-blockers, propylthiouracil, methimazole, propranolol, atenolol, metoprolol, nadolol, corticosteroids, metformin, sulfonylureas, meglitinides, thiazolidinediones, DPP-4 inhibitors, GLP-1 receptor agonists, SGLT2 inhibitors, regular insulin, insulin aspart, insulin glulisine, insulin lispro, insulin isophane, insulin degludec, insulin detemir, insulin glargine, acerbose, miglitol, acebutolol, atenolol, betaxolol, bisoprolol, cartelol, carvedilol, esmolol, labetalol, metoprolol, nadolol, nebivolol, penbutolol, pindolol, propranolol, sotalol, timolol, tomolol ophthalmic solution, sitagliptin, saxagliptin, linagliptin, alogliptin, dulaglutide, exenatide, semaglutide, liraglutide, lixisenatide, canagliflozin, dapagliflozin, empagliflozin, or any combination thereof.

Compositions and formulations can be administered with medical devices known in the art. For example, a pharmaceutical composition as provided for herein can be administered by injection with a hypodermic needle, including, e.g., a prefilled syringe or autoinjector. Examples of such devices include, but are not limited to, those disclosed in U.S. Pat. Nos. 11,571,164, 11,040,138, and U.S Patent Application Publication No. 20200061292, each of which is hereby incorporated by reference in its entirety.

The pharmaceutical compositions and formulations may also be administered with a needleless hypodermic injection device; such as the devices disclosed in U.S. Pat. Nos. 6,620,135; 6,096,002; 5,399,163; 5,383,851; 5,312,335; 5,064,413; 4,941,880; 4,790,824 or 4,596,556, each of which is hereby incorporated by reference in its entirety.

The pharmaceutical compositions and formulations may also be administered by infusion. Examples of well-known implants and modules form administering pharmaceutical compositions include: U.S. Pat. No. 4,487,603, which discloses an implantable micro-infusion pump for dispensing medication at a controlled rate; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments. Many other such implants, delivery systems, and modules are well known to those skilled in the art.

Alternately, one may administer the pharmaceutical composition in a local rather than systemic manner, for example, via injection of the antibody directly into an arthritic joint or pathogen-induced lesion characterized by immunopathology, often in a depot or sustained release pharmaceutical composition. Furthermore, one may administer the antibody in a targeted drug delivery system, for example, in a liposome coated with a tissue-specific antibody, targeting, for example, arthritic joint or pathogen-induced lesion characterized by immunopathology. The liposomes will be targeted to and taken up selectively by the afflicted tissue.

The administration regimen depends on several factors, including the serum or tissue turnover rate of the therapeutic antibody, the level of symptoms, the immunogenicity of the therapeutic antibody, and the accessibility of the target cells in the biological matrix. Preferably, the administration regimen delivers sufficient therapeutic antibody to effect improvement in the target disease state, while simultaneously minimizing undesired side effects. Accordingly, the amount of biologic delivered depends in part on the particular therapeutic antibody and the severity of the condition being treated. Guidance in selecting appropriate doses of therapeutic antibodies is available (see, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, NY; Bach (ed.) (1993) Monoclonal Antibodies and Peptide Therapy in Autoimmune Diseases, Marcel Dekker, New York, NY; Baert, et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602).

Determination of the appropriate dose is made by the clinician, e.g., using parameters or factors known or suspected in the art to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. In general, it is desirable that a biologic that will be used is derived from the same species as the animal targeted for treatment, thereby minimizing any immune response to the reagent. In the case of human subjects, for example, chimeric, humanized and fully human antibodies are desirable.

The pharmaceutical compositions provided herein can be provided by continuous infusion, or by doses administered, e.g., daily, 1-7 times per week, weekly, bi-weekly, monthly, bimonthly, quarterly, semiannually, annually etc. Doses may be provided, e.g., intravenously, subcutaneously, topically, orally, nasally, rectally, intramuscular, intracerebrally, intraspinally, or by inhalation. In some embodiments, the pharmaceutical composition is administered every three weeks, every four weeks, every five weeks, every six weeks, every seven weeks, or every eight weeks. In some embodiments, the pharmaceutical composition is administered every three weeks. In some embodiments, the pharmaceutical composition is administered every four weeks. In some embodiments, the pharmaceutical composition is administered every five weeks. In some embodiments, the pharmaceutical composition is administered every six weeks. In some embodiments, the pharmaceutical composition is administered every seven weeks. In some embodiments, the pharmaceutical composition is administered every eight weeks. In some embodiments, the pharmaceutical composition is administered for at least 21-52 weeks or longer. In some embodiments, the pharmaceutical composition is administered on such a schedule for at least 21 weeks. In some embodiments, the pharmaceutical composition is administered on such a schedule for at least 24 weeks. In some embodiments, the pharmaceutical composition is administered on such a schedule for at least 32 weeks. In some embodiments, the pharmaceutical composition is administered on such a schedule for at least 36 weeks. In some embodiments, the pharmaceutical composition is administered on such a schedule for at least 40 weeks. In some embodiments, the pharmaceutical composition is administered on such a schedule for at least 42 weeks. In some embodiments, the pharmaceutical composition is administered (e.g., infusion or subcutaneous injection) once. In some embodiments, the pharmaceutical composition is administered (e.g., infusion or subcutaneous injection) twice. In some embodiments, the pharmaceutical composition is administered (e.g., infusion or subcutaneous injection) three times. In some embodiments, the pharmaceutical composition is administered (e.g., infusion or subcutaneous injection) four times. In some embodiments, the pharmaceutical composition is administered (e.g., infusion or subcutaneous injection) five times. In some embodiments, the pharmaceutical composition is administered (e.g., infusion or subcutaneous injection) six times. In some embodiments, the pharmaceutical composition is administered (e.g., infusion or subcutaneous injection) seven times. In some embodiments, the pharmaceutical composition is administered (e.g., infusion or subcutaneous injection) eight times. In some embodiments, the antibody is administered (e.g., infusion or subcutaneous injection) nine times. In some embodiments, the pharmaceutical composition is administered (e.g., infusion or subcutaneous injection) 10 times. In some embodiments, the pharmaceutical composition is administered (e.g., infusion or subcutaneous injection) 11 times. In some embodiments, the pharmaceutical composition is administered (e.g., infusion or subcutaneous injection) 12 times. In some embodiments, the pharmaceutical composition is administered (e.g., infusion or subcutaneous injection) 13 times. In some embodiments, the pharmaceutical composition is administered (e.g., infusion or subcutaneous injection) 14 times. In some embodiments, the pharmaceutical composition is administered (e.g., infusion or subcutaneous injection) 15 times. In some embodiments, the pharmaceutical composition is administered (e.g., infusion or subcutaneous injection) 16 times. In some embodiments, the pharmaceutical composition is administered (e.g., infusion or subcutaneous injection) 17 times. In some embodiments, the pharmaceutical composition is administered (e.g., infusion or subcutaneous injection) 18 times. In some embodiments, the pharmaceutical composition is administered (e.g., infusion or subcutaneous injection) 19 times. In some embodiments, the pharmaceutical composition is administered (e.g., infusion or subcutaneous injection) 20 times. When the pharmaceutical composition is administered more than once it can be administered according to a schedule, such as the schedules provided for herein.

A total weekly dose can be as provided for herein. In some embodiments, the total weekly dose is at least 0.05 μg/kg body weight, more generally at least 0.2 μg/kg, 0.5 μg/kg, 1 μg/kg, 10 μg/kg, 100 μg/kg, 0.25 mg/kg, 1.0 mg/kg, 2.0 mg/kg, 3.0 mg/kg, 5.0 mg/kg, 10 mg/kg, 20 mg/kg, 25 mg/kg, 50 mg/kg or more (see, e.g., Yang, et al. (2003) New Engl. J. Med. 349:427-434; Herold, et al. (2002) New Engl. J. Med. 346:1692-1698; Liu, et al. (1999) J. Neurol. Neurosurg. Psych. 67:451-456; Portielji, et al. (20003) Cancer Immunol. Immunother. 52:133-144). Doses may also be provided to achieve a pre-determined target concentration of the antibody in the subject's serum, such as 0.1, 0.3, 1, 3, 10, 30, 100, 300 μg/ml or more.

In some embodiments, the antibody has a serum concentration in the subject of at least, or about, 10 μg/ml or 20 μg/ml or 50 μg/ml, 70 μg/ml, 75 μg/ml, 80 μg/ml, 85 μg/ml, 90 μg/ml, 95 μg/ml, 100 μg/ml, or 105 g/ml at least 1, 2, or 3 weeks after administration.

In some embodiments, a dose of 20 mg/kg IV is administered In some embodiments, a dosing is used to provide a Cmin of 133 μg/mL after about 5 weeks. In some embodiments, the dose of the antibody that is administered that provides a Cmin of 102 μg/mL after 6 weeks. In some embodiments, the dose of the antibody is as provided for herein, such as 10 mg/mg as a loading dose with subsequent doses being the same or lower. In some embodiments, the antibody is administered as rpvodied for herein at a dose to achieve a Cmin of at least, or about, 100 μg/mL.

### Methods of Treatment

As used herein, “inhibit” or “treat” or “treatment” includes a postponement of development of the symptoms associated with a disorder and/or a reduction in the severity of the symptoms of such disorder. The terms further include ameliorating existing uncontrolled or unwanted symptoms, preventing additional symptoms, and ameliorating or preventing the underlying causes of such symptoms. Thus, the terms denote that a beneficial result has been conferred on a vertebrate subject with a disorder, disease or symptom, or with the potential to develop such a disorder, disease or symptom.

As used herein, the terms “therapeutically effective amount”, “therapeutically effective dose” and “effective amount” refer to an amount of the antibody, or antigen binding fragment thereof, that, when administered alone or in combination with an additional therapeutic agent to a cell, tissue, or subject, is effective to cause a measurable improvement in one or more symptoms of a disease or condition or the progression of such disease or condition. A therapeutically effective dose further refers to that amount of the binding compound sufficient to result in at least partial amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously. An effective amount of a therapeutic will result in an improvement of a diagnostic measure or parameter by at least 10%; usually by at least 20%; preferably at least about 30%; more preferably at least 40%, and most preferably by at least 50%. An effective amount can also result in an improvement in a subjective measure in cases where subjective measures are used to assess disease severity. In some embodiments, an amount is a therapeutically effective amount if it is an amount that can be used to treat or ameliorate a condition as provided for herein.

The term “subject” as used throughout includes any organism, such as an animal, including a mammal (e.g., rat, mouse, dog, cat, rabbit) and, for example, a human. A subject can also be referred to as a patient. In some embodiments, the subject is a subject in need thereof. A subject that is “in need thereof” refers to a subject that has been identified as requiring treatment for the condition that is to be treated and is treated with the specific intent of treating such condition. The conditions can be, for example, any of the conditions described herein.

Whereas an isolated antibody binds an epitope on a IGF-1R protein, or other protein described herein, and displays in vitro and/or in vivo IGF-1R inhibiting or therapeutic activities, the antibodies or antigen binding fragments thereof, capable of inhibiting IGF-1R function, are suitable both as therapeutic agents for treating IGF-1R-associated conditions in humans and animals. These conditions include thyroid eye disease. According, methods of treating such conditions are also provided, wherein the method comprises administering an antibody, or antigen binding fragment thereof, to the subject with such a condition.

In some embodiments, the methods comprise administering a pharmaceutical composition described herein to a susceptible subject or to one exhibiting a condition in which IGF-1R is known or suspected to have caused the pathology observed. In some embodiments, the pharmaceutical composition comprises an active form of an anti-IGF-1R antibody. Any active form of the antibody can be administered, including, but not limited to scFV, Fab and F(a″)2 fragments and other forms of antibodies provided for herein.

As used herein, an IGF-1R associated pathology refers to conditions that are caused by the modulation of IGF-1R. These conditions include, but are not limited to, thyroid eye disease and other conditions provided for herein.

In some embodiments, the antibodies used are compatible with the recipient species such that the immune response to the mAbs does not result in an unacceptably short circulating half-life or induce an immune response to the mAbs in the subject.

Treatment of individuals may comprise the administration of a pharmaceutical composition described herein. The pharmaceutical compositions can be provided in a kit, such as those provided herein. The pharmaceutical compositions can be used or administered alone or in admixture with another therapeutic, analgesic, or diagnostic agent, such as provided for herein. In providing a patient with a pharmaceutical composition comprising an antibody, or fragment thereof, capable of binding to IGF-1R, or an antibody capable of protecting against IGF-1R, pathology in a recipient patient, the dosage of administered agent will vary depending upon such factors as the patient's age, weight, height, sex, general medical condition, previous medical history, etc.

A pharmaceutical composition comprising an antibody, capable treating a condition associated with IGF-1R activity or use to treat an IGF-1R related pathology, is intended to be provided to subjects in an amount sufficient to affect a reduction, resolution, or amelioration in the IGF-1R related symptom or pathology. Such a pathology includes thyroid eye disease and the like.

Accordingly, in some embodiments, methods of treating a subject with an IGF-1R mediated disorder are provided. In some embodiments, the method comprises administering a pharmaceutical composition comprising an antibody, or antigen binding fragment thereof, as provided herein. In some embodiments, the disorder is thyroid eye disease. A s provided for herein, the antibodies, or antigen binding fragments thereof, can be administered with other therapeutics. These can be administered simultaneously or sequentially.

In some embodiments, the pharmaceutical compositions comprising antibodies, or antigen binding fragments thereof, may be used to treat thyroid eye disease. In some embodiments, the pharmaceutical composition comprising antibodies, or antigen binding fragments thereof, may be used to treating or reduce the severity of, thyroid-associated ophthalmopathy (TAO), or a symptom thereof.

In some embodiments, methods or uses are provided to reduce proptosis in an eye in a subject with thyroid-associated ophthalmopathy (TAO).

In some embodiments, the subject is a subject how has previously been treated with a different antibody than those provided herein.

In some embodiments, methods or uses are provided to Clinical Activity Score (CAS) in subject who has or is suspected of having thyroid-associated ophthalmopathy (TAO).

In some embodiments, methods or uses are provided to reduce proptosis by at least 2 mm and b) reducing the clinical activity score (CAS) in a subject with thyroid-associated ophthalmopathy (TAO).

As used herein, the term Clinical Activity Score (CAS) refers to the protocol described and scored according to Table 8, below. According to this protocol, one point is given for the presence of each of the parameters assessed in Table 8. The sum of all points defines clinical activity and provides the CAS, where 0 or 1 constitutes inactive disease and 7 severe active ophthalmopathy.

TABLE 8
Parameters for Calculating Clinical Activity Score (CAS)

| Item No. | Parameter |
|---|---|
| 1 | Spontaneous retrobulbar pain |
| 2 | in on attempted eye movements |
| | (upward, side to side, and downward gazes; |
| | sometimes termed gaze evoked orbital pain |
| 3 | Eyelid swelling |
| 4 | Eyelid erythema (redness) |
| 5 | Conjunctival redness |
| 6 | Chemosis (swelling/edema of the conjunctiva) |
| 7 | Swelling of caruncle or pila |

As provided in Table 8, the CAS consists of seven components: spontaneous retrobulbar pain, pain on attempted eye movements (upward, side-to-side, and downward gazes), conjunctival redness, redness of the eyelids, chemosis, swelling of the caruncle/plica, and swelling of the eyelids. Each component is scored as present (1 point) or absent (0 points). The score at each efficacy assessment is the sum of all items present; giving a range of 0-7, where 0 or 1 constitutes inactive disease and 7 severe active ophthalmopathy. A change of >2 points is considered clinically meaningful.

Item 1, spontaneous orbital pain could be a painful, or oppressive feeling on, or behind, the globe. This pain may be caused by the rise in intraorbital pressure, when the orbital tissues volume increases through excess synthesis of extracellular matrix, fluid accumulation, and cellular infiltration and expansion. Item 2, gaze evoked orbital pain, could be pain in the eyes when looking, or attempting to look, up, down or sideways, i.e., pain with upward, downward, or lateral eye movement, or when attempting eye movement. This kind of pain could arise from the stretching of the inflamed muscle(s), especially on attempted upgaze. The ‘stretching pain’ cannot be provoked by digital pressing on the eyeball, as would be expected if it were a manifestation of the raised intraorbital pressure. Both kinds of pain can be reduced after anti-inflammatory treatment. These kinds of pain are therefore considered to be directly related to autoimmune inflammation in the orbit and thus useful in assessing TAO activity.

Swelling in TAO is seen as chemosis (edema of the conjunctiva), item no. 6 in Table 8, and swelling of the caruncle and/or plica semilunaris. Both are signs of TAO activity. Swollen eyelids can be caused by edema, fat prolapse through the orbital septum, or fibrotic degeneration. In addition to swelling, other symptoms indicative of active TAO includes redness and/or pain of the conjunctiva, eyelid, caruncle and/or plica semilunaris.

In some embodiments, the subject who is treated has the proptosis reduced by at least 2 mm. In some embodiments, the subject who is treated has the proptosis reduced by at least 3 mm. In some embodiments, the subject who is treated has the proptosis reduced by at least 4 mm. In some embodiments, the subject who is treated has the proptosis reduced by at least 5 mm.

In some embodiments, in the subjects who are treated the clinical activity score (CAS) of the subject is reduced by at least 2 points. In some embodiments, the clinical activity score (CAS) of the subject is reduced to one (1). In some embodiments, the clinical activity score (CAS) of the subject is reduced to zero (0).

In some embodiments, methods off treating or reducing the severity of thyroid-associated ophthalmopathy (TAO) in a subject are provided, wherein the treatment with a pharmaceutical composition (i) reduces proptosis by at least 2 mm in an eye; (ii) is not accompanied by a deterioration of 2 mm or more in the other (or fellow eye); and (iii) reduces the CAS in said subject by at least 2 points in an eye; and/or (iv) is not accompanied by an increase in CAS of 2 points or more in the other (or fellow) eye.

In some embodiments, methods of improving the quality of life in a subject with thyroid-associated ophthalmopathy (TAO, also called Grave” Ophthalmopathy/Grave” Orbitopathy) are provided. In some embodiments, the quality of life is measured by the Grave” Ophthalmopathy Quality of Life (GO-QoL) assessment, or either the Visual Functioning or Appearance subscale thereof. In some embodiments, the treatment results in an improvement of greater than or equal to 8 points on the GO-QoL. In some embodiments, the treatment results in an improvement on the Functioning subscale of the GO-QoL. In some embodiments, the treatment results in an improvement on the Appearance subscale of the GO-QOL.

In some embodiments, methods of treating or reducing the severity of diplopia in a subject with thyroid-associated ophthalmopathy (TAO) are provided. In some embodiments, the diplopia is constant diplopia. In some embodiments, the diplopia is inconstant diplopia. In some embodiments, the diplopia is intermittent diplopia. In some embodiments, the improvement in or reduction in severity of diplopia is sustained at least 20 weeks after discontinuation of pharmaceutical composition administration. In some embodiments, the improvement in or reduction in severity of diplopia is sustained at least 50 weeks after discontinuation of pharmaceutical composition administration.

The severity of the disease can be measured in the following non-limiting embodiments. For example, for lid aperture, the distance between the lid margins is measured (in mm) with the patient looking in the primary position, sitting relaxed, and with distant fixation. For swelling of the eyelids, the measure/evaluation is either “absent/equivocal,” “moderate,” or “severe.” Redness of the eyelids is either absent or present. Redness of the conjunctivae is either absent or present. In some embodiments, conjunctival edema is either absent or present. In some embodiments, inflammation of the caruncle or plica is either absent or present. Exophthalmos is measured in millimeter using the same Hertel exophthalmometer and same intercanthal distance for an individual patient. Subjective diplopia is scored from 0 to 3 (0=no diplopia; 1=intermittent, i.e., diplopia in primary position of gaze, when tired or when first awakening; 2=inconstant, i.e., diplopia at extremes of gaze; 3=constant, i.e., continuous diplopia in primary or reading position). For eye muscle involvement, the ductions are measured in degrees. Corneal involvement is either absent/punctate or keratopathy/ulcer. For optic nerve involvement, i.e., best-corrected visual acuity, color vision, optic disc, relative afferent pupillary defect, the condition is either absent or present. In addition, visual fields are checked if optic nerve compression is suspected. In some embodiments, the patient can be classified according to the following severity classification. For example, sight-Threatening Thyroid Eye Disease: Patients with dysthyroid optic neuropathy (DON) and/or corneal breakdown. This category warrants immediate intervention. Moderate-to-Severe Thyroid Eye Disease: Patients without sight-threatening disease whose eye disease have sufficient impact on daily life to justify the risks of immunosuppression (if active) or surgical intervention (if inactive). Patients with moderate-to-severe thyroid eye disease usually have any one or more of the following: lid retraction greater than or equal to 2 mm, moderate or severe soft tissue involvement, exophthalmos greater than or equal to 3 mm above normal for race and gender, inconstant or constant diplopia. Mild Thyroid Eye Disease: Patients whose features of thyroid eye disease have only a minor impact on daily life insufficient to justify immunosuppressive or surgical treatment. They usually have only one or more of the following: minor lid retraction (<2 mm), mild soft tissue involvement, exophthalmos <3 mm above normal for race and gender, transient or no diplopia, and corneal exposure responsive to lubricants.

In some embodiments, a patient can be characterized by Graves Ophthalmopathy Quality of Life (GO-QoL) score. In addition to proptosis (or exophthalmos) and CAS, quality of life is also evaluated with the use of the GO quality of life (GO-QOL) questionnaire. This questionnaire is designed to determine the improved quality of life after treatment with a method disclosed herein. In some embodiments, questionnaire may determine the decreased or lack of side effects after being treated with an antibody, or an antigen binding fragment thereof, according to a method disclosed herein as compared to treatment with glucocorticoids. The GO-QoL is a 16-item self-administered questionnaire divided into 2 subsets and used to assess the perceived effects of TED by the subjects on (i) their daily physical activity as it relates to visual function, and (ii) psychosocial functioning. Quality of life is evaluated with the use of the GO QOL questionnaire. The GO-QoL questionnaire [C. B. Terwee et al, 1998] is completed on Day 1 and Weeks 6, 12, and 24 (or PW) during the Treatment Period, and at Months 7 and 12 (or PW) during the Follow-Up Period. The GO-QoL is a 16-item self-administered questionnaire divided into two self-assessment subscales; one covering impact of visual function on daily activities, the other assesses the impact of self-perceived appearance. The visual function subscale covers activities such as driving, walking outdoors, reading, watching television. The appearance subscale asks the subject questions such as whether ophthalmopathy has altered the subject's appearance, caused other people to have a negative reaction to the subject, caused social isolation, and caused the subject to try to mask his or her appearance. Each subscale has 8 questions which are answered with: ye—very much so; ye—a little; or n—not at all. Each question is scored 0-2, respectively, and the total raw score is then mathematically transformed to a 0-100 scale, where 0 represents the most negative impact on quality of life, and 100 represents no impact. A change of > or greater than equal to 8 points on the 0-100 scale has been shown to be clinically meaningful. The combined score takes raw scores from both subscales and again transforms them to a single 0-100 scale. The questionnaire has two self-assessment subscales. Each subscale has 8 questions which are answered with: (i) ye—very much so; (ii) ye—a little; or (iii) n—not at all. Each question is scored 0-2, respectively, and the total raw score is then mathematically transformed to a 0-100 scale, where 0 represents the most negative impact on quality of life, and 100 represents no impact. A change of >8 points on the 0-100 scale is considered to be clinically meaningful. The combined score takes raw scores from both subscales and again transforms them to a single 0-100 scale.

Patients can also be assessed by the presence of absence of Gorman Grading of Diplopia. The Gorman assessment of subjective diplopia includes four categories: no diplopia (absent), diplopia when the patient is tired or awakening (intermittent), diplopia at extremes of gaze (inconstant), and continuous diplopia in the primary or reading position (constant). Patients are scored according to which grade of diplopia they are experiencing. An improvement of greater than equal or to 1 grade is considered clinically meaningful. A Gorman score of zero (0) for subjects with baseline diploplia may indicate diplopia resolution.

In some embodiments, the methods comprise administering a pharmaceutical composition comprising an anti-IGF-1R antibody, such as those provided herein. In some embodiments, the antibody is administered at a dosage of about 1 mg/kg to about 5 mg/kg antibody as a first dose. In some embodiments, the antibody is administered at a dosage of about 5 mg/kg to about 10 mg/kg antibody as a first dose. In some embodiments, the antibody is administered at a dosage of about 5 mg/kg to about 20 mg/kg antibody as a first dose.

In some embodiments, the antibody is administered in a pharmaceutical composition, such as those provided herein. In some embodiments, the pharmaceutical composition further comprises one or more pharmaceutically active compounds for the treatment of TAO. In some embodiments, the pharmaceutical composition further comprises corticosteroids; rituximab or other anti-CD20 antibodies; tocilizumab or other anti-IL-6 antibodies; or selenium, infliximab or other anti-TNFalpha antibodies or a thyroid-stimulating hormone receptor (TSHR) inhibitor.

In some embodiments, the method provided herein comprise administering to a subject an antibody, or an antigen binding fragment thereof, that specifically binds to and inhibits IGF-IR. In some embodiments, the antibody is as provided herein.

Kits are also provided which are useful for carrying out embodiments described herein. The present kits comprise a first container containing or packaged in association with the above-described antibodies. The kit may also comprise another container containing or packaged in association solutions necessary or convenient for carrying out the embodiments. The containers can be made of glass, plastic or foil and can be a vial, bottle, pouch, tube, bag, etc. The kit may also contain written information, such as procedures for carrying out the embodiments or analytical information, such as the amount of reagent contained in the first container means. The container may be in another container apparatus, e.g., a box or a bag, along with the written information.

Yet another aspect provided for herein is a kit for detecting IGF-1R protein in a biological sample. The kit includes a container holding one or more antibodies which binds an epitope of IGF-1R protein and instructions for using the antibody for the purpose of binding to IGF-1R protein to form an immunological complex and detecting the formation of the immunological complex such that the presence or absence of the immunological complex correlates with presence or absence of IGF-1R protein in the sample. Examples of containers include multiwell plates which allow simultaneous detection of IGF-1R protein in multiple samples.

In some embodiments, antibodies that bind to an IGF-1R protein are provided. In some embodiments, the antibody is isolated. In some embodiments, the antibody binds specifically. In some embodiments, the antibody binds to an IGF-1R protein that is properly folded. In some embodiments, the antibody is specific for a specific IGF-1R conformational state (open or closed). In some embodiments, the antibody binds to an IGF-1R protein in a cell membrane. In some embodiments, the antibody binds to an IGF-1R protein that is in a cell membrane in an intact cell. In some embodiments, the antibody inhibits or neutralizes the function of an IGF-1R protein. As used herein, the term “neutralize” means that the activity or function of the protein is inhibited. The inhibition can be complete or partial. In some embodiments, the activity or function of the protein is inhibited at least 10, 20, 30, 40, 50, 60, 70, 80, 90, 95, or 99%. The percent inhibition can be based upon the function or activity of the protein in the absence of the antibody. In some embodiments, the antibody inhibits the glucose transport facilitated by IGF-1R. In some embodiments, the antibody inhibits the internalization of the IGF-1R protein.

In some embodiments, the antibody comprises a sequence as provided for herein or antigen binding fragment thereof. In some embodiments, the antibody comprises a heavy chain CDR or an antigen binding fragment thereof described herein. The heavy chain may be one or more of the heavy chains described herein. In some embodiments, the antibody comprises a light chain, or an antigen binding fragment thereof as described herein.

In some embodiments, methods of treating, inhibiting or ameliorating a IGF-1R, associated pathology are provided. In some embodiments, the methods comprise administering a pharmaceutical composition described herein to a subject to treat, inhibit or ameliorate a IGF-1R associated pathology. In some embodiments, the pathology is as described herein.

### Enumerated Embodiments

Embodiment 1. A pharmaceutical composition comprising: (i) an anti-IGF-1R antibody, or antigen-binding fragment thereof; (ii) a buffer at a concentration of about 10-60 mM; (iii) sucrose at a concentration of about 1-20% (w/v); (iv) a stabilizer at a concentration of about 1-15 mM; and (v) a surfactant at a concentration of about 0.001-1% (w/v).

Embodiment 2. The pharmaceutical composition of embodiment 1, wherein the antibody, or antigen binding fragment thereof, comprises: a VL sequence as set forth in SEQ ID NO: 1, 3, 5, 7, 9, 11, 13, 15, 79, or 86; a VH sequence as set forth in SEQ ID NO: 2, 4, 6, 8, 10, 12, 14, 16, 80, or 83; a LCDR sequence as set forth in SEQ ID NO: 17, 18, 19, 23, 24, 25, 29, 30, 31, 35, 36, 37, 41, 42, 43, 47, 48, 49, 53, 54, 55, 59, 60, 61, or 81, or a HCDR sequence as set forth in SEQ ID NO: 20, 21, 22, 26, 27, 28, 32, 33, 34, 38, 39, 40, 44, 45, 46, 50, 51, 52, 56, 57, 58, 62, 63, or 64; and any combination or variant thereof.

Embodiment 3. The pharmaceutical composition of any one of embodiments 1-2, wherein the antibody, or antigen binding fragment thereof, comprises:
(i) a heavy chain variable region comprising heavy chain CDR1, CDR2, and CDR3 sequences, wherein the heavy chain CDR1 sequence has the amino acid sequence of SEQ ID NO: 20; the heavy chain CDR2 has the amino acid sequence of SEQ ID NO: 21; and the heavy chain CDR3 sequence has the amino acid sequence of SEQ ID NO: 22; or variants of any of the foregoing; and (ii) a light chain variable region comprising light chain CDR1, CDR2, and CDR3 sequences, wherein the light chain CDR1 sequence has the amino acid sequence SEQ ID NO: 17; the light chain CDR2 sequence has the amino acid sequence of SEQ ID NO: 18; and the light chain CDR3 sequence has the amino acid sequence of SEQ ID NO: 19; or variants of any of the foregoing;
(i) a heavy chain variable region comprising heavy chain CDR1, CDR2, and CDR3 sequences, wherein the heavy chain CDR1 sequence has the amino acid sequence of SEQ ID NO: 26; the heavy chain CDR2 has the amino acid sequence of SEQ ID NO: 27; and the heavy chain CDR3 sequence has the amino acid sequence of SEQ ID NO: 28; or variants of any of the foregoing; and (ii) a light chain variable region comprising light chain CDR1, CDR2, and CDR3 sequences, wherein the light chain CDR1 sequence has the amino acid sequence SEQ ID NO: 23; the light chain CDR2 sequence has the amino acid sequence of SEQ ID NO: 24; and the light chain CDR3 sequence has the amino acid sequence of SEQ ID NO: 25; or variants of any of the foregoing;
(i) a heavy chain variable region comprising heavy chain CDR1, CDR2, and CDR3 sequences, wherein the heavy chain CDR1 sequence has the amino acid sequence of SEQ ID NO: 32; the heavy chain CDR2 has the amino acid sequence of SEQ ID NO: 33; and the heavy chain CDR3 sequence has the amino acid sequence of SEQ ID NO: 34; or variants of any of the foregoing; and (ii) a light chain variable region comprising light chain CDR1, CDR2, and CDR3 sequences, wherein the light chain CDR1 sequence has the amino acid sequence SEQ ID NO: 29; the light chain CDR2 sequence has the amino acid sequence of SEQ ID NO: 30; and the light chain CDR3 sequence has the amino acid sequence of SEQ ID NO: 31; or variants of any of the foregoing;
(i) a heavy chain variable region comprising heavy chain CDR1, CDR2, and CDR3 sequences, wherein the heavy chain CDR1 sequence has the amino acid sequence of SEQ ID NO: 38; the heavy chain CDR2 has the amino acid sequence of SEQ ID NO: 39; and the heavy chain CDR3 sequence has the amino acid sequence of SEQ ID NO: 40; or variants of any of the foregoing; and (ii) a light chain variable region comprising light chain CDR1, CDR2, and CDR3 sequences, wherein the light chain CDR1 sequence has the amino acid sequence SEQ ID NO: 35; the light chain CDR2 sequence has the amino acid sequence of SEQ ID NO: 36; and the light chain CDR3 sequence has the amino acid sequence of SEQ ID NO: 37; or variants of any of the foregoing;
(i) a heavy chain variable region comprising heavy chain CDR1, CDR2, and CDR3 sequences, wherein the heavy chain CDR1 sequence has the amino acid sequence of SEQ ID NO: 44; the heavy chain CDR2 has the amino acid sequence of SEQ ID NO: 45; and the heavy chain CDR3 sequence has the amino acid sequence of SEQ ID NO: 46; or variants of any of the foregoing; and (ii) a light chain variable region comprising light chain CDR1, CDR2, and CDR3 sequences, wherein the light chain CDR1 sequence has the amino acid sequence SEQ ID NO: 41; the light chain CDR2 sequence has the amino acid sequence of SEQ ID NO: 42; and the light chain CDR3 sequence has the amino acid sequence of SEQ ID NO: 43; or variants of any of the foregoing;
(i) a heavy chain variable region comprising heavy chain CDR1, CDR2, and CDR3 sequences, wherein the heavy chain CDR1 sequence has the amino acid sequence of SEQ ID NO: 50; the heavy chain CDR2 has the amino acid sequence of SEQ ID NO: 51; and the heavy chain CDR3 sequence has the amino acid sequence of SEQ ID NO: 52; or variants of any of the foregoing; and (ii) a light chain variable region comprising light chain CDR1, CDR2, and CDR3 sequences, wherein the light chain CDR1 sequence has the amino acid sequence SEQ ID NO: 47; the light chain CDR2 sequence has the amino acid sequence of SEQ ID NO: 48; and the light chain CDR3 sequence has the amino acid sequence of SEQ ID NO: 49; or variants of any of the foregoing;
(i) a heavy chain variable region comprising heavy chain CDR1, CDR2, and CDR3 sequences, wherein the heavy chain CDR1 sequence has the amino acid sequence of SEQ ID NO: 56; the heavy chain CDR2 has the amino acid sequence of SEQ ID NO: 57; and the heavy chain CDR3 sequence has the amino acid sequence of SEQ ID NO: 58; or variants of any of the foregoing; and (ii) a light chain variable region comprising light chain CDR1, CDR2, and CDR3 sequences, wherein the light chain CDR1 sequence has the amino acid sequence SEQ ID NO: 53; the light chain CDR2 sequence has the amino acid sequence of SEQ ID NO: 54; and the light chain CDR3 sequence has the amino acid sequence of SEQ ID NO: 55; or variants of any of the foregoing;
(i) a heavy chain variable region comprising heavy chain CDR1, CDR2, and CDR3 sequences, wherein the heavy chain CDR1 sequence has the amino acid sequence of SEQ ID NO: 62; the heavy chain CDR2 has the amino acid sequence of SEQ ID NO: 63; and the heavy chain CDR3 sequence has the amino acid sequence of SEQ ID NO: 64; or variants of any of the foregoing; and (ii) a light chain variable region comprising light chain CDR1, CDR2, and CDR3 sequences, wherein the light chain CDR1 sequence has the amino acid sequence SEQ ID NO: 59; the light chain CDR2 sequence has the amino acid sequence of SEQ ID NO: 60; and the light chain CDR3 sequence has the amino acid sequence of SEQ ID NO: 61; or variants of any of the foregoing; or
(i) a heavy chain variable region comprising heavy chain CDR1, CDR2, and CDR3 sequences, wherein the heavy chain CDR1 sequence has the amino acid sequence of SEQ ID NO: 38; the heavy chain CDR2 has the amino acid sequence of SEQ ID NO: 39; and the heavy chain CDR3 sequence has the amino acid sequence of SEQ ID NO: 40; or variants of any of the foregoing; and (ii) a light chain variable region comprising light chain CDR1, CDR2, and CDR3 sequences, wherein the light chain CDR1 sequence has the amino acid sequence SEQ ID NO: 35; the light chain CDR2 sequence has the amino acid sequence of SEQ ID NO: 36; and the light chain CDR3 sequence has the amino acid sequence of SEQ ID NO: 81; or variants of any of the foregoing.

Embodiment 4. The pharmaceutical composition of any one of embodiments 1-3, wherein the antibody comprises a VL sequence as set forth in SEQ ID NO: 1, 3, 5, 7, 9, 11, 13, 15, 79, or 86, or a variant thereof.

Embodiment 5. The pharmaceutical composition of any one of embodiments 1-3, wherein the antibody comprises a VH sequence as set forth in SEQ ID NO: 2, 4, 6, 8, 10, 12, 14, 16, 80, 83, 91, 96, or 99, or a or a variant thereof.

Embodiment 6. The pharmaceutical composition of any one of embodiments 1-5, wherein the antibody comprises:
a V_{L} comprising the amino acid sequence of SEQ ID NO: 1 and a V_{H} comprising the amino acid sequence of SEQ ID NO: 2;
a V_{L} comprising the amino acid sequence of SEQ ID NO: 3 and a V_{H} comprising the amino acid sequence of SEQ ID NO: 4;
a V_{L} comprising the amino acid sequence of SEQ ID NO: 5 and a V_{H} comprising the amino acid sequence of SEQ ID NO: 6;
a V_{L} comprising the amino acid sequence of SEQ ID NO: 7 and a V_{H} comprising the amino acid sequence of SEQ ID NO: 8;
a V_{L} comprising the amino acid sequence of SEQ ID NO: 9 and a V_{H} comprising the amino acid sequence of SEQ ID NO: 10;
a V_{L} comprising the amino acid sequence of SEQ ID NO: 11 and a V_{H} comprising the amino acid sequence of SEQ ID NO: 12;
a V_{L} comprising the amino acid sequence of SEQ ID NO: 93 and a V_{H} comprising the amino acid sequence of SEQ ID NO: 92;
a V_{L} comprising the amino acid sequence of SEQ ID NO: 15 and a V_{H} comprising the amino acid sequence of SEQ ID NO: 16;
a V_{L} comprising the amino acid sequence of SEQ ID NO: 79 and a V_{H} comprising the amino acid sequence of SEQ ID NO: 80;
a V_{L} comprising the amino acid sequence of SEQ ID NO: 3 and a V_{H} comprising the amino acid sequence of SEQ ID NO: 83;
a V_{L} comprising the amino acid sequence of SEQ ID NO: 100 and a V_{H} comprising the amino acid sequence of SEQ ID NO: 101;
or a variant thereof.

Embodiment 7. The pharmaceutical composition of any one of embodiments 1-6, wherein the variant has at least 85% homology to a sequence of SEQ ID NO: 1-72, 78-83, or 85-86.

Embodiment 8. The pharmaceutical composition of any one of embodiments 1-6, wherein the variant has at least 90%, 91%, 92%, 93%, 94%, 95%, 96%, 97%, 98%, or 99% homology to a sequence of SEQ ID NO: 1-72, 78-83, or 85-86.

Embodiment 9. The pharmaceutical composition of any one of embodiments 1-8, wherein the variant has at least 85% identity to a sequence of SEQ ID NO: 1-72, 78-83, or 85-86.

Embodiment 10. The pharmaceutical composition of any one of embodiments 1-9, wherein the variant has at least 90%, 91%, 92%, 93%, 94%, 95%, 96%, 97%, 98%, or 99% identify to a sequence of SEQ ID NO: 1-72, 78-83, or 85-86.

Embodiment 11. The pharmaceutical composition of any one of embodiments 1-10, wherein the antibody, or antigen-binding fragment thereof, is present at a concentration of about 1-300 mg/mL.

Embodiment 12. The pharmaceutical composition of any one of embodiments 1-11, wherein the antibody, or antigen-binding fragment thereof, is present at a concentration of about 1-200 mg/mL.

Embodiment 13. The pharmaceutical composition of any one of embodiments 1-12, wherein the antibody, or antigen-binding fragment thereof, is present at a concentration of about 20-150 mg/mL.

Embodiment 14. The pharmaceutical composition of any one of embodiments 1-13, wherein the antibody, or antigen-binding fragment thereof, is present at a concentration of about 25 mg/mL.

Embodiment 15. The pharmaceutical composition of any one of embodiments 1-13, wherein the antibody, or antigen-binding fragment thereof, is present at a concentration of about 50 mg/mL.

Embodiment 16. The pharmaceutical composition of any one of embodiments 1-13, wherein the antibody, or antigen-binding fragment thereof, is present at a concentration of about 150 mg/mL.

Embodiment 17. The pharmaceutical composition of any one of embodiments 1-16, wherein the buffer is histidine buffer, histidine HCl buffer, glycine buffer, Tris/glycine buffer, acetate buffer, sodium acetate buffer, potassium acetate buffer, magnesium acetate buffer, phosphate buffer, citrate buffer, or succinate buffer.

Embodiment 18. The pharmaceutical composition of embodiment 17, wherein the buffer is histidine buffer.

Embodiment 19. The pharmaceutical composition of any one of embodiments 11-18, wherein the histidine buffer is at a concentration of about 10-60 mM.

Embodiment 20. The pharmaceutical composition of any one of embodiments 11-19, wherein the histidine buffer is at a concentration of about 10-40 mM.

Embodiment 21. The pharmaceutical composition of any one of embodiments 11-20, wherein the histidine buffer is at a concentration of about 15-30 mM.

Embodiment 22. The pharmaceutical composition of any one of embodiments 11-21, wherein the histidine buffer is at a concentration of about 20 mM.

Embodiment 23. The pharmaceutical composition of any one of embodiments 1-22, wherein the sucrose is at a concentration of about 1-15% (w/v).

Embodiment 24. The pharmaceutical composition of any one of embodiments 1-23, wherein the sucrose is at a concentration of about 5-10% (w/v).

Embodiment 25. The pharmaceutical composition of any one of embodiments 1-24, wherein the sucrose is at a concentration of about 8% (w/v).

Embodiment 26. The pharmaceutical composition of any one of embodiments 1-25, wherein the stabilizer, or the anti-oxidant, is methionine, L-methionine, arginine, glycine, histidine, or proline.

Embodiment 27. The pharmaceutical composition of embodiment 26, wherein the stabilizer, or the anti-oxidant, is methionine, or L-methionine.

Embodiment 28. The pharmaceutical composition of any one of embodiments 26-27, wherein the methionine, or L-methionine, is present at a concentration of about 1-15 mM.

Embodiment 29. The pharmaceutical composition of any one of embodiments 26-28, wherein the methionine, or L-methionine, is present at a concentration of about 7-13 mM.

Embodiment 30. The pharmaceutical composition of any one of embodiments 26-29, wherein the methionine, or L-methionine, is present at a concentration of about 10 mM.

Embodiment 31. The pharmaceutical composition of any one of embodiments 1-30, wherein the surfactant is a polysorbate, or a poloxamer.

Embodiment 32. The pharmaceutical composition of embodiment 31, wherein the polysorbate is polysorbate 80 (PS80), or polysorbate 20 (PS20).

Embodiment 33. The pharmaceutical composition of any one of embodiments 31-32, wherein the polysorbate is polysorbate 80 (PS80), or polysorbate 20 (PS20).

Embodiment 34. The pharmaceutical composition of any one of embodiments 31-33, wherein the polysorbate is polysorbate 80 (PS80).

Embodiment 35. The pharmaceutical composition of any one of embodiments 31-34, wherein the polysorbate 80 is at a concentration of about 0.001-1% (w/v).

Embodiment 36. The pharmaceutical composition of any one of embodiments 31-35, wherein the polysorbate 80 is at a concentration of about 0.01-0.1% (w/v).

Embodiment 37. The pharmaceutical composition of any one of embodiments 31-36, wherein the polysorbate 80 is at a concentration of about 0.01-0.05% (w/v).

Embodiment 38. The pharmaceutical composition of any one of embodiments 31-37, wherein the polysorbate 80 is at a concentration of about 0.01% (w/v).

Embodiment 39. The pharmaceutical composition of any one of embodiments 31-37, wherein the polysorbate 80 is at a concentration of about 0.02% (w/v).

Embodiment 40. The pharmaceutical composition of any one of embodiments 1-39, wherein the pH of the pharmaceutical composition is about 5.0 to about 6.5.

Embodiment 41. The pharmaceutical composition of embodiment 31, wherein the pH of the pharmaceutical composition is about 5.5.

Embodiment 42. The pharmaceutical composition of embodiment 31, wherein the pH of the pharmaceutical composition is about 6.0.

Embodiment 43. A pharmaceutical composition comprising: (i) an anti-IGF-1R antibody, or antigen-binding fragment thereof, at a concentration of about 10-300 mg/mL; (ii) a histidine buffer at a concentration of about 10-60 mM; (iii) sucrose at a concentration of about 1-20% (w/v); (iv) methionine, or L-methionine, at a concentration of about 1-15 mM; and (v) a polysorbate at a concentration of about 0.001-1% (w/v).

Embodiment 44. The pharmaceutical composition of embodiment 43, wherein the antibody, or antigen binding fragment thereof, comprises:
(i) a heavy chain variable region comprising heavy chain CDR1, CDR2, and CDR3 sequences, wherein the heavy chain CDR1 sequence has the amino acid sequence of SEQ ID NO: 20; the heavy chain CDR2 has the amino acid sequence of SEQ ID NO: 21; and the heavy chain CDR3 sequence has the amino acid sequence of SEQ ID NO: 22; or variants of any of the foregoing; and (ii) a light chain variable region comprising light chain CDR1, CDR2, and CDR3 sequences, wherein the light chain CDR1 sequence has the amino acid sequence SEQ ID NO: 17; the light chain CDR2 sequence has the amino acid sequence of SEQ ID NO: 18; and the light chain CDR3 sequence has the amino acid sequence of SEQ ID NO: 19; or variants of any of the foregoing;
(i) a heavy chain variable region comprising heavy chain CDR1, CDR2, and CDR3 sequences, wherein the heavy chain CDR1 sequence has the amino acid sequence of SEQ ID NO: 26; the heavy chain CDR2 has the amino acid sequence of SEQ ID NO: 27; and the heavy chain CDR3 sequence has the amino acid sequence of SEQ ID NO: 28; or variants of any of the foregoing; and (ii) a light chain variable region comprising light chain CDR1, CDR2, and CDR3 sequences, wherein the light chain CDR1 sequence has the amino acid sequence SEQ ID NO: 23; the light chain CDR2 sequence has the amino acid sequence of SEQ ID NO: 24; and the light chain CDR3 sequence has the amino acid sequence of SEQ ID NO: 25; or variants of any of the foregoing;
(i) a heavy chain variable region comprising heavy chain CDR1, CDR2, and CDR3 sequences, wherein the heavy chain CDR1 sequence has the amino acid sequence of SEQ ID NO: 32; the heavy chain CDR2 has the amino acid sequence of SEQ ID NO: 33; and the heavy chain CDR3 sequence has the amino acid sequence of SEQ ID NO: 34; or variants of any of the foregoing; and (ii) a light chain variable region comprising light chain CDR1, CDR2, and CDR3 sequences, wherein the light chain CDR1 sequence has the amino acid sequence SEQ ID NO: 29; the light chain CDR2 sequence has the amino acid sequence of SEQ ID NO: 30; and the light chain CDR3 sequence has the amino acid sequence of SEQ ID NO: 31; or variants of any of the foregoing;
(i) a heavy chain variable region comprising heavy chain CDR1, CDR2, and CDR3 sequences, wherein the heavy chain CDR1 sequence has the amino acid sequence of SEQ ID NO: 38; the heavy chain CDR2 has the amino acid sequence of SEQ ID NO: 39; and the heavy chain CDR3 sequence has the amino acid sequence of SEQ ID NO: 40; or variants of any of the foregoing; and (ii) a light chain variable region comprising light chain CDR1, CDR2, and CDR3 sequences, wherein the light chain CDR1 sequence has the amino acid sequence SEQ ID NO: 35; the light chain CDR2 sequence has the amino acid sequence of SEQ ID NO: 36; and the light chain CDR3 sequence has the amino acid sequence of SEQ ID NO: 37; or variants of any of the foregoing;
(i) a heavy chain variable region comprising heavy chain CDR1, CDR2, and CDR3 sequences, wherein the heavy chain CDR1 sequence has the amino acid sequence of SEQ ID NO: 44; the heavy chain CDR2 has the amino acid sequence of SEQ ID NO: 45; and the heavy chain CDR3 sequence has the amino acid sequence of SEQ ID NO: 46; or variants of any of the foregoing; and (ii) a light chain variable region comprising light chain CDR1, CDR2, and CDR3 sequences, wherein the light chain CDR1 sequence has the amino acid sequence SEQ ID NO: 41; the light chain CDR2 sequence has the amino acid sequence of SEQ ID NO: 42; and the light chain CDR3 sequence has the amino acid sequence of SEQ ID NO: 43; or variants of any of the foregoing;
(i) a heavy chain variable region comprising heavy chain CDR1, CDR2, and CDR3 sequences, wherein the heavy chain CDR1 sequence has the amino acid sequence of SEQ ID NO: 50; the heavy chain CDR2 has the amino acid sequence of SEQ ID NO: 51; and the heavy chain CDR3 sequence has the amino acid sequence of SEQ ID NO: 52; or variants of any of the foregoing; and (ii) a light chain variable region comprising light chain CDR1, CDR2, and CDR3 sequences, wherein the light chain CDR1 sequence has the amino acid sequence SEQ ID NO: 47; the light chain CDR2 sequence has the amino acid sequence of SEQ ID NO: 48; and the light chain CDR3 sequence has the amino acid sequence of SEQ ID NO: 49; or variants of any of the foregoing;
(i) a heavy chain variable region comprising heavy chain CDR1, CDR2, and CDR3 sequences, wherein the heavy chain CDR1 sequence has the amino acid sequence of SEQ ID NO: 56; the heavy chain CDR2 has the amino acid sequence of SEQ ID NO: 57; and the heavy chain CDR3 sequence has the amino acid sequence of SEQ ID NO: 58; or variants of any of the foregoing; and (ii) a light chain variable region comprising light chain CDR1, CDR2, and CDR3 sequences, wherein the light chain CDR1 sequence has the amino acid sequence SEQ ID NO: 53; the light chain CDR2 sequence has the amino acid sequence of SEQ ID NO: 54; and the light chain CDR3 sequence has the amino acid sequence of SEQ ID NO: 55; or variants of any of the foregoing;
(i) a heavy chain variable region comprising heavy chain CDR1, CDR2, and CDR3 sequences, wherein the heavy chain CDR1 sequence has the amino acid sequence of SEQ ID NO: 62; the heavy chain CDR2 has the amino acid sequence of SEQ ID NO: 63; and the heavy chain CDR3 sequence has the amino acid sequence of SEQ ID NO: 64; or variants of any of the foregoing; and (ii) a light chain variable region comprising light chain CDR1, CDR2, and CDR3 sequences, wherein the light chain CDR1 sequence has the amino acid sequence SEQ ID NO: 59; the light chain CDR2 sequence has the amino acid sequence of SEQ ID NO: 60; and the light chain CDR3 sequence has the amino acid sequence of SEQ ID NO: 61; or variants of any of the foregoing; or
(i) a heavy chain variable region comprising heavy chain CDR1, CDR2, and CDR3 sequences, wherein the heavy chain CDR1 sequence has the amino acid sequence of SEQ ID NO: 38; the heavy chain CDR2 has the amino acid sequence of SEQ ID NO: 39; and the heavy chain CDR3 sequence has the amino acid sequence of SEQ ID NO: 40; or variants of any of the foregoing; and (ii) a light chain variable region comprising light chain CDR1, CDR2, and CDR3 sequences, wherein the light chain CDR1 sequence has the amino acid sequence SEQ ID NO: 35; the light chain CDR2 sequence has the amino acid sequence of SEQ ID NO: 36; and the light chain CDR3 sequence has the amino acid sequence of SEQ ID NO: 81; or variants of any of the foregoing.

Embodiment 45. The pharmaceutical composition of any one of embodiments 43-44, wherein the antibody comprises a VL sequence as set forth in SEQ ID NO: 1, 3, 5, 7, 9, 11, 13, 15, 79, 86, 96, or 98, or a variant thereof.

Embodiment 46. The pharmaceutical composition of any one of embodiments 43-45, wherein the antibody comprises a VH sequence as set forth in SEQ ID NO: 2, 4, 6, 8, 10, 12, 14, 16, 80, 83, 91, 96, or 99 or a variant thereof.

Embodiment 47. The pharmaceutical composition of any one of claims 43-46, wherein the antibody comprises a VL and VH comprising an amino acid sequence of SEQ ID NO: 1 and SEQ ID NO: 2; SEQ ID NO: 3 and SEQ ID NO: 4; SEQ ID NO: 5 and SEQ ID NO: 6; SEQ ID NO: 7 and SEQ ID NO: 8; SEQ ID NO: 9 and SEQ ID NO: 10; SEQ ID NO: 11 and SEQ ID NO: 12; SEQ ID NO: 3 and SEQ ID NO: 83; SEQ ID NO: 13 and SEQ ID NO: 14; SEQ ID NO: 15 and SEQ ID NO: 16; SEQ ID NO: 79 and SEQ ID NO: 80; SEQ ID NO: 86 and SEQ ID NO: 14; SEQ ID NO: 98 and SEQ ID NO: 99, respectively, or a variant thereof, wherein the CDRs of the variant are constant.

Embodiment 48. The pharmaceutical composition of any one of embodiments 43-47, wherein the variant has at least 85% homology to a sequence of SEQ ID NO: 1-72, 78-83, or 85-86.

Embodiment 49. The pharmaceutical composition of any one of embodiments 43-48, wherein the variant has at least 90%, 91%, 92%, 93%, 94%, 95%, 96%, 97%, 98%, or 99% homology to a sequence of SEQ ID NO: 1-72, 78-83, or 85-86.

Embodiment 50. The pharmaceutical composition of any one of embodiments 43-49, wherein the variant has at least 85% identity to a sequence of SEQ ID NO: 1-72, 78-83, or 85-86.

Embodiment 51. The pharmaceutical composition of any one of embodiments 43-50, wherein the variant has at least 90%, 91%, 92%, 93%, 94%, 95%, 96%, 97%, 98%, or 99% identify to a sequence of SEQ ID NO: 1-72, 78-83, or 85-86.

Embodiment 52. The pharmaceutical composition of any one of embodiments 43-51, wherein the antibody, or antigen-binding fragment thereof, is present at a concentration of about 10-200 mg/mL.

Embodiment 53. The pharmaceutical composition of any one of embodiments 43-52, wherein the antibody, or antigen-binding fragment thereof, is present at a concentration of about 20-150 mg/mL.

Embodiment 54. The pharmaceutical composition of any one of embodiments 43-52, wherein the antibody, or antigen-binding fragment thereof, is present at a concentration of about 25 mg/mL.

Embodiment 55. The pharmaceutical composition of any one of embodiments 43-52, wherein the antibody, or antigen-binding fragment thereof, is present at a concentration of about 50 mg/mL.

Embodiment 56. The pharmaceutical composition of any one of embodiments 43-52, wherein the antibody, or antigen-binding fragment thereof, is present at a concentration of about 150 mg/mL.

Embodiment 57. The pharmaceutical composition of any one of embodiments 43-56, wherein the histidine buffer is at a concentration of about 10-60 mM.

Embodiment 58. The pharmaceutical composition of any one of embodiments 43-57, wherein the histidine buffer is at a concentration of about 10-40 mM.

Embodiment 59. The pharmaceutical composition of any one of embodiments 43-58, wherein the histidine buffer is at a concentration of about 15-30 mM.

Embodiment 60. The pharmaceutical composition of any one of embodiments 43-59, wherein the histidine buffer is at a concentration of about 20 mM.

Embodiment 61. The pharmaceutical composition of any one of embodiments 43-60, wherein the sucrose is at a concentration of about 1-15% (w/v).

Embodiment 62. The pharmaceutical composition of any one of embodiments 43-61, wherein the sucrose is at a concentration of about 5-10% (w/v).

Embodiment 63. The pharmaceutical composition of any one of embodiments 43-62, wherein the sucrose is at a concentration of about 8% (w/v).

Embodiment 64. The pharmaceutical composition of any one of embodiments 43-63, wherein the methionine, or L-methionine, is present at a concentration of about 1-15 mM.

Embodiment 65. The pharmaceutical composition of any one of embodiments 43-64, wherein the methionine, or L-methionine, is present at a concentration of about 7-13 mM.

Embodiment 66. The pharmaceutical composition of any one of embodiments 43-65, wherein the methionine, or L-methionine, is present at a concentration of about 10 mM.

Embodiment 67. The pharmaceutical composition of any one of embodiments 43-66, wherein the polysorbate is polysorbate 80 (PS80), or polysorbate 20 (PS20).

Embodiment 68. The pharmaceutical composition of any one of embodiments 43-67, wherein the polysorbate is polysorbate 80 (PS80).

Embodiment 69. The pharmaceutical composition of any one of embodiments 43-68, wherein the polysorbate 80 is at a concentration of about 0.001-1% (w/v).

Embodiment 70. The pharmaceutical composition of any one of embodiments 43-69, wherein the polysorbate 80 is at a concentration of about 0.01-0.1% (w/v).

Embodiment 71. The pharmaceutical composition of any one of embodiments 43-70, wherein the polysorbate 80 is at a concentration of about 0.01-0.05% (w/v).

Embodiment 72. The pharmaceutical composition of any one of embodiments 43-71, wherein the polysorbate 80 is at a concentration of about 0.01% (w/v).

Embodiment 73. The pharmaceutical composition of any one of embodiments 43-72, wherein the polysorbate 80 is at a concentration of about 0.02% (w/v).

Embodiment 74. The pharmaceutical composition of any one of embodiments 43-73, wherein the pH of the pharmaceutical composition is about 5.0 to about 6.5.

Embodiment 75. The pharmaceutical composition of embodiment 74, wherein the pH of the pharmaceutical composition is about 5.5.

Embodiment 76. The pharmaceutical composition of embodiment 74, wherein the pH of the pharmaceutical composition is about 6.0.
(5) Embodiment 77. A pharmaceutical composition comprising: an anti-IGF-1R antibody, or antigen-binding fragment thereof at a concentration of about 10-200 mg/mL, comprising:
a light chain and heavy chain comprising the amino acid sequence of SEQ ID NO: 1 and SEQ ID NO: 2; SEQ ID NO: 3 and SEQ ID NO: 4; SEQ ID NO: 5 and SEQ ID NO: 6; SEQ ID NO: 7 and SEQ ID NO: 8; SEQ ID NO: 9 and SEQ ID NO: 10; SEQ ID NO: 11 and SEQ ID NO: 12; SEQ ID NO: 3 and SEQ ID NO: 83; SEQ ID NO: 93 and SEQ ID NO: 92 SEQ ID NO: 15 and SEQ ID NO: 16; SEQ ID NO: 79 and SEQ ID NO: 80; SEQ ID NO: 86 and SEQ ID NO: 14; SEQ ID NO: 3 and SEQ ID NO: 83 SEQ ID NO: 100 and SEQ ID NO: 101, respectively, or a variant thereof;
a V_{L} and V_{H} comprising the amino acid sequence of SEQ ID NO: 1 and SEQ ID NO: 2; SEQ ID NO: 3 and SEQ ID NO: 4; SEQ ID NO: 5 and SEQ ID NO: 6; SEQ ID NO: 7 and SEQ ID NO: 8; SEQ ID NO: 9 and SEQ ID NO: 10; SEQ ID NO: 11 and SEQ ID NO: 12; SEQ ID NO: 3 and SEQ ID NO: 83; SEQ ID NO: 13 and SEQ ID NO: 14; SEQ ID NO: 15 and SEQ ID NO: 16; SEQ ID NO: 79 and SEQ ID NO: 80; SEQ ID NO: 86 and SEQ ID NO: 14; SEQ ID NO: 98 and SEQ ID NO: 99, respectively, or a variant thereof, wherein the CDRs of the variant are constant;
a V_{L} and V_{H} comprising the amino acid sequence of SEQ ID NO: 13 and SEQ ID NO: 14 or a variant thereof, wherein the CDRs of the variant are constant;
   a light chain comprising the amino acid sequence of SEQ ID NO: 93 and a heavy chain comprising the amino acid sequence of SEQ ID NO: 92; or a light chain comprising the amino acid sequence of SEQ ID NO: 93 and a heavy chain comprising the amino acid sequence of SEQ ID NO: 94;
a V_{L} and V_{H} comprising the amino acid sequence of SEQ ID NO: 98 and SEQ ID NO: 99, or a variant thereof, wherein the CDRs of the variant are constant;
   a light chain comprising the amino acid sequence of SEQ ID NO: 3 and a heavy chain comprising the amino acid sequence of SEQ ID NO: 83,
   wherein the variant has at least 90%, 91%, 92%, 93%, 94%, 95%, 96%, 97%, 98%, or 99% identity to a V_{L} and V_{H} comprising the amino acid sequence of SEQ ID NO: 1 and SEQ ID NO: 2; SEQ ID NO: 3 and SEQ ID NO: 4; SEQ ID NO: 5 and SEQ ID NO: 6; SEQ ID NO: 7 and SEQ ID NO: 8; SEQ ID NO: 9 and SEQ ID NO: 10; SEQ ID NO: 11 and SEQ ID NO: 12; SEQ ID NO: 3 and SEQ ID NO: 83; SEQ ID NO: 13 and SEQ ID NO: 14; SEQ ID NO: 15 and SEQ ID NO: 16; SEQ ID NO: 79 and SEQ ID NO: 80; SEQ ID NO: 86 and SEQ ID NO: 14; SEQ ID NO: 98 and SEQ ID NO: 99, respectively, or a variant thereof, wherein the CDRs of the variant are constant;
(ii) a histidine buffer at a concentration of about 10-40 mM;
(iii) sucrose at a concentration of about 1-10% (w/v);
(iv) methionine, or L-methionine, at a concentration of about 1-15 mM; and
(v) a polysorbate at a concentration of about 0.01-0.1% (w/v),
wherein the pH of the pharmaceutical compositions is about 5.0 to about 6.5. Embodiment 78. The pharmaceutical composition of embodiment 77, wherein the variant has at least 85% homology to a sequence of SEQ ID NO: 1-72, 78-83, or 85-86.

Embodiment 79. The pharmaceutical composition of any one of embodiments 77-78, wherein the variant has at least 90%, 91%, 92%, 93%, 94%, 95%, 96%, 97%, 98%, or 99% homology to a sequence of SEQ ID NO: 1-72, 78-83, or 85-86.

Embodiment 80. The pharmaceutical composition of any one of embodiments 77-79, wherein the variant has at least 85% identity to a sequence of SEQ ID NO: 1-72, 78-83, or 85-86.

Embodiment 81. The pharmaceutical composition of any one of embodiments 77-80, wherein the variant has at least 90%, 91%, 92%, 93%, 94%, 95%, 96%, 97%, 98%, or 99% identify to a sequence of SEQ ID NO: 1-72, 78-83, or 85-86.

Embodiment 83. The pharmaceutical composition of any one of embodiments 77-81, wherein the antibody, or antigen-binding fragment thereof, is present at a concentration of about 20-150 mg/mL.

Embodiment 84. The pharmaceutical composition of any one of embodiments 77-82, wherein the antibody, or antigen-binding fragment thereof, is present at a concentration of about 25 mg/mL.

Embodiment 85. The pharmaceutical composition of any one of embodiments 77-82, wherein the antibody, or antigen-binding fragment thereof, is present at a concentration of about 50 mg/mL.

Embodiment 86. The pharmaceutical composition of any one of embodiments 77-82, wherein the antibody, or antigen-binding fragment thereof, is present at a concentration of about 150 mg/mL.

Embodiment 87. The pharmaceutical composition of any one of embodiments 77-86, wherein the histidine buffer is at a concentration of about 15-30 mM.

Embodiment 88. The pharmaceutical composition of any one of embodiments 77-87, wherein the histidine buffer is at a concentration of about 20 mM.

Embodiment 89. The pharmaceutical composition of any one of embodiments 77-88, wherein the sucrose is at a concentration of about 5-10% (w/v).

Embodiment 90. The pharmaceutical composition of any one of embodiments 77-89, wherein the sucrose is at a concentration of about 8% (w/v).

Embodiment 91. The pharmaceutical composition of any one of embodiments 77-90, wherein the methionine, or L-methionine, is present at a concentration of about 1-15 mM.

Embodiment 92. The pharmaceutical composition of any one of embodiments 77-91, wherein the methionine, or L-methionine, is present at a concentration of about 7-13 mM.

Embodiment 93. The pharmaceutical composition of any one of embodiments 77-92, wherein the methionine, or L-methionine, is present at a concentration of about 10 mM.

Embodiment 94. The pharmaceutical composition of any one of embodiments 77-93, wherein the polysorbate is polysorbate 80 (PS80), or polysorbate 20 (PS20).

Embodiment 95. The pharmaceutical composition of any one of embodiments 77-94, wherein the polysorbate is polysorbate 80 (PS80).

Embodiment 96. The pharmaceutical composition of any one of embodiments 77-95, wherein the polysorbate 80 is at a concentration of about 0.01-0.05% (w/v).

Embodiment 97. The pharmaceutical composition of any one of embodiments 77-96, wherein the polysorbate 80 is at a concentration of about 0.01% (w/v).

Embodiment 98. The pharmaceutical composition of any one of embodiments 77-97, wherein the polysorbate 80 is at a concentration of about 0.02% (w/v).

Embodiment 99. The pharmaceutical composition of any one of embodiments 77-98, wherein the pH of the pharmaceutical composition is about 5.5 to about 6.0.

Embodiment 100. The pharmaceutical composition of embodiment 99, wherein the pH of the pharmaceutical composition is about 5.5.

Embodiment 101. The pharmaceutical composition of embodiment 99, wherein the pH of the pharmaceutical composition is about 6.0.

Embodiment 102. The pharmaceutical composition of any one of embodiments 1-101, suitable for intravenous, subcutaneous, or intramuscular administration.

Embodiment 103. The pharmaceutical composition of any one of embodiments 1-101, wherein the pharmaceutical composition is a liquid pharmaceutical composition.

Embodiment 104. The pharmaceutical composition of any one of embodiments 1-101, wherein the pharmaceutical composition is a lyophilized pharmaceutical composition.

Embodiment 105. The pharmaceutical composition of any one of embodiments 1-101, wherein the pharmaceutical composition main peak, measured by SEC-UPLC, decreases in area percent by less than 3% after 4 weeks at a temperature of 40° C., relative to the initial value.

Embodiment 106. The pharmaceutical composition of any one of embodiments 1-101, wherein the pharmaceutical composition main peak, measured by SEC-UPLC, decreases in area percent by less than 2% after 4 weeks at a temperature of 40° C., relative to the initial value.

Embodiment 107. The pharmaceutical composition of any one of embodiments 1-101, wherein the pharmaceutical composition main peak, measured by SEC-UPLC, decreases in area percent by less than 1.3% after 4 weeks at a temperature of 40° C., relative to the initial value.

Embodiment 108. A dosage form comprising the pharmaceutical composition of any one of embodiments 1-101 in a container.

Embodiment 109. The dosage form of embodiment 108, wherein the container is a plastic vial or glass vial.

Embodiment 110. The dosage form of embodiment 108, wherein the container is a glass vial with a volume of 2 mL, 6 mL, or 10 mL.

Embodiment 111. The dosage form of embodiment 108, wherein the container is a pre-filled syringe.

Embodiment 112. The dosage form of embodiment 108, wherein the container is an autoinjector.

Embodiment 113. A kit, comprising the pharmaceutical composition of any one of embodiments 1-107 or the dosage form of any one of embodiments 108-112 and instructions for use.

Embodiment 114. A method of treating thyroid associated ophthalmopathy in a subject, the method comprising administering a pharmaceutical composition of any one of embodiments 1-107 to the subject.

Embodiment 115. The method of embodiment 114, wherein the pharmaceutical composition is administered intravenously.

Embodiment 116. The method of embodiment 114, wherein the pharmaceutical composition is administered subcutaneously.

Embodiment 117. The method of any one of embodiments 114-116, wherein the pharmaceutical composition is administered in a first dose and one or more subsequent dose.

Embodiment 118. The method of embodiment 117, wherein the first dose is selected from the group consisting of: about 1 mg/kg to about 2 mg/kg, about 2 mg/kg to about 5 mg/kg, about 3 mg/kg to about 5 mg/kg, about 5 mg/kg to about 7.5 mg/kg, about 7.5 mg/kg to about 10 mg/kg, about 10 mg/kg to about 15 mg/kg, or about 15 mg/kg to about 20 mg/kg, of the anti-IGF-1R antibody to the subject.

Embodiment 119. The method of embodiment 118, wherein the first dose is about 3 mg/kg, about 10 mg/kg, or about 20 mg/kg.

Embodiment 120. The method of embodiment 117, wherein the one or more subsequent dose is selected from the group consisting of: about 1 mg/kg to about 2 mg/kg, about 2 mg/kg to about 5 mg/kg, about 3 mg/kg to about 5 mg/kg, about 5 mg/kg to about 7.5 mg/kg, about 7.5 mg/kg to about 10 mg/kg, about 10 mg/kg to about 15 mg/kg, or about 15 mg/kg to about 20 mg/kg of the anti-IGF-1R antibody to the subject.

Embodiment 121. The method of embodiment 120, wherein the one or more subsequent dose is about 3 mg/kg, about 10 mg/kg, or about 20 mg/kg.

Embodiment 122. The method of any one of embodiments 117-121, wherein the one or more subsequent dose amounts is the same as the first dose amount.

Embodiment 123 The method of any one of embodiments 117-121, wherein the one or more subsequent dose amounts is different from the first dose amount.

Embodiment 124. The method of any one of embodiments 117-123, wherein at least one subsequent dose of the one or more subsequent doses is administered one, two, three, four, five, six, or eight weeks after the first dose.

Embodiment 125. The method of embodiment 124, wherein the one or more subsequent doses of the one or more subsequent doses is administered three weeks after the first dose.

Embodiment 126. The method of embodiment 124, wherein the subsequent dose is administered every three weeks after the first dose for 4, 5, 6, 7, or 8 cycles.

Embodiment 127. The method of embodiment 124, wherein the subsequent dose is administered every three weeks after the first dose for 5 or 8 cycles.

Embodiment 128. The method of embodiment 124, wherein the method comprises administering a total of five, six, seven or eight doses to the subject.

Embodiment 129. The method of any one of embodiments 117-128, wherein after first dose of the antibody, the clinical activity score of the subject is reduced.

Embodiment 130. The method of any one of embodiments 117-129, wherein after two doses of the antibody, the clinical activity score of the subject is reduced.

Embodiment 131. The method of embodiments 117-130, wherein after the one or more subsequence dose, the clinical activity score of the subject is reduced within 6 weeks of the first dose.

Embodiment 132. The method of embodiments 117-130, wherein after the one or more subsequence dose, the clinical activity score of the subject is reduced within 3 weeks of an initial one or more subsequent doses.

Embodiment 133. The method of any one of embodiments 117-132, wherein the antibody is administered by intravenous infusion over 45 minutes to about 90 minutes, or over 60 minutes to about 90 minutes.

Embodiment 134. The method of any one of embodiments 114-133, wherein the treated subject's proptosis is reduced by at least, or about, 1-4 mm.

Embodiment 135. The method of embodiment 134, wherein the proptosis is reduced by at least, or about 2-3 mm.

Embodiment 136. The method of embodiments 134 or 135, wherein the proptosis is reduced within 3 weeks of the first dose.

Embodiment 137. The method of embodiments 134 or 135, wherein the proptosis is reduced within 6 weeks of the first dose.

Embodiment 138. The method of any one of embodiments 114-137, wherein the treated subject has reduced diplopia.

Embodiment 139. The method of embodiment 138, wherein the diplopia is reduced within 3 weeks or 6 weeks of the first dose.

Embodiment 140. The method of any one of embodiments 114-139, wherein the subject has an improvement in Clinical Activity Score (CAS) within 3 weeks or 6 weeks.

Embodiment 141. The method of embodiment 140, wherein the CAS score has an improvement of at least −2, −3, or −4.

Embodiment 142. The method of any one of embodiments 114-141, wherein the subject has a reduction in proptosis and an improvement in CAS score within 3 weeks or within 6 weeks of the first dose.

Embodiment 143. A method of treating thyroid associated ophthalmopathy in a subject, the method comprising administering a pharmaceutical composition of any one of embodiments 1-107 to the subject, wherein the administering comprises intravenously administering a dose of 3 mg/kg, 10 mg/kg, or 20 mg/kg of an anti-IGF-1R antibody to the subject at a regular interval for a period sufficient to reduce one or more symptoms associated with thyroid associated ophthalmopathy.

Embodiment 144. The method of embodiment 143, wherein the anti-IGF-1R antibody is administered by intravenous infusion.

Embodiment 145. The method of any one of embodiments 143-144, wherein the anti-IGF-1R antibody is administered every 3 weeks.

Embodiment 146. The method of any one of embodiments 143-145, wherein the anti-IGF-1R antibody is administered for a period sufficient for 5 doses.

Embodiment 147. The method of any one of embodiments 143-145, wherein the anti-IGF-1R antibody is administered for a period sufficient for 8 doses.

Embodiment 148. The method of any one of embodiments 143-147, wherein the anti-IGF-1R antibody is administered for a period selected from 3 weeks, 6 weeks, 9 weeks, 12 weeks, 15 weeks, 18 weeks, 21 weeks, 24 weeks or longer.

Embodiment 149. The method of any one of embodiments 143-148, wherein the subject's proptosis is reduced by at least, or about, 1-4 mm.

Embodiment 150. The method of embodiment 149, wherein the proptosis is reduced by at least, or about 2-3 mm.

Embodiment 151. The method of embodiments 149 or 150, wherein the proptosis is reduced within 3 weeks of the first dose.

Embodiment 152. The method of embodiments 149 or 150, wherein the proptosis is reduced within 6 weeks of the first dose.

Embodiment 153. The method of any one of embodiments 143-152, wherein the treated subject has reduced diplopia.

Embodiment 154. The method of embodiment 153, wherein the diplopia is reduced within 3 weeks or 6 weeks of the first dose.

Embodiment 155. The method of any one of embodiments 143-154, wherein the subject has an improvement in Clinical Activity Score (CAS) within 3 weeks or 6 weeks.

Embodiment 156. The method of embodiment 155, wherein the CAS score has an improvement of at least −2, −3, or −4.

Embodiment 157. The method of any one of embodiments 143-156, wherein the subject has a reduction in proptosis and an improvement in CAS score within 3 weeks or within 6 weeks of the first dose.

Embodiment 158. A method of treating thyroid associated ophthalmopathy in a subject, the method comprising administering a pharmaceutical composition of any one of embodiments 1-107 to the subject, wherein the administering comprises subcutaneously administering a dose of 3 mg/kg of an anti-IGF-1R antibody to the subject at a regular interval for a period sufficient to reduce one or more symptoms associated with thyroid associated ophthalmopathy.

Embodiment 159. The method of embodiment 158, wherein the anti-IGF-1R antibody is administered by subcutaneous infusion or injection.

Embodiment 160. The method of any one of embodiments 158-159, wherein the anti-IGF-1R antibody is administered every 3 weeks.

Embodiment 161. The method of any one of embodiments 158-159, wherein the anti-IGF-1R antibody is administered for a period sufficient for 5 doses.

Embodiment 162. The method of any one of embodiments 158-159, wherein the anti-IGF-1R antibody is administered for a period sufficient for 8 doses.

Embodiment 163. The method of any one of embodiments 158-162, wherein the anti-IGF-1R antibody is administered for a period selected from 3 weeks, 6 weeks, 9 weeks, 12 weeks, 15 weeks, 18 weeks, 21 weeks, 24 weeks or longer.

Embodiment 164. The method of any one of embodiments 158-163, wherein the subject's proptosis is reduced by at least, or about, 1-4 mm.

Embodiment 165. The method of embodiment 164, wherein the proptosis is reduced by at least, or about 2-3 mm.

Embodiment 166. The method of embodiments 164 or 165, wherein the proptosis is reduced within 3 weeks of the first dose.

Embodiment 167. The method of embodiments 164 or 165, wherein the proptosis is reduced within 6 weeks of the first dose.

Embodiment 168. The method of any one of embodiments 158-167, wherein the treated subject has reduced diplopia.

Embodiment 169. The method of embodiment 168, wherein the diplopia is reduced within 3 weeks or 6 weeks of the first dose.

Embodiment 170. The method of any one of embodiments 158-169, wherein the subject has an improvement in Clinical Activity Score (CAS) within 3 weeks or 6 weeks.

Embodiment 171. The method of embodiment 170, wherein the CAS score has an improvement of at least −2, −3, or −4.

Embodiment 172. The method of any one of embodiments 158-171, wherein the subject has a reduction in proptosis and an improvement in CAS score within 3 weeks or within 6 weeks of the first dose.

Embodiment 173. A method of treating or reducing the severity of, thyroid-associated ophthalmopathy (TAO), or a symptom thereof, comprising administering to a subject a pharmaceutical composition of any one of embodiments 1-107.

Embodiment 174. A method of reducing proptosis in an eye in a subject with thyroid-associated ophthalmopathy (TAO) comprising administering to a subject a pharmaceutical composition of any one of embodiments 1-107.

Embodiment 175. A method of treating thyroid eye disease in a subject comprising administering to a subject a pharmaceutical composition of any one of embodiments 1-107.

Embodiment 176. A method of reducing Clinical Activity Score (CAS) of thyroid-associated ophthalmopathy (TAO) in a subject comprising administering to a subject a pharmaceutical composition of any one of embodiments 1-107.

Embodiment 177. A method of a) reducing proptosis by at least 2 mm and b) reducing the clinical activity score (CAS) in a subject with thyroid-associated ophthalmopathy (TAO) comprising administering to a subject a pharmaceutical composition of any one of embodiments 1-107.

Embodiment 178. The method of any of embodiments 173-177, wherein proptosis is reduced by at least 2 mm.

Embodiment 179. The method of any of embodiments 173-177, wherein

proptosis is reduced by at least 3 mm.

Embodiment 180. The method of any of embodiments 173-177, wherein

proptosis is reduced by at least 4 mm.

Embodiment 181. The method of any of embodiments 173-177, wherein the clinical activity score (CAS) of the subject is reduced by at least 2 points.

Embodiment 182. The method of any of embodiments 173-177, wherein the clinical activity score (CAS) of the subject is reduced to one (1).

Embodiment 183. The method of any of embodiments 173-177, wherein the clinical activity score (CAS) of the subject is reduced to zero (0).

Embodiment 184. A method of treating or reducing the severity of thyroid-associated ophthalmopathy (TAO) in a subject comprising administering to a subject a pharmaceutical composition of any one of embodiments 1-107, wherein treatment with said pharmaceutical composition (i) reduces proptosis by at least 2 mm in an eye; (ii) is not accompanied by a deterioration of 2 mm or more in the other (or fellow eye); and (iii) reduces the CAS in said subject to either one (1) or zero (0).

Embodiment 185. A method of improving the quality of life in a subject with thyroid-associated ophthalmopathy (TAO, also called Grave” Ophthalmopathy/Grave” Orbitopathy) comprising administering to a subject a pharmaceutical composition of any one of embodiments 1-107.

Embodiment 186. The method of embodiment 185, wherein the quality of life is measured by the Grave” Ophthalmopathy Quality of Life (GO-QoL) assessment, or either the Visual Functioning or Appearance subscale thereof.

Embodiment 187. The method of embodiment 185, wherein the treatment results in an improvement of greater than or equal to 8 points on the GO-QoL.

Embodiment 188. The method of embodiment 185, wherein the treatment results in an improvement on the Functioning subscale of the GO-QoL.

Embodiment 189. The method of embodiment 185, wherein the treatment results in an improvement on the Appearance subscale of the GO-QoL.

Embodiment 190. A method of treating or reducing the severity of diplopia in a subject with thyroid-associated ophthalmopathy (TAO) comprising administering to a subject a pharmaceutical composition of any one of embodiments 1-107.

Embodiment 191. The method of embodiment 190, wherein the diplopia is constant diplopia.

Embodiment 192. The method of embodiment 190, wherein the diplopia is inconstant diplopia.

Embodiment 193. The method of embodiment 190, wherein the diplopia is intermittent diplopia.

Embodiment 194. The method of embodiment 190, wherein the improvement in or reduction in severity of diplopia is sustained at least 20 weeks after discontinuation of antibody administration.

Embodiment 195. The method of embodiment 190, wherein the improvement in or reduction in severity of diplopia is sustained at least 50 weeks after discontinuation of antibody administration.

Embodiment 196. The method of any one of embodiments 173-195, wherein the pharmaceutical composition is administered as a first dose and a subsequence dose, or subsequent doses.

Embodiment 197. The method of embodiment 196, wherein the subsequent dose, or subsequence doses, are administered once a week, once every two weeks, once every 3 weeks, or once every 4 weeks.

Embodiment 198. A method of increasing the internalization of IGF-1R on a cell, the method comprising contacting the cell with a pharmaceutical composition of any one of embodiments 1-107.

Embodiment 199. The method of embodiment 198, wherein the contacting comprises administering to a subject the pharmaceutical composition.

Embodiment 200. The method of embodiment 199, wherein the subject has or is at risk of thyroid eye disease (TED).

Embodiment 201. A method of inhibiting IGF-1 stimulated receptor phosphorylation on a cell, the method comprising contacting the cell with a pharmaceutical composition of any one of embodiments 1-107.

Embodiment 202. The method of embodiment 201, wherein the contacting comprises administering to a subject the pharmaceutical composition.

Embodiment 203. The method of embodiment 201, wherein the subject has or is at risk of thyroid eye disease (TED).

Embodiment 204. The method of any one of embodiments 198-203, wherein the cell is an A549 cell or a HOCF cell.

Embodiment 205. A method of inhibiting IGF-1 induced receptor autophosphorylation by at least 95%, 96%, 97%, 98%, or 99% or by 100% in a subject in need thereof, the method comprising contacting the cell with a pharmaceutical composition of any one of embodiments 1-107.

Embodiment 206. A stable formulation comprising an anti-IGF-1R antibody or antigen-binding fragment thereof at a concentration of 25-75 mg/ml, wherein the antibody comprises a heavy chain with heavy chain complementarity determining regions (CDRs) comprising an HCDR1 comprising SEQ ID NO: 53, an HCDR2 comprising SEQ ID NO: 54, an HCDR3 comprising SEQ ID NO: 55, and a light chain with light chain CDRs comprising a LCDR1 comprising SEQ ID NO: 56, a LCDR2 comprising SEQ ID NO: 57 and a LCDR3 comprising SEQ ID NO: 58, and wherein the composition has a pH of 4.5-6.0.

Embodiment 207. The stable formulation of embodiment 206, wherein the heavy chain comprises an amino acid sequence that is at least 90%, 91%, 92%, 93%, 94%, 95%, 96%, 97%, 98%, 99% or 100% identical to SEQ ID NO: 14, and the light chain comprises an amino acid sequence that is at least 90%, 91%, 92%, 93%, 94%, 95%, 96%, 97%, 98%, 99% or 100% identical to SEQ ID NO: 13; or wherein the heavy chain comprises an amino acid sequence that is at least 90%, 91%, 92%, 93%, 94%, 95%, 96%, 97%, 98%, 99% or 100% identical to SEQ ID NO: 92, and the light chain comprises an amino acid sequence that is at least 90%, 91%, 92%, 93%, 94%, 95%, 96%, 97%, 98%, 99% or 100% identical to SEQ ID NO: 93.

Embodiment 208. The stable formulation of embodiment 206, wherein the anti-IGF-1R antibody is present at a concentration of 50 mg/ml.

Embodiment 209. The stable formulation of any one of the preceding embodiments, wherein the pH is 5-6.

Embodiment 210. The stable formulation of any one of the preceding embodiments, wherein the pH is 5.5.

Embodiment 211. The stable formulation of any one of the preceding embodiments, wherein the formulation comprises sucrose.

Embodiment 212. The stable formulation of embodiment 211, wherein sucrose is present at a concentration of about 1-20% (w/v).

Embodiment 213. The stable composition of embodiment 211, wherein sucrose is present at a concentration of about 8% (w/v).

Embodiment 214. The stable formulation of any one of the preceding embodiments, wherein the formulation comprises a stabilizer.

Embodiment 215. The stable formulation of embodiment 214, wherein the stabilizer is methionine.

Embodiment 216. The stable formulation of embodiment 215, wherein methionine is present at a concentration of about 1-15 mM.

Embodiment 217. The stable formulation of embodiment 216, wherein methionine is present at a concentration of about 10 mM.

Embodiment 218. The stable formulation of any of the preceding embodiments, wherein the composition comprises a surfactant.

Embodiment 219. The stable formulation of embodiment 218, wherein the surfactant is polysorbate 80.

Embodiment 220. The stable formulation of embodiment 219, wherein polysorbate 80 is present at a concentration of about 0.001-1% (w/v).

Embodiment 221. The stable formulation of embodiment 219, wherein polysorbate 80 is present at a concentration of about 0.02% (w/v).

Embodiment 222. The stable formulation of any one of the preceding embodiments, wherein the formulation comprises a buffering agent.

Embodiment 223. The stable formulation of embodiment 222, wherein the buffering agent is histidine.

Embodiment 224. The stable formulation of embodiment 223, wherein the concentration of a histidine buffer is between about 10-60 mM.

Embodiment 225. The stable formulation of embodiment 224, wherein the concentration of a histidine buffer is about 20 mM.

Embodiment 226. A stable formulation comprising: an anti-IGF-1R antibody comprising at a concentration of 45-55 mg/ml; a histidine buffer at concentration of 20 mM; methionine at a concentration of 10 mM; polysorbate 80 at a concentration of 0.02% (w/v); sucrose at a concentration of 8% (w/v); and pH of 5.5; wherein the antibody comprises heavy chain complementarity determining regions (CDRs) comprising an HCDR1 comprising SEQ ID NO: 53, an HCDR2 comprising SEQ ID NO: 54, an HCDR3 comprising SEQ ID NO: 55, and a light chain with light chain CDRs comprising a LCDR1 comprising SEQ ID NO: 56, a LCDR2 comprising SEQ ID NO: 57 and a LCDR3 comprising SEQ ID NO: 58, and wherein the composition has a pH of 4.5-6.0.

Embodiment 227. The stable formulation of embodiment 226, wherein the heavy chain comprises an amino acid sequence that is at least 90%, 91%, 92%, 93%, 94%, 95%, 96%, 97%, 98%, 99% or 100% identical to SEQ ID NO: 14, and the light chain comprises an amino acid sequence that is at least 90%, 91%, 92%, 93%, 94%, 95%, 96%, 97%, 98%, 99% or 100% identical to SEQ ID NO: 13; or wherein the heavy chain comprises an amino acid sequence that is at least 90%, 91%, 92%, 93%, 94%, 95%, 96%, 97%, 98%, 99% or 100% identical to SEQ ID NO: 92, and the light chain comprises an amino acid sequence that is at least 90%, 91%, 92%, 93%, 94%, 95%, 96%, 97%, 98%, 99% or 100% identical to SEQ ID NO: 93.

Embodiment 228. The stable formulation of embodiment 226 or 227, wherein concentration of the anti-IGF-1R antibody is 50 mg/ml.

Embodiment 229. The stable formulation of any one of the preceding embodiments, wherein the anti-IGF-1R antibody is present in the formulation as one or more anti-IGF-1R antibody variant species.

Embodiment 230. The stable formulation of embodiment 229, wherein the anti-IGF-1R antibody variant species are characterized as monomer species, high molecular weight species (HMWS), or charge variants.

Embodiment 231. The stable formulation of embodiment 229, wherein the formulation is characterized by
(i) the amount of anti-IGF-1R antibody monomer is 95% or greater as measured by the main peak in size exclusion chromatography (SEC), and
(ii) the amount of high molecular weight species (HMWS) is 5% or less as measured by SEC.

Embodiment 232. The stable formulation of any one of embodiments 229-231, wherein the composition is characterized by a charge variant profile as measured by iCIEF comprising
(i) 25% or greater main peak species,
(ii) 70% or less acidic species, and
(iii) 15% or less basic species.

Embodiment 233. The stable formulation of any one of the preceding embodiments, wherein the composition clarity is 18 NTU or less.

Embodiment 234. The stable formulation of any one of the preceding embodiments, wherein the osmolality is between 280-380 mOsmol/kg, or between 340-420 mOsmol/kg.

Embodiment 235. The stable formulation of any one of the preceding embodiments, wherein the formulation is stored at 25 to −25° C.

Embodiment 236. The stable formulation of any one of the preceding embodiments, wherein the formulation is stored at 2-6° C.

Embodiment 237. The stable formulation of any one of embodiments 206-236, suitable for intravenous, subcutaneous, or intramuscular administration.

Embodiment 238. The stable formulation of any one of embodiments 206-236, wherein the formulation is a liquid pharmaceutical composition or a lyophilized pharmaceutical composition.

Embodiment 239. A dosage form comprising the stable formulation of any one of embodiments 206-238 in a vial, pre-filled syringe or autoinjector.

Embodiment 240. Use of the stable formulation of any one of embodiments 206-239, for treatment of thyroid associated ophthalmopathy in a subject.

Embodiment 241. The use according to embodiment 240, wherein the stable formulation is administered intravenously.

Embodiment 242. The use according to embodiment 240, wherein the stable formulation is administered subcutaneously.

Embodiment 243. Use of the stable formulation of any one of embodiments 206-236 for reducing Clinical Activity Score (CAS) of thyroid-associated ophthalmopathy (TAO) in a subject, comprising administering the stable formulation.

Embodiment 244. The use according to any one of embodiments 240-243, wherein the treated subject has reduced diplopia.

Embodiment 245. Use of the composition of any one of embodiment 206-236 for treating or reducing the severity of thyroid-associated ophthalmopathy (TAO) in a subject comprising administering the stable formulation, wherein treatment with said pharmaceutical composition reduces proptosis by at least 2 mm in an eye; is not accompanied by a deterioration of 2 mm or more in the other (or fellow eye); and reduces the CAS in said subject to either one (1) or zero (0).

Embodiment 246. Use of the composition of any one of embodiments 206-236 for improving the quality of life in a subject with thyroid-associated ophthalmopathy (TAO, also called Graves' Ophthalmopathy/Graves' Orbitopathy), the use comprising administering the stable formulation of any one of embodiments 206-236.

Embodiment 247. The use according to embodiment 246, wherein the quality of life is measured by the Graves' Ophthalmopathy Quality of Life (GO-QOL) assessment, or either the Visual Functioning or Appearance subscale thereof.

Embodiment 248. The use according to embodiment 247, wherein greater than or equal to 8 points on the GO-QoL is achieved.

Embodiment 249. The use according to embodiment 247, wherein the treatment results in an improvement on the Functioning subscale of the GO-QoL.

Embodiment 250. The use according to embodiment 247, wherein the treatment results in an improvement on the Appearance subscale of the GO-QoL.

Embodiment 251. Use of the composition of any one of embodiments 206-236 for treating or reducing the severity of diplopia in a subject with thyroid-associated ophthalmopathy (TAO), the use comprising administering the stable formulation of any one of embodiment 206-236.

Embodiment 252. The use according to embodiment 251, wherein the diplopia is constant diplopia.

Embodiment 253. The use according to embodiment 251, wherein the diplopia is inconstant diplopia.

Embodiment 254. The use according to embodiment 251, wherein the diplopia is intermittent diplopia.

Embodiment 255. The pharmaceutical composition of claim 6, wherein the antibody comprises a VL and VH comprising the amino acid sequence of SEQ ID NO: 13 and SEQ ID NO: 14 or a variant thereof, wherein the CDRs of the variant are constant.

Embodiment 256. The pharmaceutical composition of claim 6, wherein the antibody comprises a light chain comprising the amino acid sequence of SEQ ID NO: 93 and a heavy chain comprising the amino acid sequence of SEQ ID NO: 92; or a light chain comprising the amino acid sequence of SEQ ID NO: 93 and a heavy chain comprising the amino acid sequence of SEQ ID NO: 94.

Embodiment 257. The pharmaceutical composition of claim 6, wherein the antibody comprises: a VL comprising the amino acid sequence of SEQ ID NO: 98 and a VH comprising the amino acid sequence of SEQ ID NO: 99, or a variant thereof, wherein the CDRs of the variant are constant; or a light chain comprising the amino acid sequence of SEQ ID NO: 3 and a heavy chain comprising the amino acid sequence of ID NO: 83.

Embodiment 258. The pharmaceutical composition of any one of claims 1-6, wherein the variant has at least 90%, 91%, 92%, 93%, 94%, 95%, 96%, 97%, 98%, or 99% identity to a VL and VH comprising the amino acid sequence of SEQ ID NO: 1 and SEQ ID NO: 2; SEQ ID NO: 3 and SEQ ID NO: 4; SEQ ID NO: 5 and SEQ ID NO: 6; SEQ ID NO: 7 and SEQ ID NO: 8; SEQ ID NO: 9 and SEQ ID NO: 10; SEQ ID NO: 11 and SEQ ID NO: 12; SEQ ID NO: 3 and SEQ ID NO: 83; SEQ ID NO: 13 and SEQ ID NO: 14; SEQ ID NO: 15 and SEQ ID NO: 16; SEQ ID NO: 79 and SEQ ID NO: 80; SEQ ID NO: 86 and SEQ ID NO: 14; SEQ ID NO: 98 and SEQ ID NO: 99, respectively, or a variant thereof, wherein the CDRs of the variant are constant.

Embodiment 259. The pharmaceutical composition of claim 47, wherein the antibody comprises a VL and VH comprising an amino acid sequence of SEQ ID NO: 13 and SEQ ID NO: 14 or a variant thereof, wherein the CDRs of the variant are constant.

Embodiment 260. The pharmaceutical composition of claim 47, wherein the antibody comprises a light chain comprising the amino acid sequence of SEQ ID NO: 93 and a heavy chain comprising the amino acid sequence of SEQ ID NO: 92; or a light chain comprising the amino acid sequence of SEQ ID NO: 93 and a heavy chain comprising the amino acid sequence of SEQ ID NO: 94.

Embodiment 261. The pharmaceutical composition of claim 47, wherein the antibody comprises: a VL and VH comprising the amino acid sequence of SEQ ID NO: 98 and SEQ ID NO: 99, or a variant thereof, wherein the CDRs of the variant are constant; or a light chain comprising the amino acid sequence of SEQ ID NO: 3 and a heavy chain comprising the amino acid sequence of SEQ ID NO: 83.

Embodiment 262. The pharmaceutical composition of any one of claims 43-46, wherein the variant has at least 90%, 91%, 92%, 93%, 94%, 95%, 96%, 97%, 98%, or 99% identity to a VL and VH comprising the amino acid sequence of SEQ ID NO: 1 and SEQ ID NO: 2; SEQ ID NO: 3 and SEQ ID NO: 4; SEQ ID NO: 5 and SEQ ID NO: 6; SEQ ID NO: 7 and SEQ ID NO: 8; SEQ ID NO: 9 and SEQ ID NO: 10; SEQ ID NO: 11 and SEQ ID NO: 12; SEQ ID NO: 3 and SEQ ID NO: 83; SEQ ID NO: 13 and SEQ ID NO: 14; SEQ ID NO: 15 and SEQ ID NO: 16; SEQ ID NO: 79 and SEQ ID NO: 80; SEQ ID NO: 86 and SEQ ID NO: 14; SEQ ID NO: 98 and SEQ ID NO: 99, respectively, or a variant thereof, wherein the CDRs of the variant are constant.

Embodiment 263. The pharmaceutical composition of claim 77, wherein the variant has at least 85% identity to the light chain and heavy chain, provided that the CDRs of the light chain and heavy chain are constant.

Embodiment 264. The pharmaceutical composition of any one of claims 77-78, wherein the variant has at least 90%, 91%, 92%, 93%, 94%, 95%, 96%, 97%, 98%, or 99% homology to the sequence of the light chain and heavy chain, provided that the CDRs of the light chain and heavy chain are constant.

Embodiment 265. The pharmaceutical composition of any one of claims 77-79, wherein the variant has at least 85% identity to the sequence of the light chain and heavy chain, provided that the CDRs of the light chain and heavy chain are constant.

Embodiment 266. The pharmaceutical composition of any one of claims 77-80, wherein the variant has at least 90%, 91%, 92%, 93%, 94%, 95%, 96%, 97%, 98%, or 99% identity to the sequence of the light chain and heavy chain, provided that the CDRs of the light chain and heavy chain are constant.

The subject matter is now described with reference to the following examples. These examples are provided for the purpose of illustration only and the claims should in no way be construed as being limited to these examples, but rather should be construed to encompass any and all variations which become evident as a result of the teaching provided herein. Those of skill in the art will readily recognize a variety of non-critical parameters that could be changed or modified to yield essentially similar results.

### EXAMPLES

### Example 1: Analytical Methods

The following example describes analytical methods used in the studies described in Examples 2-7. The antibodies provided for herein inhibit IGF-1R and can be used for the treatment of thyroid eye disease and other associated pathologies. This data is more fully exemplified in U.S. Pat. No. 11,548,951 and U.S. Publication No. 2022/0235137, each of which is hereby incorporated by reference in its entirety.

Sample formulations were prepared containing the antibodies as described herein. The appearance of all samples, including clarity, color, and visible particles, was examined against black and white background by a lamp detector to test clarity. Protein concentration was determined via ultraviolet spectrophotometry. All measurements were repeated twice with a 2.5 μL sample volume each time and an average UV280 value was determined. The extinction coefficient used in this evaluation was 1.46 (mg/mL)⁻¹·cm⁻¹. The pH value of all solutions was measured using a pH meter with a glass electrode. All measurements were repeated twice, and an average result was reported. Osmolality was measured using an Osmometer. Before and after tests, method accuracy of the osmometer was confirmed with a Clinitrol 290™ mOsm reference solution. The sample volume for testing was 20 μL and only one test was performed for each sample.

The viscosity of samples was measured by a rheometer, and before sample testing, the accuracy of the rheometer was tested with a viscosity reference standard (Reference N35). Testing temperature was controlled at 25° C., testing volume for each sample volume 0.65 mL, and one test was performed for each sample. Differential scanning calorimetry (DSC) is a thermo-analytical technique in which the difference in the amount of heat required to increase the temperature of a sample and the reference is measured as a function of temperature. Measurements were performed on a differential scanning calorimeter (Malvern® MicroCal VP-DSC, AS12-001C). The protein samples were first diluted to 1 mg/mL with reference buffer before analysis. 400 μL of respective reference buffers were added into the odd-numbered wells of a 96-well plate and 400 μL of samples were added into the even-numbered wells of the same plate. Experimental parameters were set such that the scan temperature ramped from 10-95° C. at a rate of 90° C./h. Analysis of thermograms was performed in the MicroCal VP-DSC automated data analysis software.

Size exclusion chromatography (SEC) is a purity analysis method that separates proteins based on their size. The procedure of SEC analysis was as follows. If the sample concentration was above 10 mg/mL, the sample was diluted to 10 mg/mL with mobile phase before SEC analysis. 10 μg sample was injected into an ultra performance liquid chromatography (UPLC) system equipped with an SEC column (200 Å, 1.7 μm, 4.6 mm×150 mm) and an ultraviolet detector (detection wavelength of 280 nm). Mobile phase contained 50 mM phosphate buffer and 300 mM sodium chloride, and had a pH of 6.8±0.1. An isocratic gradient was applied for 8 minutes at a flow rate of 0.4 mL/min. All raw data was processed with Empower® 3 Chromatography Data Software.

Non-reduced capillary electrophoresis-sodium dodecyl sulfate (CE-SDS-NR), driven by a high-voltage direct current electric field, is a capillary electrophoresis method that allows protein separation by size. The medium is a continuous gel filled in the capillary which serves as a separation channel and forms a molecular sieve in the capillary. The methodology involves 60° C. heat denaturing of the 100 μg protein with of 1% (w/w) sodium dodecyl sulfate (SDS), which masks the intrinsic charge of the proteins and confers an overall net negative charge on the proteins such that they migrate to the cathode when current is applied. This also eliminates the non-covalent interaction. In the CE-SDS-NR method, the diluted protein sample are alkylated by N-ethylmaleimide (NEM) to prevent thermally induced fragmentation. Upon application of a constant electric field, components of different molecule sizes in the protein samples are detected as proteins pass through the capillary with a photodiode array (PDA) detector at 220 nm.

Reduced capillary electrophoresis in sodium dodecyl sulfate (CE-SDS-R) is driven by a high-voltage direct current electric field applied to a capillary that allows protein separation by size. The medium is a continuous gel filled in the capillary which serves as a separation channel which forms a molecular sieve in the capillary. This methodology involves 70° C. heat denaturing of the 100 μg protein with of 1% (w/w) SDS to mask the intrinsic charge of the proteins, conferring similar charge-to-mass ratios. This also decreases non-covalent interactions. In the CE-SDS-R method, the proteins are reduced by BME (2-mercaptoethanol) to break inter-chain disulfide bonds within the protein molecules before loading for separation using electrophoresis. Upon application of a constant electric field, components of different molecule sizes in the protein samples are detected as proteins pass through the capillary with a PDA detector at 220 nm.

Imaged capillary isoelectric focusing (iCIEF) is a purity analysis method used to monitor the amount of charge variant species. The isoelectric point (pI) is an intrinsic property of a specific protein molecule and represents the pH value at which the protein molecule does not carry net electrical charge. Under an external electric field, the charge variants move along a continuous pH gradient formed by ampholytes, and stop at where the pH equals its pI. An electropherogram is obtained at 280 nm. For some of the assays described herein, 20 μg sample were mixed with 80 μL master mix. The mixture was then analyzed with an iCE3® Capillary Isoelectric Focusing Analyzer equipped with a fluorocarbon (FC)-coated whole-column detection capillary. In the electropherogram, the peaks on the left of the main peak are acidic peaks representing acidic variants, and the peaks on the right of the main peak are basic peaks representing basic variants. The pI value and relative abundance of the resolved peaks can be quantitated using chromatographic software. Sub-visible particles were monitored by a micro-flow imaging system. After mixing gently, 1.5 mL of each sample was transferred to a micro-flow imaging 96-well plate in a bio-safety hood for analysis. The results were analyzed by the vendor's software (MVAS 1.4). The amount of sub-visible particles per milliliter, larger than 2 μm, 10 μm, and 25 μm, was reported.

The glass transition temperature (T′g) of the sample was measured by a differential canning calorimeter (TA Instruments® Q-2000 DSC). A sample of 20 μL was sealed in an aluminum pan and transferred into the modulate differential scanning calorimeter (mDSC). A sealed empty pan was used as reference. The samples were cooled to −60° C. and kept at this temperature for 5 minutes, then heated to 20° C. at a rate of 5° C./min. The results were recorded and analyzed by Universal Analysis software.

### Example 2: Short-Term Stability Study

The anti-IGF-1R antibody VRDN-1100 was first buffer-exchanged by ultrafiltration to have the antibody in three buffers: 20 mM histidine buffer (pH 6.0), 20 mM acetate buffer (pH 5.0) and 20 mM citrate buffer (pH 5.5). Next, they were concentrated to a protein concentration higher than the target concentration (>25 mg/mL). Stock solutions of 40% (w/w) sucrose, 25 mM methionine, 700 mM arginine monohydrochloride, and 10% (w/w) PS80 were prepared. The necessary amounts of antibody, excipient stock solutions, and surfactant stock solutions were calculated, weighed and mixed well based on the formulations listed in Table 9, below. pH was adjusted to a target value of 5.5 by using 0.5 M NaOH buffer for formulation F1-4, which shifted due to the arginine monohydrochloride. Each of the prepared samples was filtered through a 0.22 μm polyvinylidene difluoride (PVDF) filter and added to a 2 ml glass vial (1.1 mL fill volume per vial), then stoppered, capped, and labeled immediately.

TABLE 9
Short-Term Formulations

| Protein | Buffer System, | | Formulation |
|---|---|---|---|
| Concentration | pH | Excipient(s)/Surfactant | No. |
| 25 mg/mL | 20 mM | 8% Sucrose, 0.04% PS80 | F1-1 |
| | Histidine, | 8% Sucrose, 5 mM | F1-2 |
| | pH 6.0 | Methionine, | |
| | | 0.04% PS80 | |
| | 20 mM Acetate, | 8% Sucrose, 0.04% PS80 | F1-3 |
| | pH 5.0 | | |
| | 20 mM Citrate, | 140 mM Arginine-HCl, | F1-4 |
| | pH 5.5 | 0.04% PS80 | |

The sampling and testing plan for this study is shown in Table 10, below. For the agitation stress condition, samples were agitated at 25° C. with a speed of 300 rpm for up to 3 days. For the freeze-thaw stress condition, samples were completely frozen at −40° C. and thawed at room temperature (RT). For the thermal stress condition, samples were stored at 40° C. for up to 12 days. Samples were retrieved at each time point shown and kept at 2-8° C. before analysis. The specific assays performed at each time point are described by letters X, Y, and Z in Table 10, below. Letter X indicates the following assays were performed: an appearance check, pH determination, anti-IGF-1R protein concentration determination, SEC-UPLC, CE-SDS-R, CE-SDS-NR, and iCIEF. Letter code Y indicates that sub-visible particle testing was performed by micro-flow imaging. Letter code Z indicates that T′g determination by mSDC, viscosity determination, DSC, and osmolality determination were performed.

TABLE 10
Assays Performed at Specific Sampling Time Points

| Formulation | | | | | |
|---|---|---|---|---|---|
| No. | Attribute | Condition | t₀ | Sampling Points and Assay | |
| F1-1, F1-2, | Thermal | 40° C. | X, | 7 Days | 12 Days |
| F1-3, F1-4 | Stress | | Y, Z | X, Y | X, Y |
| | Freeze-Thaw | −40° C. | | 3 Cycles | 5 Cycles |
| | | to RT | | Appearance | X, Y |
| | Agitation | 300 rpm, | | 1 Day | 3 Days |
| | | 25° C. | | Appearance | X, Y |

All samples remained slightly opalescent, slightly yellow, and free of visible particles. The F1-4 formulation showed greater opalescence at to and all subsequent time points compared to the other three formulations. This greater opalescence was likely due to intermolecular attraction, which may increase the possibility of aggregations or association during long-term storage. Throughout the study, pH and protein concentration of the experimental formulations remained consistent with the target value. The osmolality of all samples was within the range of 290-308 mOsm/kg. The viscosity of all samples was within the range of 1.10-1.33 cP.

The SEC-UPLC data showed no significant increases in the percentage of high molecular weight molecules observed in formulations F1-1 to F1-4 under stress conditions (agitation for up to 3 days, 5 cycles of freeze-thawing, or 12 days at 40° C.) as compared to t₀. The 40° C. stress condition for 12 days caused the percentage of main peak molecules to decrease by an absolute value of 2.1-2.8%, along with and increased percentage of low molecular weight molecules. The CE-SDS-NR data for the 40° C., 12-day stress condition showed 0.5-1.2% decreases in purity.

The measured pI of all samples was 9.1-9.2. Analytical analysis by iCIEF showed no significant differences in the main peak percentage in formulations F1-1 to F1-4 under agitation and freeze-thaw stress conditions compared with to. The test samples stored at 40° C. for 12 days showed iCIEF main peak percentages had decreased by an absolute value of less than 20% (e.g., 16.2-19.2%) compared to t₀. No other significant differences were observed among the experimental formulations.

The non-reduced CE-SDS analysis showed no significant changes due to the agitation or freeze-thaw stress conditions compared with to. Decreased purity, by 0.5-1.2%, were observed in formulations F1-1 to F1-4 after 12 days of incubation at 40° C. The reduced CE-SDS analysis showed no significant changes after agitation or freeze-thaw stress conditions compared with to. Decreased purity, by 1.0-1.2%, were observed in formulations F1-1 to F1-4 after 12 days of incubation at 40° C. No additional significant differences were observed among the experimental formulations.

Micro-flow imaging analysis showed that the number of sub-visible particles in all test formulations was within acceptable limits, and no significant changes in the numbers of sub-visible particles (≥2 μm, ≥10 μm, or ≥25 μm) were observed after agitation for 3 days, 5 cycles of freeze-thawing, or storage at 40° C. for 12 days, compared to t₀. Analytical analysis via DSC showed that the TmOnset values of formulations F1-1 to F1-4 were within the range of 58.4-62.6° C., indicating good thermal stability of the antibody.

Glass transition temperature (T′g) is a critical parameter in determining the state of frozen antibody solution. Below the glass transition temperature, antibody is in a glassy state where potential physical or chemical changes are limited. The measured value of T′g for the antibody in formulation F1-2 was −30.8° C.

The following formulation was chosen as having the greatest stability within this study: 25 mg/mL antibody in 20 mM histidine buffer, 8% (w/v) sucrose, 5 mM L-methionine, and 0.04% (w/v) PS80, at a pH of 6.0.

### Example 3: pH Buffer Screening Based on Stability Study

To screen for an optimal pH of anti-IGF-1R antibody VDRN-1100, formulations with pH values ranging from 5.5 to 7.0 were investigated for stability. The antibody was prepared in 11 formulations as shown in Table 11-1. In all experimental conditions, the antibody was present at a concentration of 50 mg/mL, and the filling volume of each vial was 3.0 mL in 6 R vials.

TABLE 11-1
Exemplary Formulations Tested

| | | | | | Surfactant |
|---|---|---|---|---|---|
| Formulation | pH | Buffer | Sugar (w/v) | Amino Acid | (w/v) |
| F2-1 | 5.5 | 20 mM | 8% | 20 mM | 0.04% PS80 |
| | | succinate | sucrose | methionine | |
| F2-2 | 5.5 | 20 mM | 4.5% | 5 mM | 0.04% PS20 |
| | | histidine | sorbitol | methionine | |
| F2-3 | 6.0 | 20 mM | 4.5% | 20 mM | 0.04% PS20 |
| | | succinate | sorbitol | methionine | |
| F2-4 | 6.0 | 20 mM | 3% | 140 mM | 0.04% PS80 |
| | | histidine | mannitol | glycine | |
| | | | | 5 mM | |
| | | | | methionine | |
| F2-5 | 6.0 | 20 mM | 2% | 140 mM | 0.04% PS80 |
| | | histidine | sorbitol | proline | |
| | | | | 20 mM | |
| | | | | methionine | |
| F2-6 | 6.0 | 20 mM | 4% | 140 mM | 0.04% PS80 |
| | | histidine | sucrose | lysine | |
| | | | | 20 mM | |
| | | | | methionine | |
| F2-7 | 6.0 | 20 mM | 8% | 20 mM | 0.04% PS80 |
| | | histidine | sucrose | methionine | |
| F2-8 | 6.0 | 20 mM | 4.5% | 20 mM | 0.04% PS80 |
| | | histidine | mannitol | methionine | |
| F2-9 | 6.5 | 20 mM | 8% | 20 mM | 0.04% PS20 |
| | | histidine | sucrose | methionine | |
| F2-10 | 6.5 | 20 mM | 4.5% | 5 mM | 0.04% PS20 |
| | | phosphate | sorbitol | methionine | |
| F2-11 | 7.0 | 20 mM | 8% | 20 mM | 0.04% PS80 |
| | | phosphate | sucrose | methionine | |

Samples were for assessed for conformational stability, purity, and potency as measured by appearance, SEC-UPLC, CE-SDS, and DSC.

The samples were tested by appearance after (1) storage at 40° C. for two weeks, (2) storage at 40° C. for four weeks, (3) storage at 5° C. for 11 weeks, (4) 3 cycles of freeze and thaw, and (5) 5 cycles of freeze and thaw. The appearance of all the samples were slightly yellow and opalescent. As shown in Table 11-2, except formulation F2-3, all samples were free of particles under all conditions.

TABLE 11-2
Appearance of tested formulations

| # | 40° C.-2 W | 40° C.-4 W | 5° C.-11 W | FT-3 Cycles | FT-3 Cycles |
|---|---|---|---|---|---|
| F2-1 | Free of | Free of | Free of | Free of | Free of |
| | particles | particles | particles | particles | particles |
| F2-2 | Free of | Free of | Free of | Free of | Free of |
| | particles | particles | particles | particles | particles |
| F2-3 | Free of | Particles | Free of | Particles | Particles |
| | particles | observed (1) | particles | observed (1) | observed (1) |
| F2-4 | Free of | Free of | Free of | Free of | Free of |
| | particles | particles | particles | particles | particles |
| F2-5 | Free of | Free of | Particles | Free of | Free of |
| | particles | particles | observed (1) | particles | particles |
| F2-6 | Free of | Free of | Free of | Free of | Free of |
| | particles | particles | particles | particles | particles |
| F2-7 | Free of | Free of | Free of | Free of | Free of |
| | particles | particles | particles | particles | particles |
| F2-8 | Free of | Free of | Free of | Free of | Free of |
| | particles | particles | particles | particles | particles |
| F2-9 | Free of | Free of | Free of | Free of | Free of |
| | particles | particles | particles | particles | particles |
| F2- | Free of | Free of | Free of | Free of | Free of |
| 10 | particles | particles | particles | particles | particles |
| F2- | Free of | Free of | Free of | Free of | Free of |
| 11 | particles | particles | particles | particles | particles |

The amount of high molecular weight (HMW), monomer, and low molecular weight (LMW) were determined by SEC-ULPC, as shown in Table 11-3.

TABLE 11-3
Stability of formulations as determined by SEC

| | HMW/Monomer/LMW (Δmonomer %) | | | |
|---|---|---|---|---|
| No. | T0 | 40° C.-2 Weeks | 40° C.-4 Weeks | 5° C.-11 Weeks |
| F2-1 | 0.7/99.2/0.1 | 1.3/98.3/0.4 (↓ 0.9) | 1.9/97.2/0.9 (↓ 2.0) | 0.8/99.1/0.1 |
| F2-2 | 0.7/99.3/0.0 | 1.0/98.6/0.4 (↓ 0.7) | 1.3/97.8/0.9 (↓ 1.5) | 0.7/99.2/0.1 |
| F2-3 | 0.8/99.1/0.0 | 1.6/98.0/0.4 (↓ 1.0) | 2.1/97.0/0.9 (↓ 2.1) | 1.0/98.9/0.1 |
| F2-4 | 0.7/99.2/0.1 | 1.5/98.0/0.5 (↓ 1.2) | 1.9/97.0/1.0 (↓ 2.2) | 0.8/99.1/0.1 |
| F2-5 | 0.7/99.2/0.0 | 1.0/98.6/0.4 (↓ 0.6) | 1.4/97.7/0.9 (↓ 1.5) | 0.7/99.2/0.1 |
| F2-6 | 0.7/99.2/0.1 | 1.8/97.7/0.5 (↓ 1.5) | 2.5/96.5/0.9 (↓ 2.7) | 0.8/99.1/0.1 |
| F2-7 | 0.7/99.2/0.1 | 1.1/98.5/0.4 (↓ 0.7) | 1.5/97.7/0.9 (↓ 1.5) | 0.7/99.2/0.1 |
| F2-8 | 1.3/98.6/0.1 | 2.7/96.8/0.4 (↓ 1.8) | 2.5/96.6/0.9 (↓ 2.0) | 0.7/99.2/0.1 |
| F2-9 | 0.7/99.2/0.1 | 0.9/98.7/0.4 (↓ 0.5) | 1.2/97.9/0.9 (↓ 1.3) | 0.8/99.1/0.1 |
| F2-10 | 1.0/99.0/0.1 | 2.1/97.5/0.4 (↓ 1.5) | 2.8/96.3/0.9 (↓ 2.7) | 1.4/98.5/0.1 (↓ 0.5) |
| F2-11 | 0.9/99.0/0.1 | 2.0/97.5/0.5 (↓ 1.5) | 2.7/96.0/1.3 (↓ 3.0) | 1.2/98.7/0.1 |

Purities of the samples were measured by Caliper-SDS upon storage (1) at 40° C. for two weeks, (2) at 40° C. for four weeks, and (3) at 5° C. for 11 weeks. The purities of formulations F2-10 and F2-11 decreased by more than 5% upon storage at 40° C. for four weeks. As shown in Table 11-4, all the other samples maintained high purities (e.g., >93%) even after storage at 40° C. for four weeks.

TABLE 11-4
Purities of formulations as determined by Caliper-SDS

| | NR(Δ purities) | | | |
|---|---|---|---|---|
| No. | T0 | 40° C.-2 Weeks | 40° C.-4 Weeks | 5° C.-11 Weeks |
| F2-1 | 97.7 | 95.5(↓ 2.2) | 93.7(↓ 4.0) | 97.0 |
| F2-2 | 97.3 | 96.0(↓ 1.3) | 93.2(↓ 4.1) | 96.8 |
| F2-3 | 97.7 | 95.7(↓ 2.0) | 94.3(↓ 3.4) | 97.1 |
| F2-4 | 97.2 | 95.5(↓ 1.7) | 93.9(↓ 3.3) | 95.1(↓ 2.1) |
| F2-5 | 97.5 | 96.2(↓ 1.3) | 93.9(↓ 3.6) | 96.7 |
| F2-6 | 97.7 | 95.8(↓ 1.9) | 94.2(↓ 3.5) | 97.1 |
| F2-7 | 97.5 | 95.7(↓ 1.8) | 94.3(↓ 3.2) | 96.6 |
| F2-8 | 97.6 | 95.9(↓ 1.7) | 93.6(↓ 4.0) | 96.9 |
| F2-9 | 97.8 | 96.1(↓ 1.7) | 94.5(↓ 3.3) | 97.0 |
| F2-10 | 97.6 | 95.5(↓ 2.1) | 92.3(↓ 5.3) | 96.7 |
| F2-11 | 97.7 | 94.4(↓ 3.3) | 90.3(↓ 7.4) | 97.2 |

Next, samples were assessed for stability using iCIEF analysis, following storage under the following conditions: (1) 40° C. for two weeks, (2) 40° C. for four weeks, and (3) 5° C. for 11 weeks. In all experimental conditions, the antibody was present at a concentration of 50 mg/mL, and the filling volume of each vial was 3.0 mL in 6 R vials.

Analysis of shifts in charge profile of the formulations following storage at 40° C. or 5° C. generally showed a decrease in main peak percentage with a corresponding increase in acidic peak percentage. As shown in Table 11-5, below, storage at 40° C. for 4 weeks demonstrated an absolute increase of 26.6-59.1 in acidic peak percentage as measured by iCIEF. The lower pH formulations (F2-1 to F2-8 with a pH of either 5.5 or 6.0) demonstrated an absolute increase of 26.6-37.6 in acidic peak percentage, while the higher pH formulations (F2-9 to F2-11 with a pH or either 6.5 or 7.0) demonstrated a greater increase of 43.7-59.1 in acidic peak percentage.

Thus, overall, samples formulated with a pH value at or below 6.0 demonstrated higher stability upon storage at 40° C. for 4 weeks, as compared to those with pH at or above 6.5. Accordingly, formulations with a pH of greater than 6.0 (F2-9 to F2-11) were removed from further consideration.

TABLE 11-5
iCIEF Results

| | | Acidic peak %/Main peak %/Basic peak % (ΔAcid peak) | | | |
|---|---|---|---|---|---|
| No. | pH | T0 | 40° C.-2 Weeks | 40° C.-4 Weeks | 5° C.-11 Weeks |
| F2-1 | 5.5 | 34.9/44.2/20.9 | 52.7/25.1/22.2(−17.8) | 67.5/14.6/17.9(−32.6) | 35.7/44.0/20.3(+0.8) |
| F2-2 | 5.5 | 35.4/44.2/20.4 | 50.0/25.7/24.3(−14.6) | 62.0/18.0/20.1(−26.6) | 36.1/43.8/20.2(+1.7) |
| F2-3 | 6.0 | 35.1/44.0/20.9 | 55.2/23.3/21.5(−20.1) | 71.5/11.3/17.1(−36.4) | 35.2/44.8/20.0(+0.1) |
| F2-4 | 6.0 | 35.4/43.5/21.1 | 59.0/20.2/20.8(−23.6) | 73.0/12.3/14.8(−37.6) | 36.8/43.2/20.1(+1.4) |
| F2-5 | 6.0 | 35.0/44.8/20.2 | 55.9/22.9/21.2(−20.9) | 70.0/13.0/17.0(−35.0) | 36.6/43.9/19.5(+1.6) |
| F2-6 | 6.0 | 34.4/45.0/20.6 | 50.0/27.8/22.2(−15.6) | 65.9/15.6/18.6(−31.5) | 36.7/43.0/20.3(+2.3) |
| F2-7 | 6.0 | 34.8/44.1/21.1 | 53.8/25.6/20.6(−19.0) | 69.2/13.6/17.2(−34.4) | 36.0/44.0/20.0(+1.2) |
| F2-8 | 6.0 | 34.4/44.5/21.1 | 54.3/24.2/21.5(−19.9) | 69.6/14.0/16.3(−35.2) | 37.7/42.5/19.8(+3.3) |
| F2-9 | 6.5 | 34.3/44.7/21.0 | 58.7/21.2/20.1(−24.3) | 78.0/7.9/14.1(−43.7) | 38.8/42.0/19.1(+4.5) |
| F2-10 | 6.5 | 34.3/44.5/21.3 | 71.0/12.3/16.7(−36.7) | 86.3/4.0/9.7(−52.0) | 37.0/43.0/20.0(+2.7) |
| F2-11 | 7.0 | 35.2/44.1/20.7 | 81.8/6.2/12.1(−46.6) | 94.3/2.4/3.3(−59.1) | 38.7/42.0/19.3(+3.5) |

### Example 4: Additional pH Buffer Screening

To obtain a pharmaceutically acceptable composition including anti-IGF-1R antibody VRDN-1100, a variety of buffer systems and pH values for the total solution were investigated. Typically, a pH of 4.5 or higher is required to maintain an antibody in its native state (See e.g., Susumu (2014) Liquid Formulation for Antibody Drugs, Biochimica et Biophysica Acta (BBA)-Proteins and Proteomics 11:2041). In view of the data described in Example 3, formulations with a pH value within the range of 4.5-6.0 were further investigated. The stability of the antibody was determined under each set of experimental conditions. Tested buffer/pH systems are provided in Table 12-1, below.

TABLE 12-1
pH and buffer systems tested

| Formulation | pH | Buffer |
|---|---|---|
| F3-1 | 4.5 | 20M succinate |
| F3-2 | 4.8 | 20M succinate |
| F3-3 | 5.2 | 20M succinate |
| F3-4 | 5.5 | 20M succinate |
| F3-5 | 4.8 | 20M acetate |
| F3-6 | 5.2 | 20M acetate |
| F3-7 | 5.5 | 20M histidine |
| F3-8 | 6.0 | 20 mM histidine |
| F3-9 | 4.8 | 20 mM histidine and acetate |
| F3-10 | 5.5 | 20 mM histidine and acetate |
| F3-11 | 4.8 | 20 mM histidine and glutamic acid |

In all experimental conditions, the antibody was present at a concentration of 50 mg/mL, and the filling volume of each vial was 1 mL in 2 R vials.

Experimental vials, as described immediately above, were subjected to 40° C., 25° C., or 5° C. condition for 4 weeks. In some cases, samples were taken and assayed at to, 2 weeks, and 4 weeks. At t₀, the following assays were performed on all vials at all temperatures: appearance check, SEC-UPLC, caliper-SDS-NR, caliper-SDS-R, iCIEF, pH determination, anti-IGF-1R protein concentration determination, and DSC. Vials in the 40° C. and 25° C. groups were sampled and assayed at the two week time point. The following assays were performed on these samples: appearance check, SEC-UPLC, caliper-SDS-NR, and iCIEF. Vials in the 40° C. group were sampled at the 4-week time point, and the following assays were performed: appearance check, SEC-UPLC, caliper-SDS-NR, caliper-SDS-R, iCIEF, pH determination, and anti-IGF-1R protein concentration determination. Vials in the 25° C. and 5° C. groups were sampled at the 4-week time point, and the following assays were performed: appearance check, SEC-UPLC, caliper-SDS-NR, iCIEF, pH determination, and antibody concentration determination.

DSC results indicated that Tm onset was higher than 59° C. for all buffer system and final solution pH combination conditions except for 20 mM succinate with a pH of 4.5 and 20 mM succinate with a pH of 4.8. The results for these two experimental conditions indicated relatively worse conformational stability. Particles were visually observed at 2 weeks and at 4 weeks in vials with the following conditions: 20 mM succinate with a pH of 4.5 at 40° C. and 20 mM succinate with a pH of 4.8 at 40° C. Particles were first observed by eye at the 4 week time point in vials with the following conditions: 20 mM succinate buffer with a pH of 5.2 at 40° C., 20 mM acetate buffer with a pH of 5.2 at 40° C., and 20 mM histidine buffer with a pH of 6.0 at 40° C. Vials subjected to 5° C. with the following conditions contained visually observed particles at the 4-week time point: 20 mM succinate with a pH of 4.5 and 20 mM of total histidine and glutamic acid with a pH of 4.8. DSC results are further summarized in Table 12-22, below.

TABLE 12-2
DSC Results

| | | DSC (° C.) | | | |
|---|---|---|---|---|---|
| No. | Buffer, pH | T_{Onset} | Tₘ1 | Tₘ2 | Tₘ3 |
| F3-1 | Suc, 4.5 | 54.9 | 62.9 | 74.4 | 82.8 |
| F3-2 | Suc, 4.8 | 57.3 | 66.4 | 75.7 | 84.6 |
| F3-3 | Suc, 5.2 | 60.3 | 70.0 | 77.7 | 85.4 |
| F3-4 | Suc, 5.5 | 62.7 | 72.0 | 78.6 | 85.7 |
| F3-5 | Ace, 4.8 | 61.0 | 69.3 | 77.9 | 85.0 |
| F3-6 | Ace, 5.2 | 63.0 | 71.6 | 78.5 | 86.1 |
| F3-7 | His, 5.5 | 59.8 | 68.1 | 76.6 | 83.4 |
| F3-8 | His, 6.0 | 63.7 | 72.2 | 78.5 | 86.0 |
| F3-9 | His/Ace, 4.8 | 60.5 | 68.8 | 77.8 | 84.6 |
| F3-10 | His/Ace, 5.5 | 63.1 | 71.7 | 78.8 | 85.4 |
| F3-11 | His/Glu, 4.8 | 60.4 | 67.5 | 77.7 | 84.8 |
| F3-12 | His/Glu, 5.2 | 61.5 | 69.6 | 78.2 | 84.9 |

The antibody concentration determination results and the pH determination results did not appear to favor or disfavor any sets of experimental conditions with a pH value between 4.5-6.0. SEC-UPLC data indicated that the concentration of monomeric antibody decreases by greater than 2% after 4 weeks at 40° C. All other experimental conditions had a less than 1.5% decrease in intact antibody after 4 weeks at 40° C. The iCIEF results showed an absolute decrease of greater than 26% in main peak percentage of the antibody after 4 weeks at 40° C. for the following conditions: 20 mM acetate buffer with a pH of 5.2, 20 mM histidine buffer with a pH of 6.0, and 20 mM of total histidine and acetic acid with a pH of 5.5. The following conditions demonstrated a significantly lower decrease in the iCIEF main peak of anti-IGF-1R protein at either after 4 weeks at 40° C. or after 4 weeks at 25° C.: 20 mM succinate with a pH of 4.5, 20 mM acetate buffer with a pH of 4.8, 20 mM histidine buffer with a pH of 5.5, 20 mM of total histidine and acetic acid with a pH of 4.8, and 20 mM of total histidine and glutamic acid with a pH of 4.8. Protein concentration determination results are also summarized in Table 13, below.

TABLE 13
Protein Concentration and pH Determination Results

| | | Protein Concentration (mg/mL) | | | | pH | | | |
|---|---|---|---|---|---|---|---|---|---|
| | | | 40° C.- | 25° C.- | 5° C.- | | 40° C.- | 25° C.- | 5° C.- |
| No. | Buffer, pH | T0 | 4 W | 4 W | 4 W | T0 | 4 W | 4 W | 4 W |
| F3-1 | Suc, 4.5 | 51.0 | 51.3 | 51.7 | 52.4 | 4.6 | 4.6 | 4.6 | 4.6 |
| F3-2 | Suc, 4.8 | 50.8 | 51.1 | 51.5 | 52.3 | 4.8 | 4.9 | 4.9 | 4.8 |
| F3-3 | Suc, 5.2 | 51.4 | 51.6 | 52.3 | 52.7 | 5.2 | 5.2 | 5.2 | 5.2 |
| F3-4 | Suc, 5.5 | 51.3 | 51.7 | 52.2 | 52.7 | 5.5 | 5.5 | 5.5 | 5.5 |
| F3-5 | Ace, 4.8 | 51.0 | 51.4 | 52.1 | 52.3 | 5.0 | 5.1 | 5.0 | 5.0 |
| F3-6 | Ace, 5.2 | 50.9 | 51.2 | 51.7 | 52.3 | 5.3 | 5.4 | 5.4 | 5.4 |
| F3-7 | His, 5.5 | 51.2 | 52.0 | 52.5 | 52.6 | 5.5 | 5.5 | 5.5 | 5.5 |
| F3-8 | His, 6.0 | 50.8 | 51.3 | 51.9 | 52.3 | 6.0 | 6.0 | 6.0 | 6.0 |
| F3-9 | His/Ace, | 50.1 | 50.6 | 51.2 | 51.9 | 4.9 | 5.0 | 5.0 | 5.0 |
| | 4.8 | | | | | | | | |
| F3-10 | His/Ace, | 51.6 | 51.8 | 52.3 | 53.0 | 5.6 | 5.6 | 5.6 | 5.6 |
| | 5.5 | | | | | | | | |
| F3-11 | His/Glu, | 51.0 | 51.6 | 52.3 | 52.6 | 4.9 | 5.0 | 5.0 | 5.0 |
| | 4.8 | | | | | | | | |
| F3-12 | His/Glu, | 51.1 | 52.0 | 52.4 | 53.0 | 5.3 | 5.4 | 5.3 | 5.4 |
| | 5.2 | | | | | | | | |

The SC-UPLC results are summarized in Table 14, below.

TABLE 14
SC-UPLC Results

| | | SEC-UPLC | | | | | | | | |
|---|---|---|---|---|---|---|---|---|---|---|
| | | Monomer % | | | | | | HMW % | | |
| | Buffer, | | 40° C.- | 40° C.- | 25° C.- | 25° C.- | 5° C.- | | 40° C.- | 40° C.- |
| No. | pH | T0 | 2 W | 4 W | 2 W | 4 W | 4 W | T0 | 2 W | 4 W |
| F3-1 | Suc, | 99.1 | 97.9 | 96.2 | 99.0 | 98.9 | 99.3 | 0.8 | 1.3 | 2.3 |
| | 4.5 | | (−1.2) | (−2.9) | | | | | | |
| F3-2 | Suc, | 98.9 | 97.9 | 96.4 | 98.7 | 98.8 | 99.0 | 1.0 | 1.6 | 2.5 |
| | 4.8 | | (−1.0) | (−2.5) | | | | | | |
| F3-3 | Suc, | 98.9 | 97.9 | 96.7 | 98.7 | 98.6 | 99.1 | 1.0 | 1.7 | 2.6 |
| | 5.2 | | (−1.0) | (−2.2) | | | | | | |
| F3-4 | Suc, | 98.8 | 97.7 | 96.8 | 98.4 | 98.5 | 98.9 | 1.1 | 1.9 | 2.5 |
| | 5.5 | | (−1.1) | (−2.0) | | | | | | |
| F3-5 | Ace, | 98.8 | 98.3 | 97.7 | 98.3 | 98.8 | 99.1 | 1.1 | 1.3 | 1.6 |
| | 4.8 | | (−0.5) | (−1.1) | | | | | | |
| F3-6 | Ace, | 98.8 | 98.2 | 97.7 | 98.3 | 98.7 | 99.0 | 1.1 | 1.4 | 1.7 |
| | 5.2 | | (−0.6) | (−1.1) | | | | | | |
| F3-7 | His, | 99.2 | 98.5 | 97.8 | 99.2 | 99.0 | 99.3 | 0.8 | 1.1 | 1.5 |
| | 5.5 | | (−0.7) | (−1.4) | | | | | | |
| F3-8 | His, | 99.0 | 98.5 | 97.9 | 99.1 | 98.9 | 99.2 | 0.9 | 1.1 | 1.4 |
| | 6.0 | | (−0.5) | (−1.1) | | | | | | |
| F3-9 | His/ | 98.7 | 98.3 | 97.8 | 99.0 | 98.8 | 99.0 | 1.2 | 1.3 | 1.5 |
| | Ace, | | (−0.4) | (−0.9) | | | | | | |
| | 4.8 | | | | | | | | | |
| F3-10 | His/ | 98.9 | 98.4 | 98.0 | 99.1 | 99.0 | 99.2 | 1.1 | 1.3 | 1.4 |
| | Ace, | | (−0.5) | (−0.9) | | | | | | |
| | 5.5 | | | | | | | | | |
| F3-11 | His/ | 99.3 | 98.7 | 98.2 | 99.3 | 99.2 | 99.4 | 0.7 | 0.8 | 1.0 |
| | Glu, | | (−0.6) | (−1.1) | | | | | | |
| | 4.8 | | | | | | | | | |
| F3-12 | His/ | 99.3 | 98.7 | 98.3 | 99.3 | 99.2 | 99.4 | 0.6 | 0.9 | 1.0 |
| | Glu, | | (−0.6) | (−1.0) | | | | | | |
| | 5.2 | | | | | | | | | |

| | SEC-UPLC | | | | | | | | |
|---|---|---|---|---|---|---|---|---|---|
| | HMW % | | | LMW % | | | | | |
| | 25° C.- | 25° C.- | 5° C.- | | 40° C.- | 40° C.- | 25° C.- | 25° C.- | 5° C.- |
| No. | 2 W | 4 W | 4 W | T0 | 2 W | 4 W | 2 W | 4 W | 4 W |
| F3-1 | 0.9 | 0.9 | 0.6 | 0.1 | 0.8 | 1.5 | 0.2 | 0.3 | 0.1 |
| F3-2 | 1.1 | 0.9 | 0.9 | 0.1 | 0.6 | 1.1 | 0.2 | 0.2 | 0.1 |
| F3-3 | 1.2 | 1.2 | 0.8 | 0.1 | 0.4 | 0.8 | 0.1 | 0.2 | 0.1 |
| F3-4 | 1.5 | 1.4 | 1.0 | 0.1 | 0.4 | 0.7 | 0.1 | 0.2 | 0.1 |
| F3-5 | 1.5 | 1.0 | 0.8 | 0.1 | 0.4 | 0.7 | 0.1 | 0.2 | 0.1 |
| F3-6 | 1.6 | 1.1 | 1.0 | 0.1 | 0.4 | 0.6 | 0.1 | 0.2 | 0.1 |
| F3-7 | 0.7 | 0.8 | 0.6 | 0.1 | 0.4 | 0.7 | 0.1 | 0.2 | 0.1 |
| F3-8 | 0.7 | 0.9 | 0.7 | 0.1 | 0.4 | 0.7 | 0.1 | 0.2 | 0.1 |
| F3-9 | 0.9 | 1.0 | 0.9 | 0.1 | 0.4 | 0.8 | 0.1 | 0.2 | 0.1 |
| F3-10 | 0.7 | 0.9 | 0.7 | 0.1 | 0.3 | 0.6 | 0.1 | 0.2 | 0.1 |
| F3-11 | 0.6 | 0.6 | 0.5 | 0.1 | 0.4 | 0.8 | 0.1 | 0.2 | 0.1 |
| F3-12 | 0.6 | 0.6 | 0.5 | 0.1 | 0.4 | 0.7 | 0.1 | 0.2 | 0.1 |

The iCIEF results are summarized in Table 15, below.

TABLE 15
iCIEF Results

| | | iCIEF | | | | | | | | |
|---|---|---|---|---|---|---|---|---|---|---|
| | | Main peak % | | | | | | Acidic peaks % | | |
| | Buffer, | | 40° C.- | 40° C.- | 25° C.- | 25° C.- | 5° C.- | | 40° C.- | 40° C.- |
| No. | pH | T0 | 2 W | 4 W | 2 W | 4 W | 4 W | T0 | 2 W | 4 W |
| F3-1 | Suc, | 43.0 | 30.8 | 17.1 | 40.0 | 40.7 | 44.1 | 36.0 | 53.8 | 65.2 |
| | 4.5 | | (−12.2) | (−25.9) | (−3.0) | (−2.3) | | | | |
| F3-2 | Suc, | 44.3 | 31.1 | 16.4 | 43.5 | 40.3 | 43.6 | 35.5 | 54.5 | 65.3 |
| | 4.8 | | (−13.2) | (−27.9) | (−0.8) | (−4.0) | | | | |
| F3-3 | Suc, | 44.8 | 31.9 | 16.1 | 43.3 | 41.3 | 43.1 | 34.7 | 53.5 | 66.4 |
| | 5.2 | | (−12.9) | (−28.7) | (−1.5) | (−3.5) | | | | |
| F3-4 | Suc, | 43.8 | 23.3 | 15.2 | 42.6 | 40.0 | 43.4 | 35.1 | 54.6 | 66.9 |
| | 5.5 | | (−20.5) | (−28.6 | (−1.2) | (−3.8) | | | | |
| F3-5 | Ace, | 43.7 | 25.4 | 17.1 | 42.8 | 41.5 | 44.3 | 35.5 | 50.4 | 63.5 |
| | 4.8 | | (−18.3) | (−26.6) | (−0.9) | (−2.2) | | | | |
| F3-6 | Ace, | 44.4 | 24.7 | 15.3 | 42.3 | 42.5 | 43.4 | 35.3 | 52.5 | 65.6 |
| | 5.2 | | (−19.7) | (−29.1) | (−2.1) | (−1.9) | | | | |
| F3-7 | His, | 43.7 | 25.4 | 17.8 | 42.4 | 40.4 | 43.4 | 35.5 | 51.0 | 61.6 |
| | 5.5 | | (−18.3) | (−25.9) | (−1.3) | (−3.3) | | | | |
| F3-8 | His, | 43.3 | 21.5 | 14.1 | 40.5 | 38.8 | 43.7 | 35.8 | 56.1 | 67.9 |
| | 6.0 | | (−21.8) | (−29.2) | (−2.8) | (−4.5) | | | | |
| F3-9 | His/ | 42.0 | 24.9 | 17.5 | 42.1 | 41.0 | 44.1 | 37.3 | 52.1 | 62.5 |
| | Ace, | | (−17.1) | (−24.5) | (+0.1) | (−1.0) | | | | |
| | 4.8 | | | | | | | | | |
| F3-10 | His/ | 43.7 | 24.3 | 14.5 | 41.1 | 40.1 | 43.6 | 35.7 | 53.0 | 67.1 |
| | Ace, | | (−19.4) | (−29.2) | (−2.6) | (−3.6) | | | | |
| | 5.5 | | | | | | | | | |
| F3-11 | His/ | 44.1 | 26.3 | 18.2 | 41.8 | 41.5 | 44.0 | 34.8 | 49.2 | 61.0 |
| | Glu, | | (−17.8) | (−25.9) | (−2.3) | (−2.6) | | | | |
| | 4.8 | | | | | | | | | |
| F3-12 | His/ | 44.2 | 25.8 | 17.0 | 41.4 | 40.4 | 43.6 | 35.2 | 51.5 | 63.6 |
| | Glu, | | (−18.4) | (−27.2) | (−2.8) | (−3.8) | | | | |
| | 5.2 | | | | | | | | | |

| | iCIEF | | | | | | | | |
|---|---|---|---|---|---|---|---|---|---|
| | Acidic peaks % | | | Basic peaks % | | | | | |
| | 25° C.- | 25° C.- | 5° C.- | | 40° C.- | 40° C.- | 25° C.- | 25° C.- | 5° C.- |
| No. | 2 W | 4 W | 4 W | T0 | 2 W | 4 W | 2 W | 4 W | 4 W |
| F3-1 | 40.7 | 39.8 | 35.1 | 21.0 | 15.4 | 17.7 | 19.3 | 19.5 | 20.7 |
| F3-2 | 35.8 | 40.2 | 35.5 | 20.2 | 14.4 | 18.3 | 20.6 | 19.5 | 20.9 |
| F3-3 | 37.2 | 39.9 | 36.0 | 20.5 | 14.6 | 17.5 | 19.5 | 18.8 | 20.9 |
| F3-4 | 38.1 | 41.0 | 35.9 | 21.1 | 22.1 | 17.9 | 19.3 | 19.0 | 20.7 |
| F3-5 | 37.4 | 38.9 | 35.1 | 20.8 | 24.2 | 19.4 | 19.8 | 19.6 | 20.6 |
| F3-6 | 37.7 | 37.7 | 35.8 | 20.3 | 22.8 | 19.1 | 20.0 | 19.8 | 20.9 |
| F3-7 | 38.2 | 40.3 | 36.3 | 20.8 | 23.6 | 20.6 | 19.5 | 19.3 | 20.3 |
| F3-8 | 40.7 | 43.2 | 35.9 | 20.9 | 22.4 | 18.0 | 18.8 | 18.0 | 20.3 |
| F3-9 | 38.2 | 39.9 | 35.4 | 20.8 | 23.0 | 20.0 | 19.7 | 19.1 | 20.5 |
| F3-10 | 39.9 | 41.3 | 36.2 | 20.7 | 22.7 | 18.3 | 18.8 | 18.6 | 20.3 |
| F3-11 | 38.2 | 39.3 | 35.9 | 21.1 | 24.5 | 20.8 | 20.0 | 19.3 | 20.1 |
| F3-12 | 39.2 | 40.3 | 36.0 | 20.6 | 22.7 | 19.4 | 19.5 | 19.3 | 20.4 |

The results of SDS-Caliper-NR testing showed that greater decreases in purity, of greater than 4% purity lost, occurred with the following sets of conditions after 4 weeks at 40° C.: 20 mM succinate with a pH of 4.5, 20 mM succinate with a pH of 4.8, and 20 mM succinate with a pH of 5.2. The SCS-Caliper-R assays showed that the following conditions had lower decreases in purity, of less than 1.5%, compared to other conditions: 20 mM succinate at a pH of 5.2 and 20 mM of total histidine and acetic acid at a pH of 5.5. The results are also summarized in Table 16-1, below.

TABLE 16-1
SDS-Caliper-NR Results

| | | | | | | | | SDS-Caliper-R | |
|---|---|---|---|---|---|---|---|---|---|
| | | SDS-Caliper-NR | | | | | | Purity % | |
| | | Purity % | | | | | | (LC % + HC %) | |
| | Buffer, | | 40° C.- | 40° C.- | 25° C.- | 25° C.- | 5° C.- | | 40° C.- |
| No. | pH | T0 | 2 W | 4 W | 2 W | 4 W | 4 W | T0 | 4 W |
| F3-1 | Suc, 4.5 | 97.5 | 94.6 | 91.6 | 97.1 | 96.5 | 97.2 | 98.6 | 96.5 |
| | | | (−2.9) | (−5.9) | (−0.4) | (−1.0) | (−0.3) | | (−2.1) |
| F3-2 | Suc, 4.8 | 97.4 | 95.1 | 92.5 | 97.3 | 96.6 | 97.1 | 98.5 | 96.7 |
| | | | (−2.3) | (−4.9) | (−0.1) | (−0.8) | (−0.3) | | (−1.8) |
| F3-3 | Suc, 5.2 | 97.6 | 95.5 | 93.6 | 97.1 | 96.6 | 97.2 | 98.2 | 97.0 |
| | | | (−2.1) | (−4.0) | (−0.5) | (−1.0) | (−0.4) | | (−1.2) |
| F3-4 | Suc, 5.5 | 96.6 | 96.0 | 93.8 | 96.7 | 96.5 | 97.3 | 98.8 | 96.9 |
| | | | (−0.6) | (−2.8) | (−0.9) | (−0.1) | (+0.7) | | (−1.9) |
| F3-5 | Ace, 4.8 | 97.2 | 96.2 | 93.7 | 97.1 | 96.7 | 97.2 | 98.6 | 97.0 |
| | | | (−1.0) | (−3.5) | (−0.1) | (−0.5) | (0.0) | | (−1.6) |
| F3-6 | Ace, 5.2 | 97.2 | 96.1 | 94.2 | 97.1 | 96.5 | 97.2 | 98.6 | 96.8 |
| | | | (−1.1) | (−3.0) | (−0.1) | (−0.7) | (0.0) | | (−1.8) |
| F3-7 | His, 5.5 | 97.2 | 96.5 | 93.7 | 97.4 | 96.7 | 97.4 | 98.5 | 96.7 |
| | | | (−0.7) | (−3.5) | (+0.2) | (−0.5) | (+0.2) | | (−1.8) |
| F3-8 | His, 6.0 | 97.2 | 96.4 | 93.9 | 95.7 | 96.8 | 97.1 | 98.4 | 96.8 |
| | | | (−0.8) | (−3.3) | (−1.5) | (−0.4) | (−0.1) | | (−1.6) |
| F3-9 | His/Ace, | 97.2 | 95.9 | 93.3 | 96.8 | 96.5 | 97.1 | 98.5 | 96.7 |
| | 4.8 | | (−1.3) | (−3.9) | (−0.4) | (−0.7) | (−0.1) | | (−1.8) |
| F3-10 | His/Ace, | 97.3 | 96.5 | 94.6 | 97.3 | 96.7 | 96.2 | 98.0 | 97.1 |
| | 5.5 | | (−0.8) | (−2.7) | (−0.0) | (−0.6) | (−1.1) | | (−0.9) |
| F3-11 | His/Glu, | 97.5 | 95.9 | 94.3 | 97.2 | 96.4 | 96.8 | 98.6 | 96.7 |
| | 4.8 | | (−1.6) | (−3.2) | (−0.3) | (−1.1) | (−0.7) | | (−1.9) |
| F3-12 | His/Glu, | 97.4 | 96.3 | 94.6 | 97.3 | 96.4 | 97.2 | 98.5 | 96.8 |
| | 5.2 | | (−1.1) | (−2.8) | (−0.1) | (−1.0) | (−0.2) | | (−1.7) |

Table 16-2 below summarizes the pH and buffer effects illustrated in Examples 3 and 4. Formulations with pH between 4.8 and 6.5, and particularly 4.8 and 6.0, appear to be stable, with high conformational and colloidal stability, low aggregation, and high purity.

TABLE 16-2
Summary of pH and Buffer Screening Results

| | | | | | | | iCIEF | |
|---|---|---|---|---|---|---|---|---|
| | | | | | | | Main | Caliper- |
| | | | | | SEC-UPLC | SEC- | peak % | NR |
| | | | DSC | Appearance | Monomer % | UPLC | (Δmain | (Purity |
| | | | (Tm | (particle | (ΔMonomer) | HMW % | peak) | lost %) |
| | | | onset | amount at | At 40° | At 40° | At 40° | At 40° |
| No. | pH | Buffer | ° C.) | 40° C. 2 W) | C. 4 W | C. 4 W | C. 4 W | C. 4 W |
| F3-1 | 4.5 | Suc | 54.9 | >5 | 96.2 | 2.3 | 17.1 | −5.9 |
| | | | | | (−2.9) | | (−25.9) | |
| F3-2 | 4.8 | Suc | 57.3 | >5 | 96.4 | 2.5 | 16.4 | −4.9 |
| | | | | | (−2.5) | | (−27.9) | |
| F3-5 | 4.8 | Ace | 61.0 | Free of | 97.7 | 1.6 | 17.1 | −3.5 |
| | | | | particle | (−1.1) | | (−26.6) | |
| F3-9 | 4.8 | His/Ace | 60.5 | Free of | 97.8 | 1.5 | 17.5 | −3.9 |
| | | | | particle | (−0.9) | | (−24.5) | |
| F3-11 | 4.8 | His/Glu | 60.4 | Free of | 98.2 | 1.0 | 18.2 | −3.2 |
| | | | | particle | (−1.1) | | (−25.9) | |
| F3-3 | 5.2 | Suc | 60.3 | ≤5 | 96.7 | 2.6 | 16.1 | −4.0 |
| | | | | | (−2.2) | | (−28.7) | |
| F3-6 | 5.2 | Ace | 63.0 | Free of | 97.7 | 1.7 | 15.3 | −3.0 |
| | | | | particle | (−1.1) | | (−29.1) | |
| F3-12 | 5.2 | His/Glu | 61.5 | Free of | 98.3 | 1.0 | 17.0 | −2.8 |
| | | | | particle | (−1.0) | | (−27.2) | |
| F3-4 | 5.5 | Suc | 62.7 | Free of | 96.8 | 2.5 | 15.2 | −2.8 |
| | | | | particle | (−2.0) | | (−28.6) | |
| F3-7 | 5.5 | His | 59.8 | Free of | 97.8 | 1.5 | 17.8 | −3.5 |
| | | | | particle | (−1.4) | | (−25.9) | |
| F3-10 | 5.5 | His/Ace | 63.1 | Free of | 98.0 | 1.4 | 14.5 | −2.7 |
| | | | | particle | (−0.9) | | (−29.2) | |
| F3-8 | 6.0 | His | 63.7 | Free of | 97.9 | 1.4 | 14.1 | −3.3 |
| | | | | particle | (−1.1) | | (−29.2) | |
| F2-9 | 6.5 | His | 67.1 | Free of | 97.9 | 1.2 | 7.9 | −3.3 |
| | | | | particle | (−1.3) | | (−36.8) | |
| F2-10 | 6.5 | Phos | 66.2 | Free of | 96.3 | 2.8 | 4.0 | −5.3 |
| | | | | particle | (−2.7) | | (−40.5) | |
| F2-11 | 7.0 | Phos | 66.5 | Free of | 96.0 | 2.7 | 2.4 | −7.4 |
| | | | | particle | (−3.0) | | (−41.7) | |

### Example 5: Excipient and Surfactant Screening

To obtain a pharmaceutically acceptable composition including anti-IGF-1R antibody VRDN-1100, a variety of excipients and surfactant concentrations were investigated. The stability of the antibody was determined under each set of experimental conditions. In all experimental conditions, detailed below in Table 17, the antibody was present at a concentration of 50 mg/mL, and the filling volume of each vial was 3.0 mL in 6 R vials.

TABLE 16
Buffer System, Excipient, and Surfactant Combinations Tested

| Anti- | | | Label for |
|---|---|---|---|
| IGF-1R | Buffer | | Experimental |
| Protein | System | Excipient and Surfactants (w/v) | Conditions |
| 50 | 20 mM His, | 8% Sucrose, 0.02% PS80 | F4-1 |
| mg/mL | pH 5.5 | 8.8% Trehalose•2H₂O, 0.02% PS80 | F4-2 |
| | | 2% Sucrose & 4% Mannitol, 0.02% | F4-3 |
| | | PS80 | |
| | | 4.5% Sorbitol, 0.02% PS80 | F4-4 |
| | | 8% Sucrose, 0.06% PS80 | F4-5 |
| | | 8% Sucrose, 10 mM Methionine, | F4-6 |
| | | 0.02% PS80 | |
| | | 8% Sucrose, 10 mM Methionine, | F4-7 |
| | | 0.02% PS20 | |
| | 20 mM His, | 8% Sucrose, 10 mM Methionine, | F4-8 |
| | pH 6.0 | 0.02% PS80 | |
| | | 4.5% Sorbitol, 0.02% PS80 | F4-9 |

The label for each set of experimental conditions will be referred to below. Vials prepared and subject to stress condition as described in Table 18, below. Time points when samples were taken from the experimental vials are also described in Table 15, and the assays performed at each point are described by letters V, W, X, Y, Z.

Letter X indicates the following assays were performed: appearance check, antibody concentration determination, and SEC-UPLC. Letter Y indicates the following assays were performed: Caliper-SDS-NR, Caliper-SDS-R and iCIEF. Letter Z indicates that the following assays were performed: osmolality determination, pH determination, DSC, and viscosity determination. Letter V indicates that HIAC sub-visible particle analysis was performed. Letter W indicates that binding potency determination via ELISA was performed.

TABLE 18
Stress Conditions and Assays

| Stress Condition | Sampling Points and Assay | | |
|---|---|---|---|
| 40° C. | t₀ | 2 Weeks | 4 Weeks |
| | (V, W, X, Y, | X, Y | V, W, X, Y |
| Freeze/Thaw at | Z) | 3 Cycles | 5 Cycles |
| −70° C. to RT | | X | V, W, X, Y |
| Agitation at | | 1 Day | 3 Days |
| 300 rpm, 25° C. | | X | V, W, X, Y |
| 5° C. | | 4 Weeks | 3 Months |
| | | V, X, Y | V, W, X, Y |

DSC results indicated that Tm onset was higher than 60° C. for all of the experimental conditions, indicating good conformational stability. Visual observations did not detect any particles in any experimental conditions. The DSC results are summarized in Table 19, below.

TABLE 19
DSC Results

| | Excipient + Surfactant, | DSC (° C.) | | | |
|---|---|---|---|---|---|
| No. | pH | T_{Onset} | Tₘ1 | Tₘ2 | Tₘ3 |
| F4-1 | Sucr + 0.02% PS80, 5.5 | 61.6 | 68.7 | 77.5 | 85.2 |
| F4-2 | Tre + 0.02% PS80, 5.5 | 61.2 | 69.2 | 77.7 | 85.2 |
| F4-3 | Sucr + Man 0.02% PS80, | 61.2 | 68.7 | 77.5 | 85.0 |
| | 5.5 | | | | |
| F4-4 | Sor + 0.02% PS80, 5.5 | 61.3 | 68.9 | 77.3 | 84.7 |
| F4-5 | Sucr + 0.06% PS80, 5.5 | 60.9 | 68.7 | 77.5 | 85.1 |
| F4-6 | Sucr + Met + 0.02% PS80, 5.5 | 61.4 | 68.7 | 77.5 | 85.3 |
| F4-7 | Sucr + Met + 0.02% PS20, 5.5 | 60.7 | 68.7 | 77.4 | 85.1 |
| F4-8 | Sucr + Met + 0.02% PS80, 6.0 | 64.9 | 73.3 | 79.7 | 87.0 |
| F4-9 | Sor + 0.02% PS80, 6.0 | 64.5 | 72.4 | 79.4 | 86.9 |

### Stress Condition: Incubation at 40° C. for 2-4 Weeks

As shown in Table 20A, below, all samples were visually observed to be slightly yellow, slightly opalescent, and free of visible particles (SY: slightly yellow; SOL: slightly opalescent; FP: free of visible particles). No substantial changes were observed in antibody concentration in any experimental condition. As shown in Table 20A, below, HIAC analysis for all experimental conditions did not show any significant changes in particle counts from t₀ to 4 weeks. As shown in Table 20B, below, SEC-UPLC data showed that condition F4-5 underwent the largest decrease in monomer percentage, an absolute decrease of 3.0 in monomer percentage after 4 weeks at 40° C. F4-6, F4-7, and F4-8 data demonstrated the smallest decreases in monomer percentage, an absolute decrease of 1.1 or less in monomer percentage after 4 weeks at 40° C. Caliper-NR testing showed that the F4-5 condition caused a greater decrease in purity than all other conditions tested (3.3% purity loss compared to 2.2-2.7% purity losses). Caliper-R testing did not reveal any significant differences between any conditions.

TABLE 20A
HIAC Analysis

| | Excipient + | Appearance | | | Protein conc. (mg/mL) | | |
|---|---|---|---|---|---|---|---|
| | Surfactant, | | 40° C.- | 40° C.- | | 40° C.- | 40° C.- |
| No. | pH | T0 | 2 W | 4 W | T0 | 2 W | 4 W |
| F4-1 | Sucr + 0.02% | SY, | SY, | SY, | 51.8 | 54.0 | 50.1 |
| | PS80, 5.5 | SOL, | SOL, | SOL, | | | |
| | | FP | FP | FP | | | |
| F4-2 | Tre + 0.02% | SY, | SY, | SY, | 51.7 | 53.4 | 49.6 |
| | PS80, 5.5 | SOL, | SOL, | SOL, | | | |
| | | FP | FP | FP | | | |
| F4-3 | Sucr + Man | SY, | SY, | SY, | 51.1 | 52.9 | 49.7 |
| | 0.02% PS80, 5.5 | SOL, | SOL, | SOL, | | | |
| | | FP | FP | FP | | | |
| F4-4 | Sor + 0.02% | SY, | SY, | SY, | 51.4 | 51.7 | 49.7 |
| | PS80, 5.5 | SOL, | SOL, | SOL, | | | |
| | | FP | FP | FP | | | |
| F4-5 | Sucr + 0.06% | SY, | SY, | SY, | 51.8 | 52.2 | 49.9 |
| | PS80, 5.5 | SOL, | SOL, | SOL, | | | |
| | | FP | FP | FP | | | |
| F4-6 | Sucr + Met + | SY, | SY, | SY, | 51.8 | 54.2 | 49.5 |
| | 0.02% PS80, 5.5 | SOL, | SOL, | SOL, | | | |
| | | FP | FP | FP | | | |
| F4-7 | Sucr + Met + | SY, | SY, | SY, | 51.7 | 52.2 | 49.6 |
| | 0.02% PS20, 5.5 | SOL, | SOL, | SOL, | | | |
| | | FP | FP | FP | | | |
| F4-8 | Sucr + Met + | SY, | SY, | SY, | 52.0 | 53.5 | 50.0 |
| | 0.02% PS80, 6.0 | SOL, | SOL, | SOL, | | | |
| | | FP | FP | FP | | | |
| F4-9 | Sor + 0.02% | SY, | SY, | SY, | 51.3 | 51.9 | 49.7 |
| | PS80, 6.0 | SOL, | SOL, | SOL, | | | |
| | | FP | FP | FP | | | |

| | Sub-visible particles Counts (#/mL) | | | | | | | | |
|---|---|---|---|---|---|---|---|---|---|
| | T0 | | | 40° C.-4 W | | | | Osmolality | Viscosity |
| | ≥2 | ≥10 | ≥25 | ≥2 | ≥10 | ≥25 | pH | (mOsm/kg) | (cp) |
| No. | μm | μm | μm | μm | μm | μm | T0 | T0 | T0 |
| F4-1 | 254 | 20 | 0 | 259 | 22 | 0 | 5.6 | 312 | 1.8 |
| F4-2 | 261 | 10 | 0 | 309 | 8 | 1 | 5.6 | 314 | 1.8 |
| F4-3 | 315 | 7 | 0 | 175 | 32 | 1 | 5.6 | 353 | 1.7 |
| F4-4 | 151 | 10 | 1 | 109 | 3 | 0 | 5.6 | 310 | 1.6 |
| F4-5 | 162 | 12 | 1 | 108 | 6 | 1 | 5.6 | 314 | 1.7 |
| F4-6 | 49 | 5 | 3 | 55 | 6 | 0 | 5.6 | 327 | 1.7 |
| F4-7 | 37 | 3 | 1 | 174 | 10 | 1 | 5.6 | 327 | 1.7 |
| F4-8 | 796 | 35 | 3 | 195 | 15 | 2 | 6.1 | 335 | 1.7 |
| F4-9 | 206 | 10 | 1 | 70 | 5 | 1 | 6.1 | 322 | 1.6 |

TABLE 20B
SEC-UPLC Results

| | | SEC-UPLC | | | | | | | | |
|---|---|---|---|---|---|---|---|---|---|---|
| | Excipient + | Monomer % | | | HMW % | | | LMW % | | |
| | Surfactant, | | 40° C.- | 40° C.- | | 40° C.- | 40° C.- | | 40° C.- | 40° C.- |
| No. | pH | T0 | 2 W | 4 W | T0 | 2 W | 4 W | T0 | 2 W | 4 W |
| F4-1 | Sucr + 0.02% | 99.4 | 98.5 | 97.8 | 0.5 | 1.1 | 1.6 | 0.1 | 0.4 | 0.7 |
| | PS80, 5.5 | | (−0.9) | (−1.6) | | | | | | |
| F4-2 | Tre + 0.02% | 99.4 | 98.5 | 97.8 | 0.5 | 1.1 | 1.6 | 0.1 | 0.4 | 0.6 |
| | PS80, 5.5 | | (−0.9) | (−1.6) | | | | | | |
| F4-3 | Sucr + Man | 99.2 | 98.2 | 97.7 | 0.7 | 1.4 | 1.7 | 0.1 | 0.4 | 0.6 |
| | 0.02% PS80, 5.5 | | (−1.0) | (−1.5) | | | | | | |
| F4-4 | Sor + 0.02% | 99.4 | 98.4 | 97.6 | 0.5 | 1.2 | 1.7 | 0.1 | 0.4 | 0.7 |
| | PS80, 5.5 | | (−1.0) | (−1.8) | | | | | | |
| F4-5 | Sucr + 0.06% | 99.4 | 97.9 | 96.4 | 0.5 | 1.7 | 2.9 | 0.1 | 0.4 | 0.7 |
| | PS80, 5.5 | | (−1.5) | (−3.0) | | | | | | |
| F4-6 | Sucr + Met + | 99.4 | 98.8 | 98.3 | 0.5 | 0.8 | 1.1 | 0.1 | 0.4 | 0.6 |
| | 0.02% PS80, 5.5 | | (−0.6) | (−1.1) | | | | | | |
| F4-7 | Sucr + Met + | 99.4 | 99.0 | 98.5 | 0.5 | 0.7 | 0.9 | 0.1 | 0.3 | 0.5 |
| | 0.02% PS20, 5.5 | | (−0.4) | (−0.9) | | | | | | |
| F4-8 | Sucr + Met + | 99.4 | 98.8 | 98.3 | 0.6 | 0.9 | 1.1 | 0.1 | 0.4 | 0.6 |
| | 0.02% PS80, 6.0 | | (−0.6) | (−1.1) | | | | | | |
| F4-9 | Sor + 0.02% | 99.3 | 98.3 | 97.7 | 0.6 | 1.3 | 1.7 | 0.1 | 0.4 | 0.7 |
| | PS80, 6.0 | | (−1.0) | (−1.6) | | | | | | |

TABLE 20C
iCIEF Results

| | | iCIEF | | | | | | | | |
|---|---|---|---|---|---|---|---|---|---|---|
| | Excipient + | Main peak % (MP) | | | Acidic peaks % (AP) | | | Basic peaks % (BP) | | |
| | Surfactant, | | 40° C.- | 40° C.- | | 40° C.- | 40° C.- | | 40° C.- | 40° C.- |
| No. | pH | T0 | 2 W | 4 W | T0 | 2 W | 4 W | T0 | 2 W | 4 W |
| F4-1 | Sucr + 0.02% | 44.3 | 25.6 | 16.9 | 35.2 | 52.0 | 64.4 | 20.5 | 22.4 | 18.7 |
| | PS80, 5.5 | | (−18.7) | (−27.4) | | | | | | |
| F4-2 | Tre + 0.02% | 43.5 | 24.0 | 15.5 | 35.7 | 53.2 | 64.6 | 20.8 | 22.8 | 19.9 |
| | PS80, 5.5 | | (−19.5) | (−28.0) | | | | | | |
| F4-3 | Sucr + Man | 44.8 | 25.9 | 17.5 | 35.3 | 51.8 | 63.0 | 19.9 | 22.3 | 19.5 |
| | 0.02% PS80, 5.5 | | (−18.9) | (−27.3) | | | | | | |
| F4-4 | Sor + 0.02% | 43.0 | 26.1 | 15.8 | 36.5 | 51.5 | 63.9 | 20.4 | 22.3 | 20.3 |
| | PS80, 5.5 | | (−16.9) | (−27.2) | | | | | | |
| F4-5 | Sucr + 0.06% | 42.4 | 22.6 | 15.1 | 36.9 | 56.0 | 67.0 | 20.7 | 21.4 | 17.9 |
| | PS80, 5.5 | | (−19.8) | (−27.3) | | | | | | |
| F4-6 | Sucr + Met + | 44.1 | 22.1 | 16.1 | 35.6 | 53.7 | 64.1 | 20.4 | 24.2 | 19.8 |
| | 0.02% PS80, 5.5 | | (−22.0) | (−28.0) | | | | | | |
| F4-7 | Sucr + Met + | 42.9 | 21.8 | 15.9 | 36.4 | 53.3 | 63.4 | 20.7 | 24.9 | 20.8 |
| | 0.02% PS20, 5.5 | | (−21.1) | (−27.0) | | | | | | |
| F4-8 | Sucr + Met + | 43.5 | 19.2 | 12.6 | 36.2 | 58.4 | 70.1 | 20.3 | 22.5 | 17.2 |
| | 0.02% PS80, 6.0 | | (−24.3) | (−30.9) | | | | | | |
| F4-9 | Sor + 0.02% | 43.0 | 20.6 | 12.0 | 36.9 | 58.5 | 70.4 | 20.0 | 20.9 | 17.6 |
| | PS80, 6.0 | | (−22.4) | (−31.0) | | | | | | |

### Stress Condition: Incubation at 5° C. for 4 Weeks to 3 Months

As shown in Table 21A, below, all samples were visually observed to be slightly yellow, slightly opalescent, and free of visible particles (SY: slightly yellow; SOL: slightly opalescent; FP: free of visible particles). No substantial changes were observed in protein concentration in any experimental condition. As shown in Table 21A, below, HIAC analysis for all experimental conditions did not show any significant changes at up to 4 weeks. As shown in Table 21B, below, SEC-UPLC data showed no significant changes after incubation at 5° C. for 4 weeks. As shown in Table 21C, below, iCIEF analysis of the F1 condition showed an absolute decrease of 2.5 in the main peak percentage after incubation at 5° C. for 4 weeks. No other experimental conditions showed a significant change by iCIEF. As shown in Table 18D, below, testing the vials by Caliper-NR and Caliper-R showed no significant changes for any experimental conditions.

TABLE 21A
HIAC Analysis

| | | | | | | | | Sub-visible particles Counts (#/mL) | | | | | | | | |
|---|---|---|---|---|---|---|---|---|---|---|---|---|---|---|---|---|
| | Excipient + | Appearance | | | Protein conc. (mg/mL) | | | T0 | | | 5° C.-4 W | | | 5° C.-3 M | | |
| | Surfactant, | | 5° C.- | 5° C.- | | 5° C.- | 5° C.- | ≥2 | ≥10 | ≥25 | ≥2 | ≥10 | ≥25 | ≥2 | ≥10 | ≥25 |
| No. | pH | T0 | 4 W | 3 M | T0 | 4 W | 3 M | μm | μm | μm | μm | μm | μm | μm | μm | μm |
| F4-1 | Sucr + 0.02% | SY, | SY, | SY, | 51.8 | 50.3 | 50.0 | 254 | 20 | 0 | 114 | 9 | 0 | 595 | 32 | 0 |
| | PS80, 5.5 | SOL, | SOL, | SOL, | | | | | | | | | | | | |
| | | FP | FP | FP | | | | | | | | | | | | |
| F4-2 | Tre + 0.02% | SY, | SY, | SY, | 51.7 | 49.7 | 49.9 | 261 | 10 | 0 | 49 | 3 | 0 | 103 | 7 | 2 |
| | PS80, 5.5 | SOL, | SOL, | SOL, | | | | | | | | | | | | |
| | | FP | FP | FP | | | | | | | | | | | | |
| F4-3 | Sucr + Man | SY, | SY, | SY, | 51.1 | 49.4 | 50.1 | 315 | 7 | 0 | 371 | 8 | 0 | 232 | 20 | 1 |
| | 0.02% PS80, 5.5 | SOL, | SOL, | SOL, | | | | | | | | | | | | |
| | | FP | FP | FP | | | | | | | | | | | | |
| F4-4 | Sor + 0.02% | SY, | SY, | SY, | 51.4 | 49.5 | 49.6 | 151 | 10 | 1 | 189 | 7 | 0 | 233 | 13 | 0 |
| | PS80, 5.5 | SOL, | SOL, | SOL, | | | | | | | | | | | | |
| | | FP | FP | FP | | | | | | | | | | | | |
| F4-5 | Sucr + 0.06% | SY, | SY, | SY, | 51.8 | 49.7 | 49.7 | 162 | 12 | 1 | 91 | 8 | 0 | 179 | 0 | 0 |
| | PS80, 5.5 | SOL, | SOL, | SOL, | | | | | | | | | | | | |
| | | FP | FP | FP | | | | | | | | | | | | |
| F4-6 | Sucr + Met + | SY, | SY, | SY, | 51.8 | 49.9 | 49.6 | 49 | 5 | 3 | 78 | 4 | 0 | 195 | 12 | 8 |
| | 0.02% PS80, 5.5 | SOL, | SOL, | SOL, | | | | | | | | | | | | |
| | | FP | FP | FP | | | | | | | | | | | | |
| F4-7 | Sucr + Met + | SY, | SY, | SY, | 51.7 | 49.7 | 49.6 | 37 | 3 | 1 | 35 | 1 | 0 | 197 | 6 | 1 |
| | 0.02% PS20, 5.5 | SOL, | SOL, | SOL, | | | | | | | | | | | | |
| | | FP | FP | FP | | | | | | | | | | | | |
| F4-8 | Sucr + Met + | SY, | SY, | SY, | 52.0 | 50.5 | 49.8 | 796 | 35 | 3 | 41 | 1 | 0 | 276 | 4 | 0 |
| | 0.02% PS80, 6.0 | SOL, | SOL, | SOL, | | | | | | | | | | | | |
| | | FP | FP | FP | | | | | | | | | | | | |
| F4-9 | Sor + 0.02% | SY, | SY, | SY, | 51.3 | 49.7 | 49.2 | 206 | 10 | 1 | 35 | 3 | 0 | 203 | 15 | 0 |
| | PS80, 6.0 | SOL, | SOL, | SOL, | | | | | | | | | | | | |
| | | FP | FP | FP | | | | | | | | | | | | |

TABLE 21B
SEC-UPLC Results

| | Excipient + | SEC-UPLC | | | | | | | | |
|---|---|---|---|---|---|---|---|---|---|---|
| | Surfactant, | Monomer % | | | HMW % | | | LMW % | | |
| No. | pH | T0 | 5-4 W | 5-3 M | T0 | 5-4 W | 5-3 M | T0 | 5-4 W | 5-3 M |
| F4-1 | Sucr + 0.02% | 99.4 | 99.3 | 99.2 | 0.5 | 0.6 | 0.7 | 0.1 | 0.1 | 0.1 |
| | PS80, 5.5 | | | | | | | | | |
| F4-2 | Tre + 0.02% | 99.4 | 99.3 | 99.2 | 0.5 | 0.6 | 0.7 | 0.1 | 0.1 | 0.1 |
| | PS80, 5.5 | | | | | | | | | |
| F4-3 | Sucr + Man | 99.2 | 99.3 | 99.1 | 0.7 | 0.6 | 0.8 | 0.1 | 0.1 | 0.1 |
| | 0.02% PS80, 5.5 | | | | | | | | | |
| F4-4 | Sor + 0.02% | 99.4 | 99.3 | 99.2 | 0.5 | 0.6 | 0.8 | 0.1 | 0.1 | 0.1 |
| | PS80, 5.5 | | | | | | | | | |
| F4-5 | Sucr + 0.06% | 99.4 | 99.3 | 99.2 | 0.5 | 0.6 | 0.7 | 0.1 | 0.1 | 0.1 |
| | PS80, 5.5 | | | | | | | | | |
| F4-6 | Sucr + Met + | 99.4 | 99.4 | 99.4 | 0.5 | 0.5 | 0.6 | 0.1 | 0.1 | 0.1 |
| | 0.02% PS80, 5.5 | | | | | | | | | |
| F4-7 | Sucr + Met + | 99.4 | 99.4 | 99.4 | 0.5 | 0.5 | 0.5 | 0.1 | 0.1 | 0.1 |
| | 0.02% PS20, 5.5 | | | | | | | | | |
| F4-8 | Sucr + Met + | 99.4 | 99.3 | 99.3 | 0.6 | 0.6 | 0.7 | 0.1 | 0.1 | 0.0 |
| | 0.02% PS80, 6.0 | | | | | | | | | |
| F4-9 | Sor + 0.02% | 99.3 | 99.2 | 99.2 | 0.6 | 0.7 | 0.7 | 0.1 | 0.1 | 0.1 |
| | PS80, 6.0 | | | | | | | | | |

TABLE 21C
iCIEF Results

| | | iCIEF | | | | | | | | |
|---|---|---|---|---|---|---|---|---|---|---|
| | Excipient + | Main peak % (MP) | | | Acidic peaks % (AP) | | | Basic peaks % (BP) | | |
| No. | Surfactant, pH | T0 | 5° C.-4 W | 5° C.-3 M | T0 | 5° C.-4 W | 5° C.-3 M | T0 | 5° C.-4 W | 5° C.-3 M |
| F4-1 | Sucr + 0.02% | 44.3 | 41.8 | 44.2 | 35.2 | 36.8 | 35.3 | 20.5 | 21.3 | 20.6 |
| | PS80, 5.5 | | | | | | | | | |
| F4-2 | Tre + 0.02% | 43.5 | 44.1 | 43.5 | 35.7 | 35.4 | 35.5 | 20.8 | 20.5 | 21.0 |
| | PS80, 5.5 | | | | | | | | | |
| F4-3 | Sucr + Man | 44.8 | 43.1 | 43.6 | 35.3 | 36.4 | 35.7 | 19.9 | 20.5 | 20.7 |
| | 0.02% PS80, 5.5 | | | | | | | | | |
| F4-4 | Sor + 0.02% | 43.0 | 42.7 | 43.0 | 36.5 | 36.9 | 36.5 | 20.4 | 20.4 | 20.5 |
| | PS80, 5.5 | | | | | | | | | |
| F4-5 | Sucr + 0.06% | 42.4 | 42.3 | 43.9 | 36.9 | 36.8 | 35.4 | 20.7 | 20.9 | 20.7 |
| | PS80, 5.5 | | | | | | | | | |
| F4-6 | Sucr + Met + | 44.1 | 43.4 | 43.0 | 35.6 | 35.8 | 36.1 | 20.4 | 20.8 | 20.9 |
| | 0.02% PS80, 5.5 | | | | | | | | | |
| F4-7 | Sucr + Met + | 42.9 | 43.0 | 43.6 | 36.4 | 36.9 | 35.5 | 20.7 | 20.1 | 20.8 |
| | 0.02% PS20, 5.5 | | | | | | | | | |
| F4-8 | Sucr + Met + | 43.5 | 41.2 | 42.9 | 36.2 | 39.0 | 36.8 | 20.3 | 19.8 | 20.3 |
| | 0.02% PS80, 6.0 | | | | | | | | | |
| F4-9 | Sor + 0.02% | 43.0 | 43.5 | 42.4 | 36.9 | 36.2 | 37.2 | 20.0 | 20.2 | 20.4 |
| | PS80, 6.0 | | | | | | | | | |

TABLE 21D
Caliper-NR and Caliper-R Results

| | | Caliper-NR | | | Caliper-R | | |
|---|---|---|---|---|---|---|---|
| | Excipient + | Purity % | | | Purity % | | |
| No. | Surfactant, pH | T0 | 5° C.-4 W | 5° C.-3 M | T0 | 5° C.-4 W | 5° C.-3 M |
| F4-1 | Sucr + 0.02% | 97.5 | 97.5 | 98.0 | 99.2 | 99.2 | 99.4 |
| | PS80, 5.5 | | | | | | |
| F4-2 | Tre + 0.02% | 97.3 | 97.3 | 97.9 | 99.2 | 99.2 | 99.4 |
| | PS80, 5.5 | | | | | | |
| F4-3 | Sucr + Man | 97.3 | TBD | 98.1 | 99.2 | 99.1 | 99.4 |
| | 0.02% PS80, 5.5 | | | | | | |
| F4-4 | Sor + 0.02% | TBD | 97.5 | 98.0 | 99.1 | 99.2 | 99.4 |
| | PS80, 5.5 | | | | | | |
| F4-5 | Sucr + 0.06% | 97.5 | 97.7 | 98.1 | 99.1 | 99.1 | 99.3 |
| | PS80, 5.5 | | | | | | |
| F4-6 | Sucr + Met + | 97.8 | 97.4 | 98.1 | 99.1 | 99.2 | 99.3 |
| | 0.02% PS80, 5.5 | | | | | | |
| F4-7 | Sucr + Met + | 97.7 | 97.5 | 98.2 | 99.1 | 99.2 | 99.4 |
| | 0.02% PS20, 5.5 | | | | | | |
| F4-8 | Sucr + Met + | 97.4 | 97.5 | 98.0 | 99.1 | 99.2 | 99.4 |
| | 0.02% PS80, 6.0 | | | | | | |
| F4-9 | Sor + 0.02% | 97.4 | 97.4 | 97.9 | 99.1 | 99.2 | 99.4 |
| | PS80, 6.0 | | | | | | |

### Stress Condition: Agitation at 300 Rpm and 25° C. for 1-3 Days

As shown in Table 22A, below, all samples were visually observed to be slightly yellow, slightly opalescent, and free of visible particles (SY: slightly yellow; SOL: slightly opalescent; FP: free of visible particles). No substantial changes were observed in protein concentration in any experimental condition. As shown in Table 22A, below, HIAC analysis for all experimental conditions did not show any significant changes at up to 3 days. As shown in Table 22B, below, no significant change in SEC-UPLC, iCIEF, Caliper-NR, or Caliper-R data was observed for any conditions after 1 or 3 days of agitation.

TABLE 22A
HIAC Analysis

| | | | | | | | | Sub-visible particles Counts (#/mL) | | | | | |
|---|---|---|---|---|---|---|---|---|---|---|---|---|---|
| | Excipient + | Appearance | | | Protein conc. (mg/mL) | | | T0 | | | A-3 D | | |
| No. | Surfactant, pH | T0 | A-1 D | A-3 D | T0 | A-1 D | A-3 D | ≥2 μm | ≥10 μm | ≥25 μm | ≥2 μm | ≥10 μm | ≥25 μm |
| F4-1 | Sucr + 0.02% | SY, | SY, | SY, | 51.8 | 50.2 | 49.8 | 254 | 20 | 0 | 109 | 6 | 2 |
| | PS80, 5.5 | SOL, | SOL, | SOL, | | | | | | | | | |
| | | FP | FP | FP | | | | | | | | | |
| F4-2 | Tre + 0.02% | SY, | SY, | SY, | 51.7 | 49.6 | 51.6 | 261 | 10 | 0 | 95 | 1 | 0 |
| | PS80, 5.5 | SOL, | SOL, | SOL, | | | | | | | | | |
| | | FP | FP | FP | | | | | | | | | |
| F4-3 | Sucr + Man | SY, | SY, | SY, | 51.1 | 49.5 | 50.6 | 315 | 7 | 0 | 57 | 3 | 0 |
| | 0.02% PS80, 5.5 | SOL, | SOL, | SOL, | | | | | | | | | |
| | | FP | FP | FP | | | | | | | | | |
| F4-4 | Sor + 0.02% | SY, | SY, | SY, | 51.4 | 49.5 | 50.7 | 151 | 10 | 1 | 53 | 0 | 0 |
| | PS80, 5.5 | SOL, | SOL, | SOL, | | | | | | | | | |
| | | FP | FP | FP | | | | | | | | | |
| F4-5 | Sucr + 0.06% | SY, | SY, | SY, | 51.8 | 49.8 | 50.5 | 162 | 12 | 1 | 249 | 4 | 0 |
| | PS80, 5.5 | SOL, | SOL, | SOL, | | | | | | | | | |
| | | FP | FP | FP | | | | | | | | | |
| F4-6 | Sucr + Met + | SY, | SY, | SY, | 51.8 | 49.7 | 50.5 | 49 | 5 | 3 | 111 | 3 | 0 |
| | 0.02% PS80, 5.5 | SOL, | SOL, | SOL, | | | | | | | | | |
| | | FP | FP | FP | | | | | | | | | |
| F4-7 | Sucr + Met + | SY, | SY, | SY, | 51.7 | 49.7 | 50.0 | 37 | 3 | 1 | 44 | 0 | 0 |
| | 0.02% PS20, 5.5 | SOL, | SOL, | SOL, | | | | | | | | | |
| | | FP | FP | FP | | | | | | | | | |
| F4-8 | Sucr + Met + | SY, | SY, | SY, | 52.0 | 49.9 | 50.4 | 796 | 35 | 3 | 46 | 1 | 0 |
| | 0.02% PS80, 6.0 | SOL, | SOL, | SOL, | | | | | | | | | |
| | | FP | FP | FP | | | | | | | | | |
| F4-9 | Sor + 0.02% | SY, | SY, | SY, | 51.3 | 49.6 | 49.9 | 206 | 10 | 1 | 179 | 9 | 0 |
| | PS80, 6.0 | SOL, | SOL, | SOL, | | | | | | | | | |
| | | FP | FP | FP | | | | | | | | | |

TABLE 22B
SEC-UPLC, iCIEF, Caliper-NR, and Caliper-R Results

| | | SEC-UPLC | | | | | | | | |
|---|---|---|---|---|---|---|---|---|---|---|
| | Excipient + | Monomer % | | | HMW % | | | LMW % | | |
| | Surfactant, pH | T0 | A-1 D | A-3 D | T0 | A-1 D | A-3 D | T0 | A-1 D | A-3 D |
| F4-1 | Sucr + 0.02% | 99.4 | 99.4 | 99.4 | 0.5 | 0.5 | 0.5 | 0.1 | 0.1 | 0.1 |
| | PS80, 5.5 | | | | | | | | | |
| F4-2 | Tre + 0.02% | 99.4 | 99.4 | 99.4 | 0.5 | 0.5 | 0.5 | 0.1 | 0.1 | 0.1 |
| | PS80, 5.5 | | | | | | | | | |
| F4-3 | Sucr + Man | 99.2 | 99.3 | 99.3 | 0.7 | 0.6 | 0.6 | 0.1 | 0.1 | 0.1 |
| | 0.02% PS80, 5.5 | | | | | | | | | |
| F4-4 | Sor + 0.02% | 99.4 | 99.4 | 99.4 | 0.5 | 0.5 | 0.6 | 0.1 | 0.1 | 0.1 |
| | PS80, 5.5 | | | | | | | | | |
| F4-5 | Sucr + 0.06% | 99.4 | 99.4 | 99.4 | 0.5 | 0.6 | 0.6 | 0.1 | 0.1 | 0.1 |
| | PS80, 5.5 | | | | | | | | | |
| F4-6 | Sucr + Met + | 99.4 | 99.4 | 99.4 | 0.5 | 0.5 | 0.5 | 0.1 | 0.1 | 0.1 |
| | 0.02% PS80, 5.5 | | | | | | | | | |
| F4-7 | Sucr + Met + | 99.4 | 99.4 | 99.4 | 0.5 | 0.5 | 0.5 | 0.1 | 0.1 | 0.1 |
| | 0.02% PS20, 5.5 | | | | | | | | | |
| F4-8 | Sucr + Met + | 99.4 | 99.4 | 99.4 | 0.6 | 0.6 | 0.6 | 0.1 | 0.1 | 0.1 |
| | 0.02% PS80, 6.0 | | | | | | | | | |
| F4-9 | Sor + 0.02% | 99.3 | 99.3 | 99.3 | 0.6 | 0.6 | 0.6 | 0.1 | 0.1 | 0.1 |
| | PS80, 6.0 | | | | | | | | | |

| | iCIEF | | | | | | Caliper-NR | | Caliper-R | |
|---|---|---|---|---|---|---|---|---|---|---|
| | Main peak % | | Acidic peaks % | | Basic peaks % | | Purity % | | Purity % | |
| | T0 | A-3 D | T0 | A-3 D | T0 | A-3 D | T0 | A-3 D | T0 | A-3 D |
| F4-1 | 44.3 | 43.2 | 35.2 | 36.1 | 20.5 | 20.6 | 97.5 | 97.3 | 99.2 | 99.2 |
| F4-2 | 43.5 | 43.2 | 35.7 | 36.3 | 20.8 | 20.5 | 97.3 | 97.5 | 99.2 | 99.2 |
| F4-3 | 44.8 | 43.8 | 35.3 | 35.8 | 19.9 | 20.3 | 97.3 | 97.4 | 99.2 | 99.1 |
| F4-4 | 43.0 | 42.6 | 36.5 | 37.0 | 20.4 | 20.4 | TBD | 97.5 | 99.1 | 99.2 |
| F4-5 | 42.4 | 43.3 | 36.9 | 36.7 | 20.7 | 20.0 | 97.5 | 97.4 | 99.1 | 99.1 |
| F4-6 | 44.1 | 43.1 | 35.6 | 36.2 | 20.4 | 20.6 | 97.8 | 97.5 | 99.1 | 99.1 |
| F4-7 | 42.9 | 42.5 | 36.4 | 37.3 | 20.7 | 20.1 | 97.7 | 97.4 | 99.1 | 99.1 |
| F4-8 | 43.5 | 42.4 | 36.2 | 37.5 | 20.3 | 20.1 | 97.4 | 97.4 | 99.1 | 99.1 |
| F4-9 | 43.0 | 43.8 | 36.9 | 36.3 | 20.0 | 19.9 | 97.4 | 97.3 | 99.1 | 99.2 |

### Stress Condition: Freeze-Thawing at −70° C. to Room Temperature for 3-5 Cycles

As shown in Table 23A, below, all samples were visually observed to be slightly yellow, slightly opalescent, and free of visible particles (SY: slightly yellow; SOL: slightly opalescent; FP: free of visible particles). No substantial changes were observed in protein concentration in any experimental condition. As shown in Table 23A, below, HIAC analysis for all experimental conditions did not show any significant changes at up to 4 freeze-thaw cycles. As shown in Table 23B, below, Caliper-R analysis showed that the F4-9 condition displayed a purity decrease of 1.6% after 5 cycles of freeze-thawing. Analysis of freeze-thaw tested vials by SEC-UPLC, iCIEF, and Caliper-NR did not show any significant differences between the experimental conditions.

TABLE 23A
HIAC Analysis

| | | Appearance | | | Protein conc. (mg/mL) | | | Sub-visible particles Counts (#/mL) | | | | | |
|---|---|---|---|---|---|---|---|---|---|---|---|---|---|
| | Excipient + | | FT-3 | FT-5 | | FT-3 | FT-5 | T0 | | | FT-5 Cys | | |
| No. | Surfactant, pH | T0 | Cys | Cys | T0 | Cys | Cys | ≥2 μm | ≥10 μm | ≥25 μm | ≥2 μm | ≥10 μm | ≥25 μm |
| F4-1 | Sucr + 0.02% | SY, | SY, | SY, | 51.8 | 50.4 | 49.9 | 254 | 20 | 0 | 117 | 9 | 1 |
| | PS80, 5.5 | SOL, | SOL, | SOL, | | | | | | | | | |
| | | FP | FP | FP | | | | | | | | | |
| F4-2 | Tre + 0.02% | SY, | SY, | SY, | 51.7 | 50.9 | 49.9 | 261 | 10 | 0 | 89 | 5 | 0 |
| | PS80, 5.5 | SOL, | SOL, | SOL, | | | | | | | | | |
| | | FP | FP | FP | | | | | | | | | |
| F4-3 | Sucr + Man | SY, | SY, | SY, | 51.1 | 50.4 | 50.1 | 315 | 7 | 0 | 77 | 3 | 0 |
| | 0.02% PS80, 5.5 | SOL, | SOL, | SOL, | | | | | | | | | |
| | | FP | FP | FP | | | | | | | | | |
| F4-4 | Sor + 0.02% | SY, | SY, | SY, | 51.4 | 50.7 | 49.9 | 151 | 10 | 1 | 135 | 9 | 1 |
| | PS80, 5.5 | SOL, | SOL, | SOL, | | | | | | | | | |
| | | FP | FP | FP | | | | | | | | | |
| F4-5 | Sucr + 0.06% | SY, | SY, | SY, | 51.8 | 50.4 | 49.9 | 162 | 12 | 1 | 80 | 1 | 0 |
| | PS80, 5.5 | SOL, | SOL, | SOL, | | | | | | | | | |
| | | FP | FP | FP | | | | | | | | | |
| F4-6 | Sucr + Met + | SY, | SY, | SY, | 51.8 | 51.8 | 50.1 | 49 | 5 | 3 | 43 | 2 | 0 |
| | 0.02% PS80, 5.5 | SOL, | SOL, | SOL, | | | | | | | | | |
| | | FP | FP | FP | | | | | | | | | |
| F4-7 | Sucr + Met + | SY, | SY, | SY, | 51.7 | 51.3 | 49.7 | 37 | 3 | 1 | 41 | 6 | 0 |
| | 0.02% PS20, 5.5 | SOL, | SOL, | SOL, | | | | | | | | | |
| | | FP | FP | FP | | | | | | | | | |
| F4-8 | Sucr + Met + | SY, | SY, | SY, | 52.0 | 51.7 | 50.5 | 796 | 35 | 3 | 56 | 0 | 0 |
| | 0.02% PS80, 6.0 | SOL, | SOL, | SOL, | | | | | | | | | |
| | | FP | FP | FP | | | | | | | | | |
| F4-9 | Sor + 0.02% | SY, | SY, | SY, | 51.3 | 51.1 | 49.9 | 206 | 10 | 1 | 32 | 3 | 0 |
| | PS80, 6.0 | SOL, | SOL, | SOL, | | | | | | | | | |
| | | FP | FP | FP | | | | | | | | | |

TABLE 23B
SEC-UPLC, iCIEF, Caliper-NR, Caliper-R Results

| | | SEC-UPLC | | | | | | | | |
|---|---|---|---|---|---|---|---|---|---|---|
| | | Monomer % | | | HMW % | | | LMW % | | |
| | Excipient + | | FT-3 | FT-5 | | FT-3 | FT-5 | | FT-3 | FT-5 |
| No. | Surfactant, pH | T0 | Cys | Cys | T0 | Cys | Cys | T0 | Cys | Cys |
| F4-1 | Sucr + 0.02% | 99.4 | 99.4 | 99.4 | 0.5 | 0.6 | 0.6 | 0.1 | 0.1 | 0.1 |
| | PS80, 5.5 | | | | | | | | | |
| F4-2 | Tre + 0.02% | 99.4 | 99.4 | 99.4 | 0.5 | 0.6 | 0.6 | 0.1 | 0.1 | 0.1 |
| | PS80, 5.5 | | | | | | | | | |
| F4-3 | Sucr + Man | 99.2 | 99.2 | 99.2 | 0.7 | 0.7 | 0.7 | 0.1 | 0.1 | 0.1 |
| | 0.02% PS80, 5.5 | | | | | | | | | |
| F4-4 | Sor + 0.02% | 99.4 | 99.4 | 99.4 | 0.5 | 0.6 | 0.6 | 0.1 | 0.1 | 0.1 |
| | PS80, 5.5 | | | | | | | | | |
| F4-5 | Sucr + 0.06% | 99.4 | 99.4 | 99.4 | 0.5 | 0.6 | 0.6 | 0.1 | 0.1 | 0.1 |
| | PS80, 5.5 | | | | | | | | | |
| F4-6 | Sucr + Met + | 99.4 | 99.4 | 99.4 | 0.5 | 0.5 | 0.5 | 0.1 | 0.1 | 0.1 |
| | 0.02% PS80, 5.5 | | | | | | | | | |
| F4-7 | Sucr + Met + | 99.4 | 99.4 | 99.4 | 0.5 | 0.5 | 0.5 | 0.1 | 0.1 | 0.1 |
| | 0.02% PS20, 5.5 | | | | | | | | | |
| F4-8 | Sucr + Met + | 99.4 | 99.3 | 99.3 | 0.6 | 0.6 | 0.6 | 0.1 | 0.1 | 0.1 |
| | 0.02% PS80, 6.0 | | | | | | | | | |
| F4-9 | Sor + 0.02% | 99.3 | 99.3 | 99.3 | 0.6 | 0.6 | 0.6 | 0.1 | 0.1 | 0.1 |
| | PS80, 6.0 | | | | | | | | | |

| | iCIEF | | | | | | Caliper-NR | | Caliper-R | |
|---|---|---|---|---|---|---|---|---|---|---|
| | Main peak % | | Acidic peaks % | | Basic peaks % | | Purity % | | Purity % | |
| | | FT-5 | | FT-5 | | FT-5 | | FT-5 | | FT-5 |
| No. | T0 | Cys | T0 | Cys | T0 | Cys | T0 | Cys | T0 | Cys |
| F4-1 | 44.3 | 43.6 | 35.2 | 35.8 | 20.5 | 20.6 | 97.5 | 97.5 | 99.2 | 99.2 |
| F4-2 | 43.5 | 43.5 | 35.7 | 35.7 | 20.8 | 20.8 | 97.3 | 97.4 | 99.2 | 99.1 |
| F4-3 | 44.8 | 43.0 | 35.3 | 36.3 | 19.9 | 20.6 | 97.3 | 97.2 | 99.2 | 99.2 |
| F4-4 | 43.0 | 42.8 | 36.5 | 37.3 | 20.4 | 19.9 | TBD | 97.2 | 99.1 | 99.1 |
| F4-5 | 42.4 | 43.0 | 36.9 | 36.1 | 20.7 | 21.0 | 97.5 | 97.4 | 99.1 | 99.2 |
| F4-6 | 44.1 | 43.7 | 35.6 | 35.5 | 20.4 | 20.8 | 97.8 | 97.5 | 99.1 | 99.2 |
| F4-7 | 42.9 | 42.9 | 36.4 | 36.7 | 20.7 | 20.4 | 97.7 | 97.4 | 99.1 | 99.2 |
| F4-8 | 43.5 | 43.6 | 36.2 | 36.2 | 20.3 | 20.2 | 97.4 | 97.5 | 99.1 | 99.1 |
| F4-9 | 43.0 | 42.9 | 36.9 | 36.9 | 20.0 | 20.1 | 97.4 | 97.4 | 99.1 | 97.5 |

The data obtained from these investigations indicated that combinations of excipients and surfactants with sucrose, methionine, polysorbate 20 or 80, with a pH of about 4.0-6.0 had superior properties, which could not have been predicted. In some embodiments, these combinations were: 8% Sucrose, 10 mM Methionine, 0.02% PS80, with 20 mM histidine buffer, at a pH of 5.5; and 8% Sucrose, 10 mM Methionine, 0.02% PS20, with 20 mM histidine buffer, at a pH of 5.5.

### Example 6: Formulation Confirmation Study

To obtain a pharmaceutically acceptable composition including anti-IGF-1R antibody VRDN-1100, the stability of a preferred formulation was tested while the formulation was housed in specific container closure systems. In all experimental conditions, described below in Table 24, the following formulations were tested: antibody at 50 mg/mL, 20 mM histidine buffer, 8% sucrose, 10 mM Methionine, and 0.02% PS80, at a pH of 5.5. The container closure systems for this study are as follows: 2 R vials from Schott® (Cat. No. 1626151) with a stopper from West Pharmaceuticals Services™ (Cat. No. 1970-0173) and an aluminum-plastic cap from West Pharmaceuticals Services™ (Cat. No. 5413-2150), and 10 R vials from Schott (Cat. No. 1531542) with a stopper from West Pharmaceuticals Services™ (Cat. No. 1970-0376) and an aluminum-plastic cap from West Pharmaceutical Services™ (Cat. No. 5420-2960).

TABLE 24
Stress Conditions and Assays

| Conditions | t₀ | Sampling Points and Assay | | |
|---|---|---|---|---|
| 2-8° C. (Inverted) | X, Y, Z, Q | 2 Weeks | 4 Weeks | 3 Months |
| | | X, Y | None | X, Y, Z |
| 25° C. (Inverted) | | 2 Weeks | 4 Weeks | 3 Months |
| | | X, Y | None | X, Y, Z |
| 40° C. (Upright) | | 2 Weeks | 4 Weeks | 3 Months |
| | | X, Y | X, Y, Z | None |

| | | |
|---|---|---|
| Agitation at 100 rpm, | 1 Day | 3 Days |
| 25° C. (Upright) | X | X, Y |

The target filling volumes of the vials for the 2-8° C., 25° C., and 40° C. stress conditions were 2 mL within the 2 R vials and 10 mL within the 10 R vials. The target filling volumes of the vials for the agitation stress condition were 2.26 mL within the 2 R vials and 10.88 mL within the 10 R vials.

Vials containing the formulation above were subjected to the stress conditions described in Table 24, above. The time points when samples were taken from the experimental vials are also described in Table 24, and the assays performed at each point are described by the letters Q, X, Y, and Z. Letter X indicates the following assays were performed: appearance check, pH determination, anti-IGF-1R protein concentration determination, SEC-UPLC, CE-SDS (NR and R), and ICIEF. Letter Y indicates that HIAC sub-visible particle analysis was performed. Letter Z indicates that binding potency determination via ELISA was performed. Letter Q indicates that the glass transition temperature was determined by mDSC.

All samples were visually observed to be slightly yellow, slightly opalescent, and free of visible particles. No substantial changes were observed in protein concentration in any experimental condition. HIAC analysis for all experimental conditions did show not any significant changes in from t₀ to 2 weeks, or from t₀ to three days in the case of the agitation stress condition. The HIAC results met the USP <788> criteria. The pH of all vials did not change significantly from to t₀ 2 weeks, or from t₀ to three days in the case of the agitation stress condition.

The results of the SEC-UPLC, iCIEF, CE-NR, CE-R, and potency testing for the 2 R vials were as follows: The vials that underwent agitation at 100 rpm for up to 3 days displayed no significant changes. No significant change was observed after incubation at 2-8° C. for up to 3 months. Vials incubated at 25° C. for 2 weeks showed a small absolute decrease of 2.4 in main peak percentage. The other analytical techniques did not show any changes within the 25° C. group. Vials incubated at 40° C. for 2 weeks showed an absolute decrease of 0.6 in monomer percentage via SEC-UPLC, an absolute decrease of 14.8 in main peak percentage as measured via iCIEF, and an absolute decrease of 1.9 in main peak percentage as measured via CE-NR. These results are summarized in Table 25, below.

TABLE 25
SEC-UPLC, iCIEF, SDS-CE-NR, SDS-CE-R, and Potency Results

| | | | | | iCIEF | | | | | |
|---|---|---|---|---|---|---|---|---|---|---|
| | | SEC-UPLC | | | Main | Acidic | Basic | SDS-CE-NR | SDS-CE-R | Potency |
| Sampling Points | | Monomer % | HMW % | LMW % | peak % | peaks % | peaks % | Purity % | Purity % | (%) |
| T0 | | 99.1 | 0.8 | 0.1 | 50.3 | 46.2 | 3.5 | 96.6 | 98.1 | 97 |
| Agitation | 1 D | 99.1 | 0.7 | 0.1 | 49.5 | 47.6 | 2.9 | 96.6 | 98.0 | NT |
| 100 rpm | 3 D | 99.2 | 0.7 | 0.1 | 48.7 | 47.8 | 3.4 | 96.6 | 98.1 | NT |
| 2-8° C. | 2 W | 99.1 | 0.8 | 0.1 | 48.9 | 47.6 | 3.4 | 96.4 | 98.1 | NT |
| (Inverted) | 3 M | 99.2 | 0.8 | 0.1 | 49.0 | 48.4 | 2.6 | 96.5 | 98.1 | 98 |
| 25° C. | 2 W | 99.1 | 0.7 | 0.2 | 47.9 | 49.1 | 3.0 | 96.3 | 97.6 | NT |
| (Inverted) | 3 M | 98.7 | 1.0 | 0.4 | 33.5 | 63.6 | 2.9 | 95.3 | 97.5 | 93 |
| 40° C. | 2 W | 98.5 | 0.9 | 0.7 | 35.5 | 62.1 | 2.3 | 94.7 | 97.2 | NT |
| (Upright) | 4 W | 96.7 | 2.0 | 1.3 | 23.7 | 73.6 | 2.7 | 93.4 | 95.6 | 91 |

The results of the SEC-UPLC, iCIEF, CE-NR, CD-R, and potency testing for the 10 R vials were as follows. Vials that underwent agitation at 100 rpm for up to 3 days displayed no significant changes. No significant change was observed after incubation at 2-8° C. for up to 3 months. Vials incubated at 25° C. for 2 weeks showed an absolute decrease of 2.8 in main peak percentage as measured via iCIEF. The other analytical techniques did not show any changes within the 25° C. group. Vials incubated at 40° C. for 2 weeks showed an absolute decrease of 0.8 in monomer percentage as measured via SE-UPLC, an absolute decrease of 15.6 in main peak percentage as measured via iCIEF, and an absolute decrease of 1.8 in main peak percentage as measured via CE-NR. These results are also summarized in Table 26, below.

TABLE 26
SEC-UPLC, iCIEF, SDS-CE-NR, SDS-CE-R, Potency Results

| | | | | | iCIEF | | | | | |
|---|---|---|---|---|---|---|---|---|---|---|
| | | SE-UPLC | | | Main | Acidic | Basic | SDS-CE-NR | SDS-CE-R | Potency |
| Sampling Points | | Monomer % | HMW % | LMW % | peak % | peaks % | peaks % | Purity % | Purity % | (%) |
| T0 | | 99.1 | 0.8 | 0.1 | 49.4 | 47.4 | 3.3 | 96.6 | 98.3 | 94 |
| Agitation | 1 D | 99.2 | 0.7 | 0.1 | 49.5 | 47.2 | 3.3 | 96.6 | 97.8 | NT |
| 100 rpm | 3 D | 99.2 | 0.7 | 0.1 | 50.0 | 47.2 | 2.7 | 96.5 | 98.2 | NT |
| 2-8° C. | 2 W | 99.1 | 0.8 | 0.1 | 48.1 | 48.5 | 3.5 | 96.3 | 97.7 | NT |
| (Inverted) | 3 M | 99.1 | 0.7 | 0.1 | 49.7 | 47.9 | 2.4 | 96.3 | 98.2 | 91 |
| 25° C. | 2 W | 99.1 | 0.7 | 0.2 | 46.6 | 50.0 | 3.4 | 96.2 | 97.9 | NT |
| (Inverted) | 3 M | 98.7 | 0.9 | 0.4 | 33.3 | 64.1 | 2.7 | 95.3 | 97.5 | 90 |
| 40° C. | 2 W | 98.3 | 1.0 | 0.7 | 33.8 | 63.6 | 2.7 | 94.8 | 97.5 | NT |
| (Upright) | 4 W | 96.7 | 2.0 | 1.3 | 23.1 | 73.8 | 3.0 | 93.3 | 96.3 | 93 |

### Example 7: Stable Formulation of VRDN-01100 Treats Thyroid Associated Ophthalmopathy in a Subject with a Reduction in Proptosis Occurring within 3 Weeks of a First Dose

Anti-IGF-1R antibody VRDN-01100 was evaluated in participants with Thyroid Eye Disease (TED). Two infusions three weeks apart of 3 mg/kg, 10 mg/kg, or 20 mg/kg were administered to human patients with TED. The antibody was administered in a stable formulation of 50 mg/mL VRDN-01100, 20 mM histidine buffer, 8% sucrose, 10 mM Methionine, and 0.02% PS80, at a pH of 5.5. Proptosis response, CAS response, CAS score of 0 or 1, overall response, and diplopia resolution were assessed in the patients at week six following two administrations of VRDN-01100. Results observed are as noted in Table 27. Week 6 efficacy results in human patients with TED after two infusions of VRDN-01100.

TABLE 27

| Efficacy | 3 mg/kg | 10 mg/kg | 20 mg/kg |
|---|---|---|---|
| Endpoint/Dose | Cohort | Cohort | Cohort |
| Proptosis Response | 6/9 (67%) | 5/6 (83%) | 4/6 (67%) |
| CAS Response | 8/9 (67%) | 6/6 (100%) | 6/6 (100%) |
| CAS Score of 0 or 1 | 6/9 (67%) | 5/6 (83%) | 2/6 (33%) |
| Overall Response | 5/9 (56%) | 5/6 (83%) | 4/6 (67%) |
| Diplopia Resolution | 1/5 (20%) | 3/4 (75%) | 3/4 (75%) |

After only two infusions at 3 mg/kg, 10 mg/kg, or 20 mg/kg, VRDN-01100 achieved significant clinical efficacy endpoints. As compared to the published data for anti-IGF-1R antibody Teprotumumab, VRDN-01100 achieved significantly faster results in mean reduction in proptosis and improvement in diplopia (See, e.g., Smith et al., Teprotumumab for Thyroid-Associated Ophthalmopathy, N Engl J Med 2017; 376:1748-61; Douglas et al., Teprotumumab for the Treatment of Active Thyroid Eye Disease, N. Engl J Med 2020; 382:341-52; and Douglas et al., Teprotumumab Efficacy, Safety, and Durability in Longer-Duration Thyroid Eye Disease and Re-treatment, Ophthalmology 2022, Vol. 129, No. 4). Patients treated with VRDN-011000 had, on average, a reduction or improvement in at least two of proptosis, diplopia, and CAS score; these results were not observed in the placebo cohort.

### Example 8: High Concentration Formulation Study

This study illustrates that the anti-IGF-1R antibody can be concentrated to high concentration (i.e., up to 230 mg/ml) in the exemplary formulation described in the above examples.

As one of ordinary skill in the art would appreciate, high concentration antibody formulations are challenging to develop due to issues such as physical and chemical instability, high viscosity, and delivery volume limitations. High concentrations can lead to protein aggregation, opalescence, and phase separation, which affect stability and efficacy. Aggregates can be immunogenic, potentially reducing efficacy and safety. Additionally, increased viscosity can complicate manufacturing and administration. Furthermore, issues like deamination and isomerization can occur, impacting the formulation's shelf life and performance. These challenges require robust formulation and process development strategies. Despite these difficulties, high concentration formulations are increasingly popular due to their advantages in subcutaneous administration, which allows for self-administration and reduces the need for hospital visits.

Evaluation of 150 mg/ml Concentration

In this study, a formulation comprising 20 mM histidine buffer at pH5.5, 8% sucrose, 10 mM methionine, and 0.04% PS80 was prepared for 150 mg/ml of VRDN-1100. The formulation was stored at 5° C. and 25° C. for up to three months and at 40° C. for up to one month. Various analytical methods were used to determine stability, purity, and potency of the formulation.

The formulation was free of visible particles upon storage for at least 3 months at 5° C. and 25° C., and for at least one month at 40° C. Additionally, SEC, iCIEF, and CE-NR were used to determine stability/aggregation, purity, and potency as shown in Table 28. The results show that a formulation comprising histidine buffer at pH 5.5, methionine, sucrose, and surfactant was stable even when the antibody was at 150 mg/ml.

TABLE 28
Summary of SE-UPLC/iCIEF/CE-NR of 150 mg/ml Formulation

| | | | | | iCIEF | | | |
|---|---|---|---|---|---|---|---|---|
| | | SE-UPLC | | | Main | Acidic | Basic | SDS-CE-NR |
| Sampling Points | | Monomer % | HMW % | LMW % | peak % | peaks % | peaks % | Purity % |
| T0 | | 98.8 | 1.1 | 0.1 | 49.2 | 48.1 | 2.6 | 96.7 |
| Agitation | 1 D | 98.7 | 1.2 | 0.1 | 49.0 | 48.7 | 2.2 | 96.6 |
| 100 rpm | 3 D | 98.7 | 1.2 | 0.1 | 48.3 | 49.3 | 2.3 | 96.5 |
| Freeze/thaw | 5 C | 98.8 | 1.2 | 0.1 | 49.1 | 48.6 | 2.2 | 96.6 |
| −20° C. to RT | | | | | | | | |
| 5° C. | 1 M | 98.7 | 1.3 | 0.1 | 49.3 | 48.6 | 2.1 | 96.6 |
| | 3 M | 98.6 | 1.3 | 0.1 | 50.5 | 47.1 | 2.4 | 96.2 |
| 25° C. | 2 W | 98.3 (↓0.5) | 1.5 | 0.1 | 47.1 | 51.1 | 1.8 | 96.4 |
| | 1 M | 98.1 (↓0.7) | 1.7 | 0.2 | 43.5 (↓5.7) | 54.2 | 2.3 | 96.0 |
| | 3 M | 97.6 (↓1.2) | 2.0 | 0.4 | 35.5 (↓13.7) | 61.6 | 3.0 | 94.6 (↓2.1) |
| 40° C. | 1 M | 95.7 (↓3.1) | 2.9 | 1.4 | 23.0 (↓26.2) | 75.8 | 1.2 | 92.5 (↓4.2) |

Evaluation of Concentrations Greater than 150 mg/ml

At a concentration of greater than 150 mg/ml, liquid antibody formulations run into two major challenges: high rates of aggregation and increased viscosity. As antibody concentrations increase, self-association leads to higher viscosity, complicating manufacturing, stability, and delivery process. This is particularly problematic for subcutaneous administration, where high viscosity can impede injection. Strategies to prepare high concentration formulations often require tailored approaches for each antibody, complicating standardization. To examine whether the exemplary formulation of 20 mM histidine buffer at pH 5.5, sugar, 10 mM methionine, and 0.04% PS80 can be used to achieve an antibody concentration of greater than 150 mg/ml, the following study was performed.

UF/DF pool material in the formulation of 80 mg/mL protein, 20 mM histidine buffer, at pH 5.5 was used in this study. Firstly, 20 mM histidine buffer solution (pH 5.5), 40% (w/v) sucrose stock solution, 30% (w/v) sorbitol stock solution, 200 mM L-methionine stock solution and 5% (w/w) PS80 stock solution were prepared. The pre-formulation buffer of 20 mM histidine buffer, 8% (w/v) sucrose, 10 mM L-methionine, pH 5.5 and 20 mM histidine buffer, 4.5% (w/v) sorbitol, 10 mM L-methionine, pH 5.5 were prepared by compounding the histidine buffer with the corresponding stock solutions. The UF/DF pool material was condensed to about 231.3 mg/mL and compounded and diluted to get the final formulations at 165, 175, 185 and 200 mg/mL. The samples were filtered with 0.22 μm PVDF filter, filled into 2 mL glass vials (2 mL per vial), then stoppered, capped, and labeled immediately.

Appearance and viscosity at 25° C. and 5° C. were evaluated. As shown in Table 29, all formulation samples were well below the maximum viscosity for subcutaneous delivery (e.g., 50 cp), except for the 230 mg/ml formulation at 5° C.

TABLE 29
High Concentration Formulations

| | Ab | | Viscosity (cp) | |
|---|---|---|---|---|
| Formulation | Concentration | Appearance | 25° C. | 5° C. |
| 20 mM Histidine, pH 5.5 (UF/DF | 230 mg/ml | Not tested | 19.13 | 52.93 |
| sample) | | | | |
| 20 mM Histidine, 8% Sucrose, 10 | 165 mg/ml | Not tested | 6.76 | 14.78 |
| mM Methionine, 0.04% PS80, pH | 175 mg/ml | Free of particles | 7.94 | 17.83 |
| 5.5 | 185 mg/ml | Not tested | 8.89 | 21.43 |
| | 200 mg/ml | Not tested | 13.57 | 33.89 |
| 20 mM Histidine, 4.5% Sorbitol, 10 | 165 mg/ml | Not tested | 5.94 | 12.91 |
| mM Methionine, 0.04% PS80, pH | 175 mg/ml | Not tested | 7.01 | 15.67 |
| 5.5 | 185 mg/ml | Not tested | 7.67 | 17.56 |
| | 200 mg/ml | Not tested | 11.2 | 27.72 |

Overall, the high concentration formulation feasibility study confirmed that the anti-IGF-1R antibody (VRDN-1100) can be concentrated up to 230 mg/mL in exemplary formulations such as 20 mM histidine buffer and 200 mg/mL in the two selected formulations with 8% (w/v) sucrose or 4.5% (w/v) sorbitol. The viscosity values of two selected formulations were about 7-8 cP, which were acceptable for manufacturing and injection.

### Example 9: Stability of 175 mg/ml Formulation

Stability study was to confirm the stability of high concentration formulation (e.g., 175 mg/ml) in the selected primary CCS (2 mL vial (Schott-1626151), 13 mm stopper (Cat. No.: 1970-0173) and 13 mm cap (Cat. No.: 5413-2150)) with target fill volume (2 mL per vial) for liquid DP. The 175 mg/ml of the anti-IGF-1R antibody was prepared in 20 mM histidine buffer, 8% (w/v) sucrose, 10 mM L-methionine, 0.04% (w/v) PS80, pH 5.5. Appearance, sub-visible particles, monomer, HMW, LMW, iCIEF, purity, and potency were evaluated.

As shown in Table 30-1, all the samples remained yellow, slightly opalescent, and free of visible particles at T0, after agitation for up to 3 days, freeze/thaw for up to 5 cycles, incubation at 5° C. or 25° C. for up to 3 months and incubation at 40° C. for up to 1 month.

TABLE 30-1
Appearance of 175 mg/ml formulation

| Sampling point | | Appearance |
|---|---|---|
| T0 | | Free of particles |
| Agitation | 1D | Free of particles |
| | 3D | Free of particles |
| Freeze/thaw | 3 Cycles | Free of particles |
| | 5 Cycles | Free of particles |
| 5° C. | 1M | Free of particles |
| | 3M | Free of particles |
| 25° C. | 1M | Free of particles |
| | 3M | Free of particles |
| 40° C. | 1M | Free of particles |

As shown in Table 30-2, no substantial changes of sub-visible particles were observed in the samples after agitation for 3 days, freeze/thaw for up to 5 cycles, incubation at 5° C. or 25° C. for up to 3 months or incubation at 40° C. for 1 month when compared with T0.

Additionally, no substantial changes of potency were observed in the samples after incubation at 5° C. or 25° C. for up to 3 months or incubation at 40° C. for 1 month when compared with T0. No substantial change of CE-SDS-NR purity was observed in the samples after agitation for up to 3 days, freeze/thaw for up to 5 cycles or incubation at 5° C. for up to 3 months when compared with T0. 1.5% and 4.4% decreases of CE-SDS-NR purity were observed after incubation at 25° C. for 3 months and 40° C. for 1 month respectively, which were acceptable under the accelerated or stressed conditions.

TABLE 30-2
Sub-visible particles, potency and purity of 175 mg/ml formulation

| | | Sub-visible particles (HIAC) | | | ELISA | CE-SDS-NR |
|---|---|---|---|---|---|---|
| Sampling point | | ≥2 μm | ≥10 μm | ≥25 μm | Potency % | Purity % |
| T0 | | 183 | 15 | 0 | 95 | 96.6 |
| Agitation | 1 D | 126 | 11 | 0 | n.t. | 96.5 |
| | 3 D | 8 | 0 | 0 | n.t. | 96.4 |
| Freeze/thaw | 3 Cycles | 93 | 8 | 0 | n.t. | 96.6 |
| | 5 Cycles | 99 | 8 | 0 | n.t. | 96.3 |
| 5° C. | 1 M | 179 | 6 | 0 | n.t. | 96.4 |
| | 3 M | 299 | 9 | 0 | 88 | 96.5 |
| 25° C. | 1 M | 15 | 0 | 0 | 95 | 95.9 |
| | 3 M | 234 | 17 | 0 | 90 | 95.1 (↓1.5) |
| 40° C. | 2 W | 148 | 4 | 0 | n.t. | 94.4 (↓2.2) |
| | 1 M | 115 | 0 | 0 | 87 | 92.2 (↓4.4) |

As shown in Table 30-3, no substantial changes of SE-UPLC monomer, HMW and LMW were observed in the samples after agitation for up to 3 days, freeze/thaw for up to 5 cycles or incubation at 5° C. for 3 months when compared with T0. 1.6% and 2.5% decreases of SE-UPLC monomer with the increase mainly in HMW were observed after incubation at 25° C. for 3 months and 40° C. for 1 month respectively, which were acceptable under the accelerated or stressed condition. Additionally, no substantial changes of iCIEF main peak, acidic peaks and basic peaks were observed in the samples after incubation at 5° C. for 3 months, agitation for up to 3 days or freeze/thaw for up to 5 cycles when compared with T0. 17.0% decrease of iCIEF main peak with the increase both in acidic peaks and basic peaks was observed after incubation at 25° C. for 3 months. 28.7% decrease of iCIEF main peak with the increase mainly in acidic peaks was observed after incubation at 40° C. for 1 month. The changes were acceptable under the accelerated or stressed conditions.

TABLE 30-3
Sub-visible particles and potency of 175 mg/ml formulation

| | | SE-ULPC | | | iCIEF | | |
|---|---|---|---|---|---|---|---|
| Sampling point | | Monomer % | HMW % | LMW % | Main % | Acidic % | Basic % |
| T0 | | 98.9 | 1.1 | 0.1 | 53.0 | 44.7 | 2.3 |
| Agitation | 1 D | 98.8 | 1.1 | 0.1 | 52.7 | 44.7 | 2.6 |
| | 3 D | 98.8 | 1.1 | 0.1 | 52.1 | 45.0 | 2.8 |
| Freeze/thaw | 3 Cycles | 98.8 | 1.1 | 0.1 | 53.3 | 44.0 | 2.7 |
| | 5 Cycles | 98.8 | 1.1 | 0.1 | 52.8 | 44.2 | 3.0 |
| 5° C. | 1 M | 98.8 | 1.1 | 0.1 | 52.5 | 44.9 | 2.6 |
| | 3 M | 98.5 | 1.5 | 0.1 | 50.4 | 45.8 | 3.7 |
| 25° C. | 1 M | 98.2 (↓0.7) | 1.5 | 0.3 | 47.1 (↓5.9) | 49.7(↑5.0) | 3.2(↑0.9) |
| | 3 M | 97.3 (↓1.6) | 2.2 | 0.6 | 36.0 (↓17.0) | 60.3(↑15.6) | 3.8(↑1.5) |
| 40° C. | 2 W | 97.5 (↓1.4) | 1.9 | 0.7 | 34.6 (↓18.4) | 61.8(↑17.1) | 3.6(↑1.3) |
| | 1 M | 96.4 (↓2.5) | 2.3 | 1.3 | 24.3 (↓28.7) | 72.5(↑27.8) | 3.2(↑0.9) |

The formulation of 175 mg/mL the IGF-1R antibody (VRDN-1100) in the exemplary formulation of 20 mM histidine buffer, 8% (w/v) sucrose, 10 mM methionine, 0.04% (w/v) PS80, pH 5.5 was stable enough to be developed as liquid DP with long-term storage condition of 2-8° C. in the evaluated CCS (2 mL Schott vial (Cat. No.: 1626151), 13 mm stopper (Cat. No.: 1970-0173), 13 mm cap (Cat. No.: 5413-2150)).

### Example 10: Long-Term Stability of 150 mg/ml Formulation

This example illustrates that the 150 mg/mL antibody (VRDN-1100) formulation is stable in another primary container closure system (the prefilled syringe; PFS) in addition to the vial and stopper presentation used previously. This is notable because some protein formulations do not like (i.e. are not stable in PFS) due to the silicone oil that is sprayed and coated on the inside of the syringe barrel (which is for easing gliding force during administration) or the residual tungsten oxide that can be present in PFS. This residual tungsten oxide from this process step has been shown to be deleterious to some protein's stability.

In this study, stability of the antibody in an exemplary formulation was evaluated in two types of PFS: BD syringe (Cat. No.: 47510610), and Ompi syringe (Cat. No.: 7600003.8400). The 50 mg/ml of the anti-IGF-1R antibody (VRDN-1100) was prepared in 20 mM histidine buffer, 8% (w/v) sucrose, 10 mM L-methionine, 0.04% (w/v) PS80, pH 5.5. PFS sizes were 2.25 ml, and the fill volume was 2.1 ml. Appearance, sub-visible particles, monomer, HMW, LMW, iCIEF, purity, and potency were evaluated.

All samples were yellow, slightly opalescent and free of visible particles. Additionally, no substantial changes of protein concentration and pH were observed after incubation at 5° C. for at least up to 12 months. As shown in Tables 31-1 and 31-2, all samples were stable at 5° C. for at least up to 12 months with minimal decrease in monomer and purity and changes in main, acidic, and basic peaks, for both BD and Ompi syringes. Similar results were obtained for storage at 25° C. (Tables 31-3 and 31-4). At 40° C., 5 of monomer decreased by about 5% after 1 month, but the changes were acceptable under the accelerated or stressed conditions.

TABLE 31-1
Stability and Purity of Formulation in BD syringe at 5° C.

| | | BD | | | | | | | |
|---|---|---|---|---|---|---|---|---|---|
| | | SEC-UPLC | | | iCIEF | | | CE-SDS-NR | CE-SDS-R |
| Sampling Points | | LMW % | HMW % | Monomer % | Main % | Acidic % | Basic % | Purity % | Purity % |
| T0 | | 0.1 | 0.7 | 99.2 | 55.3 | 42.3 | 2.5 | 97.0 | 98.1 |
| 5 C. | 2 W | 0.0 | 0.8 | 99.2 | 54.8 | 42.2 | 3.0 | 97.1 | 98.2 |
| | 1 M | 0.1 | 0.8 | 99.1 | 54.5 | 42.7 | 2.7 | 97.0 | 98.0 |
| | 2 M | 0.1 | 0.8 | 99.1 | 52.7 | 44.5 | 2.8 | 96.8 | 98.0 |
| | 3 M | 0.0 | 1.0 | 98.9 | 54.2 | 42.5 | 3.4 | 96.8 | 97.7 |
| | 6 M | 0.1 | 1.1 | 98.9 | 55.9 | 41.6 | 2.5 | 96.5 | 97.8 |
| | 12 M | 0.8 | 1.2 | 98.0(↓1.2) | 52.7 | 44.0 | 3.4 | 96.3 | 97.1 |

TABLE 31-2
Stability and Purity of Formulation in Ompi syringe at 5° C.

| | | Ompi | | | | | | | |
|---|---|---|---|---|---|---|---|---|---|
| | | SEC-UPLC | | | iCIEF | | | CE-SDS-NR | CE-SDS-R |
| Sampling Points | | LMW % | HMW % | Monomer % | Main % | Acidic % | Basic % | Purity % | Purity % |
| T0 | | 0.0 | 0.7 | 99.2 | 55.6 | 42.0 | 2.4 | 97.0 | 98.2 |
| 5 C. | 2 W | 0.0 | 0.8 | 99.1 | 55.0 | 42.4 | 2.6 | 97.0 | 98.1 |
| | 1 M | 0.1 | 0.8 | 99.1 | 54.2 | 42.9 | 2.9 | 96.9 | 98.1 |
| | 2 M | 0.1 | 0.8 | 99.1 | 54.5 | 42.5 | 2.9 | 96.7 | 98.1 |
| | 3 M | 0.0 | 1.0 | 99.0 | 55.3 | 41.7 | 3.0 | 96.7 | 97.6 |
| | 6 M | 0.1 | 1.1 | 98.9 | 55.6 | 41.6 | 2.8 | 96.5 | 97.7 |
| | 12 M | 0.8 | 1.1 | 98.1(↓1.1) | 52.4(↓3.2) | 44.3 | 3.3 | 96.4 | 97.6 |

TABLE 31-3
Stability and Purity of Formulation in BD syringe at 25° C.

| | | | BD | | | | | | | |
|---|---|---|---|---|---|---|---|---|---|---|
| | | | SEC-UPLC | | | iCIEF | | | CE-SDS-NR | CE-SDS-R |
| Sampling Points | | | LMW % | HMW % | Monomer % | Main % | Acidic % | Basic % | Purity % | Purity % |
| T0 | | | 0.1 | 0.7 | 99.2 | 55.3 | 42.3 | 2.5 | 97.0 | 98.1 |
| 25 C. | 2 | W | 0.1 | 1.0 | 98.9 | 52.9 | 44.6 | 2.5 | 96.7 | 97.9 |
| | 1 | M | 0.2 | 1.1 | 98.7 | 48.8 (↓6.5) | 48.5 | 2.7 | 96.4 | 97.9 |
| | 2 | M | 0.2 | 1.2 | 98.6 (↓0.6) | 46.3 (↓9.0) | 51.4 | 2.3 | 95.8 (↓1.2) | 97.6 |
| | 3 | M | 0.3 | 1.6 | 98.1 (↓1.1) | 41.5 (↓13.8) | 55.0 | 3.5 | 95.7 (↓1.3) | 96.4 (↓1.7) |
| | 4.5 | M | 2.0 | 1.6 | 96.4 (↓2.8) | 34.3 (↓21.0) | 63.5 | 2.2 | 95.2 (↓1.8) | 96.4 (↓1.70) |
| | 6 | M | 2.6 | 1.9 | 95.6 (↓3.6) | 30.6 (↓24.7) | 66.9 | 2.5 | 94.1 (↓2.9) | 96.1 (↓2.0) |
| | 12 | M | 3.4 | 2.3 | 94.3 (↓4.9) | 18.2 (↓37.1) | 78.9 | 2.9 | 92.0 (↓5.0) | 95.1 (↓3.0) |

TABLE 31-4
Stability and Purity of Formulation in Ompi syringe at 25° C.

| | | | Ompi | | | | | | | | |
|---|---|---|---|---|---|---|---|---|---|---|---|
| | | | SEC-UPLC | | | iCIEF | | | | CE-SDS-NR | CE-SDS-R |
| Sampling Points | | | LMW % | HMW % | Monomer % | Main % | | Acidic % | Basic % | Purity % | Purity % |
| T0 | | | 0.0 | 0.7 | 99.2 | 55.6 | | 42 | 2.4 | 97 | 98.2 |
| 25 C. | 2 | W | 0.1 | 0.9 | 98.9 | 52.4 | (↓3.2) | 44.9 | 2.8 | 96.7 | 98 |
| | 1 | M | 0.1 | 1.1 | 98.8 | 48.8 | (↓6.8) | 48.6 | 2.6 | 96.3 | 98 |
| | 2 | M | 0.2 | 1.2 | 98.6 (↓0.6) | 45.8 | (↓9.8) | 50.9 | 3.4 | 95.8 (↓1.2) | 97.7 |
| | 3 | M | 0.3 | 1.6 | 98.1 (↓1.1) | 41.4 | (↓14.2) | 55.1 | 3.5 | 95.6 (↓1.4) | 96.4 (↓2.2) |
| | 4.5 | M | 2 | 1.6 | 96.4 (↓2.8) | 33.1 | (↓22.5) | 64.9 | 2 | 95.2 (↓1.8) | 96.4 (↓1.8) |
| | 6 | M | 2.6 | 1.9 | 95.6 (↓3.6) | 30.9 | (↓24.7) | 66.6 | 2.6 | 94.1 (↓2.9) | 96.3 (↓1.9) |
| | 12 | M | 3.4 | 2.2 | 94.4 (↓4.8) | 18.2 | (↓37.4) | 78.9 | 2.9 | 92.0 (↓5.0) | 95.0 (↓3.2) |

Potency was also evaluated upon at 5° C. for 12M, 25° C. for 12M and 40° C. for 2M. All values were within the acceptable ranges, as shown in Table 31-5.

TABLE 31-5
Potency results summary

| | | Potency (%) | |
|---|---|---|---|
| Sampling Points | | BD | Ompi |
| T0 | | 102 | 97 |
| 5 C | 2M | 93 | 91 |
| | 6M | 97 | 91 |
| | 12M | 96 | 96 |
| 25 C | 2M | 100 | 102 |
| | 6M | 85 | 83 |
| | 12M | 87 | 87 |
| 40 C | 1M | 95 | 92 |
| | 2M | 77 | 76 |

All references cited herein are incorporated by reference to the same extent as if each individual publication, database entry (e.g., Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g., Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present embodiments are not to be limited in scope by the specific embodiments described herein. Indeed, various modifications in addition to those described herein will become apparent to those skilled in the art from the foregoing description. Such modifications are intended to fall within the scope of the embodiments and any appended claims.

The present specification is considered to be sufficient to enable one skilled in the art to practice the embodiments. Various modifications in addition to those shown and described herein will become apparent to those skilled in the art from the foregoing description and fall within the scope of the present disclosure and any appended claims.

1. A stable formulation comprising an anti-IGF-1R antibody or antigen-binding fragment thereof at a concentration of 25-250 mg/ml,
wherein the antibody comprises a heavy chain variable region comprising HCDR1 of SEQ ID NO: 53, an HCDR2 of SEQ ID NO: 54, an HCDR3 of SEQ ID NO: 55, a light chain variable region comprising a LCDR1 of SEQ ID NO: 56, a LCDR2 of SEQ ID NO: 57 and a LCDR3 of SEQ ID NO: 58, and
wherein the formulation has a pH of 4.8-6.5.

2. The stable formulation of claim 1, wherein the heavy chain variable region is at least 90% identical to SEQ ID NO: 14 and the light chain variable region is at least 90% identical to SEQ ID NO: 13.

3. (canceled)

4. The stable formulation of claim 1, wherein the antibody comprises a heavy chain of SEQ ID NO: 92 or SEQ ID NO: 94, and a light chain of SEQ ID NO: 93.

5. The stable formulation of claim 1, wherein the anti-IGF-1R antibody is present at a concentration of between 25-200 mg/ml, 50-200 mg/ml, 75-180 mg/ml, or 100-150 mg.

6-7. (canceled)

8. The stable formulation of claim 1, wherein the pH is between 5.0-6.0, 5.2-6.0 or 5.2-6.5.

9. (canceled)

10. The stable formulation of claim 1, wherein the formulation further comprises sucrose.

11-12. (canceled)

13. The stable formulation of claim 1, wherein the formulation further comprises a stabilizer.

14-16. (canceled)

17. The stable formulation of claim 1, wherein the composition further comprises a surfactant.

18-21. (canceled)

22. The stable formulation of claim 1, wherein the formulation further comprises a buffering agent.

23-25. (canceled)

26. The stable formulation of claim 1, wherein the buffering agent does not comprise succinate.

27. A stable formulation comprising:
an anti-IGF-1R antibody comprising at a concentration of 50 mg/ml;
a histidine buffer at concentration of 20 mM;
methionine at a concentration of 10 mM;
polysorbate 80 at a concentration of 0.02% (w/v);
sucrose at a concentration of 8% (w/v); and
pH of 5.5;
wherein the antibody comprises an HCDR1 of SEQ ID NO: 53, a HCDR2 of SEQ ID NO: 54, an HCDR3 of SEQ ID NO: 55, a LCDR1 of SEQ ID NO: 56, a LCDR2 of SEQ ID NO: 57 and a LCDR3 of SEQ ID NO: 58.

28. A stable formulation comprising:
an anti-IGF-1R antibody comprising at a concentration of 150 mg/ml;
a histidine buffer at concentration of 20 mM;
methionine at a concentration of 10 mM;
polysorbate 80 at a concentration of 0.04% (w/v);
sucrose at a concentration of 8% (w/v); and
pH of 5.5;
wherein the antibody comprises an HCDR1 of SEQ ID NO: 53, an HCDR2 of SEQ ID NO: 54, an HCDR3 of SEQ ID NO: 55, a LCDR1 of SEQ ID NO: 56, a LCDR2 of SEQ ID NO: 57 and a LCDR3 of SEQ ID NO: 58.

29. The stable formulation of claim 1, wherein antibody comprises a heavy chain variable region that is at least 90% identical to SEQ ID NO: 14 and a light chain variable region that is at least 90% identical to SEQ ID NO: 13.

30. (canceled)

31. The stable formulation of claim 1, wherein anti-IGF-1R antibody comprises a heavy chain comprising SEQ ID NO: 92, and a light chain comprising SEQ ID NO: 93.

32. (canceled)

33. The stable formulation of claim 1, wherein less than 5%, less than 4%, less than 3%, less than 2.5%, less than 2%, or less than 1.5% of the IGF-1R antibody exists as HMW species in the formulation upon storage at 40° C. for at least 4 weeks.

34-44. (canceled)

45. The stable formulation of claim 1, wherein the osmolality is between 280-380 mOsmol/kg or between 340-420 mOsmol/kg.

46. The stable formulation of claim 1, wherein the formulation is stored at 25 to 25° C.

47. (canceled)

48. The stable formulation of claim 1, wherein the formulation is suitable for intravenous, subcutaneous, or intramuscular administration.

49-50. (canceled)

51. A method of treating thyroid associated ophthalmopathy in a subject comprising administering the stable formulation of claim 1.

52-55. (canceled)

56. A method of treating or reducing the severity of thyroid-associated ophthalmopathy (TAO) in a subject comprising administering the stable formulation of claim 1,
wherein treatment with said pharmaceutical composition reduces proptosis by at least 2 mm in an eye; or
is not accompanied by a deterioration of 2 mm or more in the other (or fellow eye); and/or
reduces the CAS in said subject to either one (1) or zero (0).

57-64. (canceled)


SEQUENCE LISTING (118 sequences)

SEQ ID NO: 1 - protein, 215 aa, synthetic construct
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 448 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 219 aa, synthetic construct
DIVMTQSPLSLPVTPGEPASISCRSSQSIVHSNGNTYLQWYLQKPGQSPQLLIYKVSNRLYGVPDRFSGSGSGTDFTLKISRVEAEDVGVYYCFQGSHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 447 aa, synthetic construct
QVQLQESGPGLVKPSETLSLTCTVSGYSITGGYLWNWIRQPPGKGLEWIGYISYDGTNNYKPSLKDRVTISRDTSKNQFSLKLSSVTAADTAVYYCARYGRVFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 214 aa, synthetic construct
SSELTQDPAVSVALGQTVRITCQGDSLRSYYATWYQQKPGQAPILVIYGENKRPSGIPDRFSGSSSGNTASLTITGAQAEDEADYYCKSRDGSGQHLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPAECS

SEQ ID NO: 6 - protein, 460 aa, synthetic construct
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARAPLRFLEWSTQDHYYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 214 aa, synthetic construct
DIQMTQFPSSLSASVGDRVTITCRASQGIRNDLGWYQQKPGKAPKRLIYAASRLHRGVPSRFSGSGSGTEFTLTISSLQPEDFATYYCLQHNSYPCSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 450 aa, synthetic construct
EVQLLESGGGLVQPGGSLRLSCTASGFTFSSYAMNWVRQAPGKGLEWVSAISGSGGTTFYADSVKGRFTISRDNSRTTLYLQMNSLRAEDTAVYYCAKDLGWSDSYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 9 - protein, 219 aa, synthetic construct
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGTHWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 449 aa, synthetic construct
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARWTGRTDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 214 aa, synthetic construct
EIVLTQSPGTLSVSPGERATLSCRASQSIGSSLHWYQQKPGQAPRLLIKYASQSLSGIPDRFSGSGSGTDFTLTISRLEPEDFAVYYCHQSSRLPHTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 448 aa, synthetic construct
EVQLVQSGGGLVKPGGSLRLSCAASGFTFSSFAMHWVRQAPGKGLEWISVIDTRGATYYADSVKGRFTISRDNAKNSLYLQMNSLRAEDTAVYYCARLGNFYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 113 aa, synthetic construct
DVVMTQTPLSLPVSLGDPASISCRSSQSIVHSNVNTYLEWYLQKPGQSPRLLIYKVSNRFSGVPDRFSGSGAGTDFTLRISRVEAEDLGIYYCFQGSHVPPTFGGGTKLEIKR

SEQ ID NO: 14 - protein, 124 aa, synthetic construct
QVQLVQSGAEVVKPGASVKLSCKASGYTFTSYWMHWVKQRPGQGLEWIGEINPSNGRTNYNQKFQGKATLTVDKSSSTAYMQLSSLTSEDSAVYYFARGRPDYYGSSKWYFDVWGQGTTVTVSS

SEQ ID NO: 15 - protein, 107 aa, synthetic construct
DIQMTQSPLSLSASVGDRVTITCQASRDIRNYLNWYQQKPGKAPKLLIYDASSLQTGVPSRFGGSGSGTDFSFTIGSLQPEDIATYYCQQFDSLPHTFGQGTKLEIK

SEQ ID NO: 16 - protein, 120 aa, synthetic construct
EVQLLESGGGLVQPGGSLRLSCAASGFTFSIYRMQWVRQAPGKGLEWVSGISPSGGTTWYADSVKGRFTISRDNSKNTLYLQMNSLRAEDTAVYYCARWSGGSGYAFDIWGQGTMVTVSS

SEQ ID NO: 17 - protein, 11 aa, synthetic construct
RASQSVSSYLA

SEQ ID NO: 18 - protein, 7 aa, synthetic construct
DASKRAT

SEQ ID NO: 19 - protein, 10 aa, synthetic construct
QQRSKWPPWT

SEQ ID NO: 20 - protein, 5 aa, synthetic construct
SYGMH

SEQ ID NO: 21 - protein, 17 aa, synthetic construct
IIWFDGSSTYYADSVRG

SEQ ID NO: 22 - protein, 9 aa, synthetic construct
ELGRRYFDL

SEQ ID NO: 23 - protein, 20 aa, synthetic construct
RSSQSIVHSNGNTYLQWYLQ

SEQ ID NO: 24 - protein, 7 aa, synthetic construct
KVSNRLY

SEQ ID NO: 25 - protein, 9 aa, synthetic construct
FQGSHVPWT

SEQ ID NO: 26 - protein, 6 aa, synthetic construct
GGYLWN

SEQ ID NO: 27 - protein, 16 aa, synthetic construct
YISYDGTNNYKPSLKD

SEQ ID NO: 28 - protein, 8 aa, synthetic construct
YGRVFFDY

SEQ ID NO: 29 - protein, 11 aa, synthetic construct
QGDSLRSYYAT

SEQ ID NO: 30 - protein, 7 aa, synthetic construct
GENKRPS

SEQ ID NO: 31 - protein, 11 aa, synthetic construct
KSRDGSGQHLV

SEQ ID NO: 32 - protein, 5 aa, synthetic construct
SYAIS

SEQ ID NO: 33 - protein, 17 aa, synthetic construct
GIIPIFGTANYAQKFQG

SEQ ID NO: 34 - protein, 21 aa, synthetic construct
APLRFLEWSTQDHYYYYYMDV

SEQ ID NO: 35 - protein, 11 aa, synthetic construct
RASQGIRNDLG

SEQ ID NO: 36 - protein, 7 aa, synthetic construct
AASRLHR

SEQ ID NO: 37 - protein, 9 aa, synthetic construct
LQHNSYPCS

SEQ ID NO: 38 - protein, 5 aa, synthetic construct
SYAMN

SEQ ID NO: 39 - protein, 17 aa, synthetic construct
AISGSGGTTFYADSVKG

SEQ ID NO: 40 - protein, 16 aa, synthetic construct
DLGWSDSYYYYYGMDV

SEQ ID NO: 41 - protein, 16 aa, synthetic construct
RSSQSLLHSNGYNYLD

SEQ ID NO: 42 - protein, 6 aa, synthetic construct
LGSNRA

SEQ ID NO: 43 - protein, 9 aa, synthetic construct
MQGTHWPLT

SEQ ID NO: 44 - protein, 7 aa, synthetic construct
SSSNWWS

SEQ ID NO: 45 - protein, 16 aa, synthetic construct
EIYHSGSTNYNPSLKS

SEQ ID NO: 46 - protein, 10 aa, synthetic construct
WTGRTDAFDI

SEQ ID NO: 47 - protein, 11 aa, synthetic construct
RASQSIGSSLH

SEQ ID NO: 48 - protein, 7 aa, synthetic construct
YASQSLS

SEQ ID NO: 49 - protein, 9 aa, synthetic construct
HQSSRLPHT

SEQ ID NO: 50 - protein, 5 aa, synthetic construct
SFAMH

SEQ ID NO: 51 - protein, 16 aa, synthetic construct
VIDTRGATYYADSVKG

SEQ ID NO: 52 - protein, 10 aa, synthetic construct
LGNFYYGMDV

SEQ ID NO: 53 - protein, 16 aa, synthetic construct
RSSQSIVHSNVNTYLE

SEQ ID NO: 54 - protein, 7 aa, synthetic construct
KVSNRFS

SEQ ID NO: 55 - protein, 9 aa, synthetic construct
FQGSHVPPT

SEQ ID NO: 56 - protein, 5 aa, synthetic construct
SYWMH

SEQ ID NO: 57 - protein, 18 aa, synthetic construct
GEINPSNGRTNYNQKFQG

SEQ ID NO: 58 - protein, 15 aa, synthetic construct
GRPDYYGSSKWYFDV

SEQ ID NO: 59 - protein, 11 aa, synthetic construct
QASRDIRNYLN

SEQ ID NO: 60 - protein, 7 aa, synthetic construct
DASSLQT

SEQ ID NO: 61 - protein, 9 aa, synthetic construct
QQFDSLPHT

SEQ ID NO: 62 - protein, 5 aa, synthetic construct
IYRMQ

SEQ ID NO: 63 - protein, 16 aa, synthetic construct
GISPSGGTTWYADSVK

SEQ ID NO: 64 - protein, 11 aa, synthetic construct
WSGGSGYAFDI

SEQ ID NO: 65-74 - skipped (10 entries)

SEQ ID NO: 75 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 215 aa, synthetic construct
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 78 - skipped

SEQ ID NO: 79 - protein, 107 aa, synthetic construct
DIQMTQFPSSLSASVGDRVTITCRASQGIRNDLGWYQQKPGKAPKRLIYAASRLHRGVPSRFSGSGSGTEFTLTISSLQPEDFATYYCLQHNSYPSSFGQGTKLEIK

SEQ ID NO: 80 - protein, 125 aa, synthetic construct [sequence omitted]

SEQ ID NO: 81 - protein, 9 aa, synthetic construct
LQHNSYPSS

SEQ ID NO: 82 - skipped

SEQ ID NO: 83 - protein, 446 aa, synthetic construct
QVQLQESGPGLVKPSETLSLTCTVSGYSITGGYLWNWIRQPPGKGLEWIGYISYDGTNNYKPSLKDRVTISRDTSKNQFSLKLSSVTAADTAVYYCARYGRVFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 84 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 85 - skipped

SEQ ID NO: 86 - protein, 113 aa, synthetic construct
DVVMTQTPLSLPVSLGDPASISCRSSQSIVHSNVNTYLEWYLQKPGQSPKLLIYKVSNRFSGVPDRFSGSGAGTDFTLRISRVEAEDLGIYYCFQGSHVPPTFGGGTKLEIKR

SEQ ID NO: 87 - protein, 330 aa, synthetic construct [sequence omitted]

SEQ ID NO: 88 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 89 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 90 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 124 aa, synthetic construct
QVQLVQSGAEVVKPGASVKLSSKASGYTFTSYWMHWVKQRPGQGLEWIGEINPSNGRTNYNQKFQGKATLTVDKSSSTAYMQLSSLTSEDSAVYYFARGRPDYYGSSKWYFDVWGQGTTVTVSS

SEQ ID NO: 92 - protein, 454 aa, synthetic construct
QVQLVQSGAEVVKPGASVKLSCKASGYTFTSYWMHWVKQRPGQGLEWIGEINPSNGRTNYNQKFQGKATLTVDKSSSTAYMQLSSLTSEDSAVYYFARGRPDYYGSSKWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 219 aa, synthetic construct
DVVMTQTPLSLPVSLGDPASISCRSSQSIVHSNVNTYLEWYLQKPGQSPRLLIYKVSNRFSGVPDRFSGSGAGTDFTLRISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 453 aa, synthetic construct
QVQLVQSGAEVVKPGASVKLSCKASGYTFTSYWMHWVKQRPGQGLEWIGEINPSNGRTNYNQKFQGKATLTVDKSSSTAYMQLSSLTSEDSAVYYFARGRPDYYGSSKWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 95 - protein, 453 aa, synthetic construct
QVQLVQSGAEVVKPGASVKLSSKASGYTFTSYWMHWVKQRPGQGLEWIGEINPSNGRTNYNQKFQGKATLTVDKSSSTAYMQLSSLTSEDSAVYYFARGRPDYYGSSKWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 96 - protein, 124 aa, synthetic construct; identical to SEQ ID NO: 91

SEQ ID NO: 97 - protein, 112 aa, synthetic construct
DVVMTQTPLSLPVSLGDPASISCRSSQSIVHSNVNTYLEWYLQKPGQSPRLLIYKVSNRFSGVPDRFSGSGAGTDFTLRISRVEAEDLGIYYCFQGSHVPPTFGGGTKLEIK

SEQ ID NO: 98 - protein, 113 aa, synthetic construct
DIVMTQSPLSLPVTPGEPASISCRSSQSIVHSNGNTYLQWYLQKPGQSPQLLIYKVSNRLYGVPDRFSGSGSGTDFTLKISRVEAEDVGVYYCFQGSHVPWTFGQGTKVEIKR

SEQ ID NO: 99 - protein, 117 aa, synthetic construct [sequence omitted]

SEQ ID NO: 100-101 - skipped (2 entries)

SEQ ID NO: 102 - protein, 11 aa, synthetic construct
QSIVHSNVNTY

SEQ ID NO: 103 - skipped

SEQ ID NO: 104 - protein, 8 aa, synthetic construct
GYTFTSYW

SEQ ID NO: 105 - protein, 8 aa, synthetic construct
INPSNGRT

SEQ ID NO: 106 - protein, 17 aa, synthetic construct
ARGRPDYYGSSKWYFDV

SEQ ID NO: 107 - protein, 11 aa, synthetic construct
QSIVHSNGNTY

SEQ ID NO: 108 - protein, 9 aa, synthetic construct
GYSITGGYL

SEQ ID NO: 109 - protein, 7 aa, synthetic construct
ISYDGTN

SEQ ID NO: 110 - protein, 10 aa, synthetic construct
ARYGRVFFDY

SEQ ID NO: 111 - protein, 7 aa, synthetic construct
GYTFTSY

SEQ ID NO: 112 - protein, 6 aa, synthetic construct
NPSNGR

SEQ ID NO: 113 - protein, 8 aa, synthetic construct
YKVSNRFS

SEQ ID NO: 114 - protein, 13 aa, synthetic construct
KASGYTFTSYWMH

SEQ ID NO: 115 - protein, 10 aa, synthetic construct
EINPSNGRTN

SEQ ID NO: 116 - protein, 14 aa, synthetic construct
TVSGYSITGGYLWN

SEQ ID NO: 117 - protein, 9 aa, synthetic construct
YISYDGTNN

SEQ ID NO: 118 - protein, 8 aa, synthetic construct
YKVSNRLY